(19) United States
(12) Patent Application Publication
(10) Pub. No.: US 2022/0226288 A1
(43) Pub. Date: Jul. 21, 2022

(54) DEXMEDETOMIDINE TREATMENT REGIMENS

(71) Applicant: BioXcel Therapeutics, Inc., New Haven, CT (US)

(72) Inventors: Adedayo Adedoyin, New Haven, CT (US); Jeffrey R. Sabados, New Haven, CT (US); Lavanya Rajachandran, New Haven, CT (US); Robert Risinger, New Haven, CT (US)

(21) Appl. No.: 17/568,474

(22) Filed: Jan. 4, 2022

Related U.S. Application Data
(60) Provisional application No. 63/218,965, filed on Jul. 7, 2021.
(60) Provisional application No. 63/180,284, filed on Apr. 27, 2021.
(60) Provisional application No. 63/168,995, filed on Mar. 31, 2021.
(60) Provisional application No. 63/156,703, filed on Mar. 4, 2021.
(60) Provisional application No. 63/133,593, filed on Jan. 4, 2021.

(51) Int. Cl. A61K 31/4174; A61K 9/00; A61P 25/28

(52) U.S. Cl. CPC A61K 31/4174; A61P 25/28; A61K 9/006

(57) ABSTRACT

Disclosed herein are methods of administering dexmedetomidine or a pharmaceutically acceptable salt thereof to a human subject. The disclosed methods are particularly suitable for the treatment of agitation, especially when associated with neurodegenerative and/or neuropsychiatric diseases or disorders such as dementia and delirium.

### CROSS REFERENCE TO RELATED APPLICATIONS

This application claims the benefit of priority under 35 U.S.C. 119 (e) to U.S. Provisional Patent Application No. 63/133,593, which was filed on Jan. 4, 2021; U.S. Provisional Patent Application No. 63/156,703, which was filed on Mar. 4, 2021; U.S. Provisional Patent Application No. 63/168,995, which was filed on Mar. 31, 2021; U.S. Provisional Patent Application No. 63/180,284, which was filed on Apr. 27, 2021; U.S. Provisional Patent Application No 63/218,965, which was filed on Jul. 7, 2021; the disclosures of each of which are incorporated herein by reference in their entireties.

### BACKGROUND OF THE INVENTION

On Dec. 17, 1999, the U.S. Food and Drug Administration approved a dexmedetomidine product, PRECEDEX®, formulated as an intravenous solution for continuous infusion, and indicated as a sedative agent for initially intubated and mechanically ventilated patients during treatment in an intensive care setting. PRECEDEX® was later approved as a sedative agent for non-intubated patients prior to and/or during surgical and other procedures.

Dexmedetomidine has also been administered intravenously and via other routes to treat a range of conditions, often peri- or post-surgery, including the treatment of pain, anxiety, delirium, withdrawal symptoms, sleep disorders and agitation. However, administration of dexmedetomidine in an appropriate dosage form to provide effective, rapid, relief for the subject without also causing significant sedation is a challenging task. The utilization of dexmedetomidine has also been limited in clinical practice due to its common side effects, such as hypotension and bradycardia. For example, significant cardiovascular side-effects have occurred at therapeutic doses following administration of dexmedetomidine hydrochloride via a sublingual spray or tablets, or intravenously. Thus, a continuing, unmet need exists for an effective dexmedetomidine product which does not cause significant sedation, and desirably is effective without also producing significant adverse effects, such as cardiovascular events. The unmet need is particularly acute for non-addictive medicines that can effectively treat agitation or signs of agitation without also producing the aforementioned adverse effects and sedation.

### SUMMARY

The inventors of the present application have surprisingly found that relatively low doses of dexmedetomidine or a pharmaceutically acceptable salt thereof are efficacious in treating agitation or signs of agitation in dementia patients. For example, administering dexmedetomidine or a pharmaceutically acceptable salt thereof to dementia patients results in about 38% higher Cmax and about 55% higher AUC, when compared to the same dose administered to schizophrenia and bipolar disorder patients. The inventors also surprisingly found that pharmacokinetic effects following administration of dexmedetomidine or a pharmaceutically acceptable salt thereof varied in treating agitation in patients with different underlying conditions. For example, administering a dose of about 60 μg of dexmedetomidine or a pharmaceutically acceptable salt thereof (e.g., dexmedetomidine hydrochloride) sublingually or buccally to patients with dementia, produces similar pharmacokinetic effects as a dose of about 90 μg of dexmedetomidine hydrochloride, administered sublingually or buccally to patients with schizophrenia or bipolar disorder.

In other embodiments, the present disclosure provides methods of treating agitation or signs of agitation in a human subject suffering from dementia, without also inducing significant sedation, comprising administering about 30 μg to about 180 μg of dexmedetomidine or a pharmaceutically acceptable salt thereof.

In embodiments, the present disclosure provides methods of treating agitation or signs of agitation in elderly patients (e.g, 65 years old or older) having dementia comprising administering dexmedetomidine or a pharmaceutically acceptable salt thereof to the patient in an agitated state at a dose sufficient to provide a dexmedetomidine Cₘₐₓ from about 50 ng/L to about 300 ng/L; wherein the route of administration is to the oral mucosa, preferably sublingually, buccally, or gingivally. In embodiments, the patient is an elderly patient, for example, about 65 years old or older.

In embodiments, dexmedetomidine or a pharmaceutically acceptable salt thereof is administered to an agitated patient suffering from dementia at a dose of about 30 μg to about 90 μg. In embodiments, the unit dose comprising about 30 μg to about 90 μg of dexmedetomidine or a pharmaceutically acceptable salt thereof is administered one to six times a day at an interval of at least 2 hours (e.g., about 2, 4, 6, 8, 10, or 12 hours) in the event of persistent or recurrent agitation. In embodiments, the dexmedetomidine or a pharmaceutically acceptable salt thereof is administered at a dose of about 30 μg, about 40 μg, about 50 μg, about 60 μg, about 70 μg, about 80 μg, or about 90 μg. In embodiments, the dexmedetomidine or a pharmaceutically acceptable salt thereof is administered at a dose of about 30 μg. In embodiments, the dexmedetomidine or a pharmaceutically acceptable salt thereof is administered at a dose of about 40 μg. In embodiments, the dexmedetomidine or a pharmaceutically acceptable salt thereof is administered at a dose of about 50 μg. In embodiments, the dexmedetomidine or a pharmaceutically acceptable salt thereof is administered at a dose of about 60 μg. In embodiments, the dexmedetomidine or a pharmaceutically acceptable salt thereof is administered at a dose of about 70 μg. In embodiments, the dexmedetomidine or a pharmaceutically acceptable salt thereof is administered at a dose of about 80 μg. In embodiments, the dexmedetomidine or a pharmaceutically acceptable salt thereof is administered at a dose of about 90 μg.

In embodiments, the dexmedetomidine or a pharmaceutically acceptable salt thereof is administered to an agitated patient suffering from dementia at a dose of about 30 μg to about 90 μg, and the patient has not received treatment for hypertension prior to the administration of dexmedetomidine or a pharmaceutically acceptable salt thereof. In embodiments, the patient has not received treatment for hypertension within about 10 hours, within about 1 day, within about 1 week prior to administration of the dexmedetomidine or a pharmaceutically acceptable salt thereof. In embodiments, the patient is not sedated following administration of dexmedetomidine or a pharmaceutically acceptable salt thereof. In embodiments, the route of administration is to the oral mucosa, wherein the oromucosal administration includes sublingual, buccal or gingival. In embodiments, the AUC₀₋₈ is in the range of about 200 hr*ng/L to about 1500 hr*ng/L. In embodiments, the AUC_{0-inf} is in the range of about 200 hr*ng/L to about 2200 hr*ng/L. In embodiments, the agitation is acute agitation. In embodiments, the agitation is chronic agitation. In embodiments, the AUC values and Cₘₐₓ values are within the range of about 80% to about 125% of the given values. In embodiments, dexmedetomidine or a pharmaceutically acceptable salt thereof (e.g., a hydrochloride salt) is administered oromucosally (e.g., sublingually or buccally) as a film. In embodiments, the patient achieves a mean change in PEC score of greater than −2 relative to baseline within 2 hours of administering the composition. In embodiments, the patient achieves a mean change in PAS score of greater than −2 relative to baseline within 2 hours of administering the composition. In embodiments, the patient achieves a mean change in Mod-CMAI score of greater than −7 relative to baseline 2 hours after administering the composition. In embodiments, the patient achieves a CGI-I score improvement to about a 1 (very much improved) or about a 2 (much improved). In embodiments, the agitation is reduced to a 2 (moderate agitation), 3 (mild agitation) or 4 (normal behavior) within about 2 hours after administering the dexmedetomidine or a pharmaceutically acceptable salt thereof, as measured by the Agitation-Calmness Evaluation Scale (ACES). In embodiments, the elderly patient is about 70 years old or older. In embodiments, the elderly patient is about 75 to about 80 years old. In embodiments, the elderly patient is about 80 years old or older.

In other embodiments, the present disclosure provides a method of reducing a period of opioid withdrawal in a human subject in need thereof, comprising administering oromucosally (e.g., sublingually, buccally, or gingivally), to said subject about 30 μg to about 600 μg of dexmedetomidine or a pharmaceutically acceptable salt thereof. In embodiments, the period of withdrawal is up to about 14 days. In embodiments, dexmedetomidine or a pharmaceutically acceptable salt thereof is oromucosally administered at a unit dose containing about 30 μg, about 60 μg, about 90 μg, about 120 μg, about 150 μg, about 180 μg, about 240 μg or about 300 μg twice daily. In embodiments, the period of withdrawal may be 13 days, 12 days, 11 days, 10 days, 9 days, 8 days, 7 days, 6 days, 5 days, 4 days, or 3 days. In other embodiments, the present disclosure provides a method of reducing a period of opioid withdrawal in a human subject in need thereof, comprising administering oromucosally (e.g., sublingually, buccally, or gingivally), to said subject about 30 μg to about 600 μg of dexmedetomidine or a pharmaceutically acceptable salt thereof. In embodiments, the period of withdrawal is up to about 60 days. In embodiments, the period of withdrawal may be 59 days, 58 days, 57 days, 56 days, 55 days, 54 days, 53 days, 52 days, 51 days, 50 days, 49 days, 48 days, 47 days, 46 days, 45 days, 44 days, 43 days, 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days or 3 days. In embodiments the human subject is an adult (e.g., 18 years or older). In embodiments, dexmedetomidine or a pharmaceutically acceptable salt is administered oromucosally (e.g., sublingually, buccally, gingivally), orally, intranasally or parenterally. In embodiments, dexmedetomidine or a pharmaceutically acceptable salt thereof (e.g., hydrochloride) is administered sublingually as a film. In embodiments, dexmedetomidine or a pharmaceutically acceptable salt (e.g., dexmedetomidine hydrochloride) is administered buccally or sublingually as a film. In embodiments, the opioid withdrawal is withdrawal from use of fentanyl, morphine, codeine, heroin, oxycodone, hydrocodone, alfentanil, carfentanil, tramadol, hydromorphone, buprenorphine, naloxone, naltrexone, remifentanil, butorphanol, meperidine, methadone, dextropropoxyphene (propoxyphene) thebaine, sufentanil, or pentazocine or combinations thereof.

In embodiments, the disclosure provides methods of reducing a period of opioid withdrawal in a human subject in need thereof comprising administering dexmedetomidine or a pharmaceutically acceptable salt (e.g., dexmedetomidine hydrochloride) to the oral mucosa (i.e. sublingually, buccally, or gingivally) of said subject in an amount of about 30 μg to about 600 μg. In embodiments, the mean plasma concentrations is in the range of about 40 ng/L to about 500 ng/L after 2 hours following administration of dexmedetomidine or a pharmaceutically acceptable salt (e.g., dexmedetomidine hydrochloride). In embodiments, the mean plasma concentrations are in the range of about 20 ng/L to about 150 ng/L after 12 hours of administration of dexmedetomidine or a pharmaceutically acceptable salt (e.g. hydrochloride). In embodiments, the mean plasma concentrations are in the range of about 50 ng/L to about 500 ng/L after 2 hours of administration of dexmedetomidine or a pharmaceutically acceptable salt (e.g. hydrochloride). In embodiments, the mean plasma concentrations are in the range of about 10 ng/L to 150 ng/L after 12 hours of administration of dexmedetomidine or a pharmaceutically acceptable salt.

In other embodiments, the present disclosure provides a method of treating agitation in an agitated dementia patient in need thereof, comprising administering a mucoadhesive oromucosal (e.g., sublingually, buccally, or gingivally) composition, to said patient in an amount of about 30 μg to about 120 μg of dexmedetomidine or a pharmaceutically acceptable salt thereof. In embodiments, dexmedetomidine or a pharmaceutically acceptable salt thereof is oromucosally administered at a unit dose containing about 20 μg, about 30 μg, about 40 μg, about 50 μg, about 60 μg, about 70 μg, about 80 μg, about 90 μg, about 100 μg, about 110 μg, or about 120 μg once or twice daily. In embodiments, the the patient has Alzheimer's disease. In embodiments, the patient is 65 to 80 years old. In embodiments, the dose is about 30 mcg and the administration to the oral mucosa results in a Cₘₐₓ from about 36 ng/L to about 147 ng/L and an AUC_{0-inf} of from about 200 hr*ng/L to about 1500 hr*ng/L. In embodiments, the dose is about 40 mcg and the administration to the oral mucosa results in a Cₘₐₓ from about 50 ng/L to about 300 ng/L and an AUC_{0-inf} of from about 200 hr*ng/L to about 1500 hr*ng/L.

The present disclosure also provides methods of managing or treating agitation in subjects with delirium, comprising administering to said subject about 20 μg to about 300 μg of dexmedetomidine or a pharmaceutically acceptable salt thereof. In embodiments, the subject is hospitalized. In embodiments, the subject is hospitalized in the intensive care unit. In embodiments, dexmedetomidine or a pharmaceutically acceptable salt thereof is administered oromucosally (e.g., sublingually, buccally, or gingivally) at a dose of about 20 μg, about 30 μg, about 60 μg, about 80 μg, about 90 μg, about 100 μg, about 120 μg, about 150 μg, about 180 μg, about 210 μg, about 240 μg, about 270 μg, or about 300 μg. In embodiments, dexmedetomidine or a pharmaceutically acceptable salt thereof is administered one to six times a day. In embodiments, dexmedetomidine or a pharmaceutically acceptable salt thereof is administered twice a day. In order to achieve the desired dose, about may be oromucosally administered as a single unit dose, as multiple unit doses, or as a fraction of one or more unit doses (e.g. half of a unit dose), or a combination thereof. By way of example, to administer 120 μg of dexmedetomidine or a pharmaceutically acceptable salt thereof, the subject may be administered for example, a single unit dose of 120 μg of dexmedetomidine or a pharmaceutically acceptable salt thereof, two unit doses of 60 μg of dexmedetomidine or a pharmaceutically acceptable salt thereof, or three unit doses of 40 μg dexmedetomidine or a pharmaceutically acceptable salt thereof. In embodiments, dexmedetomidine or a pharmaceutically acceptable salt thereof is administered as a film. Thus, half doses can be achieved by cutting the film in half, for example, cutting a 120 μg or 180 μg film in half to achieve a 60 μg dose and a 90 μg dose, respectively. In embodiments, the dose may be administered multiple times (e.g. one to four times) at an appropriate dosing interval (e.g. every 0.5 hours) to produce a desired effect; for example, a 20 μg unit dose or a 60 μg unit dose can be administered four times at a dosing interval of every 0.5 hours within 6 hours of the first dose to produce the effect of a 80 μg dose and a 240 μg dose, respectively. In embodiments, each dosage unit may be administered one to two times at an appropriate dosing interval (every 12 hours) to produce a desired effect; for example, a 120 μg unit is administered two times in a day at an interval of 12 hours to produce the effect of a 240 μg dose. In embodiments, each dosage unit may be administered one to ten times at an appropriate dosing interval (e.g. every 1 to 6 hours) to produce a desired effect; for example, a 120 μg dose (starting dose) is administered followed by an additional seven doses in a day at an interval of about 1 to about 6 hours to produce the maximum cumulative dose of a 960 μg dose. In embodiments, each dosage unit may be administered one to ten times at an appropriate dosing interval (e.g. every 1 to 6 hours) to produce a desired effect; for example, a 180 μg dose (starting dose) is administered followed by an additional six doses of 120 μg in a day at an interval of about 1 to about 6 hours to produce the maximum cumulative dose of a 900 μg dose. In embodiments, each dosage unit may be administered one to ten times at an appropriate dosing interval (for e.g. of at least 1 to 6 hours) to produce a desired effect; for example, a 240 μg dose (starting dose) is administered followed by an additional six doses of 120 μg in a day at an interval of about 1 to about 6 hours to produce the maximum cumulative dose of a 960 μg dose. In embodiments, each dosage unit may be administered one to ten times at an appropriate dosing interval (for e.g. of at least 1 to 6 hours) to produce a desired effect; for example, a 300 μg dose (starting dose) is administered followed by additional five doses of 120 μg in a day at an interval of about 1 to about 6 hours to produce the maximum cumulative dose of a 900 μg dose. In embodiments, dexmedetomidine or a pharmaceutically acceptable salt thereof is administered oromucosally (e.g. sublingually or buccally) as a film. In embodiments, the subject is 18-64 years old. In embodiments, the subject is over 65 years old. In embodiments, the dexmedetomidine is administered at a dose of about 60 μg, 90 μg, 120 μg and 150 μg one to six times a day (e.g. for patients that are 65 years old or older). In embodiments, the dexmedetomidine is administered at a dose of about 120 μg, 180 μg, 240 μg and 300 μg one to six times a day (e.g. for patients that are less than 65 years old). In embodiments, the subject is treated without experiencing clinically significant cardiovascular effects.

In embodiments, the disclosure provides methods of managing or treating agitation or signs of agitation in subjects with delirium, comprising administering a dose of about 20 μg, about 40 μg, about 60 μg, about 90 μg, about 120 μg or about 150 μg of dexmedetomidine or a pharmaceutically acceptable salt thereof wherein the subject's age is 65 years old or older. In embodiments, one to ten doses can be administered at an appropriate dosing interval (e.g. 1 to 6 hours) to produce a desired effect; for example, about 60 μg, 90 μg, 120 μg, or 150 μg dose (starting dose) is administered followed by an additional 5-7 doses of 60 μg in a day at an interval ranging from about 1 to about 6 hours to produce the maximum cumulative dose of a 480 μg dose.

The disclosure also provides a pharmaceutical composition comprising from about 20 μg to about 300 μg dexmedetomidine or a pharmaceutically acceptable salt thereof (e.g. dexmedetomidine hydrochloride). In embodiments, the dose of dexmedetomidine is about 30 μg. In embodiments, the dose of dexmedetomidine is about 40 μg. In embodiments, the dose of dexmedetomidine is about 60 μg. In embodiments, the dose of dexmedetomidine is about 90 μg. In embodiments, the dose of dexmedetomidine is about 120 μg. In embodiments, the dose of dexmedetomidine is about 150 μg. In embodiments, the dose of dexmedetomidine is about 180 μg. In embodiments, the dose of dexmedetomidine is about 240 μg. In embodiments, the dose of dexmedetomidine is about 300 μg. In embodiments, the dose can be taken one to ten times a day.

### BRIEF DESCRIPTION OF THE DRAWINGS

FIG. 1 depicts change in PEC score from baseline in elderly dementia patients until 8 hours post-dose of 30 μg and 60 μg dexmedetomidine oromucosal thin film compared to placebo.

FIG. 2 depicts change in PAS score from baseline in elderly dementia patients until 8 hours post-dose of 30 μg and 60 μg dexmedetomidine oromucosal thin film compared to placebo.

FIG. 3 depicts change in Mod-CMAI score from baseline in elderly dementia patients at 2 hours post-dose of 30 μg and 60 μg dexmedetomidine oromucosal thin film compared to placebo.

FIG. 4 depicts percent response in elderly dementia patients at 1, 2, 4 and 8 hours following administration of 30 μg (middle bar) and 60 μg dexmedetomidine (right bar) oromucosal film compared to placebo (left bar), as measured by Clinical Global Impression-Improvement (CGI).

FIG. 5 depicts calming improvement in elderly dementia patients at 2 hours following administration of 30 μg (middle bar) and 60 μg dexmedetomidine (right bar) oromucosal film compared to placebo (left bar), as measured by Agitation and Calmness Evaluation Scale (ACES).

FIG. 6 depicts the Cₘₐₓ (top) and AUC₀₋₈ (bottom) dose relationship in elderly dementia patients.

FIG. 7 depicts reduced acute opioid withdrawal symptoms compared to placebo as measured by the COWS after administration of dexmedetomidine oromucosal film 120 mcg, 180 μg and 240 μg BID according to Example 5.

FIG. 8 depicts reduced acute opioid withdrawal symptoms compared to placebo as measured by the SOWS after administration of dexmedetomidine oromucosal film 120 μg, 180 μg and 240 μg BID according to Example 5.

FIG. 9 depicts higher study retention after administration of dexmedetomidine oromucosal film 120 μg, 180 μg and 240 μg BID as compared to placebo according to Example 5.

FIG. 10 depicts the Score Simulations of Placebo and SL administration Regimens

FIG. 11 depicts the PEC Change from Baseline (%) Simulations of Placebo and SL administration Regimens

FIG. 12 depicts the PAS Score Simulations of Placebo and SL administration Regimens

FIG. 13 depicts the PAS Change from Baseline (%) Simulations of Placebo and SL administration Regimens

FIG. 14 depicts the CMAI Score Simulations of Placebo and SL administration Regimens

FIG. 15 depicts the CMAI Change from Baseline (%) Simulations of Placebo and SL administration Regimens

### DETAILED DESCRIPTION

### Abbreviations

ACES: Agitation-Calmness Evaluation Scale

AD: Alzheimer disease

AE: Adverse event

AUC: Area under the curve

AUC₀₋₈: Area under the plasma concentration-time curve from time of administration to 8 hours

AUC_{0-inf}: Area under the plasma concentration-time curve from time of administration to infinity

AUD: alcohol use disorder

BAC/BrAC: Breath Alcohol concentration

BAES: Biphasic Alcohol Effects Scale

BID: twice a day

BMI: Body mass index

CAPS-5: Clinician-Administered PTSD Scale for DSM-5

CGI-I: Clinical Global Impression-Improvement

CGI-S: Clinical Global Impression-Severity

CIWA-AR: Clinical Institute Withdrawal Assessment for Alcohol Scale

CLIA: Clinical Laboratory Improvement Amendments

Cₘₐₓ: maximum plasma concentration

COWS: Clinical Opiate Withdrawal Scale

CMAI: Cohen Mansfield Agitation Inventory

CMC: Carboxy methylcellulose

C-SSRS: Columbia Suicide Severity Rating Scale

CT: Computed tomography

DBP: Diastolic Blood Pressure

DEQ: Drug Effects questionnaire

DES-R: Differential Emotions Scale

Dex or DEX: Dexmedetomidine

DSM: Diagnostic and Statistical Manual of Mental Disorders

DSMB: Drug Safety Monitoring Board

DT: Disintegration time

ECG: Electrocardiogram

FTD: Fronto temporal disease

HPC: Hydroxypropyl cellulose

HPMC: Hydroxyl propyl methyl cellulose

HR: Heart rate

HVLT-R: Hopkins Verbal Learning Test

ICH: International Conference on Harmonisation

ICU—Intensive care unit

IUD: intrauterine device

ITT: Intent to treat Population

LAR: Legally authorized representative

LEC-5 Life Event Checklist for DSM-5

LS: Least square

MedDRA: Medical Dictionary for Regulatory Activities

MINI-5: Mini-International Neuropsychiatric Interview for DSM-5

MMRM: Mixed model repeated measures

MMSE: Mini-Mental State Examination

MW: Molecular weight

mm: Millimeter

mcg: Microgram

mg: Milligrams

μg: Microgram

ml: Milliliter

mmHg: Millimeters of mercury

msec: Millisecond

NDS: Number of Drinks Scale

ng: Nanogram

PANSS: Positive and Negative Syndrome Scale

PAS: Pittsburgh Agitation Scale

PCL-5: PTSD Checklist for DSM-5

PCRS: Placebo-Control Reminder Script

PEC: PANSS Excitement Component

PEO: Polyethylene oxide

PD: Pharmacodynamic

PK: Pharmacokinetics

PTSD: posttraumatic stress disorder

PVA: Polyvinyl alcohol

QTcF: QT interval corrected for heart rate using Fridericia's formula

QID: Quater in die

RASS: Richmond Agitation Sedation Scale

RVIP: Rapid Information Processing Task

SAE: Serious adverse event

SOWS-Gossop: Short Opiate Withdrawal Scale of Gossop

SAP: Statistical Analysis Plan

SBP: Systolic Blood Pressure

SD: Standard deviation

SE: Standard error

SL: Sublingual

STAI: State Trait Anxiety Inventory

T_{1/2}: Elimination half-life

TEAE: Treatment emergent adverse event

TLFB: Timeline Follow Back

Tmax: Time of maximum plasma concentration

Wt %: Weight percentage

ULN: Upper limit of normal

VAS: Visual Analog Scale

YCS: Yale craving scale

YMRS: Young Mania Rating Scale

### Definitions

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity. As such, the terms “a” (or “an”), “one or more” and “at least one” are used interchangeably herein. In addition, reference to “an agent” by the indefinite article “a” or “an” does not exclude the possibility that more than one of the agents are present, unless the context clearly requires that there is one and only one of the agents.

As used herein, “about” means plus or minus 10% of the indicated numerical value.

The term “agitation”, as used herein, means irritability, emotional outburst, impaired thinking, or excess motor and verbal activity that may occur due to either dysfunction of specific brain regions such as frontal lobes or due to dysfunction of neurotransmitter systems such as dopamine and nor-epinephrine. In the present disclosure, agitation also includes aggression and hyper-arousal in post-traumatic stress disorder. The agitation may be acute or chronic.

The term “buccal” means administration of the dosage form against the gum and the inner lip or cheek.

As used herein, the term “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

The term “clinically significant cardiovascular effects” means herein a lowering in blood pressure (hypotension) and/or heart rate (bradycardia) to the extent that medical intervention is required to address the cardiovascular side effects, where the term “medical intervention” means an intervention that more serious than administering fluids, such as an energy drink.

As used herein, the phrase “deposited on the surface of a polymer matrix” means that dexmedetomidine or a pharmaceutically acceptable salt thereof is formulated as liquid composition separate from the preparation of the solid polymer matrix, and deposited onto the solid polymer, e.g. as one or more micro-deposits, where it dries. The dried product is sometimes referred to herein as the “micro-deposited matrix film”. The drug liquid formulation may be in any form, including as a solution, emulsion, suspension, or dispersion.

As used herein, the phrase “disposed within a polymer matrix” means that dexmedetomidine or a pharmaceutically acceptable salt thereof is incorporated directly into the polymer solution prior to the formation of the solid polymer matrix film composition.

The term “dissolvable” means the films herein are readily disintegrated, e.g. at least within about 20 minutes, following administration to the oral mucosa. Disintegration is achieved by saliva and/or other aqueous materials on the mucosal surface.

The term “an effective amount” is interchangeable with “therapeutically effective dose,” or “therapeutically effective amount,” and refers to an amount sufficient to produce the desired effect. An effective amount is sufficient to cause an improvement in a condition (e.g. agitation) of the subject.

The term “film” herein includes thin films, in any shape, including rectangular, square, or other desired shape. The film may be of any desired thickness and size, such that it can be conveniently placed oromucosally in the patient. For example, the film may be a relatively thin film having a thickness of from about 20 micrometers to about 200 micrometers or may be a somewhat thicker film having a thickness of from about 20 micrometers to about 1000 micrometers. In embodiments, the film may be even thicker, e.g., having a thickness greater than about 30 millimeters.

The terms “formulation” and “composition” are used interchangeably, except where otherwise clearly intended to have different meanings.

The term “intranasal administration” means administration by the nasal route, whereby a drug is insufflated through the nose. The administration can be either topical or systemic, meaning the locally delivered drug can go on to exhibit either purely local or systemic effects.

The term “mucoadhesion” is used herein to refer to adhesion to mucosal membranes, such as those in the oral cavity.

The term “mucoadhesive” refers to the property of adhering to a mucosal tissue surface in vivo. Such adhesion adherently localizes the dosage form onto the mucus membrane and requires the application of a force to separate the mucoadhesive material from the mucus membrane.

“Opioid or alcohol or substance withdrawal” refers to a variety of signs and complaints appearing with the abrupt removal of, or a rapid decrease in the regular dosage of opioids or alcohol or other substance. Physical manifestations may include sweating, nausea, yawning, chills, diarrhea, papillary dilation, piloerection, tachycardia, increased blood pressure, hypersensitivity to pain, stomach cramps, and muscle cramps. Opioid or alcohol or substance withdrawal is a set of symptoms (a syndrome) arising from the sudden withdrawal or reduction of opioids alcohol or other substance where previous usage has been heavy and prolonged. Signs and symptoms of withdrawal can include drug craving, anxiety, restless legs, nausea, vomiting, diarrhea, sweating, and an increased heart rate. Psychological manifestations of opioid withdrawal may include agitation, dysphoria, restlessness, irritability, anxiety, and depression. In embodiments, the opioid withdrawal symptom is agitation. In embodiments, treating or ameliorating opioid withdrawal refers to the treatment or lessening of one or more of the aforementioned symptoms.

The term “oromucosal” means administration to the oral mucosa, specifically the oral cavity and/or the pharynx. Oromucosal administration includes administration by sublingual, buccal, or gingival routes.

The term “parenteral” refers to administration of a drug by injection under one or more layer of skin or mucous membrane, and can include, for example, subcutaneous, intravenous, intraperitoneal or intramuscular injection.

The term “pharmaceutically acceptable carrier” refers to a pharmacologically inert substance to be used as a carrier. As used herein, the phrase “carrier” and “excipients” are used interchangeably, except where otherwise clearly intended to have different meanings.

The term “pharmaceutically acceptable salt” refers to a salt known to be non-toxic and commonly used in the pharmaceutical literature. Typical inorganic acids used to form such salt include hydrochloric, hydrobromic, hydroiodic, nitric, sulfuric, phosphoric, hypophosphoric, and the like. Salts derived from organic acids, such as aliphatic mono and dicarboxylic acids, phenyl substituted alkanoic acids, hydroxyalkanoic and hydroxyl alkandioic acids, aromatic acids, aliphatic and aromatic sulfonic acids may also be used. A preferred salt is the hydrochloride salt.

The term “self-supporting” means the films herein maintain structural integrity upon handling without the need for a backing layer. Some flexibility in the film is contemplated and may be desirable.

The term “signs of agitation” includes excessive motor activity (examples include: pacing, rocking, gesturing, pointing fingers, restlessness, performing repetitious mannerisms), verbal aggression (e.g. yelling, speaking in an excessively loud voice, using profanity, screaming, shouting, threatening other people), physical aggression (e.g. grabbing, shoving, pushing, clenching hands into fists, resisting, hitting others, kicking objects or people, scratching, biting, throwing objects, hitting self, slamming doors, tearing things), and destroying property.

As used herein, the term “subject” preferably refers to a human patient. In embodiments, the subject can be any animal, including non-human mammals, such as mice, rats, other rodents, rabbits, dogs, cats, swine, cattle, sheep, horses, or primates.

The term “significantly reduced” refers to a reduction level by at least 10% or higher, preferably 20% or higher, more preferably 40% or higher, even more preferably 60% or higher, still more preferably 80% or higher, and 90% or higher, as compared to a control. For example, in the context of agitation, the skilled artisan will readily understand that the reduction can be measured in terms of well-known agitation scales, such as PEC score and CGI-I As an example, when agitation is significantly reduced in a patient, the reduction may be interpreted as as those who achieve at least a 10%, 20%, 30%, 40%, 50%, 60%, 70%, 80%, 90%, or 100% or greater reduction in PEC total score from baseline (e.g. measured at 2 hours post-dose). In embodiments, significantly reduced agitation refers to at least a 40% reduction in PEC total score from baseline. Similarly, a significant reduction in agitation may be measured on the CGI-I scale and may refer to a patient that has a score of 1 or 2 on the CGI-I scale (e.g. measured at 1, 2, or 4 hours post-dose) or the Agitation-Calmness Evaluation Scale (ACES) scale and may refer to a patient that has a score of e.g. 3 or higher.

The term “sublingual” means “under the tongue” and refers to a method of administering substances via the blood vessels under the tongue. Sublingual absorption occurs through the highly vascularized sublingual mucosa, which allows a substance direct access to the blood circulation, thereby providing for direct systemic administration independent of gastrointestinal influences and avoiding undesirable first-pass hepatic metabolism.

“Therapeutic” as used herein, refer to treatment and/or prophylaxis, depending on context.

The terms “treat”, “treating,” and “treatment,” as used herein refer to a particular disease or disorder includes lessening, improving, ameliorating or abrogating the symptoms and/or pathology of the disease or disorder. The term “prevention “means preventing the occurrence of a disease or condition, or associated symptoms or preventing the recurrence of the same, for example after a period of improvement.

The term “unit dose,” “unit dosage,” or “unit dosage form” means a physically discrete unit that contains a predetermined quantity of dexmedetomidine or a pharmaceutically acceptable salt thereof.

As used herein, the phrase “water-soluble polymer” refers to (i) a polymer that is at least partially soluble in water, and desirably fully or predominantly soluble in water, and/or (ii) a polymer that absorbs water. Polymers that absorb water are referred to herein as water-swellable polymers.

The term “without significant sedation” and the like means that the patient experiences a level of sedation not greater than Level 3 on the Ramsay Sedation Scale. Level 3 means sedated but responds to commands. In embodiments, the dexmedetomidine may be dosed to achieve a Richmond Agitation Sedation Scale (RASS) of −1 (“light sedation”).

The term AUC_{0-inf} represents the total drug exposure across time. AUC_{0-inf} is calculated as the sum of AUCₗₐₛₜ and AUCₑₓₜ. AUCₗₐₛₜ is calculated by integration of the concentration-time data using the trapezoidal rule up to the last quantifiable concentration. AUCₑₓₜ is calculated by dividing the last quantifiable concentration by the elimination rate constant.

### Active Agent

Dexmedetomidine has the IUPAC name (+) 4-(S)-[1-(2,3-dimethylphenyl)ethyl]-1H-imidazole. As the monohydrochloride salt, it is predominantly used as a medication for the sedation of patients during treatment in an intensive care setting or to sedate patients prior to and/or during surgical and other procedures. Such medication is currently sold under the registered trade name “PRECEDEX”.

Pharmaceutically acceptable salts of dexmedetomidine that may be included herein generally include any suitable salt that has been or may be approved by the U.S. FDA or other appropriate foreign or domestic agency for administration to a human. Non-limiting examples of suitable pharmaceutically acceptable salts include salts of inorganic acids such as hydrochloric, hydrobromic, nitric, carbonic, monohydrocarbonic, phosphoric, monohydrogen phosphoric, dihydrogen phosphoric, sulfuric, hydrogen sulfuric, and hydroiodic acid. Other examples include salts derived from non-toxic organic acids, including acetic, propionic, isobutyric, maleic, malonic, benzoic, succinic, suberic, fumaric, lactic, mandelic, phthalic, benzenesulfonic, p-toluenesulfonic, citric, tartaric, and methanesulfonic acids, or combinations of these acid salts. Exemplary salts include dexmedetomidine hydrochloride, dexmedetomidine hydrobromide, dexmedetomidine sulfate, dexmedetomidine sulfonate, dexmedetomidine phosphate, dexmedetomidine nitrate, dexmedetomidine formate, dexmedetomidine citrate, dexmedetomidine tartrate, dexmedetomidine malate, dexmedetomidine benzoate, dexmedetomidine salicylate, dexmedetomidine ascorbate or the like. In embodiments, deuterated forms of dexmedetomidine or a pharmaceutically acceptable salt thereof may be included.

### Methods and Administration

In embodiments, the disclosure encompasses methods of treating agitation or signs of agitation in a subject comprising oromucosally administering dexmedetomidine or a pharmaceutically acceptable salt thereof to the subject in an agitated state. In embodiments, the disclosure includes a method of treating agitation or signs of agitation in a subject with dementia comprising oromucosally administering dexmedetomidine or a pharmaceutically acceptable salt thereof to the subject in an agitated state. In embodiments, the disclosure provides methods of treating agitation in elderly patients having dementia comprising oromucosally administering dexmedetomidine or a pharmaceutically acceptable salt thereof to the patient in an agitated state. In embodiments, the disclosure provides methods of managing or treating agitation in subjects with delirium, comprising oromucosally administering dexmedetomidine or a pharmaceutically acceptable salt thereof to the patient in an agitated state. In embodiments, the disclosure also provides methods of treating or ameliorating opioid withdrawal or related symptoms, comprising oromucosally administering dexmedetomidine or a pharmaceutically acceptable salt thereof (e.g., dexmedetomidine hydrochloride) to a patient in need thereof. In embodiments, the disclosure provides a method of treating cocaine toxicity and/or symptoms associated with cocaine toxicity comprising oromucosally administering an effective amount of dexmedetomidine or a pharmaceutically acceptable salt thereof. In embodiments, the disclosure provides a method of treating agitation or signs of agitation in a pediatric subject comprising oromucosally administering dexmedetomidine or a pharmaceutically acceptable salt thereof to the subject in an agitated state.In embodiments, the agitation is acute agitation. In embodiments, the agitation is chronic agitation. In embodiments, the disclosure is a method of treating agitation without also inducing significant sedation. In embodiments, the treatment is effective without causing clinically significant cardiovascular effects.

The exemplary dosage of dexmedetomidine or a pharmaceutically acceptable salt thereof to be administered to a particular patient in the various methods of the invention will depend on the type and extent of the condition, the overall health status of the particular patient, the particular form of dexmedetomidine or a pharmaceutically acceptable salt thereof being administered, and the particular formulation used to treat the patient. In embodiments, one or more units (e.g., film composition) is administered to deliver the dose. In embodiments, the dose can be given by combining two or more dose units, for example, 60 μg (30 μg unit+30 μg unit), 90 μg (30 μg unit+60 μg unit), 120 μg (60 μg unit+60 μg unit), 150 μg (half of 120 μg unit+half of 180 μg unit), 240 μg (180 μg unit+half of 120 μg unit), 300 μg (120 μg unit+180 μg unit) and so on. The dose may be administered one or more times a day including twice, three times, four times, five times or six times per day.

In embodiments, the dosage of dexmedetomidine or a pharmaceutically acceptable salt thereof administered is between about 0.5 μg to about 1200 μg. Examples of suitable dosages include, e.g., about 0.5 μg to about 1200 μg, about 0.5 μg to about 500 μg, about 0.5 μg to about 450 μg, about 0.5 μg to about 405 μg, about 0.5 μg to about 360 μg, about 0.5 μg to about 270 μg, about 0.5 μg to about 180 μg, and about 0.5 μg to about 120 μg.

In embodiments, dexmedetomidine or a pharmaceutically acceptable salt thereof may be administered at a dose of about 10 μg to about 300 μg, e.g., about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, including all values and ranges in between.

In embodiments, dexmedetomidine or a pharmaceutically acceptable salt thereof may be administered at a dose of about 120 μg to about 405 μg, e.g. about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, or about 405 μg, including all values and ranges in between. In embodiments, dexmedetomidine or a pharmaceutically acceptable salt thereof may be administered at a dose of about 120 μg to about 405 μg, e.g. about 120 μg to about 270 μg, about 120 μg and about 180 μg.

In embodiments, dexmedetomidine or a pharmaceutically acceptable salt thereof may be administered oromucosally (e.g. sublingually or buccally) at a dose of about 10 μg to about 300 μg, e.g., about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, including all values and ranges in between. In embodiments, dexmedetomidine or a pharmaceutically acceptable salt thereof may be administered oromucosally (e.g. sublingually or buccally) at a dose of about 10 μg to about 300 μg, e.g. about 10 μg to 270 μg, about 20 μg to about 240 μg, about 30 μg to about 180 μg, about 40 μg to about 140 μg, about 50 μg to about 120 μg , about 60 μg to about 120 μg, about 70 μg to about 100 μg, about 80 μg to about 100 μg.

In embodiments, dexmedetomidine or a pharmaceutically acceptable salt thereof (e.g. dexmedetomidine hydrochloride) is oromucosally administered in an amount of about 300 μg. In embodiments, dexmedetomidine or a pharmaceutically acceptable salt thereof (e.g. dexmedetomidine hydrochloride) is oromucosally administered in an amount of about 270 μg. In embodiments, dexmedetomidine or a pharmaceutically acceptable salt thereof (e.g. dexmedetomidine hydrochloride) is oromucosally administered in an amount of about 240 μg. In embodiments, dexmedetomidine or a pharmaceutically acceptable salt thereof (e.g. dexmedetomidine hydrochloride) is oromucosally administered in an amount of about 210 μg. In embodiments, dexmedetomidine or a pharmaceutically acceptable salt thereof (e.g. dexmedetomidine hydrochloride) is oromucosally administered in an amount of about 180 μg. In embodiments, dexmedetomidine or a pharmaceutically acceptable salt thereof (e.g. dexmedetomidine hydrochloride) is oromucosally administered in an amount of about 150 μg. In embodiments, dexmedetomidine or a pharmaceutically acceptable salt thereof (e.g. dexmedetomidine hydrochloride) is oromucosally administered in an amount of about 120 μg. In embodiments, dexmedetomidine or a pharmaceutically acceptable salt thereof (e.g. dexmedetomidine hydrochloride) is oromucosally administered in an amount of about 90 μg. In embodiments, dexmedetomidine or a pharmaceutically acceptable salt thereof (e.g. dexmedetomidine hydrochloride) is oromucosally administered in an amount of about 60 μg. In embodiments, dexmedetomidine or a pharmaceutically acceptable salt thereof (e.g. dexmedetomidine hydrochloride) is oromucosally administered in an amount of about 40 μg. In embodiments, dexmedetomidine or a pharmaceutically acceptable salt thereof (e.g. dexmedetomidine hydrochloride) is oromucosally administered in an amount of about 30 μg. In embodiments, the dosages of dexmedetomidine or a pharmaceutically acceptable salt thereof are administered at a dose of about 30 μg to about 180 μg (e.g., 30 μg, 40 μg, 45 μg, 60 μg, 90 μg, 120 μg or 180 μg) In embodiments, one or more units is administered to deliver the dose.

In embodiments, dexmedetomidine or a pharmaceutically acceptable salt thereof is administered intramuscularly at a dose of about 100 μg to about 200 μg, e.g. about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, or about 200 μg, including all values and ranges in between. In embodiments, dexmedetomidine or a pharmaceutically acceptable salt thereof is administered intramuscularly at a dose of about 100 μg to about 200 μg, e.g. about 120 μg to about 190 μg.

In embodiments, dexmedetomidine or a pharmaceutically acceptable salt thereof may be administered orally at a dose of about 500 μg to about 1500 μg based on total weight of the oral composition, e.g., about 500 μg, about 510 μg, about 520 μg, about 530 μg, about 540 μg, about 550 μg, about 560 μg, about 570 μg, about 580 μg, about 590 μg, about 600 μg, about 610 μg, about 620 μg, about 630 μg, about 640 μg, about 650 μg, about 660 μg, about 670 μg, about 680 μg, about 690 μg, about 700 μg, about 710 μg, about 720 μg, about 730 μg, about 740 μg, about 750 μg, about 760 μg, about 770 μg, about 780 μg, about 790 μg, about 800 μg, about 810 μg, about 820 μg, about 830 μg, about 840 μg, about 850 μg, about 860 μg, about 870 μg, about 880 μg, about 890 μg, about 900 μg, about 910 μg, about 920 μg, about 930 μg, about 940 μg, about 950 μg, about 960 μg, about 970 μg, about 980 μg, about 990 μg, about 1000 μg, about 1010 μg, about 1020 μg, about 1030 μg, about 1040 μg, about 1050 μg, about 1060 μg, about 1070 μg, about 1080 μg, about 1090 μg, about 1100 μg, about 1110 μg, about 1120 μg, about 1130 μg, about 1140 μg, about 1150 μg, about 1160 μg, about 1170 μg, about 1180 μg, about 1190 μg, about 1200 μg, about 1210 μg, about 1220 μg, about 1230 μg, about 1240 μg, about 1250 μg, about 1260 μg, about 1270 μg, about 1280 μg, about 1290 μg, about 1300 μg, about 1310 μg, about 1320 μg, about 1330 μg, about 1340 μg, about 1350 μg, about 1360 μg, about 1370 μg, about 1380 μg, about 1390 μg, about 1400 μg, about 1410 μg, about 1420 μg, about 1430 μg, about 1440 μg, about 1450 μg, about 1460 μg, about 1470 μg, about 1480 μg, about 1490 μg, or about 1500 μg, including all values and ranges in between.

In embodiments, the dose is about 30 μg and the AUC₀₋₈ range is between about 200 hr*ng/L to about 600 hr*ng/L, for example, about 200 hr*ng/L to about 400 hr*ng/L, about 300 hr*ng/L to about 600 hr*ng/L, about 300 hr*ng/L to about 500 hr*ng/L, about 350 hr*ng/L to about 450 hr*ng/L.

In embodiments, the dose is about 30 μg and the AUC_{0-inf} is about 200 hr*ng/L to about 1700 hr*ng/L, e.g., about 200 hr*ng/L, about 225 ng*hr/mL, about 250 ng*hr/mL, about 275 ng*hr/mL, about 300 ng*hr/mL, about 325 ng*hr/mL, about 350 ng*hr/mL, about 375 ng*hr/mL, about 400 ng*hr/mL, about 425 ng*hr/mL, about 450 ng*hr/mL, about 475 ng*hr/mL, about 500 ng*hr/mL, about 525 ng*hr/mL, about 550 ng*hr/mL, about 575 ng*hr/mL, about 600 ng*hr/mL, about 625 ng*hr/mL, about 650 ng*hr/mL, about 675 ng*hr/mL, about 700 ng*hr/mL, about 725 ng*hr/mL, about 750 ng*hr/mL, about 775 ng*hr/mL, about 800 ng*hr/mL, about 825 ng*hr/mL, about 850 ng*hr/mL, about 875 ng*hr/mL, about 900 ng*hr/mL, about 925 ng*hr/mL, about 950 ng*hr/mL, about 975 ng*hr/mL, about 1000 ng*hr/mL, about 1025 ng*hr/mL, about 1050 ng*hr/mL, about 1075 ng*hr/mL, about 1100 ng*hr/mL, about 1125 ng*hr/mL, about 1150 ng*hr/mL, about 1175 ng*hr/mL, about 1200 ng*hr/mL, about 1225 ng*hr/mL, about 1250 ng*hr/mL, about 1275 ng*hr/mL, about 1300 ng*hr/mL, about 1325 ng*hr/mL, about 1350 ng*hr/mL, about 1375 ng*hr/mL, about 1400 ng*hr/mL, about 1425 ng*hr/mL, about 1450 ng*hr/mL, about 1475 ng*hr/mL, about 1500 ng*hr/mL, about 1525 ng*hr/mL, about 1550 ng*hr/mL, about 1575 ng*hr/mL, about 1600 ng*hr/mL, about 1625 ng*hr/mL, about 1650 ng*hr/mL, about 1675 ng*hr/mL, or about 1700 ng*hr/mL, including all values and ranges in between.

In embodiments, the dose is about 30 μg, the AUC₀₋₈ range is about 80% to 125% of about 200 hr*ng/L to about 600 hr*ng/L and and the AUC_{0-inf} range is about 80% to 125% of about 200 hr*ng/L to about 1700 hr*ng/L.

In embodiments, the dose is about 40 μg and the AUC_{0-inf} range is about 80% to 125% of about 300 hr*ng/L to about 2200 hr*ng/L, about 500 hr*ng/L to about 1500 hr*ng/L , about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L

In embodiments, the dose is about 40 μg and the AUC_{0-inf} is about 300 hr*ng/L to about 2200 hr*ng/L, e.g., about 300 hr*ng/L, about 325 ng*hr/mL, about 350 ng*hr/mL, about 375 ng*hr/mL, about 400 ng*hr/mL, about 425 ng*hr/mL, about 450 ng*hr/mL, about 475 ng*hr/mL, about 500 ng*hr/mL, about 525 ng*hr/mL, about 550 ng*hr/mL, about 575 ng*hr/mL, about 600 ng*hr/mL, about 625 ng*hr/mL, about 650 ng*hr/mL, about 675 ng*hr/mL, about 700 ng*hr/mL, about 725 ng*hr/mL, about 750 ng*hr/mL, about 775 ng*hr/mL, about 800 ng*hr/mL, about 825 ng*hr/mL, about 850 ng*hr/mL, about 875 ng*hr/mL, about 900 ng*hr/mL, about 925 ng*hr/mL, about 950 ng*hr/mL, about 975 ng*hr/mL, about 1000 ng*hr/mL, about 1025 ng*hr/mL, about 1050 ng*hr/mL, about 1075 ng*hr/mL, about 1100 ng*hr/mL, about 1125 ng*hr/mL, about 1150 ng*hr/mL, about 1175 ng*hr/mL, about 1200 ng*hr/mL, about 1225 ng*hr/mL, about 1250 ng*hr/mL, about 1275 ng*hr/mL, about 1300 ng*hr/mL, about 1325 ng*hr/mL, about 1350 ng*hr/mL, about 1375 ng*hr/mL, about 1400 ng*hr/mL, about 1425 ng*hr/mL, about 1450 ng*hr/mL, about 1475 ng*hr/mL, about 1500 ng*hr/mL, about 1525 ng*hr/mL, about 1550 ng*hr/mL, about 1575 ng*hr/mL, about 1600 ng*hr/mL, about 1625 ng*hr/mL, about 1650 ng*hr/mL, about 1675 ng*hr/mL, about 1700 ng*hr/mL, about 1725 ng*hr/mL, about 1750 ng*hr/mL, about 1775 ng*hr/mL, about 1800 ng*hr/mL, about 1825 ng*hr/mL, about 1850 ng*hr/mL, about 1875 ng*hr/mL, about 1900 ng*hr/mL, about 1925 ng*hr/mL, about 1950 ng*hr/mL, about 1975 ng*hr/mL, about 2000 ng*hr/mL, about 2025 ng*hr/mL, about 2050 ng*hr/mL, about 2075 ng*hr/mL, about 2100 ng*hr/mL, about 2125 ng*hr/mL, about 2150 ng*hr/mL, about 2175 ng*hr/mL, or about 2200 ng*hr/mL, including all values and ranges in between. In embodiments, the dose is about 40 μg and the AUC_{0-inf} range is between about about 300 hr*ng/L to about 2200 hr*ng/L, for example, about 400 hr*ng/L to about 2000 hr*ng/L, about 400 hr*ng/L to about 1800 hr*ng/L, about 500 hr*ng/L to about 1500 hr*ng/L, for example, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L.

In embodiments, the dose is about 45 μg and the AUC₀₋₈ range is about 80% to 125% of about 500 hr*ng/L to about 900 hr*ng/L; about 500 hr*ng/L to about 800 hr*ng/L, about 600 hr*ng/L to about 900 hr*ng/L, about 600 hr*ng/L to about 800 hr*ng/L, about 650 hr*ng/L to about 750 hr*ng/L

In embodiments, the dose is about 45 μg and the AUC₀₋₈ range is between about 500 hr*ng/L to about 900 hr*ng/L, for example, about 500 hr*ng/L to about 800 hr*ng/L, about 600 hr*ng/L to about 900 hr*ng/L, about 600 hr*ng/L to about 800 hr*ng/L, about 650 hr*ng/L to about 750 hr*ng/L.

In embodiments, the dose is about 60 μg, AUC₀₋₈ range is about 80% to 125% of about 500 hr*ng/L to about 1500 hr*ng/L and the AUC_{0-inf} range is about 80% to 125% of about 500 hr*ng/L to about 2000 hr*ng/L.

In embodiments, the dose is about 60 μg and the AUC_{0-inf} is about 500 hr*ng/L to about 3500 hr*ng/L, e.g., about 500 hr*ng/L, about 550 ng*hr/mL, about 600 ng*hr/mL, about 650 ng*hr/mL, about 700 ng*hr/mL, about 750 ng*hr/mL, about 800 ng*hr/mL, about 850 ng*hr/mL, about 900 ng*hr/mL, about 950 ng*hr/mL, about 1000 ng*hr/mL, about 1050 ng*hr/mL, about 1100 ng*hr/mL, about 1150 ng*hr/mL, about 1200 ng*hr/mL, about 1250 ng*hr/mL, about 1300 ng*hr/mL, about 1350 ng*hr/mL, about 1400 ng*hr/mL, about 1450 ng*hr/mL, about 1500 ng*hr/mL, about 1550 ng*hr/mL, about 1600 ng*hr/mL, about 1650 ng*hr/mL, about 1700 ng*hr/mL, about 1750 ng*hr/mL, about 1800 ng*hr/mL, about 1850 ng*hr/mL, about 1900 ng*hr/mL, about 1950 ng*hr/mL, about 2000 ng*hr/mL, about 2050 ng*hr/mL, about 2100 ng*hr/mL, about 2150 ng*hr/mL, about 2200 ng*hr/mL, about 2250 ng*hr/mL, about 2300 ng*hr/mL, about 2350 ng*hr/mL, about 2400 ng*hr/mL, about 2450 ng*hr/mL, about 2500 ng*hr/mL, about 2550 ng*hr/mL, about 2600 ng*hr/mL, about 2650 ng*hr/mL, about 2700 ng*hr/mL, about 2750 ng*hr/mL, about 2800 ng*hr/mL, about 2850 ng*hr/mL, about 2900 ng*hr/mL, about 2950 ng*hr/mL, about 3000 ng*hr/mL, about 3050 ng*hr/mL, about 3100 ng*hr/mL, about 3150 ng*hr/mL, about 3200 ng*hr/mL, about 3250 ng*hr/mL, about 3300 ng*hr/mL, about 3350 ng*hr/mL, about 3400 ng*hr/mL, about 3450 ng*hr/mL, or about 3500 ng*hr/mL, including all values and ranges in between. In embodiments, the dose is about 60 and the AUC_{0-inf} range is between about 500 hr*ng/L to about 3500 hr*ng/L, about 500 hr*ng/L to about 3000 hr*ng/L, about 500 hr*ng/L to about 2500 hr*ng/L, about 500 hr*ng/L to about 2000 hr*ng/L, for example, about 600 hr*ng/L to about 1900 hr*ng/L, about 700 hr*ng/L to about 1800 hr*ng/L, about 700 hr*ng/L to about 1700 hr*ng/L, about 700 hr*ng/L to about 1600 hr*ng/L, about 700 hr*ng/L to about 1500 hr*ng/L, about 800 hr*ng/L to about 1500 hr*ng/L, about 900 hr*ng/L to about 1500, about 1000 hr*ng/L to about 1500 hr*ng/L, about 1100 hr*ng/L to about 1500 hr*ng/L, about 1200 hr*ng/L to about 1500 hr*ng/L, about 1300 hr*ng/L to about 1500 hr*ng/L, or about 1400 hr*ng/L to about 1500 hr*ng/L.

In embodiments, the dose is about 60 μg and the AUC₀₋₈ range is between about 500 hr*ng/L to about 1500 hr*ng/L, for example, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L.

In embodiments, the dose is about 90 μg, the AUC₀₋₈ range is about 80% to 125% of about 500 hr*ng/L to about 1500 hr*ng/L, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L.

In embodiments, the dose is about 90 μg and the AUC_{0-inf} is about 500 hr*ng/L to about 5000 hr*ng/L, e.g., about 500 hr*ng/L, about 550 ng*hr/mL, about 600 ng*hr/mL, about 650 ng*hr/mL, about 700 ng*hr/mL, about 750 ng*hr/mL, about 800 ng*hr/mL, about 850 ng*hr/mL, about 900 ng*hr/mL, about 950 ng*hr/mL, about 1000 ng*hr/mL, about 1050 ng*hr/mL, about 1100 ng*hr/mL, about 1150 ng*hr/mL, about 1200 ng*hr/mL, about 1250 ng*hr/mL, about 1300 ng*hr/mL, about 1350 ng*hr/mL, about 1400 ng*hr/mL, about 1450 ng*hr/mL, about 1500 ng*hr/mL, about 1550 ng*hr/mL, about 1600 ng*hr/mL, about 1650 ng*hr/mL, about 1700 ng*hr/mL, about 1750 ng*hr/mL, about 1800 ng*hr/mL, about 1850 ng*hr/mL, about 1900 ng*hr/mL, about 1950 ng*hr/mL, about 2000 ng*hr/mL, about 2050 ng*hr/mL, about 2100 ng*hr/mL, about 2150 ng*hr/mL, about 2200 ng*hr/mL, about 2250 ng*hr/mL, about 2300 ng*hr/mL, about 2350 ng*hr/mL, about 2400 ng*hr/mL, about 2450 ng*hr/mL, about 2500 ng*hr/mL, about 2550 ng*hr/mL, about 2600 ng*hr/mL, about 2650 ng*hr/mL, about 2700 ng*hr/mL, about 2750 ng*hr/mL, about 2800 ng*hr/mL, about 2850 ng*hr/mL, about 2900 ng*hr/mL, about 2950 ng*hr/mL, about 3000 ng*hr/mL, about 3050 ng*hr/mL, about 3100 ng*hr/mL, about 3150 ng*hr/mL, about 3200 ng*hr/mL, about 3250 ng*hr/mL, about 3300 ng*hr/mL, about 3350 ng*hr/mL, about 3400 ng*hr/mL, about 3450 ng*hr/mL, about 3500 ng*hr/mL, about 3550 ng*hr/mL, about 3600 ng*hr/mL, about 3650 ng*hr/mL, about 3700 ng*hr/mL, about 3750 ng*hr/mL, about 3800 ng*hr/mL, about 3850 ng*hr/mL, about 3900 ng*hr/mL, about 3950 ng*hr/mL, about 4000 ng*hr/mL, about 4050 ng*hr/mL, about 4100 ng*hr/mL, about 4150 ng*hr/mL, about 4200 ng*hr/mL, about 4250 ng*hr/mL, about 4300 ng*hr/mL, about 4350 ng*hr/mL, about 4400 ng*hr/mL, about 4450 ng*hr/mL, about 4500 ng*hr/mL, about 4550 ng*hr/mL, about 4600 ng*hr/mL, about 4650 ng*hr/mL, about 4700 ng*hr/mL, about 4750 ng*hr/mL, about 4800 ng*hr/mL, about 4850 ng*hr/mL, about 4900 ng*hr/mL, about 4950 ng*hr/mL, or about 5000 ng*hr/mL, including all values and ranges in between. In embodiments, the dose is about 90 μg and the AUC_{0-inf} range is between about 500 hr*ng/L to about 5000 hr*ng/L, for example, about 500 hr*ng/L to about 4500 hr*ng/L, about 500 hr*ng/L to about 4000 hr*ng/L, about 500 hr*ng/L to about 3500 hr*ng/L, about 500 hr*ng/L to about 3000 hr*ng/L, about 500 hr*ng/L to about 2500 hr*ng/L, about 500 hr*ng/L to about 2000 hr*ng/L, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L.

In embodiments, the dose is about 90 μg and the AUC₀₋₈ range is between about 500 hr*ng/L to about 1500 hr*ng/L, for example, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L.

Advantageously, the dose provides a Cₘₐₓ in a range of about 50 ng/L to about 500 ng/L; for example, about 50 ng/L to about 400 ng/L, preferably about 50 ng/L to about 300 ng/L.

Advantageously, the dose provides a Cₘₐₓ in a range of about 50 ng/L to about 500 ng/L; for example, about 50 ng/L to about 400 ng/L, preferably about 50 ng/L to about 300 ng/L. Advantageously, the dose provides an AUC₀₋₈ in a range of about 200 hr*ng/L to about 1500 hr*ng/L; for example, about 200 hr*ng/L to about 1250 hr*ng/L, about 200 hr*ng/L to about 1000 hr*ng/L, about 200 hr*ng/L to about 750 hr*ng/L, about 200 hr*ng/L to about 500 hr*ng/L, about 500 hr*ng/L to about 1500 hr*ng/L, about 500 hr*ng/L to about 1250 hr*ng/L, about 500 hr*ng/L to about 1000 hr*ng/L, about 500 hr*ng/L to about 750 hr*ng/L, about 750 hr*ng/L to about 1500 hr*ng/L, about 750 hr*ng/L to about 1250 hr*ng/L, about 750 hr*ng/L to about 1000 hr*ng/L, about 1000 hr*ng/L to about 1500 hr*ng/L. In embodiments, the dose provides an AUC₀₋₈ of about 200 hr*ng/L, about 300 hr*ng/L, about 400 hr*ng/L, about 500 hr*ng/L, about 600 hr*ng/L, about 700 hr*ng/L, about 800 hr*ng/L, about 900 hr*ng/L, about 1000 hr*ng/L, about 1100 hr*ng/L, about 1200 hr*ng/L, about 1300 hr*ng/L, about 1400 hr*ng/L, or about 1500 hr*ng/L. Advantageously, the dose provides an AUC_{0-inf} in a range of about 200 hr*ng/L to about 2200 hr*ng/L, about 200 hr*ng/L to about 2000 hr*ng/L; for example, about 300 hr*ng/L to about 1900 hr*ng/L, about 400 hr*ng/L to about 1800 hr*ng/L, about 500 hr*ng/L to about 1700 hr*ng/L, about 500 hr*ng/L to about 1600 hr*ng/L, about 500 hr*ng/L to about 1500 hr*ng/L, about 600 hr*ng/L to about 1500 hr*ng/L, about 700 hr*ng/L to about 1500 hr*ng/L. In embodiments, the dose provides an AUC_{0-inf} of about 200 hr*ng/L, about 300 hr*ng/L, about 400 hr*ng/L, about 500 hr*ng/L, about 600 hr*ng/L, about 700 hr*ng/L, about 750 hr*ng/L, about 800 hr*ng/L, about 850 hr*ng/L, about 900 hr*ng/L, about 950 hr*ng/L, about 1000 hr*ng/L, about 1050 hr*ng/L, about 1100 hr*ng/L, about 1150 hr*ng/L, about 1200 hr*ng/L, about 1250 hr*ng/L, about 1300 hr*ng/L, about 1350 hr*ng/L, about 1400 hr*ng/L, about 1450 hr*ng/L, or about 1500 hr*ng/L. In embodiments, the Cmax, AUC_{0-inf}, and AUC₀₋₈ is preferably 80% to 125% of these ranges and values.

In embodiments, the dose is about 30 μg, and the Cₘₐₓ is about 30 ng/L to about 150 ng/L, e.g., about 30 ng/L, about 35 ng/L, about 40 ng/L, about 45 ng/L, about 50 ng/L, about 55 ng/L, about 60 ng/L, about 65 ng/L, about 70 ng/L, about 75 ng/L, about 80 ng/L, about 85 ng/L, about 90 ng/L, about 95 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, or about 150 ng/L, including all values and ranges in between.

In embodiments, the dose is about 30 μg, the Cₘₐₓ range is about 80% to 125% of about 50 ng/L to about 150 ng/L, about 50 ng/L to about 125 ng/L, about 50 ng/L to about 100 ng/L, about 50 ng/L to about 85 ng/L, about 75 ng/L to about 150 ng/L, about 75 ng/L to about 125 ng/L, about 75 ng/L to about 100 ng/L, about 100 ng/L to about 150 ng/L

In embodiments, the dose is about 30 μg and the Cₘₐₓ range is between about 50 ng/L to about 150 ng/L, for example, about 50 ng/L to about 125 ng/L, about 50 ng/L to about 100 ng/L, about 50 ng/L to about 75 ng/L, about 75 ng/L to about 150 ng/L, about 75 ng/L to about 125 ng/L, about 75 ng/L to about 100 ng/L, about 100 ng/L to about 150 ng/L. In embodiments, the Cₘₐₓ and AUC₀₋₈ is preferably 80% to 125% of these ranges.

In embodiments, the dose is about 30 μg and the Cₘₐₓ range is between about 50 ng/L to about 150 ng/L, for example, about 50 ng/L to about 125 ng/L, about 50 ng/L to about 100 ng/L, about 50 ng/L to about 85 ng/L, about 75 ng/L to about 150 ng/L, about 75 ng/L to about 125 ng/L, about 75 ng/L to about 100 ng/L, about 100 ng/L to about 150 ng/L. In embodiments, the Cₘₐₓ and AUC_{0-inf} is preferably 80% to 125% of these ranges.

In embodiments, the dose is about 40 μg, and the Cₘₐₓ is about 40 ng/L to about 200 ng/L, e.g., about 40 ng/L, about 45 ng/L, about 50 ng/L, about 55 ng/L, about 60 ng/L, about 65 ng/L, about 70 ng/L, about 75 ng/L, about 80 ng/L, about 85 ng/L, about 90 ng/L, about 95 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 155 ng/L, about 160 ng/L, about 165 ng/L, about 170 ng/L, about 175 ng/L, about 180 ng/L, about 185 ng/L, about 190 ng/L, about 195 ng/L, or about 200 ng/L, including all values and ranges in between.

In embodiments, the dose is about 40 μg and the Cₘₐₓ range is about 80% to 125% of 50 ng/L to about 250 ng/L, for example, about 50 ng/L to about 225 ng/L, about 50 ng/L to about 200 ng/L, about 100 ng/L to about 180 ng/L, about 100 ng/L to about 150 ng/L, about 150 ng/L to about 200 ng/L.

In embodiments, the dose is about 40 μg and the Cₘₐₓ range is between about 50 ng/L to about 250 ng/L, for example, about 50 ng/L to about 225 ng/L, about 50 ng/L to about 200 ng/L, about 100 ng/L to about 180 ng/L, about 100 ng/L to about 150 ng/L, about 150 ng/L to about 200 ng/L. In embodiments, the Cₘₐₓ and AUC_{0-inf} is preferably 80% to 125% of these ranges and values.

In embodiments, the dose is about 45 μg, Cₘₐₓ range is about 80% to 125% of about 75 ng/L to about 175 ng/L, about 75 ng/L to about 150 ng/L, about 75 ng/L to about 125 ng/L, about 75 ng/L to about 100 ng/L, about 100 ng/L to about 150 ng/L.

In embodiments, the dose is about 45 μg and the Cₘₐₓ range is between about 75 ng/L to about 175 ng/L, for example, about 75 ng/L to about 150 ng/L, about 75 ng/L to about 125 ng/L, about 75 ng/L to about 100 ng/L, about 100 ng/L to about 150 ng/L. In embodiments, the Cₘₐₓ and AUC₀₋₈ is preferably 80% to 125% of these ranges and values.

In embodiments, the dose is about 60 μg, and the Cₘₐₓ is about 70 ng/L to about 300 ng/L, e.g., about 70 ng/L, about 75 ng/L, about 80 ng/L, about 85 ng/L, about 90 ng/L, about 95 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 155 ng/L, about 160 ng/L, about 165 ng/L, about 170 ng/L, about 175 ng/L, about 180 ng/L, about 185 ng/L, about 190 ng/L, about 195 ng/L, about 200 ng/L, about 205 ng/L, about 210 ng/L, about 215 ng/L, about 220 ng/L, about 225 ng/L, about 230 ng/L, about 235 ng/L, about 240 ng/L, about 245 ng/L, about 250 ng/L, about 255 ng/L, about 260 ng/L, about 265 ng/L, about 270 ng/L, about 275 ng/L, about 280 ng/L, about 285 ng/L, about 290 ng/L, about 295 ng/L, or about 300 ng/L, including all values and ranges in between.

In embodiments, the dose is about 60 μg , Cₘₐₓ range is about 80% to 125% of about 100 ng/L to about 300 ng/L, about 100 ng/L to about 250 ng/L, about 100 ng/L to about 225 ng/L, about 100 ng/L to about 200 ng/L, about 100 ng/L to about 150 ng/L, about 150 ng/L to about 250 ng/L, about 150 ng/L to about 200 ng/L.

In embodiments, the dose is about 60 μg and the Cₘₐₓ range is between about 100 ng/L to about 250 ng/L, for example, about 100 ng/L to about 225 ng/L, about 100 ng/L to about 200 ng/L, about 150 ng/L to about 250 ng/L, about 150 ng/L to about 200 ng/L. In embodiments, the Cmax and AUC₀₋₈ is preferably 80% to 125% of these ranges and values.

In embodiments, the dose is about 60 μg and the Cₘₐₓ range is between about 100 ng/L to about 300 ng/L, for example, about 100 ng/L to about 250 ng/L, about 100 ng/L to about 225 ng/L, about 100 ng/L to about 200 ng/L, about 100 ng/L to about 150 ng/L, about 150 ng/L to about 250 ng/L, about 150 ng/L to about 200 ng/L. In embodiments, the Cₘₐₓ and AUC_{0-inf} is preferably 80% to 125% of these ranges and values.

In embodiments, the dose is about 90 μg, the Cₘₐₓ range is about 80% to 125% of about 100 ng/L to about 400 ng/L, 100 ng/L to about 350 ng/L, about 100 ng/L to about 300 ng/L, about 200 ng/L to about 400 ng/L, about 200 ng/L to about 350 ng/L.

In embodiments, the dose is about 90 μg and the Cₘₐₓ range is between about 100 ng/L to about 400 ng/L, for example, about 100 ng/L to about 350 ng/L, about 100 ng/L to about 300 ng/L, about 200 ng/L to about 400 ng/L, about 200 ng/L to about 350 ng/L. In embodiments, the Cₘₐₓ and AUC₀₋₈ is preferably 80% to 125% of these ranges and values.

In embodiments, when the dose is 30μg, the mean plasma concentrations values are in the range of about 20 ng/L to about 50 ng/L (for example about 25 ng/L, about 30 ng/L, about 35 ng/L, about 40 ng/L and about 45 ng/L) post administration of dexmedetomidine or pharmaceutically acceptable salt thereof on day 6. In embodiments, the Cₘₐₓ values are preferably 80% to 125% of these ranges and values.

In embodiments, when the dose is 60μg, the mean plasma concentrations are in the range of about 25 ng/L to about 150 ng/L (for example about 25 ng/L, about 30 ng/L, about 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L) post administration of dexmedetomidine or pharmaceutically acceptable salt thereof on day 6. In embodiments, the mean plasma concentrations are preferably 80% to 125% of these ranges and values.

In embodiments, the dose is about 90 μg, and the Cₘₐₓ is about 100 ng/L to about 500 ng/L, e.g., about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 155 ng/L, about 160 ng/L, about 165 ng/L, about 170 ng/L, about 175 ng/L, about 180 ng/L, about 185 ng/L, about 190 ng/L, about 195 ng/L, about 200 ng/L, about 205 ng/L, about 210 ng/L, about 215 ng/L, about 220 ng/L, about 225 ng/L, about 230 ng/L, about 235 ng/L, about 240 ng/L, about 245 ng/L, about 250 ng/L, about 255 ng/L, about 260 ng/L, about 265 ng/L, about 270 ng/L, about 275 ng/L, about 280 ng/L, about 285 ng/L, about 290 ng/L, about 295 ng/L, about 300 ng/L, about 305 ng/L, about 310 ng/L, about 315 ng/L, about 320 ng/L, about 325 ng/L, about 330 ng/L, about 335 ng/L, about 340 ng/L, about 345 ng/L, about 350 ng/L, about 355 ng/L, about 360 ng/L, about 365 ng/L, about 370 ng/L, about 375 ng/L, about 380 ng/L, about 385 ng/L, about 390 ng/L, about 395 ng/L, about 400 ng/L, about 405 ng/L, about 410 ng/L, about 415 ng/L, about 420 ng/L, about 425 ng/L, about 430 ng/L, about 435 ng/L, about 440 ng/L, about 445 ng/L, about 450 ng/L, about 455 ng/L, about 460 ng/L, about 465 ng/L, about 470 ng/L, about 475 ng/L, about 480 ng/L, about 485 ng/L, about 490 ng/L, about 495 ng/L, or about 500 ng/L, including all values and ranges in between.

In embodiments, when the dose is 90μg, the mean plasma concentrations are in the range of about 30 ng/L to about 150 ng/L (for example about about 30 ng/L, 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L or about 145 ng/L, including all values and ranges in between) post administration of dexmedetomidine or pharmaceutically acceptable salt thereof on day 6. In embodiments, the Cₘₐₓ values are preferably 80% to 125% of these ranges and values.

In embodiments, when the dose is 120 μg, the mean plasma concentrations are in the range of about 50 ng/L to about 200 ng/L (for example, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L about 195 ng/L, or about 200 ng/L, including all values and ranges in between) post administration of dexmedetomidine or pharmaceutically acceptable salt thereof on day 6. In embodiments, the mean plasma concentrations values are preferably 80% to 125% of these ranges and values.

In embodiments, when the dose is 180 μg, the mean plasma concentrations are in the range of about 100 ng/L to about 450 ng/L (for example, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, about 400 ng/L, about 425 ng/L, or about 450 ng/L, including all values and ranges in between) post administration of dexmedetomidine or pharmaceutically acceptable salt thereof on day 6. In embodiments, the Cₘₐₓ values are preferably 80% to 125% of these ranges and values.

In embodiments, when the dose is 240 μg, the mean plasma concentrations are in the range of about 100 ng/L to about 400 ng/L (for example, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, about 395 ng/L, about 400 ng/L, including all values and ranges in between) post administration of dexmedetomidine or pharmaceutically acceptable salt thereof on day 6. In embodiments, the Cₘₐₓ values are preferably 80% to 125% of these ranges and values.

In embodiments, when the dose is 30 μg, the mean plasma concentrations are in the range of about 10 ng/L to about 100 ng/L (for example about 10 ng/L, about 15 ng/L, about 20 ng/L, about 25 ng/L, about 30 ng/L, about 35 ng/L, about 40 ng/L, about 45 ng/L, about 50 ng/L, about 60 ng/L, about 70 ng/L, about 75 ng/L, about 80 ng/L, about 90 ng/L, about 95 ng/L, or about 95 ng/L, including all values and ranges in between) post administration of dexmedetomidine or pharmaceutically acceptable salt thereof on day 12.

In embodiments, when the dose is 60μg, the mean plasma concentrations are in the range of about 10 ng/L to about 150 ng/L (for example about 10 ng/L, about 15 ng/L, about 20 ng/L, about 25 ng/L, about 30 ng/L, about 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, or about 150 ng/L, including all values and ranges in between) post administration of dexmedetomidine or pharmaceutically acceptable salt thereof on day 12.

In embodiments, when the dose is 90μg, the mean plasma concentrations are in the range of about 25 ng/L to about 150 ng/L (for example about 25 ng/L, about 30 ng/L, about 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, or about 150 ng/L, including all values and ranges in between) post administration of dexmedetomidine or pharmaceutically acceptable salt thereof on day 12.

In embodiments, when the dose is 120 μg, the mean plasma concentrations are in the range of about 50 ng/L to about 200 ng/L (for example, about 55 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L and about 195 ng/L, or about 200 ng/L, including all values and ranges in between) post administration of dexmedetomidine or pharmaceutically acceptable salt thereof on day 12.

In embodiments, when the dose is 180 μg, the mean plasma concentrations are in the range of about 100 ng/L to about 400 ng/L (for example, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, or about 400 ng/L, including all values and ranges in between) post administration of dexmedetomidine or pharmaceutically acceptable salt thereof on day 12.

In embodiments, when the dose is 240 μg, the mean plasma concentrations are in the range of about 50 ng/L to about 500 ng/L (for example, about 55 ng/L, about 60 ng/L, about 70 ng/L, about 75 ng/L, about 80 ng/L, about 90 ng/L, about 95 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, about 395 ng/L, about 400 ng/L, about 425 ng/L, about 450 ng/L, about 460 ng/L, about 465 ng/L, about 475 ng/L, about 480 ng/L, about 485 ng/L, about 490 ng/L, about 495 ng/L, or about 500 ng/L, including all values and ranges in between) post administration of dexmedetomidine or pharmaceutically acceptable salt thereof on day 12.

In embodiments, the subject is about 1 to about 100 years old, e.g., about 1 year old, about 2 years old, about 3 years old, about 4 years old, about 5 years old, about 6 years old, about 7 years old, about 8 years old, about 9 years old, about 10 years old, about 11 years old, about 12 years old, about 13 years old, about 14 years old, about 15 years old, about 16 years old, about 17 years old, about 18 years old, about 19 years old, about 20 years old, about 25 years old, about 30 years old, about 35 years old, about 40 years old, about 45 years old, about 50 years old, about 55 years old, about 60 years old, about 65 years old, about 70 years old, about 75 years old, about 80 years old, about 85 years old, about 90 years old, about 95 years old, about 100 years old, including all values and ranges in between.

In embodiments, the subject is about 1 to about 18 years old, e.g., about 1 year old, about 2 years old, about 3 years old, about 4 years old, about 5 years old, about 6 years old, about 7 years old, about 8 years old, about 9 years old, about 10 years old, about 11 years old, about 12 years old, about 13 years old, about 14 years old, about 15 years old, about 16 years old, about 17 years old, about 18 years old, including all values and ranges in between.

In embodiments, the subject is about 10 to about 17 years old, e.g., about 10 years old, about 11 years old, about 12 years old, about 13 years old, about 14 years old, about 15 years old, about 16 years old, about 17 years, including all values and ranges in between.

In embodiments, the subject is about 13 to about 17 years old, e.g., about 13 years old, about 14 years old, about 15 years old, about 16 years old, about 17 years old, including all values and ranges in between.

In embodiments, the subject is about 18 years old to about 64 years old, e.g., about 18 years old, about 19 years old, about 20 years old, about 21 years old, about 22 years old, about 23 years old, about 24 years old, about 25 years old, about 26 years old, about 27 years old, about 28 years old, about 29 years old, about 30 years old, about 31 years old, about 32 years old, about 33 years old, about 34 years old, about 35 years old, about 36 years old, about 37 years old, about 38 years old, about 39 years old, about 40 years old, about 41 years old, about 42 years old, about 43 years old, about 44 years old, about 45 years old, about 46 years old, about 47 years old, about 48 years old, about 49 years old, about 50 years old, about 51 years old, about 52 years old, about 53 years old, about 54 years old, about 55 years old, about 56 years old, about 57 years old, about 58 years old, about 59 years old, about 60 years old, about 61 years old, about 62 years old, about 63 years old, about 64 years old, including all values and ranges in between.

In embodiments, the subject is about 21 years old to about 50 years old, e.g., about 21 years old, about 22 years old, about 23 years old, about 24 years old, about 25 years old, about 26 years old, about 27 years old, about 28 years old, about 29 years old, about 30 years old, about 31 years old, about 32 years old, about 33 years old, about 34 years old, about 35 years old, about 36 years old, about 37 years old, about 38 years old, about 39 years old, about 40 years old, about 41 years old, about 42 years old, about 43 years old, about 44 years old, about 45 years old, about 46 years old, about 47 years old, about 48 years old, about 49 years old, about 50 years old, including all values and ranges in between.

In embodiments, the subject is older than 64 years old. In embodiments, the subject is about 65 years old to about 80 years old, e.g., about 65 years old, about 66 years old, about 67 years old, about 68 years old, about 69 years old, about 70 years old, about 71 years old, about 72 years old, about 73 years old, about 74 years old, about 75 years old, about 76 years old, about 77 years old, about 78 years old, about 79 years old, about 80 years old, including all values and ranges in between. In embodiments, the subject is about 75 years old to about 80 years old, e.g., about 75 years old, about 76 years old, about 77 years old, about 78 years old, about 79 years old, about 80 years old, including all values and ranges in between. In embodiments, the subject is older than 80 years old, e.g., about 81 years old, about 82 years old, about 83 years old, about 84 years old, about 85 years old, about 86 years old, about 87 years old, about 88 years old, about 89 years old, about 90 years old, about 91 years old, about 92 years old, about 93 years old, about 94 years old, about 95 years old, about 96 years old, about 97 years old, about 98 years old, about 99 years old, about 100 years old, including all values and ranges in between.

In order to achieve the desired dose, dexmedetomidine may be oromucosally administered as a single unit dose, as multiple unit doses, or as a fraction of one or more unit doses (e.g. half of a unit dose), or a combination thereof. By way of example, to administer 120 μg of dexmedetomidine, a patient may be administered a single unit dose of 120 μg of dexmedetomidine, two unit doses of 60 μg of dexmedetomidine, or one half of a unit dose of 240 μg of dexmedetomidine. In embodiments, dexmedetomidine is administered as a film. Thus, half doses can be achieved by cutting the film in half, for example, cutting a 120 μg or 180 μg film in half to achieve a 60 μg dose and a 90 μg dose, respectively.

In embodiments, the dose may be administered multiple times (e.g. one to four times) at an appropriate dosing interval (e.g. every 0.5 hours) to produce a desired effect; for example, a 20 μg unit dose or a 60 μg unit dose can be administered four times at a dosing interval of every 0.5 hours within 6 hours of the first dose to produce the effect of a 80 μg dose and a 240 μg dose, respectively. In embodiments, each dosage unit may be administered one to two times at an appropriate dosing interval (every 12 hours) to produce a desired effect; for example, a 120 μg unit is administered two times in a day at an interval of 12 hours to produce the effect of a 240 μg dose. In embodiments, each dosage unit may be administered one to ten times at an appropriate dosing interval (e.g. every 1 to 6 hours) to produce a desired effect; for example, a 120 μg dose (starting dose) is administered followed by an additional seven doses in a day at an interval of about 1 to about 6 hours to produce the maximum cumulative dose of a 960 μg dose. In embodiments, each dosage unit may be administered one to ten times at an appropriate dosing interval (e.g. every 1 to 6 hours) to produce a desired effect; for example, a 180 μg dose (starting dose) is administered followed by an additional six doses of 120 μg in a day at an interval of about 1 to about 6 hours to produce the maximum cumulative dose of a 900 μg dose. In embodiments, each dosage unit may be administered one to ten times at an appropriate dosing interval (for e.g. of at least 1 to 6 hours) to produce a desired effect; for example, a 240 μg dose (starting dose) is administered followed by an additional six doses of 120 μg in a day at an interval of about 1 to about 6 hours to produce the maximum cumulative dose of a 960 μg dose. In embodiments, each dosage unit may be administered one to ten times at an appropriate dosing interval (for e.g. of at least 1 to 6 hours) to produce a desired effect; for example, a 300 μg dose (starting dose) is administered followed by additional five doses of 120 μg in a day at an interval of about 1 to about 6 hours to produce the maximum cumulative dose of a 900 μg dose. In embodiments, dexmedetomidine or a pharmaceutically acceptable salt thereof is administered oromucosally (e.g. sublingually or buccally) as a film.

In embodiments, the dosage of dexmedetomidine or a pharmaceutically acceptable salt thereof may be administered one to six times a day.

In embodiments, the dosage of dexmedetomidine or a pharmaceutically acceptable salt thereof may be administered one to four times a day. In embodiments, the dosage of dexmedetomidine or a pharmaceutically acceptable salt thereof may be administered twice a day.

In embodiments, dexmedetomidine is oromucosally (e.g. sublingually or buccally) administered one to ten times a day at an appropriate dosing interval (e.g. after every 30 minutes) within 6 hours of first dose to produce a desired effect; for example, a 20 μg unit dose is administered four times at a dosing interval of every 30 minutes within 6 hours of the first dose to produce the effect of a 80 μg dose; or a 60 μg unit dose is administered four times at a dosing interval of every 30 minutes within 6 hours of the first dose to produce the effect of a 240 μg. In embodiments, each dosage unit may be administered one to two times at an appropriate dosing interval (e.g. at least 12 hours apart) to produce a desired effect; for example, a 120 μg unit is administered two times in a day at an interval of 12 hours apart. In embodiments, each dosage unit may be administered one to ten times at an appropriate dosing interval (e.g. of at least 1 to 6 hours) to produce a desired effect; for example, a 120 μg dose (starting dose) is administered an additional seven times in a day at an interval of about 1 to about 6 hours to produce a maximum cumulative dose of a 960 μg. In embodiments, each dosage unit may be administered one to ten times at an appropriate dosing interval (e.g. at least 1 to 6 hours) to produce a desired effect; for example, a 180 μg dose (starting dose) is administered followed by an additional six doses of 120 μg at an interval of about 1 to about 6 hours to produce the maximum cumulative dose of a 900 μg. In embodiments, each dosage unit may be administered one to ten times at an appropriate dosing interval (e.g. of at least 1 to 6 hours) to produce a desired effect; for example, a 240 μg dose (starting dose) is administered followed by an additional six doses of 120 μg in a day at an interval of about 1 to about 6 hours to produce the maximum cumulative dose of a 960 μg. In embodiments, each dosage unit may be administered one to ten times at an appropriate dosing interval (e.g. of at least 1 to 6 hours) to produce a desired effect; for example, a 300 μg dose (starting dose) is administered followed by an additional five doses of 120 μg in a day at an interval of about 1 to about 6 hours to produce the maximum cumulative dose of 900 μg.

In embodiments, a lower dosage is administered in the morning (for example, from about 10 μg to about 60 μg) and a higher dosage is administered in the evening or night (for example, the doses above 60 μg).

In embodiments, dexmedetomidine or a pharmaceutically acceptable salt thereof may be administered orally, oromucosally (e.g. sublingually, buccally), intravenously, intramuscularly, subcutaneously, topically, transdermally, intratracheally, intraperitoneally, intraorbitally, by implantation, by inhalation, intrathecally, intraventricularly or intranasally.

In embodiments, dexmedetomidine or a pharmaceutically acceptable salt thereof is administered to the subject by the sublingual, buccal, oral, intranasal or parenteral route. In embodiments, dexmedetomidine or a pharmaceutically acceptable salt thereof is administered by the sublingual or buccal route.

In embodiments, the present disclosure is a method of treating agitation or signs of agitation in a human subject with dementia. In embodiments, the present disclosure is a method of treating agitation or signs of agitation in a human subject with dementia, without also inducing significant sedation.

In embodiments, the present disclosure is a method of treating agitation or signs of agitation in a human subject with dementia, without also inducing significant sedation, comprising administering from about 30 μg to about 180 μg of dexmedetomidine or a pharmaceutically acceptable salt thereof. In embodiments, the subject with dementia has Alzheimer's disease. In embodiments, 30 μg, 40 μg, 60 μg or 90 μg of dexmedetomidine or a pharmaceutically acceptable salt thereof is administered as a single dose per day. In embodiments, an additional dose (e.g. 10 μg, 20 μg, 30 μg or 40 μg) is administered after a suitable period of time (e.g. 2, 4, 6, 8, or 12 hours) in the event of persistent or recurrent agitation. In embodiments, dexmedetomidine or a pharmaceutically acceptable salt thereof is oromucosally administered as a unit dose comprising about 30 μg to about 90 μg from 1 to 6 times per day. For example, dexmedetomidine or a pharmaceutically acceptable salt thereof is oromucosally administered 1, 2, 3, 4, 5, or 6 times every 2 hours, every 4 hours, every 6 hours, every 8 hours, every 10 hours, or every 12 hours. In embodiments, a unit dose comprising about 30 μg to 60 μg of dexmedetomidine or a pharmaceutically acceptable salt thereof is administered one to six times per day at an interval of 2 hours with the provision of maximum of three doses within 12 hours of first dose. In embodiments, each unit dose containing about 90 μg of dexmedetomidine or a pharmaceutically acceptable salt thereof may be taken one to four times per day at an interval of 2 hours with the provision of maximum of two doses within 12 hours of first dose. In embodiments, dexmedetomidine or a pharmaceutically acceptable salt thereof is administered oromucosally (e.g. sublingually or buccally) as a film. In embodiments, the dose is achieved by cutting a film in half to deliver a half-dose, e.g. a 60 μg dose may be administered with half of a second 60 μg dose (30 μg) to make a 90 μg dose.

In embodiments, a repeat dose cannot be administered until 2 or more hours have passed after the initial dose and after the collection of the 2 hour initial dose assessments. In embodiments, arepeat dose must occur within 12 hours of the initial dose and be based on a PEC change from baseline of <40%. In some embodiments, the maximum number of repeat doses per subject is 1.

In embodiments, the present disclosure is a method of treating agitation or signs of agitation in a human subject with dementia, without also inducing significant sedation, comprising administering from about 30 μg to about 180 μg of dexmedetomidine or a pharmaceutically acceptable salt thereof, wherein the agitiation is acute agitation. In embodiments, the present disclosure is a method of treating agitation or signs of agitation in a human subject with dementia, without also inducing significant sedation, comprising administering from about 30 μg to about 180 μg of dexmedetomidine or a pharmaceutically acceptable salt thereof, wherein the agitiation is chronic agitation.

The present disclosure provides methods of treating agitation in elderly patients having dementia comprising administering dexmedetomidine or a pharmaceutically acceptable salt thereof to the patient in an agitated state.

The data showed that doses higher than about 90 μg have negative consequences in elderly dementia patients and are not used; for example, higher doses may interact with blood pressure medications to cause unacceptable drops in blood pressure, brachycardia, and dizziness; syncope can occur in severe cases. In certain circumstances, however, doses above about 90 μg may be used in elderly dementia patients when agitated provided that the patient is not at risk for hypotension side effects. This disclosure thus provides for administering doses higher than about 90 μg to patients who have not received a hypertension treatment for at least 10 hours, at least 24 hours, at least 48 hours, or at least a week, prior to administering dexmedetomidine. Such higher doses may be about 100 μg to about 130 μg; for example, about 100 μg, about 110 μg, about 120 μg, or about 130 μg. Advantageously, dexmedetomidine may be used to replace the hyper-tension-treating drug while treating both agitation and the hyper-tension.

In embodiments, the dexmedetomidine may be administered to the agitated elderly dementia patients via a route that avoids first-pass metabolism; for example, intravenous, intramuscular, subcutaneous, transdermal. Preferably delivery is oromucosal; for example, buccal or sub-lingual. In embodiments, the dosage form is a film. Suitable films are described in U.S. Pat. No. 10,792,246, which is hereby incorporated by reference in its entirety for all purposes. In embodiments, the elderly patient is 55 years old or older, e.g., about 55 years old, about 56 years old, about 57 years old, about 58 years old, about 59 years old, about 60 years old, about 61 years old, about 62 years old, about 63 years old, about 64 years old, about 65 years old, about 66 years old, about 67 years old, about 68 years old, about 69 years old, about 70 years old, about 71 years old, about 72 years old, about 73 years old, about 74 years old, about 75 years old, about 76 years old, about 77 years old, about 78 years old, about 79 years old, about 80 years old, about 81 years old, about 82 years old, about 83 years old, about 84 years old, about 85 years old, about 86 years old, about 87 years old, about 88 years old, about 89 years old, about 90 years old, about 91 years old, about 92 years old, about 93 years old, about 94 years old, about 95 years old, about 96 years old, about 97 years old, about 98 years old, about 99 years old, about 100 years old. In embodiments, the elderly patient is about 65 years old or older. In embodiments, the elderly patient is about 70 years old or older. In embodiments, the elderly patient is about 75 to about 80 years old. In embodiments, the elderly patient is about 80 years old or older. In embodiments, the elderly patient has both dementia and Alzheimer's disease.

In embodiments, the agitation is acute agitation. In embodiments, the agitation is chronic agitation.

In embodiments, the present disclosure provides methods of treating agitation in elderly patients having dementia comprising administering dexmedetomidine or a pharmaceutically acceptable salt thereof to the patient in an agitated state, wherein the dose of dexmedetomidine is about 30 μg to about 90 μg (e.g. 40 μg); wherein the Cₘₐₓ is about 50 ng/L to about 300 ng/L; wherein the route of administration is oromucosal, intravenous, intramuscular, subcutaneous, or transdermal; and wherein the elderly patient is 65 years old or older. For example, the dose may be about 30 μg to about 90 μg; for example, about 30 μg to about 60 μg; about 60 μg to about 90 μg, about 30 μg to about 45 μg or about 30 μg to about 40 μg. In embodiments, the dose may be about 30 μg, about 40 μg, about 45 μg, about 50 μg, about 60 μg, about 75 μg, about 80 μg or about 90 μg.

In embodiments , the dose provides an AUC₀₋₈ in a range of about 200 hr*ng/L to about 1500 hr*ng/L; for example, about 200 hr*ng/L to about 1250 hr*ng/L, about 200 hr*ng/L to about 1000 hr*ng/L, about 200 hr*ng/L to about 750 hr*ng/L, about 200 hr*ng/L to about 500 hr*ng/L, about 500 hr*ng/L to about 1500 hr*ng/L, about 500 hr*ng/L to about 1250 hr*ng/L, about 500 hr*ng/L to about 1000 hr*ng/L, about 500 hr*ng/L to about 750 hr*ng/L, about 750 hr*ng/L to about 1500 hr*ng/L, about 750 hr*ng/L to about 1250 hr*ng/L, about 750 hr*ng/L to about 1000 hr*ng/L, or about 1000 hr*ng/L to about 1500 hr*ng/L. In embodiments, the dose provides an AUC₀₋₈ of about 200 hr*ng/L, about 300 hr*ng/L, about 400 hr*ng/L, about 500 hr*ng/L, about 600 hr*ng/L, about 700 hr*ng/L, about 800 hr*ng/L, about 900 hr*ng/L, about 1000 hr*ng/L, about 1100 hr*ng/L, about 1200 hr*ng/L, about 1300 hr*ng/L, about 1400 hr*ng/L, or about 1500 hr*ng/L.

In embodiments, the dose provides an AUC_{0-inf} in a range of about 200 hr*ng/L to about 5000 hr*ng/L, about 200 hr*ng/L to about 3500 hr*ng/L, about 200 hr*ng/L to about 2200 hr*ng/L, about 200 hr*ng/L to about 2000 hr*ng/L; for example, about 300 hr*ng/L to about 1900 hr*ng/L, about 400 hr*ng/L to about 1800 hr*ng/L, about 500 hr*ng/L to about 1700 hr*ng/L, about 500 hr*ng/L to about 1600 hr*ng/L, about 500 hr*ng/L to about 1500 hr*ng/L, about 600 hr*ng/L to about 1500 hr*ng/L, about 700 hr*ng/L to about 1500 hr*ng/L. In embodiments, the dose provides an AUC_{0-inf} of about 200 hr*ng/L, about 300 hr*ng/L, about 400 hr*ng/L, about 500 hr*ng/L, about 600 hr*ng/L, about 700 hr*ng/L, about 750 hr*ng/L, about 800 hr*ng/L, about 850 hr*ng/L, about 900 hr*ng/L, about 950 hr*ng/L, about 1000 hr*ng/L, about 1050 hr*ng/L, about 1100 hr*ng/L, about 1150 hr*ng/L, about 1200 hr*ng/L, about 1250 hr*ng/L, about 1300 hr*ng/L, about 1350 hr*ng/L, about 1400 hr*ng/L, about 1450 hr*ng/L, about 1500 hr*ng/L, about 1600 hr*ng/L, about 1700 hr*ng/L, about 1800 hr*ng/L, about 1900 hr*ng/L, about 2000 hr*ng/L, about 2100 hr*ng/L, about 2200 hr*ng/L, about 2300 hr*ng/L, about 2400 hr*ng/L, about 2500 hr*ng/L, about 2600 hr*ng/L, about 2700 hr*ng/L, about 2800 hr*ng/L, about 2900 hr*ng/L, about 3000 hr*ng/L, about 3100 hr*ng/L, about 3200 hr*ng/L, about 3300 hr*ng/L, about 3400 hr*ng/L, about 3500 hr*ng/L, about 2200 hr*ng/L, about 3600 hr*ng/L, about 3700 hr*ng/L, about 3800 hr*ng/L, about 3900 hr*ng/L, about 4000 hr*ng/L, about 4100 hr*ng/L, about 4200 hr*ng/L, about 4300 hr*ng/L, about 4400 hr*ng/L, about 4500 hr*ng/L, about 4600 hr*ng/L, about 4700 hr*ng/L, about 4800 hr*ng/L, about 4900 hr*ng/L or about 5000 hr*ng/L, including all values and ranges in between. In embodiments, the Cₘₐₓ and AUC₀₋₈ is preferably 80% to 125% of these ranges and values. In embodiments, the Cₘₐₓ and AUC_{0-inf} is preferably 80% to 125% of these ranges and values. The ranges obtained using these doses of dexmedetomidine were not expected based on prior studies. Indeed, compared to schizophrenia patients, the Cₘₐₓ data is about 38% higher and the AUC data is about 55% higher.

In embodiments, the present disclosure provides methods of treating an acute agitation episode in an elderly dementia patient, comprising administering dexmedetomidine, or a pharmaceutically acceptable salt thereof, to the agitated patient at a dose sufficient to provide a dexmedetomidine AUC₀₋₈ to a range between about 200 hr*ng/L to about 1500 hr*ng/L; wherein the Cₘₐₓ is about 50 ng/L to about 300 ng/L; wherein the patient is 65 years old or older; wherein the route of administration is selected from oromucosal, intravenous, intramuscular, subcutaneous, and transdermal. In embodiments, the present disclosure provides methods of treating an acute agitation episode in an elderly dementia patient, comprising administering dexmedetomidine, or a pharmaceutically acceptable salt thereof, to the agitated patient at a dose sufficient to provide a dexmedetomidine AUC_{0-inf} to a range between about 200 hr*ng/L to about 2200 hr*ng/L; wherein the Cₘₐₓ is about 50 ng/L to about 300 ng/L; wherein the patient is 65 years old or older; wherein the route of administration is selected from oromucosal, intravenous, intramuscular, subcutaneous, and transdermal. In embodiments, the method of treating an acute agitation episode in an elderly dementia patient, comprises administering to the patient about 30 μg to about 90 μg dexmedetomidine, or a pharmaceutically acceptable salt thereof, e.g. about 30 μg, about 40 μg, about 45 μg, about 50 μg, about 60 μg, about 75 μg, about 80 μg or about 90 μg. In embodiments, the method of treating an acute agitation episode in an elderly dementia patient, comprises administering to the patient about 30 μg dexmedetomidine, or a pharmaceutically acceptable salt thereof. In embodiments, the method of treating an acute agitation episode in an elderly dementia patient, comprises administering to the patient about 40 μg dexmedetomidine, or a pharmaceutically acceptable salt thereof. In embodiments, the method of treating an acute agitation episode in an elderly dementia patient, comprises administering to the patient about 60 μg dexmedetomidine, or a pharmaceutically acceptable salt thereof. In embodiments, the patient is not significantly sedated within about 60 minutes after administration.

In embodiments, the disclosure provides methods of treating chronic agitation in an elderly dementia patient, comprising administering dexmedetomidine, or a pharmaceutically acceptable salt thereof, to the agitated patient at a dose sufficient to provide a dexmedetomidine AUC₀₋₈ to a range between about 200 hr*ng/L to about 1500 hr*ng/L; wherein the Cₘₐₓ is about 50 ng/L to about 300 ng/L; wherein the patient is 65 years old or older; wherein the route of administration is selected from oromucosal, intravenous, intramuscular, subcutaneous, and transdermal. In embodiments, the present disclosure provides methods of treating chronic agitation in an elderly dementia patient, comprising administering dexmedetomidine, or a pharmaceutically acceptable salt thereof, to the agitated patient at a dose sufficient to provide a dexmedetomidine AUC_{0-inf} to a range between about 200 hr*ng/L to about 2200 hr*ng/L; wherein the Cₘₐₓ is about 50 ng/L to about 300 ng/L; wherein the patient is 65 years old or older; wherein the route of administration is selected from oromucosal, intravenous, intramuscular, subcutaneous, and transdermal. In embodiments, the method of treating chronic agitation in an elderly dementia patient, comprises administering to the patient about 30 μg to about 90 μg dexmedetomidine, or a pharmaceutically acceptable salt thereof, e.g. about 30 μg, about 40 μg, about 45 μg, about 50 μg, about 60 μg, about 75 μg, about 80 μg or about 90 μg. In embodiments, the method of treating chronic agitation in an elderly dementia patient, comprises administering to the patient about 30 μg dexmedetomidine, or a pharmaceutically acceptable salt thereof. In embodiments, the patient is not significantly sedated within about 60 minutes after administration.

In embodiments, the present disclosure provides methods of treating chronic agitation in an elderly dementia patient, comprising administering dexmedetomidine, or a pharmaceutically acceptable salt thereof, to the agitated patient at a dose sufficient to provide a dexmedetomidine AUC_{0-inf} to a range between about 200 hr*ng/L to about 3500 hr*ng/L; wherein the Cₘₐₓ is about 50 ng/L to about 300 ng/L; wherein the patient is 65 years old or older; wherein the route of administration is selected from oromucosal, intravenous, intramuscular, subcutaneous, and transdermal. In embodiments, the present disclosure provides methods of treating chronic agitation in an elderly dementia patient, comprising administering dexmedetomidine, or a pharmaceutically acceptable salt thereof, to the agitated patient at a dose sufficient to provide a dexmedetomidine AUC_{0-inf} to a range between about 200 hr*ng/L to about 5000 hr*ng/L; wherein the Cₘₐₓ is about 50 ng/L to about 500 ng/L; wherein the patient is 65 years old or older; wherein the route of administration is selected from oromucosal, intravenous, intramuscular, subcutaneous, and transdermal.

In embodiments, the dexmedetomidine or a pharmaceutically acceptable salt thereof is administered at a dose of about 30 μg to about 130 μg, and wherein the patient has not received treatment for hypertension within about 10 hours prior to dexmedetomidine administration.

In embodiments, the present disclosure also provides methods of managing or treating agitation in subjects with delirium, comprising administering about 20 μg to about 300 μg of dexmedetomidine or a pharmaceutically acceptable salt thereof. In embodiments, the subject is hospitalized. In embodiments, the subject is hospitalized in the intensive care unit. In embodiments, dexmedetomidine or a pharmaceutically acceptable salt thereof is oromucosally administered at a dose of about 20 μg, about 30 μg, about 40 μg, about 50 μg, about 60 μg, about 70 μg, about 80 μg, about 90 μg, about 100 μg, about 120 μg, about 150 μg, about 180 μg, about 210 μg, about 240 μg, about 270 μg, or about 300 μg.

In embodiments, the present disclosure provides methods of managing or treating agitation in subjects with delirium, comprising administering a dose of about 40 μg, about 60 μg, about 90 μg, about 120 μg or about 150 μg of dexmedetomidine or a pharmaceutically acceptable salt thereof wherein the subject's age is above 64 years old. In embodiments, one to ten doses can be administered at an appropriate dosing interval (e.g. 1 to 6 hours) to produce a desired effect; for example, about 60 μg, about 90 μg, about 120 μg, or about 150 μg dose (starting dose) is administered followed by an additional 5-7 doses of about 60 μg in a day at an interval ranging from about 1 to about 6 hours to produce the maximum cumulative dose of a about 480 μg dose.

In embodiments, the present disclosure provides a method of treating agitation or signs of agitation in a pediatric subject without also inducing significant sedation, comprising oromucosally administering about 10 μg to about 90 μg of dexmedetomidine or a pharmaceutically acceptable salt thereof, wherein the agitation is associated with schizophrenia, e.g. about 10 μg, about 15 μg, about 20 μg, about 30 μg, about 40 μg, about 50 μg, about 60 μg, about 70 μg, about 80 μg, or about 90 μg. In embodiments, the present disclosure provides a method of treating agitation or signs of agitation in a pediatric subject without also inducing significant sedation, comprising oromucosally administering a single dose containing about 10 μg of dexmedetomidine or a pharmaceutically acceptable salt thereof, wherein the agitation is associated with schizophrenia. In embodiments, the present disclosure provides a method of treating agitation or signs of agitation in a pediatric subject without also inducing significant sedation, comprising oromucosally administering a single dose containing about 20 μg of dexmedetomidine or a pharmaceutically acceptable salt thereof, wherein the agitation is associated with schizophrenia. In embodiments, the present disclosure provides a method of treating agitation or signs of agitation in a pediatric subject without also inducing significant sedation, comprising oromucosally administering a single dose containing about 30 μg of dexmedetomidine or a pharmaceutically acceptable salt thereof, wherein the agitation is associated with schizophrenia. In embodiments, the present disclosure provides a method of treating agitation or signs of agitation in a pediatric subject without also inducing significant sedation, comprising oromucosally administering a single dose containing about 80 μg of dexmedetomidine or a pharmaceutically acceptable salt thereof, wherein the agitation is associated with schizophrenia. In embodiments, the present disclosure provides a method of treating agitation or signs of agitation in a pediatric subject without also inducing significant sedation, comprising oromucosally administering a single dose containing about 120 μg of dexmedetomidine or a pharmaceutically acceptable salt thereof, wherein the agitation is associated with schizophrenia. In embodiments, the present disclosure provides a method of treating agitation or signs of agitation in a pediatric subject without also inducing significant sedation, comprising oromucosally administering a single dose containing about 80 μg of dexmedetomidine or a pharmaceutically acceptable salt thereof, wherein the agitation is associated with bipolar disorder. In embodiments, the present disclosure provides a method of treating agitation or signs of agitation in a pediatric subject without also inducing significant sedation, comprising oromucosally administering a single dose containing about 120 μg of dexmedetomidine or a pharmaceutically acceptable salt thereof, wherein the agitation is associated with bipolar disorder. In embodiments, the subject is about 13-17 years old. In embodiments, the subject is about 10-17 years old. In embodiments, the agitation is acute. In embodiments, the subject is diagnosed with Attenuated Psychosis Syndrome DSM-5 298.8 (F28). In embodiments, the subject has a score of ≥4 on at least 1 of the 5 items on the PEC at baseline. In embodiments, the dexmedetomidine or a pharmaceutically acceptable salt thereof is administered sublingually or buccally as a film. In embodiments, the reduction in agitation or signs of agitation is measured by relative change in PEC score after administration of dexmedetomidine or a pharmaceutically acceptable salt thereof. In embodiments, the clinical improvement in agitation is measured using PANSS, ACES, and/or CGI-I scales.

In embodiments, the pediatric subject is about 1 to about 18 years old, e.g., about 1 year old, about 2 years old, about 3 years old, about 4 years old, about 5 years old, about 6 years old, about 7 years old, about 8 years old, about 9 years old, about 10 years old, about 11 years old, about 12 years old, about 13 years old, about 14 years old, about 15 years old, about 16 years old, about 17 years old, about 18 years old, including all values and ranges in between.

In embodiments, the pediatric subject is about 10 to about 17 years old, e.g., about 10 years old, about 11 years old, about 12 years old, about 13 years old, about 14 years old, about 15 years old, about 16 years old, about 17 years, including all values and ranges in between.

In embodiments, the pediatric subject is about 13 to about 17 years old, e.g., about 13 years old, about 14 years old, about 15 years old, about 16 years old, about 17 years old, including all values and ranges in between.

In embodiments, the present disclosure also provides methods of treating or ameliorating opioid withdrawal symptoms, comprising administering a composition comprising dexmedetomidine or a pharmaceutically acceptable salt thereof (e.g., dexmedetomidine hydrochloride) to a human patient in need thereof. In embodiments, onset of opioid withdrawal begins within 6-24 hours from last opioid use.

In embodiments, the present disclosure provides methods of treating or ameliorating opioid withdrawal symptoms, comprising administering a composition comprising dexmedetomidine or a pharmaceutically acceptable salt thereof (e.g., dexmedetomidine hydrochloride) to a human patient in need thereof, wherein the dexmedetomidine or a pharmaceutically acceptable salt thereof is administered at a dose of about 20 μg to about 600 μg, e.g., about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, or about 600 μg, including all values and ranges in between. In embodiments, the dexmedetomidine or a pharmaceutically acceptable salt thereof is administered at a dose of about 240 μg twice a day (in an interval of 12 hours). In embodiments, the patient is at least 18 years old. In embodiments, the period of withdrawal is up to 14 days. In embodiments, the period of withdrawal is up to 12 days. In embodiments, the period of withdrawal is up to 10 days. In embodiments, the period of withdrawal is up to 6 days.

It was unexpectedly discovered that dexmedetomidine is effective at reducing the period of opioid withdrawal in an subject. This is surprising because opioids (e.g. fentanyl) become localized in body fat over time and are released intermittently and have unpredictable effects on patients during the withdrawal process. Due to the high degree of variability and intermittent release of opioids, a clinician would not have expected repeated administration of dexmedetomidine to be an effective therapy.

In embodiments, the present disclosure provides a method of reducing a period of opioid withdrawal in a human subject in need thereof, comprising administering to said subject dexmedetomidine or a pharmaceutically acceptable salt thereof once daily. In embodiments, the period of withdrawal is 1 day to 14 days, e.g. 14 days, 13 days, 12 days, 11 days, 10 days, 9 days, 8 days, 7 days, 6 days, 5 days, 4 days, or 3 days, including all values and ranges in between.

In embodiments, the present disclosure provides a method of reducing a period of opioid withdrawal in a human subject in need thereof, comprising administering to said subject dexmedetomidine or a pharmaceutically acceptable salt thereof twice daily. In embodiments, the period of withdrawal is 1 day to 14 days, e.g. 14 days, 13 days, 12 days, 11 days, 10 days, 9 days, 8 days, 7 days, 6 days, 5 days, 4 days, or 3 days, including all values and ranges in between.

In other embodiments, the present disclosure provides a method of reducing a period of opioid withdrawal in a human subject in need thereof, comprising administering to said subject dexmedetomidine or a pharmaceutically acceptable salt thereof twice daily. In embodiments, the period of withdrawal is up to about 60 days. In embodiments, the period of withdrawal may be 59 days, 58 days, 57 days, 56 days, 55 days, 54 days, 53 days, 52 days, 51 days, 50 days, 49 days, 48 days, 47 days, 46 days, 45 days, 44 days, 43 days, 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days or 3 days, including all values and ranges in between.

In embodiments, the present disclosure provides a method of reducing a period of opioid withdrawal in a human subject in need thereof, comprising administering to said subject dexmedetomidine or a pharmaceutically acceptable salt thereof twice daily, wherein the period of withdrawal is up to 14 days. In embodiments, the period of withdrawal is up to 12 days. In other embodiments, the present disclosure provides a method of reducing a period of opioid withdrawal in a human subject in need thereof, comprising administering to said subject dexmedetomidine or a pharmaceutically acceptable salt thereof twice daily where the period of withdrawal is up to about 60 days. In embodiments, the human subject is an adult (e.g. at least 18 years old) and suffering with opioid use disorder who is physically dependent on opioids. In embodiments, dexmedetomidine or a pharmaceutically acceptable salt is administered sublingually, buccally, orally, intranasally or parenterally. In embodiments, dexmedetomidine or a pharmaceutically acceptable salt (e.g. hydrochloride) is administered sublingually as a film. In embodiments, dexmedetomidine or a pharmaceutically acceptable salt thereof is oromucosally administered at a dose range of about 30 μg to about 600 μg, e.g., about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, about 500 μg, about 510 μg, about 520 μg, about 530 μg, about 540 μg, about 550 μg, about 560 μg, about 570 μg, about 580 μg, about 590 μg, or about 600 μg, including all values and ranges in between. In embodiments, dexmedetomidine or a pharmaceutically acceptable salt thereof is oromucosally administered at a dose range of about 30 μg to about 600 μg as a single dose. In embodiments, dexmedetomidine is administered as a dose of about 30 μg, about 60 μg, about 90 μg, about 120 μg, about 180 μg, about 240 μg, or about 300 μg twice daily approximately 12 hours apart for a period of at least 3 days (e.g. 3 days, 4 days, 5 days, 6 days, 7 days, 8 days, 9 days, 10 days, 11 days, 12 days or 13 days). In embodiments, each unit may be administered at an appropriate dosing interval (e.g. about 12 hours between doses) or can be administered concurrently, for example two units of 30 μg can be administered concurrently to produce the effect of a 60 μg dose or three units of 60 μg can be administered concurrently to produce the effect of a 180 μg dose.

In embodiments, the opioid may be selected from the group consisting of, but are not limited to fentanyl, morphine, codeine, heroin, oxycodone, hydrocodone, alfentanil, carfentanil, tramadol, hydromorphone, buprenorphine, naloxone, naltrexone, remifentanil butorphanol, meperidine, methadone, dextropropoxyphene (propoxyphene) thebaine, sufentanil or pentazocine. In embodiments, the opioid had been administered for the amount of time longer than neonate treatment prior to withdrawal.

In embodiments, the dose range of dexmedetomidine or a pharmaceutically acceptable salt thereof of between about 30 μg and about 600 μg. For example, the composition comprises a unit dose of about 30 μg, about 60 μg, about 90 μg, about 120 μg, 150 μg, 180 μg, 240 μg, or 300 μg, of dexmedetomidine or a pharmaceutically acceptable salt thereof. In embodiments, a single dose of about 180 μg dexmedetomidine or a pharmaceutically acceptable salt thereof is effective for up to at least about 24 hours. In embodiments, the dose is administered twice daily. In embodiments, the composition is administered twice daily for 7 days.

In embodiments, the mean plasma concentrations required to achieve the reduction in opioid withdrawal symptoms on day 6 after 2 hours of administration of dexmedetomidine or a pharmaceutically acceptable salt (e.g. hydrochloride) are in the range of about 40 ng/L to about 500 ng/L (for example about 40 ng/L to about 450 ng/L; about 40 ng/L to about 400 ng/L; about 40 ng/L to about 350 ng/L; about 40 ng/L to about 300 ng/L; about 40 ng/L to about 250 ng/L; about 40 ng/mL to about 200 ng/L; about 50 ng/L to about 150 ng/L; about 60 ng/L to about 150 ng/L; about 70 ng/L to about 100 ng/L including about 75 ng/L; about 80 ng/L, about 90 ng/L, about 95 ng/L, about 105 ng/L, about 115 ng/L, about 120 ng/L),

In embodiments, the mean plasma concentrations required to achieve the reduction in opioid withdrawal symptoms on day 6 after 6 hours of administration of dexmedetomidine or a pharmaceutically acceptable salt (e.g. hydrochloride) are in the range of about 20 ng/L to about 200 ng/L (for example about 20 ng/L to about 180 ng/L; about 20 ng/L to about 175 ng/L ; about 30 ng/L to about 170 ng/L; about 35 ng/L to about 165 ng/L; about 40 ng/L to about 160 ng/L; about 50 ng/L to about 150 ng/L; about 65 ng/L to about 125 ng/L; about 60 ng/L to about 100 ng/L; including about 70 ng/L; about 75 ng/L, about 80 ng/L, about 85 ng/L, about 90 ng/L, about 95 ng/L.

In embodiments, the mean plasma concentrations required to achieve the reduction in opioid withdrawal symptoms on day 6 after 12 hours of administration of dexmedetomidine or a pharmaceutically acceptable salt (e.g. hydrochloride).are in the range of about 20 ng/L to about 150 ng/L (for example : about 20 ng/L to about 125 ng/L; about 20 ng/L to about 100 ng/L; about 30 ng/L to about 90 ng/L, about 30 ng/L to about 75 ng/L).

In embodiments, the mean plasma concentrations required to achieve the reduction in opioid withdrawal symptoms on day 12 after 2 hours of administration of dexmedetomidine or a pharmaceutically acceptable salt (e.g. hydrochloride) are in the range of about 50 ng/L to about 500 ng/L (for example : about 50 ng/L to about 450 ng/L; about 50 ng/L to about 400 ng/L; about 75 ng/L to about 350 ng/L; about 75 ng/mL to about 300 ng/L; about 90 ng/L to about 250 ng/L ; about 90 ng/L to about 200 ng/L; about 100 ng/L to about 150 ng/L including about 140 ng/L, about 130 ng/L, about 125 ng/L , about 120 ng/L, about 110 ng/L).

In embodiments, the mean plasma concentrations required to achieve the reduction in opioid withdrawal symptoms on day 12 after 6 hours of administration of dexmedetomidine or a pharmaceutically acceptable salt (e.g. hydrochloride) are in the range of about 20 ng/L to about 250 ng/L (for example : about 20 ng/L to about 225 ng/L, about 20 ng/L to about 200 ng/L, about 20 ng/L to about 180 ng/L; about 20 ng/L to about 175 ng/L; about 30 ng/L to about 170 ng/mL; about 35 ng/L to about 165 ng/L; about 40 ng/L to about 160 ng/L; about 50 ng/L to about 150 ng/L; about 65 ng/L to about 125 ng/L; about 60 ng/L to about 100 ng/L including about 70 ng/L; about 75 ng/L, about 80 ng/L, about 85 ng/L, about 90 ng/L, about 95 ng/L).

In embodiments, the mean plasma concentrations required to achieve the reduction in opioid withdrawal symptoms on day 12 after 12 hours of administration of dexmedetomidine or a pharmaceutically acceptable salt are in the range of about 10 ng/L to 150 ng/L (for example: about 10 ng/L to 140 ng/L; about 20 ng/L to about 130 ng/L; about 30 ng/L to about 120 ng/L; about 40 ng/L to about 100 ng/L; about 50 ng/L to about 90 ng/L; about 75 ng/L to about 90 ng/L).

In embodiments, the mean plasma concentrations values are preferably 80% to 125% of these ranges and values.

In embodiments, the present disclosure provides a method of treating cocaine toxicity and/or symptoms associated with cocaine toxicity comprising administering oromucosally an effective amount of dexmedetomidine or a pharmaceutically acceptable salt thereof. In embodiments, dexmedetomidine or a pharmaceutically acceptable salt thereof is administered as an oromucosal film at a dose range of about 30 μg to about 400 μg as a single dose or as a multi-dose therapy. In embodiments, the present disclosure provides a pharmaceutical composition comprising from about 20 μg to about 600 μg dexmedetomidine or a pharmaceutically acceptable salt thereof (e.g. dexmedetomidine hydrochloride). In embodiments, the dose of dexmedetomidine is about 120 μg. In embodiments, the dose of dexmedetomidine is about 180 μg. In embodiments, the dose of dexmedetomidine is about 150 μg. In embodiments, the dose of dexmedetomidine is about 240 μg. In embodiments, the dose of dexmedetomidine is about 300 μg.

### Clinical Endpoints

The reduction of agitation in the elderly dementia patients may be assessed using various measurements: PEC, PAS, ACES, Mod-CMAI, and/or CGI-I.

In embodiments, the patient achieves a mean change in PEC score of greater than −2, −3, −4, −5, −6, −7, −8, −9, or −10 relative to baseline within 2 hours of administering the composition. In embodiments, the dose of dexmedetomidine is 30 μg and patient achieves a mean change in PEC score of greater than −4 relative to baseline within 2 hours of administering the composition. In embodiments, the dose of dexmedetomidine is 40 μg and patient achieves a mean change in PEC score of greater than −5 relative to baseline within 2 hours of administering the composition. In embodiments, the dose of dexmedetomidine is 60 μg and patient achieves a mean change in PEC score of greater than −7 relative to baseline within 2 hours of administering the composition. In embodiments, the decrease in PEC score is maintained for at least 2, 3, 4, 5, 6, 7, 8, 9, 10, 11, or 12, hours following administration of the composition.

In embodiments, the patient achieves a mean change in PAS score of greater than −2, −3, −4, −5, −6, −7, −8, −9, or −10 relative to baseline within 2 hours of administering the composition. In embodiments, the dose of dexmedetomidine is 30 μg and patient achieves a mean change in PAS score of greater than −3 relative to baseline within 2 hours of administering the composition.

In embodiments, the dose of dexmedetomidine is 40 μg and patient achieves a mean change in PAS score of greater than −4 relative to baseline within 2 hours of administering the composition. In embodiments, the dose of dexmedetomidine is 60 μg and patient achieves a mean change in PEC score of greater than −5 relative to baseline within 2 hours of administering the composition.

In embodiments, the decrease in PAS score is maintained for at least 2, 3, 4, 5, 6, 7, 8, 9, 10, 11, or 12, hours following administration of the composition.

In embodiments, the patient achieves a mean change in Mod-CMAI score of greater than −7, −8, −9, −10, −11, −12, −13, −14, −15, −16, −17, or −18 relative to baseline 2 hours after administering the composition. In embodiments, the dose of dexmedetomidine is 30 μg and patient achieves a mean change in Mod-CMAI score of greater than −7 relative to baseline within 2 hours of administering the composition. In embodiments, the dose of dexmedetomidine is 40 μg and patient achieves a mean change in Mod-CMAI score of greater than −10 relative to baseline within 2 hours of administering the composition. In embodiments, the dose of dexmedetomidine is 60 μg and patient achieves a mean change in Mod-CMAI score of greater than −13 relative to baseline within 2 hours of administering the composition. In embodiments, the decrease in Mod-CMAI score is maintained for at least 2, 3, 4, 5, 6, 7, 8, 9, 10, 11, or 12, hours following administration of the composition.

In embodiments, the patient achieves a CGI-I score improvement to about a 1 (very much improved) or about a 2 (much improved). In embodiments, the score improvement is sustained for a period of about 2 hours to about 6 hours. In embodiments, the dose of dexmedetomidine is 30 μg. In embodiments, the dose of dexmedetomidine is 40 μg. In embodiments, the dose of dexmedetomidine is 60 μg. In embodiments, the score is sustained for a period of about 2, 3, 4, 5, 6, 7, 8, 9, 10, 11, or about 12 hours.

In embodiments, the agitation is reduced to a 2 (moderate agitation), 3 (mild agitation) or 4 (normal behavior) 2 hours after administering the composition, as measured by the Agitation-Calmness Evaluation Scale (ACES). In embodiments, the dose of dexmedetomidine is 60μg. In embodiments, the dose of dexmedetomidine is 40 μg. In embodiments, the agitation is reduced to a 3 (mild agitation).

In embodiments, following administering of about 20 μg to about 300 μg of dexmedetomidine or a pharmaceutically acceptable salt thereof (e.g. dexmedetomidine hydrochloride) to an agitated human subject with delirium hospitalized (e.g. in an ICU), the agitation or signs of agitation and delirium severity are significantly reduced as measured by the RASS and DRS-R-98 respectively. For example, the agitation or signs of agitation and delirium severity are significantly reduced as measured by the RASS and DRS-R-98 just prior to and after every 30 minutes, 1 hour, 2 hours, 3, hours, 4 hours, 5 hours, or 6 hours post-administration of dexmedetomidine. In embodiments, the subject experiences a 2-point or greater drop in RASS at 2 hours after administration of about 20 μg to about 300 μg of dexmedetomidine or a pharmaceutically acceptable salt thereof. In embodiments, the subject experiences a 2-point or greater drop in RASS at 2 hours after administration of about 30 μg, about 60 μg, about 90 μg, about 120 μg, about 180 μg, about 240 μg, or about 300 μg of dexmedetomidine or a pharmaceutically acceptable salt thereof. In embodiments, the subject experiences a 2-point or greater drop in RASS at 2 hours after administration of about 20 μg to about 300 μg of dexmedetomidine or a pharmaceutically acceptable salt thereof, wherein the subject's initial RASS was not less than or equal to −3.

The treating or ameliorating opioid withdrawal symptoms may be measured by a variety of well-known means in the art, including but not limited to, the Clinical Opiate Withdrawal Scale (COWS) and/or Short Opiate Withdrawal Scale of Gossop (SOWS-Gossop) score.

In embodiments, the withdrawal symptoms following the treatment are assessed using the Clinical Opiate Withdrawal Scale and/or the Short Opiate Withdrawal Scale of Gossop (e.g. over a 10-day period).

In embodiments, following administering a unit dose of about 30 μg, about 60 μg, about 90 μg, about 120 μg, about 180 μg , about 240 μg, or about 300 μg of dexmedetomidine or a pharmaceutically acceptable thereof in a human subject experiencing opioid withdrawal symptoms (e.g. agitation or signs of agitation), the withdrawal symptoms are significantly reduced as measured by the relative COWS and/or the SOWS-Gossop scores just prior to and 2 hour post administration of dexmedetomidine or a pharmaceutically acceptable salt thereof. In embodiments, each unit may be administered twice daily over an appropriate period of withdrawal (e.g. for at least 3, 4, 5, 6, 7, 8, 9, 10, 11, 12, 13, or 14 days).

### Pharmaceutical Compositions

### Dosage Forms/Administration Methods

In embodiments, dexmedetomidine or a pharmaceutically acceptable salt thereof is administered oromucosally in the form of a tablet, film, spray, gel or drops, particularly a film. In embodiments, the film is placed under the tongue, close to the base of the tongue, on the left or right side. In embodiments, dexmedetomidine or a pharmaceutically acceptable salt thereof is administered buccally in the form of a film, patch or tablet, particularly a film. In embodiments, the film is placed against the inner lip or check, close to the jaw line. In embodiments, dexmedetomidine or a pharmaceutically acceptable salt thereof is administered parenterally to the subject in the form of an intramuscular injection. In embodiments, dexmedetomidine or a pharmaceutically acceptable salt thereof is administered to the subject by oral route. In embodiments, dexmedetomidine or a pharmaceutically acceptable salt thereof is administered orally in the form of tablets, orally disintegrating tablets (ODTs), effervescent tablets, capsules, pellets, pills, lozenges or troches, powders, dispersible granules, catchets, aqueous solutions, syrups, emulsions, suspensions, solutions, soft gels, dispersions and the like. In embodiments, dexmedetomidine or a pharmaceutically acceptable salt thereof is administered orally to the subject in the form of an orally disintegrating tablet

According to the present disclosure, dexmedetomidine or a pharmaceutically acceptable salt thereof can be administered to the human subject through various routes, including oromucosal (e.g. sublingual, buccal), oral, parenteral and the like. Formulations suitable for use according to the present disclosure are outlined below. Additional formulations suitable for use according to the present disclosure are described in US 2020/0000717, which is hereby incorporated by reference in its entirety for all purposes.

Oromucosal Formulations (Sublingual and/or Buccal Formulations)

Dexmedetomidine or a pharmaceutically acceptable salt thereof can be formulated, according to the present disclosure, into dosage forms suitable for oromucosal (e.g. sublingual or buccal) administration. Such dosage forms include tablets, powders, pills, films, capsules, liquids, gels, syrups, slurries, suspensions, and the like. In embodiments, dexmedetomidine or a pharmaceutically acceptable salt thereof is formulated as a film product.

Carriers suitable for inclusion in oromucosal (e.g. sublingual or buccal) formulations include, but are not limited to, sugars, starches, cellulose and its derivatives, malt, gelatin, talc, calcium sulphate, vegetable oils, synthetic oils, polyols, alginic acid, phosphate buffered solutions, emulsifiers, isotonic saline, pyrogen-free water and combinations thereof. Carriers which readily dissolve in saliva may be preferred.

Oromucosal (e.g. sublingual or buccal) formulations may also include other pharmaceutically acceptable carriers and/or excipients such as binders, lubricants, diluents, coatings, disintegrants, barrier layer components, glidants, coloring agents, solubility enhancers, gelling agents, fillers, proteins, co-factors, emulsifiers, solubilizing agents, suspending agents and mixtures thereof. Particular excipients, which may be used according to this disclosure, are known in the art, for example as described in Handbook of Pharmaceutical Excipients, fifth edition, 2005 edited by Rowe et al., Mcgraw Hill.

### Films

Suitable films for sublingual or buccal administration (i.e. oromucosal administration) according to the present disclosure comprise dexmedetomidine or a pharmaceutically acceptable salt thereof either (i) disposed within a polymer matrix or (ii) deposited on the surface of a polymer matrix, e.g., on the surface of a “placebo” film.

### Polymer Component of Film

The polymer component consists of one or more water-soluble polymers within the film matrix and/or as part of the drug-containing deposit (e.g. one or more droplets) on the surface of the polymer. In embodiments of the disclosure, the polymer component consists of a single water-soluble polymer. In embodiments, the polymer component consists of two or more water-soluble polymers, including two or more of the same water-soluble polymers having different molecular weights.

The polymer component in the film matrix is of a suitable composition and present in a sufficient amount to ensure rapid disintegration of the film matrix in the oral mucosa. For example, the presence of the polymer component may allow the film matrix to disintegrate completely oromucosally in about 15 seconds to about 180 seconds, for example, about 30 seconds to about 180 seconds, including about 120 seconds. The polymer component in the film matrix also provides the film with sufficient strength (i.e. the film is self-supporting).

When present in one or more droplets of the dexmedetomidine composition deposited onto the surface of the polymer matrix/substrate, the polymer component may, for example, consist of the water-soluble polymer hydroxypropyl cellulose, although different water-soluble polymers are also contemplated as described hereinafter under the definition “first water-soluble polymer” and “second water soluble polymer”. For example, the polymer component may consist of one, two or three hydroxypropyl celluloses having different molecular weights. The molecular weights of the different hydroxypropyl celluloses may conveniently range from (i) less than about 60,000 Daltons (e.g. about 5,000 Daltons to about 49,000 Daltons) (ii) about 90,000 Daltons to about 200,000 Daltons and (iii) about 200,000 Daltons to about 500,000 Daltons. The two or more hydroxypropyl celluloses may be mixed in any suitable ratio to achieve the desired droplet viscosity. The viscosity of the dexmedetomidine composition solution or suspension can be measured using a Brookfield viscometer with a small sample adapter at a temperature of 25° C. and may range from about 5 cps to about 3700 cps. For example, it may range from about 5 cps to about 500 cps, about 6 cps to about 200 cps, about 6 cps to about 100 cps or about 6 cps to about 50 cps. In some embodiments of the present disclosure, the viscosity of the dexmedetomidine composition solution or suspension is from about 6 cps to about 20 cps at 25° C. and a shear rate of about 7 (1/s).

When present in a monolithic (i.e. placebo or drug-containing) film, the polymer component may, for example, consist of one water soluble polymer or two different water-soluble polymers. When two different water-soluble polymers are present, one of the water-soluble polymers may include the same polymer but present in the polymer component as a combination of different molecular weights. For example, the polymer component may consist of one, two or three hydroxypropyl celluloses having different molecular weights, although different water-soluble polymers are also contemplated as described hereinafter under the definition “first water-soluble polymer” and “second water soluble polymer” such as polyethylene oxide. The molecular weights of the different hydroxypropyl celluloses may conveniently range from (i) less than about 60,000 Daltons (e.g. about 5000 Daltons to about 49000 Daltons) (ii) about 90000 Daltons to about 200000 Daltons and (iii) about 200,000 Daltons to about 500,000 Daltons (e.g. about 300000 Daltons to about 450000 Daltons). The two or more hydroxypropyl celluloses (e.g. low and high molecular weight hydroxypropyl celluloses) may be mixed in any suitable ratio to achieve the desired film properties. When present in a monolithic (i.e. placebo or drug-containing) film or micro-deposited film matrix composition, the polymer component may conveniently consist of one or more water-soluble polymers having a molecular weight less than about 60,000 Daltons (e.g. about 5,000 Daltons to about 49,000 Daltons), and/or from about 90000 Daltons to about 200,000 Daltons and/or about 200,000 Daltons to about 500,000 Daltons (e.g. about 300000 Daltons to about 450000 Daltons). When a structurally different water-soluble polymer is also present, it may conveniently have a higher molecular weight, for example a molecular weight greater than about 500,000 Daltons.

In embodiments, the disclosure provides pharmaceutical film compositions, comprising: (i) dexmedetomidine or a pharmaceutically acceptable salt thereof; (ii) a polymer component consisting of a first water-soluble polymer having a molecular weight less than about 60,000 Daltons (e.g. about 5,000 Daltons to about 49,000 Daltons), and one or more second-water soluble polymers having a molecular weight greater than about 60,000 Daltons; and, optionally, (iii) one or more pharmaceutically acceptable carriers.

In embodiments, the disclosure provides pharmaceutical film compositions comprising: (i) dexmedetomidine or a pharmaceutically acceptable salt thereof; (ii) a polymer component consisting of a first water-soluble polymer having a molecular weight less than about 60,000 Daltons (e.g. about 5,000 Daltons to about 49,000 Daltons), and one or more second-water soluble polymers having a molecular weight greater than about 60,000 Daltons; and, optionally, (iii) one or more pharmaceutically acceptable carriers.

In embodiments, the disclosure provides pharmaceutical film compositions consisting of: (i) dexmedetomidine or a pharmaceutically acceptable salt thereof; (ii) a polymer component consisting of a first water-soluble polymer having a molecular weight less than about 60,000 Daltons (e.g. about 5,000 Daltons to about 49,000 Daltons), and one or more second water-soluble polymers having a molecular weight greater than about 60,000 Daltons; and, optionally, (iii) one or more pharmaceutically acceptable carriers.

In embodiments, one or more first water-soluble polymers are selected from the group consisting of hydroxypropyl cellulose (HPC), hydroxyethyl cellulose, hydroxypropyl methylcellulose (HPMC), carboxymethyl cellulose, methyl cellulose and mixtures thereof, including mixtures of the same polymer having different molecular weights.

In embodiments, one or more second water-soluble polymers are selected from the group consisting of hydroxypropyl cellulose, hydroxypropyl methylcellulose, hydroxyethyl cellulose, carboxy methylcellulose, methylcellulose and mixtures thereof, including mixtures of the same polymer having different molecular weights. Polyethylene oxide (PEO) may also be present herein as a second water-soluble polymer or may be described separately hereinafter in the pharmaceutical film compositions as an example of a pharmaceutically acceptable carrier, or more particularly, as a mucoadhesive agent.

In embodiments, the weight ratio of said first water-soluble polymer to said second water-soluble polymer(s) (including PEO when present in the film) in the entire film composition is from about 2:1 to about 1:50, e.g., about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, including all values and ranges in between.

In embodiments, the weight ratio of said first water-soluble polymer to said second water-soluble polymer(s) (including PEO when present in the film) in the entire film composition is from about 1:10 to about 1:30, about 1:15 to about 1:25 or about 1:15 to about 1:20. In embodiments, a ratio of about 1:15 to about 1:20 provides beneficial functional effects.

In embodiments, other water-soluble polymers which may be included in the film with the first water-soluble polymer/second water-soluble polymer or replace such polymer(s). In embodiments other water-soluble polymers include povidone (polyvinylpyrrolidone), copovidone (copolymers of N-vinyl-2-pyrrolidone and vinyl acetate), polyvinyl alcohol, polyethylene glycol, polyacrylic acid, methylmethacrylate copolymer, carboxyvinyl copolymers, polydextrose, pullulan, carboxymethyl cellulose, sodium alginate, chitosan, xanthan gum, tragacanth gum, guar gum, acacia gum, arabic gum, starch, carrageenan, gelatin and mixtures thereof. The water-soluble polymer component, including water-soluble polymer carriers when present, may conveniently comprise about 40% to about 99.8%, about 50% to about 99.7%, about 60% to about 99.6% of the film composition, based on the weight of the film on a dry weight basis.

In embodiments, the polymer component for the film composition comprises a first water-soluble polymer present in an amount of from about 2% to about 15% on a dry weight basis of the polymer component (e.g. about 3% to about 8% w/w of the total film weight). This water-soluble polymer may conveniently have a molecular weight from about 5,000 Daltons to about 49,000 Daltons. Examples of suitable such water-soluble polymers include those selected from the group consisting of hydroxypropyl cellulose, hydroxyethyl cellulose, hydroxypropyl methylcellulose, carboxymethyl cellulose, methyl cellulose, and mixtures thereof.

In embodiments, low molecular weight hydroxypropyl cellulose may be present in the film at about 3% to about 8% w/w of the total film weight.

In embodiments, one or more second water-soluble polymers (e.g. polyethylene oxide) are present in an amount of from about 50 to about 98 weight percent on dry weight basis of the polymer component. In embodiments, the one or more second water-soluble polymers each has a molecular weight greater than 60,000 Daltons, for example, from about 90,000 Daltons to about 1,500,000 Daltons, especially when the polymer is selected from the group consisting of polyethylene oxide, hydroxypropyl cellulose, hydroxypropyl methylcellulose, hydroxyethyl cellulose, carboxy methylcellulose, methylcellulose, and mixtures thereof.

In embodiments, the one or more second water-soluble polymers are present in the film from about 25% w/w to about 40% w/w of the total film weight; the one or more second water-soluble polymers each has a molecular weight from about 90,000 Daltons to about 200,000 Daltons and/or from about 200,000 Daltons to about 500,000 Daltons, and the polymer is selected from the group consisting of hydroxypropyl cellulose, hydroxypropyl methylcellulose, hydroxyethyl cellulose, carboxy methylcellulose, methylcellulose, and mixtures thereof.

In embodiments, polyethylene oxide is present in the film at about 50% to about 60% w/w of the total film weight.

In embodiments, the polymer component of the film composition consists of a low molecular weight, water-soluble polymer (e.g., having a molecular weight less than about 60,000 Daltons) and one or more high molecular weight polymers (e.g., having a molecular weight greater about 60,000, up to about 1,500,000 Daltons when polyethylene oxide is included in the polymer mixture or up to about 500,000 Daltons when polyethylene oxide is not included in the polymer mixture). This polymer combination, especially when the polymers are a combination of hydroxypropyl cellulose and polyethylene oxide, lends certain advantages to the tensile strength and pharmacokinetics of the film composition.

In embodiments, the present disclosure provides a film composition comprising a therapeutically effective amount of dexmedetomidine or a pharmaceutically acceptable salt thereof; a polymer component comprising a water-soluble polymer and a pharmaceutically acceptable carrier.

In embodiments, the present disclosure provides a film composition comprising a therapeutically effective amount of dexmedetomidine or a pharmaceutically acceptable salt thereof; a polymer component comprising (a) a first water-soluble polymer (e.g. hydroxypropyl cellulose, hydroxypropyl methylcellulose, hydroxyethyl cellulose, carboxy methylcellulose, methylcellulose, and mixtures thereof) having a molecular weight from about 5,000 Daltons to about 49,000 Daltons, for example, in about 2 to about 15 weight percent on dry weight basis of the total polymer component; and (b) a second water-soluble polymers (e.g. polyethylene oxide, hydroxypropyl cellulose, hydroxypropyl methylcellulose, hydroxyethyl cellulose, carboxy methylcellulose, methylcellulose, and mixtures thereof) having a molecular weight greater than 60,000 Daltons (e.g. greater than 100000 Daltons), in about 50 to about 98 weight percent on dry weight basis of the total polymer component; and a pharmaceutically acceptable carrier.

The molecular weight of hydroxypropyl cellulose, when present in the film of the present disclosure, may be varied, and may be present as both a low molecular weight, water-soluble polymer and as one or more high molecular weight, water-soluble polymers. In embodiments, the molecular weight is less than about 60,000 Daltons (e.g. about 5,000 Daltons to about 49,000 Daltons). In embodiments the molecular weight of hydroxypropyl cellulose is about 90,000 Daltons to about 200,000 Daltons. In embodiments, the molecular weight of hydroxypropyl cellulose is about 200,000 Daltons to about 500,000 Daltons.

In embodiments, the composition comprises hydroxypropyl cellulose, in the range of about 10% to about 90% by weight on a dry weight basis of the polymer component, e.g. about 20% to about 80%, e.g. about 20% to about 50%, e.g. about 25% to about 45%.

The molecular weight of polyethylene oxide, in the film of the present disclosure, may also be varied. In embodiments, the composition comprises a water-soluble, high molecular weight polyethylene oxide, to increase muco-adhesivity of the film. In embodiments, the molecular weight of polyethylene oxide is about 100,000 Daltons to about 1,500,000 Daltons, e.g., about 100,000 Daltons, about 200,000 Daltons, about 300,000 Daltons, about 600,000 Daltons, about 900,000 Daltons or 1,000,000 Daltons. In embodiments, the composition comprises a combination of polyethylene oxide having a molecular weight of about 600,000 Daltons to about 900,000 Daltons and polyethylene oxide having a molecular weight of about 100,000 Daltons to about 300,000 Daltons in the polymer component.

In embodiments, the composition contains, about 30% to about 90% polyethylene oxide by weight on a dry weight basis of the total polymer component, e.g. about 40% to about 85%, and about 55% to about 80% polyethylene oxide by weight on a dry weight basis of the polymer component.

Such film compositions may contain the drug dispersed within the film, or micro-deposited onto a surface of the film. When micro-deposited on the surface of a “placebo” film, the drug may conveniently be added as part of a dexmedetomidine composition as one or more droplets in a liquid carrier, such as a solvent (e.g. an alcohol such as ethanol), optionally together with one or more (e.g. two) water-soluble polymers and/or pharmaceutically acceptable carriers. Suitable water-soluble polymers include (1) a low molecular weight, water-soluble polymer, for example a low molecular weight, water-soluble polymer having a molecular weight of less than about 60,000 Daltons (e.g. a molecular weight of about 5,000 Daltons to about 49,000 Daltons and optionally (2) one or more (e.g. one or two) high molecular weight, water-soluble polymers, for example a high molecular weight, water-soluble polymer having a molecular weight of greater than about 60,000 Daltons (e.g. a molecular weight of from about 60,000 Daltons to about 150,000 Daltons such as hydroxypropyl cellulose (77,000 MW), hydroxypropyl cellulose (80,000 MW), hydroxypropyl cellulose (90,000 MW), or hydroxypropyl cellulose (140,000 MW)) and/or a high molecular weight, water-soluble polymer having a molecular weight of greater than about 60,000 Daltons (e.g. a molecular weight of from about 200,000 Daltons to about 900,000 Daltons such as hydroxypropyl cellulose (about 340,000 MW), hydroxypropyl cellulose (about 370,000 MW), polyethylene oxide (about 200,000 MW) or polyethylene oxide (about 600,000 MW)). Each water-soluble polymer may independently be selected from the group consisting of hydroxypropyl cellulose, hydroxypropyl methylcellulose, hydroxyethyl cellulose, carboxymethyl cellulose, polyethylene oxide and methyl cellulose, e.g. hydroxypropyl cellulose and/or polyethylene oxide.

In embodiments, the composition comprises dexmedetomidine hydrochloride, a low molecular weight polymer which is hydroxypropyl cellulose and one or two high molecular weight polymers which are each hydroxypropyl cellulose in an ethanol solvent.

In embodiments, the composition comprises dexmedetomidine or a pharmaceutically acceptable salt thereof (e.g. dexmedetomidine hydrochloride), hydroxypropyl cellulose (about 40,000 MW) and one or both of hydroxypropyl cellulose (about 140,000 MW) and hydroxypropyl cellulose (about 370,000 MW).

In embodiments, the composition comprises dexmedetomidine or a pharmaceutically acceptable salt thereof (e.g. dexmedetomidine hydrochloride), and only two hydroxypropyl celluloses, namely hydroxypropyl cellulose (about 40,000 MW) and hydroxypropyl cellulose (about 140,000 MW).

In embodiments, the deposition composition may be in any form, including as a solution, emulsion, suspension or dispersion. For example, the dexmedetomidine composition may be added as one or more droplets in an ethanol-based solution, optionally containing a pH-neutralizing agent such as sodium hydroxide. In embodiments, the film substrate surface contains two or more micro-deposited spots of dexmedetomidine hydrochloride (e.g. two microdeposited spots) in a polymer matrix. The viscosity of deposition solution/suspension may range from about 6 cps to about 3700 cps as measured at 25° C. using a Brookfield viscometer with a small sample adapter. As an example, it may range from about 5 cps to about 500 cps, about 6 cps to about 200 cps, about 6 cps to about 100 cps or about 6 cps to about 50 cps.

In embodiments of the present disclosure, the viscosity of the dexmedetomidine composition is from about 6 cps to about 20 cps at 25° C. and a shear rate of about 7 (1/s).

Following drying to remove the solvent, the film comprises a film substrate (e.g. a placebo) with the dexmedetomidine composition as previously described but absent the solvent deposited (e.g. micro-deposited) on the surface of the film substrate. The dried composition containing dexmedetomidine or a pharmaceutically acceptable salt thereof (e.g. dexmedetomidine hydrochloride) may cover the whole of the film substrate surface or only part of the film substrate surface.

In embodiments, the dried dexmedetomidine composition appears as one or more discrete drug-containing droplets on the film substrate surface. Alternatively, stenciling may be used to achieve a one or more defined and discrete regions of drug-containing composition on the surface of the film substrate.

In embodiments, the disclosure provides a dry film product comprising a film substrate with one or more discrete drug-containing droplets on the film substrate surface, wherein each such drug-containing droplet comprises dexmedetomidine or a pharmaceutically acceptable salt thereof, and hydroxypropyl cellulose of two molecular weights: hydroxypropyl cellulose (40,000 MW) available as HPC-SSL, and hydroxypropyl cellulose (140,000 MW) marketed under the tradename of Klucel™ Type JF NF, and wherein the film substrate comprises hydroxypropyl cellulose of three molecular weights: hydroxypropyl cellulose (40,000 MW), hydroxypropyl cellulose (140,000 MW), and hydroxypropyl cellulose (370,000 MW) marketed under the tradename of Klucel™ Type GF NF. In some embodiments, the film substrate also comprises polyethylene oxide (600,000 MW) available under the name of Sentry Polyox WSR 205 LEO NF.

In embodiments, the dry film product comprises a deposition composition (also referred to herein as a “dexmedetomidine composition”) comprising: (i) dexmedetomidine hydrochloride, present at about 9% to about 50% w/w of the deposition composition, e.g. about 15% to about 25% w/w of the deposition composition; (ii) hydroxypropyl cellulose (40,000 MW), present at about 5% to about 85% w/w of the deposition composition; (iii) hydroxypropyl cellulose (140,000 MW) present at about 5% to 85% w/w of the deposition composition; and (iv) hydroxypropyl cellulose (370,000 MW) present at about 0% to about 65% w/w of the deposition composition. The film also comprises a polymer matrix, wherein the polymer matrix comprises: (i) hydroxypropyl cellulose (40,000 MW) present at about 3% to about 40% w/w of the polymer matrix; (ii) hydroxypropyl cellulose (140,000 MW) present at about 3% to about 40% w/w of the polymer matrix; (iii) hydroxypropyl cellulose (370,000 MW) present at about 0% to about 30% w/w of the polymer matrix, and (iv) polyethylene oxide (600,000 MW) present at about 55% to about 75% w/w of the polymer matrix.

In embodiments, the dry film product (e.g. a micro-deposited film product) comprises(i) dexmedetomidine hydrochloride, present at about 1% to about 50% w/w of the total film weight; (ii) hydroxypropyl cellulose (40,000 MW), present at about 2% to about 30% w/w of the total film weight ; (iii) hydroxypropyl cellulose (140,000 MW) present at about 2% to about 30% w/w of the total film weight ; (iv) hydroxypropyl cellulose (370,000 MW) present at about 10% to about 50% w/w of the total film weight , (v) polyethylene oxide (600,000 MW) present at about 40% to about 75% w/w of the total film weight and (vi) optionally other pharmaceutically acceptable carriers.

In embodiments, the films disclosed herein combine several types of hydroxypropyl cellulose (HPC) to provide a film with advantageous properties. For example, the film composition may contain two or three of hydroxypropyl cellulose (40,000 MW), hydroxypropyl cellulose (140,000 MW) and hydroxypropyl cellulose (370,000 MW) in combination. In some embodiments, polyethylene oxide (600,000 MW) is included with these types of HPC when part of a monolithic film.

In certain film compositions of the present disclosure, a low molecular weight hydroxypropyl cellulose (e.g. 40,000 MW) is present at about 3% to about 8% (e.g. about 5%) w/w of the total film weight, a high molecular weight hydroxypropyl cellulose (e.g. 140,000 MW) is present at about 3% to about 8% (e.g. about 5%) w/w of the total film weight, a high molecular weight hydroxypropyl cellulose (e.g. 370,000 MW) is present at about 20% to about 40% w/w of the total film weight, and a polyethylene oxide (e.g. 600,000 MW) is present at about 40% to about 70%, (e.g. about 50% to about 60%) w/w of the total film weight. In some embodiments, the two high molecular weight, water-soluble polymers are together present at about 25% to about 40% w/w of the total film weight.

The selection and ratio of water-soluble polymers can be made to effect complete dissolution of the film composition in oral mucosal fluids within seconds to minutes, e.g. in about 0.25 minutes to about 15 minutes, thus ensuring delivery of a therapeutically effective amount of dexmedetomidine or a pharmaceutically acceptable salt thereof via the oral mucosa. For example, the film compositions may reside in the sublingual or buccal region of the mouth up to about 15 minutes, up to about 10 minutes, or up to about 5 minutes, including for a period of from about 30 seconds to about 15 minutes, about 1 minute to about 10 minutes, or about 1 minute to about 5 minutes.

The standard basket or paddle apparatus described in any pharmacopoeia can be used for in vitro dissolution testing. The selection of dissolution medium will essentially depend as per the sink conditions and highest dose of drug. The temperature of dissolution medium should be maintained at 37±0.5° C. and rpm at 50 (see Bala et al., in Int J Pharm Investigation, vol. 3(2), pages 67-76).

Films disclosed herein have several functional advantages to promote rapid onset of drug effect. In some embodiments, thin films compositions of the disclosure have a disintegration time (DT) of about 15 seconds to about 180 seconds, about 15 seconds to about 160 seconds, about 25 seconds to about 150 seconds, about 15 seconds to about 140 seconds, about 15 seconds to about 120 seconds, about 40 seconds to about 120 seconds, about 50 seconds to about 120 seconds, for example about 120 seconds, when applied sublingually or buccally. A disintegration time in this time-frame provides optimal onset of drug effects.

In embodiments, thin film compositions of the invention have mucoadhesion properties that provide practical benefits of localizing the film to the oromucosal (e.g. buccal or sublingual) location and reducing, or preventing, effective removal prior to dissolution. This quality is particularly advantageous in a clinical setting with an agitated subject. Thus, in embodiments, thin film compositions have a mucoadhesion force (the mucoadhesion strength or shear strength) of about 50 g or above, about 100 g or above, about 200 g or above, about 300 g or above, about 400 g or above, about 500 g or above, about 600 g or above, about 700 g or above, about 800 g or above, about 900 g or above, about 1000 g or above. In embodiments, the mucoadhesion force is in a range of about 300 g to about 4000 g, about 500 g to about 3000 g, or about 1000 g to about 2000 g.

Burst strength of the film also contributes to drug delivery. Certain thin film compositions of the invention have a burst strength at or above 50 g, 100 g, 200 g, 300 g, 400 g, 500 g, 600 g, 700 g, 800 g, 900 g, 1000 g, 1100 g, 1200 g, 1300 g, 1400 g, 1500 g, 1600 g, 1700 g, 1800 g, 1900 g, 2,000 g, 2,500 g, 3,000 g, 3,500 g, 4,000 g, 4,500 g, 5,000 g, 5,500 g, 6,000 g, 6,500 g, 7,000 g, 7,500 g, 8,000 g, 8,500 g, 9,000 g, 9,500 g, 10,000 g or 15,000 g. For example, the burst strength may be in a range of about 200 g to about 15000 g, about 300 g to about 10,000 g, or about 400 g to about 5,000 g.

### Pharmaceutically Acceptable Carriers

The film compositions may further comprise one or more pharmaceutically acceptable carriers that includes, but is not limited to, liquid carriers, flavors, sweeteners, refreshing agents, antioxidants, pH adjusting agents, permeation enhancers, mucoadhesive agents, plasticizers, bulking agents, surfactants/non-ionic solubilizers, stabilizers, anti-foam agents, colors or the like. In embodiments, the film compositions are substantially free of acidic buffer or other acidic agents.

### Liquid Carriers

According to embodiments, the pharmaceutically acceptable carrier includes a liquid carrier. The liquid carrier comprises one or more solvents useful in the preparation of the polymer matrix (drug containing or placebo) and deposition composition on the polymer matrix. In embodiments, the solvent may be water. In embodiments, the solvent may a polar organic solvent including, but are not limited to, ethanol, isopropanol, acetone, butanol, benzyl alcohol and mixtures thereof. In embodiments, the solvent may be a non-polar organic solvent, such as methylene chloride, toluene, ethyl acetate and mixtures thereof. Certain solvents are alcohols, especially ethanol, water and mixtures thereof. Desirably, the solvent content in the wet polymer matrix is at least about 30% by weight of the total wet weight of the total film composition prior to drying. The subsequent dried film composition will desirably contain less than about 10% by weight of solvent, more desirably less than about 8% by weight of solvent, even more desirably less than about 6% by weight of solvent and most desirably less than about 2% by weight of solvent.

### Flavors/Sweeteners/Refreshing Agents

It may be beneficial to add a sweetener, flavoring agent, refreshing agent, taste-masking agent or a combination thereof to the film compositions to improve the film composition taste. Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. Non-limiting flavor oils include: spearmint oil, cinnamon oil, peppermint oil, clove oil, bay oil, thyme oil, cedar leaf oil, oil of nutmeg, oil of sage, and oil of bitter almonds. In some embodiments, the flavor is a peppermint oil flavor available as peppermint oil, NF.

The amount may be varied in order to obtain the result desired in the final product. Such variations are within the capabilities of those skilled in the art without the need for undue experimentation. In general, amounts of about 0.1% to about 30 wt % may be used in the films to supply flavoring. Suitable sweeteners include both natural and artificial sweeteners. Non-limiting examples of suitable sweeteners include, e.g.: water-soluble sweetening agents such as monosaccharides, disaccharides and polysaccharides such as xylose, ribose, glucose (dextrose), mannose, galactose, fructose (levulose), sucrose (sugar), high fructose corn syrup, maltose, invert sugar (a mixture of fructose and glucose derived from sucrose), partially hydrolyzed starch, corn syrup solids, and dihydrochalcones; water-soluble artificial sweeteners such as the soluble saccharin salts, i.e., sodium or calcium saccharin salts, cyclamate salts and water-soluble sweeteners derived from naturally occurring water-soluble sweeteners, such as a chlorinated derivatives of ordinary sugar (sucrose), known, for example, as sucralose. In some embodiments, the sweetener is sucralose.

Flavoring agents, sweeteners and refreshing agents can be added in conventional quantities, generally up to a total amount of about 0.01% to about 10% of the weight of the film on a dry weight basis, e.g. from about 0.1% to about 7% of the weight of the film on a dry weight basis, e.g. about 0.1% to about 5% based on the weight of the film on a dry weight basis.

Other taste-masking agents include, for example polymers, oils, or waxes. In embodiments, dexmedetomidine or a pharmaceutically acceptable salt thereof is coated with a taste-masking agent prior to formulation of the film compositions. In embodiments, if a taste-masking agent is used to coat the active ingredient, it may be present in an amount of from about 5% to about 80% by weight of the particle or granule containing the active ingredient. In embodiments, the taste-masking agent is present in an amount from about 25% to about 35% by weight of the particle or granule containing the active ingredient.

### Antioxidants

Examples of oxygen scavengers or antioxidants that substantially improve long-term stability of the film composition against oxidative degradation include sulfite salts, such as sodium sulfite, sodium bisulfite, sodium metabisulfite and analogous salts of potassium and calcium. A suitable amount of the sulfite salt (e.g., sodium sulfite) is up to about 5%, e.g. about 0.001% to about 2% based on the weight of the film composition on a dry weight basis.

pH-Adjusting Agents/pH-Neutralizing Agents

The absorption of dexmedetomidine or a pharmaceutical acceptable salt thereof through the oral mucosa may increase in an alkaline microenvironment. As an example, this may be achieved when the film compositions are maintained at a pH of above 6, from about 6 to about 9, or about 6.5 to about 8. In embodiments, the film may include an alkaline substance that increases the pH of the film product. Non-limiting examples of pH-adjusting/pH-neutralizing agents include bicarbonates (e.g., sodium bicarbonate), citrates (e.g., potassium citrate), carbonates (e.g., calcium carbonate), lactates (e.g., sodium lactate), acetates (e.g., calcium acetate), alkaline buffer (e.g. glycine), sodium hydroxide, sodium chloride or the like. An alkaline buffer, such as glycine, is one example of a pH-neutralizing agent. A suitable amount of pH-adjusting/pH-neutralizing agent present in the film composition includes, for example, up to about 10%, e.g. about 1% to about 5% based on the weight of the film composition on a dry weight basis

### Permeation Enhancer Agents

Certain effective penetration enhancers that promote absorption of dexmedetomidine or a pharmaceutically acceptable salt thereof across the oral mucosa include alcohols. An alcohol penetration enhancer, such as butanol, can conveniently be added to the film composition in an amount of up to about 10%, e.g. about 0.1% to about 5%, e.g. about 1% to about 3% based on the weight of the film composition on a dry weight basis.

### Mucoadhesive Agents

Examples of mucoadhesive agents that can be added to the film composition include, but are not limited to, sodium alginate, sodium carboxymethyl cellulose, guar gum, polyethylene oxide, hydroxyethyl cellulose, hydroxypropyl cellulose, hydroxypropylmethyl cellulose, karaya gum, methylcellulose, retene, tragacanth and the like. One mucoadhesive agent is polyethylene oxide, which may conveniently be added to the film composition in an amount of from about 20% to about 90%, e.g. about 40% to about 70% based on the total weight of the film composition on a dry weight basis.

### Plasticizers

Plasticizers that can be effectively employed herein include polyethylene glycol, propylene glycol, tributyl citrate, triethyl citrate and glycerol. Depending on the selected film-forming polymer(s) and other components of the film formulation, a suitable amount of plasticizer included in the film composition may typically be up to about 10%, e.g. about 0.1% to about 5%, e.g. about 0.5% to about 5% based on the weight of the film on a dry weight basis. For certain applications, higher molecular weight polyethylene glycols may be utilized, including polyethylene oxide

### Fillers

Suitable fillers that can be added to a film composition of include starch, calcium salts, such as calcium carbonate, and sugars, such as lactose, glucose, sucrose, mannose, sorbitol, mannitol, galactitol, sucralose, trehalose and combinations thereof. The amount of filler that can conveniently be added to the film formulation is typically up to about 25%, e.g. about 0.5% to about 20%, e.g. about 1% to about 15%, e.g. about 2% to about 10%, based on the weight of the film composition on a dry weight basis.

### Surfactants/Non-Ionic Solubilizers

The film typically incorporates at least one surfactant/non-ionic solubilizer including, for example, but are not limited to, a poloxamer, polyoxyl hydrogenated castor oil, glyceryl polyethylene glycol oxystearates, fatty acid glyceryl polyglyceryl esters, polyglyceryl esters, and combinations thereof. The amount of surfactant(s) that can be added to the film composition is typically up to about 5%, e.g. about 0.5% to about 3%, e.g. about 1% to about 3% based on the weight of the film composition on a dry weight basis.

### Anti Foaming Components

Simethicone is an example of a useful anti-foaming and/or de-foaming agent, although other anti-foaming and/or de-foaming agents may suitable be used. An anti-foaming and/or de-foaming agent such as simethicone may be added to the film composition in an amount from about 0.01% to about 5.0%, more desirably from about 0.05% to about 2.5%, and most desirably from about 0.1% to about 1.0% based on the weight of the film composition on a dry weight basis.

### Colorants

Color additives that may be included in a film composition include food, drug and cosmetic colors (FD&C), drug and cosmetic colors (D&C), or external drug and cosmetic colors (Ext. D&C). These colors are dyes, their corresponding lakes, and certain natural and derived colorants. Certain examples of color additives are inorganic pigments, such as oxides of iron or titanium, added in concentrations ranging from about 0.001% to about 10%, e.g. about 0.01% to about 3%, based on the weight of the film composition on a dry weigh basis. In embodiment, the color used for the dexmedetomidine composition (i.e. the deposit composition) is different from the color used for the film substrate (e.g. the placebo film). One color of the monolithic film and the film substrate of the micro-deposited film is emerald green, and available as Fast Emerald Green Shade (06507). One color of the dexmedetomidine composition (i.e. the deposit composition) is a different color from the color of the film substrate, e.g. blue (available as FD&C Blue No. 1). In embodiments of the film embodiments of the present disclosure, for example, as described in aspects and embodiments hereinabove, is a film comprising about 180 μg of dexmedetomidine or a pharmaceutically acceptable salt thereof containing two blue color microdeposited spots of dexmedetomidine hydrochloride on the green color film substrate.

In embodiments of the film embodiments of the present disclosure, for example, as described in aspects and embodiments hereinabove, is a film comprising about 120 μg of dexmedetomidine or a pharmaceutically acceptable salt thereof.

In one embodiment (A), there is provided a self-supporting, dissolvable, film, comprising:
(i) about 180 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In another embodiment (B), there is provided a self-supporting, dissolvable, film, comprising:
(i) about 120 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In another embodiment (C), there is provided a self-supporting, dissolvable, film, comprising:
(i) about 90 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In another embodiment (D), there is provided a self-supporting, dissolvable, film, comprising:
(i) about 80 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In another embodiment (E), there is provided a self-supporting, dissolvable, film, comprising:
(i) about 60 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In another embodiment (F), there is provided a self-supporting, dissolvable, film, comprising:
(i) about 40 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In another embodiment (G), there is provided a self-supporting, dissolvable, film, comprising:
(i) about 20 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In another embodiment (H), there is provided a self-supporting, dissolvable, film, comprising:
(i) about 10 μg of dexmedetomidine or a pharmaceutically acceptable salt thereof (e.g. the hydrochloride salt);
(ii) one or more water-soluble polymers;
(iii) a polyethylene oxide and, optionally,
(iv) one or more pharmaceutically acceptable carriers.

In a particular embodiment, the one or more water-soluble polymers (ii) of embodiments (A)-(H) above comprises a low molecular weight, water-soluble polymer and two high molecular weight, water-soluble polymers, for example wherein the low molecular weight, water-soluble polymer has a molecular weight from about 5,000 Daltons to about 49,000 Daltons (e.g. about 40,000 Daltons), and each high molecular weight, water-soluble polymer has a molecular weight of greater than about 60,000 Daltons (e.g. where one of the two high molecular weight, water-soluble polymers has a molecular weight of about 140,000 Daltons, and the other high molecular weight, water-soluble polymer has a molecular weight of about 370,000 Daltons). Each water-soluble polymer is, in some embodiments, hydroxypropyl cellulose. The polyethylene oxide, in some embodiments, has a molecular weight of about 600,000 Daltons.

In certain embodiments, there is provided a pharmaceutical film composition comprising or consisting essentially of therapeutically effective amount of dexmedetomidine or pharmaceutically acceptable salt thereof and one or more excipients selected from polyethylene oxide, hydroxypropyl cellulose, sucralose, peppermint oil, Emerald green colorant, and FD&C blue colorant.

In another embodiment (I), there is provided a self-supporting, dissolvable, film, comprising:
(i) about 180 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In another embodiment (J), there is provided a self-supporting, dissolvable, film, comprising:
(i) about 120 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In another embodiment (K), there is provided a self-supporting, dissolvable, film, comprising:
(i) about 90 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In another embodiment (L), there is provided a self-supporting, dissolvable, film, comprising:
(i) about 80 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In another embodiment (M), there is provided a self-supporting, dissolvable, film, comprising:
(i) about 60 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In another embodiment (N), there is provided a self-supporting, dissolvable, film, comprising:
(i) about 40 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In another embodiment (0), there is provided a self-supporting, dissolvable, film, comprising:
(i) about 20 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In another embodiment (P), there is provided a self-supporting, dissolvable, film, comprising:
(i) about 10 μg of dexmedetomidine or a pharmaceutically acceptable salt thereof (e.g. the hydrochloride salt);
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

In a particular embodiment of the just-mentioned films of embodiments (I) and (P), the film components excluding dexmedetomidine or a pharmaceutically acceptable salt thereof form a single layer film substrate, and dexmedetomidine or a pharmaceutically acceptable salt thereof is present on the surface of the film substrate (e.g., within a composition comprising dexmedetomidine or a pharmaceutically acceptable salt thereof, a low molecular weight, water-soluble polymer having a molecular weight of about 40,000 Daltons, and a high molecular weight, water-soluble polymer having a molecular weight of about 140,000 Daltons). Each water-soluble polymer is, in some embodiments, hydroxypropyl cellulose.

In embodiment (Q), there is provided a self-supporting, dissolvable, film, comprising:
(a) a composition consisting essentially of:
   (i) about 180 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In embodiment (R), there is provided a self-supporting, dissolvable, film, comprising:
(a) a composition consisting essentially of:
   (i) about 120 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In embodiment (S), there is provided a self-supporting, dissolvable, film, comprising:
(a) a composition consisting essentially of:
   (i) about 90 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In embodiment (T), there is provided a self-supporting, dissolvable, film, comprising:
(a) a composition consisting essentially of:
   (i) about 80 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In embodiment (U), there is provided a self-supporting, dissolvable, film, comprising:
(a) a composition consisting essentially of:
   (i) about 60 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In embodiment (V), there is provided a self-supporting, dissolvable, film, comprising:
(a) a composition consisting essentially of:
   (i) about 40 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In embodiment (W), there is provided a self-supporting, dissolvable, film, comprising:
(a) a composition consisting essentially of:
   (i) about 20 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In embodiment (X), there is provided a self-supporting, dissolvable, film, comprising:
(a) a composition consisting essentially of:
   (i) about 10 μg of dexmedetomidine hydrochloride;
   (ii) hydroxypropyl cellulose (40,000 MW); and
   (iii) hydroxypropyl cellulose (140,000 MW); and
(b) a film substrate consisting essentially of:
   (i) hydroxypropyl cellulose (40,000 MW);
   (ii) hydroxypropyl cellulose (140,000 MW);
   (iii) hydroxypropyl cellulose (370,000 MW); and
   (iv) polyethylene oxide (600,000 MW);

wherein the composition of part (a) is present on the surface of the film substrate (b).

In a particular embodiment of the just-mentioned films of embodiments (Q) and (X), dexmedetomidine hydrochloride is present at about 0.1% to about 2% w/w of the total film weight, hydroxypropyl cellulose (40,000 MW) is present at about 4% to about 8% w/w of the total film weight, hydroxypropyl cellulose (140,000 MW) is present at about 4% to about 8% w/w of the total film weight, hydroxypropyl cellulose (370,000 MW) is present at about 25% to about 30% w/w of the total film weight, and polyethylene oxide (600,000 MW) is present at about 50% to about 60% w/w of the total film weight.

In embodiments, the pharmaceutical composition of the present disclosure provides detectable Cₘₐₓ of dexmedetomidine in human plasma concentration after single dose administration and multiple dose administrations of the pharmaceutical composition of the present disclosure. In embodiments, the pharmaceutical composition of the present disclosure provides a Tmax of dexmedetomidine in human plasma concentration after a single dose administration or multiple dose administrations of the pharmaceutical composition of the present disclosure. In embodiments, pharmaceutical compositions of the present disclosure provide detectable Area Under the Curve (AUC) of dexmedetomidine and its metabolites in human plasma concentration after single dose administration or multiple dose administrations. In some embodiments, the AUC of dexmedetomidine (or its metabolites) is measured from time 0 (the time of administration) to 24 hours from the time 0 and is expressed as AUC_{0-24 h.}. In embodiments, the AUC of dexmedetomidine (or its metabolites) is measured from time 0 (the time of administration) to time extrapolated to infinity and is expressed as AUC_{0-Inf}. In embodiments, the ranges and values for AUC₀₋ₗₐₛₜ and AUC_{0-Inf} for dexmedetomidine (or its metabolites) are similar to the ranges and values for AUC_{0-6 h} for dexmedetomidine (or its metabolites). In embodiments, the present disclosure provides pharmaceutical buccal film compositions comprising or consisting essentially of therapeutically effective amount of dexmedetomidine or pharmaceutically acceptable salt thereof, one or more mucoadhesive polymers and optional excipients selected from one or more of plasticizers, penetration enhancers, coloring agents, sweetening agents, flavoring agents, taste-making agents or salivary stimulants. Mucoadhesive polymers may be selected from hydrophilic polymers and hydrogels. Examples of hydrophilic polymers include polyvinyl alcohol [PVA], sodium carboxy methylcellulose, hydroxyl propyl methyl cellulose [HPMC], hydroxyl ethyl cellulose and hydroxypropyl cellulose [HPC]. Examples of hydrogels include anionic polymers like Carbopol, polyacrylates, cationic polymers like chitosan and non-ionic polymers like Eudragit analogues.

### Sprays, Drops or Gels

In embodiments, the present disclosure provides pharmaceutical spray compositions or drop compositions suitable for sublingual or buccal administration comprising or consisting essentially of a therapeutically effective amount of dexmedetomidine or pharmaceutically acceptable salt thereof and one or more pharmaceutically acceptable liquids (from about 1% to about 99.995% by weight). Such liquids may be solvents, co-solvents, or non-solvents for dexmedetomidine or a pharmaceutically acceptable salt thereof. Examples of pharmaceutically acceptable liquids include water, ethanol, dimethyl sulfoxide, propylene glycol, polyethylene glycol, propylene carbonate, glycerin, N-methylpyrrolidone, pharmaceutically acceptable oils (e.g., soybean, sunflower, peanut, etc.) or the like. The pharmaceutically acceptable liquid is selected either to dissolve dexmedetomidine or pharmaceutically acceptable salt thereof, to produce a stable, homogenous suspension of it, or to form any combination of a suspension or solution. In addition to these ingredients, spray or drop formulations of dexmedetomidine or pharmaceutically acceptable salt thereof may include one or more excipients such as viscosity modulating materials (e.g. polymers, sugars, sugar alcohols, gums, clays, silicas, and the like, such as polyvinylpyrrolidone (PVP)); preservatives (e.g., ethanol, benzyl alcohol, propylparaben and methylparaben); flavoring agents (e.g. peppermint oil), sweeteners (e.g., sugars such as sucrose, glucose, dextrose, maltose, fructose, etc.), artificial sweeteners (e.g. saccharin, aspartame, acesulfame, sucralose), or sugar alcohols (e.g. mannitol, xylitol, lactitol, maltitol syrup); buffers and pH-adjusting agent (e.g., sodium hydroxide, citrate, and citric acid); coloring agents; fragrances, chelating agents (e.g., EDTA); UV absorbers and antifoam agents (e.g., low molecular weight alcohols, dimethicone). In addition to one or more of the aforementioned ingredients suitable for sublingual or buccal sprays or drops, gel formulations of dexmedetomidine or pharmaceutically acceptable salt thereof may include one or more excipients such as viscosity modulating materials (e.g. water soluble or water swellable polymers such as carbopol, hydroxypropyl cellulose, hydroxypropyl methyl cellulose, carboxymethyl cellulose).

Sprays, drops, and gels may be made by mixing appropriate quantities of the foregoing ingredients in accordance with standard good manufacturing practices. Such excipients may be included in the formulation to improve patient or subject acceptance or taste, to improve bioavailability, to increase shelf-life, to reduce manufacturing and packaging costs, to comply with requirements of governmental regulatory agencies, and for other purposes. The relative amounts of each ingredient should not interfere with the desirable pharmacological and pharmacokinetic properties of the resulting formulation.

In embodiments, there is provided an oromucosal spray composition comprising or consisting essentially of therapeutically effective amount of dexmedetomidine or pharmaceutically acceptable salt thereof and one or more pharmaceutically acceptable carrier or excipients.

In embodiments, a patient is treated by sublingually or buccally administering 1 to 2 actuations from a spray pump. An advantage of spray delivery is the ability to easily titrate patients by 1 or 2 doses as required by a single actuation.

Pump action sprays are characterized in requiring the application of external pressure for actuation, for example, external manual, mechanical or electrically initiated pressure. This is in contrast to pressurized systems, e.g., propellant-driven aerosol sprays, where actuation is typically achieved by controlled release of pressure e.g., by controlled opening of a valve.

Various oromucosal spray formulations comprising dexmedetomidine hydrochloride at doses of about 10 μg, about 20 μg, about 30 μg, about 40 μg, about 60 μg, about 80 μg, about 90 μg, about 120 μg, about 180 μg and about 240 μg and excipients as described in table 1.

TABLE 1
Oromucosal spray formulation embodiments
according to the disclosure

| | Oromucosal Spray Formulation Embodiment No. | | | |
|---|---|---|---|---|
| Ingredients | 1 | 2 | 3 | 4 |
| N-methylpyrrolidone | ✓ | | | |
| Propylene Glycol | | ✓ | | |
| Polyethylene Glycol | | | ✓ | |
| Glycerine | | | | ✓ |
| Ethanol | ✓ | ✓ | ✓ | ✓ |
| Sucralose | ✓ | ✓ | ✓ | ✓ |
| Peppermint Oil | ✓ | ✓ | ✓ | ✓ |
| Purified water | ✓ | ✓ | ✓ | ✓ |
| Optionally other | ✓ | ✓ | ✓ | ✓ |
| pharmaceutically | | | | |
| acceptable excipients | | | | |

Various oromucosal drop compositions comprising dexmedetomidine hydrochloride at doses of about 10 μg, about 20 μg, about 30 μg, about 40 μg, about 60 μg, about 90 μg, about 120 μg, 180 μg, and about 240 μg and excipients as described in table 2.

TABLE 2
Oromucosal drop formulations embodiments according to the disclosure

| | Oromucosal Drop Formulation Embodiment No. | | | | | | | | | | | | | |
|---|---|---|---|---|---|---|---|---|---|---|---|---|---|---|
| Ingredients | 1 | 2 | 3 | 4 | 5 | 6 | 7 | 8 | 9 | 10 | 11 | 12 | 13 | 14 |
| Povidone | ✓ | ✓ | ✓ | ✓ | ✓ | | | | | | | | | |
| N-methylpyrrolidone | ✓ | | | | | ✓ | | | | ✓ | | | | |
| Hydroxypropyl | | | | | | ✓ | ✓ | ✓ | ✓ | | | | | |
| methylcellulose | | | | | | | | | | | | | | |
| Carbopol | | | | | | | | | | ✓ | ✓ | ✓ | ✓ | ✓ |
| Polyethylene glycol | | | ✓ | | | | | | | | | ✓ | | |
| Propylene glycol | | ✓ | | | | | ✓ | | | | ✓ | | | |
| Glycerine | | | | ✓ | | | | | ✓ | | | | ✓ | |
| Ethanol | | | | | ✓ | | | ✓ | | | | | | ✓ |
| Sucralose | ✓ | ✓ | ✓ | ✓ | ✓ | ✓ | ✓ | ✓ | ✓ | ✓ | ✓ | ✓ | ✓ | ✓ |
| Peppermint Oil | ✓ | ✓ | ✓ | ✓ | ✓ | ✓ | ✓ | ✓ | ✓ | ✓ | ✓ | ✓ | ✓ | ✓ |
| Purified water | ✓ | ✓ | ✓ | ✓ | ✓ | ✓ | ✓ | ✓ | ✓ | ✓ | ✓ | ✓ | ✓ | ✓ |
| Optionally other | ✓ | ✓ | ✓ | ✓ | ✓ | ✓ | ✓ | ✓ | ✓ | ✓ | ✓ | ✓ | ✓ | ✓ |
| pharmaceutically | | | | | | | | | | | | | | |
| acceptable excipients | | | | | | | | | | | | | | |

Various oromucosal gel compositions comprising dexmedetomidine hydrochloride at doses of 20 μg, 30 μg, 40 μg, 60 μg, 90 μg, 120 μg, 180 μg and 240 μg, and excipients as described in table 3.

TABLE 3
Oromucosal gel formulations embodiments according to the disclosure.

| | Oromucosal Gel Formulation Embodiment Nos. | | | | | | | | | | | | | | |
|---|---|---|---|---|---|---|---|---|---|---|---|---|---|---|---|
| Ingredients | 1 | 2 | 3 | 4 | 5 | 6 | 7 | 8 | 9 | 10 | 11 | 12 | 13 | 14 | 15 |
| Carbopol | ✓ | | | ✓ | | | ✓ | | ✓ | | | | ✓ | | |
| Hydroxypropyl | | ✓ | | | ✓ | | | ✓ | | | ✓ | | | ✓ | |
| methylcellulose | | | | | | | | | | | | | | | |
| Hydroxypropyl | | | | | | | | | | | | | | | |
| cellulose | | | | | | | | | | | | | | | |
| Carboxymethyl | | | ✓ | | | ✓ | | | | ✓ | | ✓ | | | ✓ |
| cellulose | | | | | | | | | | | | | | | |
| N-Methylpyrrolidone | | | | ✓ | ✓ | ✓ | | | | | | | | | |
| Propylene glycol | | | | | | | ✓ | ✓ | | ✓ | | | | | |
| Polyethylene glycol | | | | | | | | | ✓ | | ✓ | ✓ | | | |
| Glycerine | | | | | | | | | | | | | ✓ | ✓ | ✓ |
| Ethanol | ✓ | ✓ | ✓ | | | | | | | | | | | | |
| Sucralose | ✓ | ✓ | ✓ | ✓ | ✓ | ✓ | ✓ | ✓ | ✓ | ✓ | ✓ | | ✓ | ✓ | ✓ |
| Peppermint oil | | ✓ | ✓ | ✓ | ✓ | ✓ | ✓ | ✓ | ✓ | ✓ | ✓ | | ✓ | ✓ | ✓ |
| Purified water | ✓ | ✓ | ✓ | ✓ | ✓ | ✓ | ✓ | ✓ | ✓ | ✓ | ✓ | ✓ | ✓ | ✓ | ✓ |
| Optionally other | ✓ | ✓ | ✓ | ✓ | ✓ | ✓ | ✓ | ✓ | ✓ | ✓ | ✓ | ✓ | ✓ | ✓ | ✓ |
| pharmaceutically | | | | | | | | | | | | | | | |
| acceptable excipients | | | | | | | | | | | | | | | |

### Tablets

In embodiments, the present disclosure provides tablet formulations suitable for oromucosal administration(e.g. sublingual or buccal administration) comprising or consisting essentially of therapeutically effective amount of dexmedetomidine or pharmaceutically acceptable salt thereof and one or more pharmaceutically acceptable carrier (from about 1% to about 99.995% by weight). Such carriers may be taste masking agents, diluents, disintegrants, binders, lubricants, glidants, flavoring agents or liquid solvents. Examples of pharmaceutically acceptable liquids include water, ethanol, dimethyl sulfoxide, propylene glycol, polyethylene glycol, propylene carbonate, glycerine, N-methylpyrrolidone, pharmaceutically acceptable oils (e.g., soybean, sunflower, peanut, etc.) or the like. Taste masking agents include, for example, amberlite, Opadry® AMB TAN, polymethacrylates (especially Eudragit® L100), sodium starch glycolate (Primojel), carbopol polymers, PEG-5M, sodium acetate, ethylcellulose, betacyclodextrin. Flavouring agents may be, for example, mint powder, menthol, vanillin, aspartame, acesulfame potassium, saccharin. Disintegrants include, for example, sodium starch glycolate, low-substituted hydroxy propyl cellulose, alginic acid, carbon dioxide, carboxymethylcellulose calcium, carboxymethylcellulose sodium, croscarmellose sodium, guar gum, methylcellulose, polacrilin potassium, poloxamer, sodium alginate. Diluents may be, for example, microcrystalline cellulose, dextrates, dextrose, fructose, mannitol, sucralose, sorbitol, starch, pregelatinized starch, sucrose, xylitol, maltose, maltodextrin, maltitol. Binders may be, for example, alginic acid, carbomer, ethyl cellulose, gelatine, liquid glucose, guar gum, hydroxyethyl cellulose, methylcellulose, polydextrose, polyethylene oxide, hydroxypropyl methylcellulose, hydroxypropyl cellulose, sodium alginate. At least one lubricant may conveniently be incorporated into the formulation to prevent the powder from adhering to tablet punches during the compression procedure. Lubricants may be, for example, talc, magnesium stearate, calcium stearate, glyceryl behenate, hydrogenated castor oil, stearic acid, sodium lauryl sulphate. Glidants are used to promote powder flow by reducing interparticle friction and cohesion. These are used in combination with lubricants as they have no ability to reduce die wall friction. Glidants, may be, for example, colloidal silicon dioxide, calcium silicate, calcium phosphate tribasic.

Various buccal tablet formulations comprising dexmedetomidine hydrochloride at doses of 20 μg, 30 μg, 40 μg, 60 μg, 90 μg, 120 μg, 180 μg and 240 μg and excipients as described in table 4.

TABLE 4
Buccal tablet formulation embodiments according to the disclosure.

| | Buccal Tablet Formulation Embodiment No. | | | | |
|---|---|---|---|---|---|
| Ingredients | 1 | 2 | 3 | 4 | 5 |
| Lactose monohydrate | ✓ | ✓ | ✓ | ✓ | ✓ |
| Polyethylene oxide | ✓ | | | | |
| Hydroxypropyl | | ✓ | | | |
| cellulose | | | | | |
| Hydroxypropyl | | | | | ✓ |
| methylcellulose | | | | | |
| Sodium alginate | | | | ✓ | |
| Xanthan gum | | | ✓ | | |
| Sucralose | ✓ | ✓ | ✓ | ✓ | ✓ |
| Magnesium stearate | ✓ | ✓ | ✓ | ✓ | ✓ |
| Talc | | ✓ | ✓ | ✓ | ✓ |
| Optionally other | ✓ | ✓ | ✓ | ✓ | ✓ |
| pharmaceutically | | | | | |
| acceptable excipients | | | | | |

Various oromucosal tablet compositions comprising dexmedetomidine hydrochloride at doses of 20 μg, 30 μg, 40 μg, 60 μg, 90 μg, 120 μg, 180 μg and 240 μg and excipients as described in table 5.

TABLE 5
Oromucosal tablet formulation embodiments
according to the disclosure.

| | Oromucosal Tablet Formulation Embodiment No. | | | | | | | | | |
|---|---|---|---|---|---|---|---|---|---|---|
| Ingredients | 1 | 2 | 3 | 4 | 5 | 6 | 7 | 8 | 9 | 10 |
| Lactose | ✓ | ✓ | ✓ | ✓ | ✓ | ✓ | ✓ | ✓ | ✓ | ✓ |
| Monohydrate | | | | | | | | | | |
| Hydroxypropyl | ✓ | ✓ | | | | | | | | |
| methylcellulose | | | | | | | | | | |
| Hydroxypropyl | | | ✓ | ✓ | | | | | | |
| cellulose | | | | | | | | | | |
| Croscarmellose | ✓ | | ✓ | | ✓ | | ✓ | | ✓ | |
| Sodium | | | | | | | | | | |
| Sodium starch | | ✓ | | ✓ | | ✓ | | ✓ | | ✓ |
| glycolate | | | | | | | | | | |
| Polyethylene oxide | | | | | ✓ | ✓ | | | | |
| Xanthan gum | | | | | | | ✓ | ✓ | | |
| Sodium alginate | | | | | | | | | ✓ | ✓ |
| Sucralose | ✓ | ✓ | ✓ | ✓ | ✓ | ✓ | ✓ | ✓ | ✓ | ✓ |
| Magnesium stearate | ✓ | ✓ | ✓ | ✓ | ✓ | ✓ | ✓ | ✓ | ✓ | ✓ |
| Optionally other | ✓ | ✓ | ✓ | ✓ | ✓ | ✓ | ✓ | ✓ | ✓ | ✓ |
| pharmaceutically | | | | | | | | | | |
| acceptable excipients | | | | | | | | | | |

### Intranasal Formulations

The compositions of the disclosure may be administered to the nasal cavity in any suitable form. For example, the composition may be administered to the nasal cavity in the form of a spray emulsion, suspension or solution, as drops or as a powder.

A powder blend according to the present disclosure may be prepared by mixing dexmedetomidine or a pharmaceutically acceptable salt thereof with inert ingredients that are standard in the art. Such inert ingredients include, but are not limited to diluents such as calcium phosphate, lactose, sugars such as dextrose and sucrose, polyols such as mannitol and sorbitol, and microcrystalline cellulose, glidants such as colloidal silica and lubricants such as magnesium stearate and hydrogenated vegetable oil and surfactants such as polysorbates; and polyethylene glycol. For preparing a uniform powder blend on a small scale, a pestle and mortar and/or sieve may be appropriate whereas mechanical mixers are required for larger scale manufacture. There are numerous types of mixers available and these are widely described in the literature, for example Chapter 37, Remington: The Science and Practice of Pharmacy, 20 Edition, Lipincott, Williams and Wilkins, Baltimore, 2000.

If the powder composition of the disclosure comprises granules, these granules may be produced by techniques well known to those skilled in the art such as wet granulation, dry granulation (slugging), extrusion/spheronisation, fluid bed granulation and spray congealing. Further details on granulation processes may be found in the literature, for example Chapter 6, Pharmaceutical Principles of Solid Dosage Forms, J. T. Carstensen, Technomic, Lancaster, Pa., 1993.

In addition to dexmedetomidine or a pharmaceutically acceptable salt thereof, other ingredients may be incorporated into the granules. Such other ingredients include, but are not limited to diluents such as calcium phosphate, lactose, dextrose, mannitol and microcrystalline cellulose, binders such as povidone (polyvinylpyrrolidone), methylcellulose, polyethylene glycol, gelatin and acacia, disintegrants such as starch, croscarmellose and crospovidone, glidants such as colloidal silica, and lubricants such as magnesium stearate and hydrogenated vegetable oil. Methods for preparation of microspheres are well known to those skilled in the art and include, but are not limited to, spray drying, interfacial polymerisation, coarcervation/phase separation and solvent evaporation. Methods for producing microspheres are described in, for example, Physicochemical Principles of Pharmacy, 3rd Edition, pages 357 to 360, A T Florence and D Attwood, Macmillan, London, 1998 and Physical Pharmacy, 4th Edition, pages 516 to 519, A Martin, Wilkins and Wilkins, Baltimore, 1993. The microspheres may alternatively be produced using the methods described in W098/30207 and the documents cited therein.

The powder compositions of the present disclosure may be administered to the subject in aerosolized form whereby energy from patient inhalation (sniffing) is used to aerosolize the powder into the nasal cavity or where the device itself provides the aerosolization energy, such as via compressed air. An example of the former device is manufactured by Pfeiffer and an example of the latter is the “Monopowder” manufactured by Valois. The present disclosure also provides a nasal drug delivery device or a dose cartridge for use in a nasal delivery device loaded with a composition as defined above.

In embodiments, the compositions of the disclosure also disclose the process for preparing the solutions of the disclosure comprises mixing the components in a suitable solvent such as water, ethanol, propylene glycol, polyethylene glycol, glycofurol, benzyl benzoate and polyoxyethylene castor oil derivatives. The compositions may be prepared using methods known in the art.

The solutions of the present disclosure may also contain other pharmaceutically acceptable ingredients well known in the art. Such ingredients include, but are not limited to, thickening, adhesive or gelling agents, such as, but are not limited to, celluloses (e.g. hydroxypropyl methylcellulose, methylcellulose, hydroxypropyl cellulose and microcrystalline cellulose), carbomers, polyethylene oxide, poloxamers or polyethylene glycols, antioxidants (for example sodium metabisulphite), chelating agents (such as edetic acid or one of its salts), preservatives (such as potassium sorbate, parabens, phenylethyl alcohol or benzalkonium chloride), flavors, sweeteners, thickening, adhesive or gelling agents, including, but are not limited to, celluloses such as hydroxypropyl methylcellulose, methylcellulose, hydroxypropyl cellulose, sodium carboxyl cellulose and microcrystalline cellulose, poloxamers, polyethylene glycols, carbomers or polyethylene oxide.

The solutions of the disclosure may contain a preservative and/or are sterile. If preservatives are omitted from the compositions, microorganisms may be removed using any suitable method known in the art, for example by making the compositions aseptically or by terminally sterilizing them. In some embodiments, the compositions of the invention are non-pyrogenic.

In embodiments, intranasal compositions of the present disclosure comprise aqueous suspension, solution, or emulsion containing materials in addition to the active ingredient, such as suitable dispersant and/or wetting agent, for example propylene glycol or polyethylene glycol, emulsifier, suspending agent, surfactant, solubilizer, vehicle etc.

The pharmaceutical composition may also be formulated as liposomes, microcapsules or centrosomes, with one or more suitable pharmaceutically acceptable carrier.

In addition to dexmedetomidine or a pharmaceutically acceptable salt thereof, microspheres used in the present disclosure may include ingredients that are known in the art to be suitable to be included in microspheres such as, but are not limited to, starches, dextrans, gelatin, albumin, collagen, hyaluronic acid, chitosan, lactose, sucrose, dextrose, mannitol, methacrylate copolymers such as the Eudragit® polymers (Degussa, Germany), celluloses such as methylcellulose, and polyesters such as poly(lactide-co-glycolide).

Any device that is suitable for intranasal administration can be used. In embodiments, the device is a metered dose device. Examples of a metered dose device include, but are not limited to, a spray pump, a pre-compression nasal spray pump, a metered valve device, an actuated spray device, a side actuated spray device, a syringe nasal spray device (e.g. a syringe that has an atomizer to deliver a spray to the nasal cavity), a mucosal atomization device, an electromechanical pump device (with and without a counter), and the like. Examples of metered dose devices also include, but are not limited to, devices manufactured by Aptar Pharma (Congers, N.Y.) and are commercially available. Examples of metered dose devices also include, but are not limited to, UDS (Aptar Pharma), BDS (Aptar Pharma), eDevices (Aptar Pharma), Equadel (Aptar Pharma), Latitude (Aptar Pharma), DF30 (Aptar Pharma), VP7 (Aptar Pharma), Classic Nasal Device (Aptar Pharma), MAD Nasal Drug Device (Wolf Tory Medical, Inc.), BD Accuspray SCF™ (Becton Dickinson), and the like. Another example includes, but is not limited to, an Aptar Unitdose Intranasal System.

### Parenteral Formulations

Liquid pharmaceutical compositions for parenteral administration may be formulated for administration by injection or continuous infusion. Routes of administration by injection or infusion can include, but are not limited to, intravenous, intraperitoneal, intramuscular, intrathecal, and subcutaneous. In embodiments, parenteral formulations can include prefilled syringes, vials, powder for infusion for reconstitution, concentrate for infusion to be diluted before delivery (ready to dilute) or solutions (ready to use).

Injectable pharmaceutical compositions can be aqueous isotonic solutions or suspensions, and suppositories can be prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

In embodiments, the pharmaceutical compositions of the present disclosure include biodegradable subcutaneous implant, osmotically controlled device, subcutaneous implant, subcutaneous sustained release injection, lipid nanoparticles, liposomes, and the like. Liquid preparations can include, but are not limited to, solutions, suspensions and emulsions. Such preparations are exemplified by water or water/propylene glycol solutions for parenteral injection. Liquid preparations may also include solutions for intranasal administration.

For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled to render the preparation isotonic.

The liquid vehicle used for the preparation of the intramuscular injection may be, for example, water, a saline solution, another aqueous liquid (aqueous solvent) or non-aqueous liquid (non-aqueous solvent).

The parenteral formulations of the present disclosure can be sterilized. Non-limiting examples of sterilization techniques include filtration through a bacterial-retaining filter, terminal sterilization, incorporation of sterilizing agents, irradiation, and heating.

Administration of the above-described parenteral formulations may be by periodic injections of a bolus of the preparation, or may be administered by intravenous or intraperitoneal administration from a reservoir which is external (e.g., an intravenous bag) or internal (e.g., a bioerodable implant, a bioartificial or organ). See, e.g., U.S. Pat. Nos. 4,407,957 and 5,798,113, each incorporated herein by reference in their entireties. Intrapulmonary delivery methods and apparatus are described, for example, in U.S. Pat. Nos. 5,654,007, 5,780,014, and 5,814,607, each incorporated herein by reference in their entireties. Other useful parenteral delivery systems include ethylene-vinyl acetate copolymer particles, osmotic pumps, implantable infusion systems, pump delivery, encapsulated cell delivery, liposomal delivery, needle-delivered injection, needle-less injection, nebulizer, aerosolizer, electroporation, and transdermal patch. Needle-less injector devices are described in U.S. Pat. Nos. 5,879,327; 5,520,639; 5,846,233 and 5,704,911, the specifications of which are herein incorporated herein by reference in their entireties. Any of the formulations described herein can be administered in these methods. Further injectable formulations of dexmedetomidine are disclosed in U.S. Pat. Nos. 8,242,158, 9,649,296, JP. Patent No. 5,921, 928, JP. Pat. Appl. No. 2016154598, CN Pat. Appl. No. 103284945, CN Pat. Appl. No. 104161760, CN Pat. Appl. No. 105168122, CN Pat. Appl. No. 105534891, CN Pat. Appl. No. 106038538, U.S. Pat. Appl. No. 20170128421, CN Pat. Appl. No. 107028880, CN Pat. Appl. No. 107412152, CN Pat. Appl. No. 108498469, EP Patent. No. 2252290, JP. Pat. Appl. No. 2019048091 and U.S. Pat. Appl. No. 20190183729.

The present disclosure includes intramuscular compositions comprising: dexmedetomidine, or a pharmaceutically acceptable salt thereof, at a concentration of between about 0.05 μg/mL and about 15 μg/mL, sodium chloride at a concentration of between about 0.01 and about 2.0 weight percent and pH in the range of about 1 to about 10.

### Oral Formulations

The present disclosure includes oral formulations that can be used for delivering dexmedetomidine. Examples of oral formulations includes tablets, orally disintegrating tablets, mouth dissolving tablets, wafers, solution, suspension, emulsions, and capsules.

The disclosure encompasses oral disintegrating tablets comprising dexmedetomidine or a pharmaceutically acceptable salt thereof and at least one orally disintegrating carrier, wherein the oral disintegrating tablet disintegrates in about 0.5 to about 120 seconds and/or a therapeutically effective amount of the dexmedetomidine is absorbed into the bloodstream within about 1 to about 5 minutes. In embodiments, a therapeutically effective amount of the dexmedetomidine is absorbed into the bloodstream within about 3 minutes.

In embodiments, the at least one orally disintegrating carrier is selected from the group consisting of water-soluble sugars or sugar alcohol, crospovidone, (low-substituted) hydroxypropyl cellulose, croscarmellose sodium, microcrystalline cellulose, lactose, pregelatinized starch, sodium starch glycolate, sodium lauryl sulphate, crystalline cellulose and the combination thereof. The water-soluble sugars or sugar alcohol is selected from the group consisting of sucrose, sorbitol, mannitol, xylitol, erythritol, isomalt and fructose. In embodiments, the orally disintegrating carriers together constitute at least 50 wt. %, for example at least 80 wt. % or at least 85 wt. % of the orally disintegrating carriers. The aforementioned carriers are in the form of particles typically have a volume weighted mean particle size of 50-300 micrometers, for example of 70-200 micrometers. F-Melt® (Fuji Chemical Industry Co.) is an example of a commercially available particulate material that contains a disintegrating agent dispersed in a matrix containing C4-C6 sugar alcohol (mannitol and xylitol). Ludiflash® (BASF) is another example of a commercially available particulate material that contains a disintegrating agent dispersed in a matrix of C4-C6 sugar alcohol (mannitol).

The orally disintegrating tablet as used herein may be prepared by mixing the dexmedetomidine with water-soluble diluents and compressed in a tablet. A suspension comprising dexmedetomidine may be prepared with appropriate excipients and the dexmedetomidine suspension may be dispensed into blister packs and freeze-dried. An exemplary freeze-dried preparation platform that could be used for the dexmedetomidine ODT is the ZYDIS® (Catalent, Somerset, N.J., USA) formulation. In particular, the excipients, including water, are blended and the dexmedetomidine is separately milled to size and mixed with the excipients. The suspension then undergoes lyophilization by flash freezing and freeze drying. Other methods of preparing ODTs may be used without limitation, and detailed description of general methods thereof have been disclosed, for example, in U.S. Pat. Nos. 5,631,023; 5,837,287; 6,149,938; 6,212,791; 6,284,270; 6,316,029; 6,465,010; 6,471,992; 6,471,992; 6,509,040; 6,814,978; 6,908,626; 6,908,626; 6,982,251; 7,282,217; 7,425,341; 7,939,105; 7,993,674; 8,048,449; 8,127,516; 8,158,152; 8,221,480; 8,256,233; and 8,313,768, each of which is incorporated herein by reference in its entirety.

### Specific Embodiments

Embodiment 1. A method of treating agitation or signs of agitation in elderly patients having dementia, comprising administering dexmedetomidine or a pharmaceutically acceptable salt thereof to the patient in an agitated state at a dose sufficient to provide a Cₘₐₓ in a range of about 80% to about 125% of about 50 ng/L to about 500 ng/L, wherein the patient is 65 years old or older.

Embodiment 2. A method of treating an acute agitation episode in an elderly dementia patient, comprising administering dexmedetomidine, or a pharmaceutically acceptable salt thereof, to the agitated patient at a dose sufficient to provide AUC_{0-inf} in a range about 80% to about 125% of about 200 hr*ng/L to about 2200 hr*ng/L, wherein the Cₘₐₓ is in a range of about 80% to about 125% of about 50 ng/L to about 300 ng/L and the patient is 65 years old or older.

Embodiment 3. A method of treating an acute agitation episode in an elderly dementia patient, comprising administering dexmedetomidine, or a pharmaceutically acceptable salt thereof, to the agitated patient at a dose sufficient to provide AUC₀₋₈ in a range about 80% to about 125% of about 200 hr*ng/L to about 1500 hr*ng/L; wherein the Cₘₐₓ is in a range of about 80% to about 125% of about 50 ng/L to about 300 ng/L; and the patient is 65 years old or older.

Embodiment 4. The method of any of embodiments 1 to 3, wherein the dose of dexmedetomidine or a pharmaceutically acceptable salt thereof is about 30 μg to about 90 μg (for example, about 30 μg to about 60 μg; 60 μg to about 90 μg, or 30 μg to about 45 μg).

Embodiment 5. The method of embodiment 4, wherein the dose of dexmedetomidine or a pharmaceutically acceptable salt thereof is about 30 μg, about 40 μg, about 45 μg, about 50 μg, about 60 μg, about 75 μg, about 80 μg or about 90 μg.

Embodiment 6. The method of any of embodiments 1 to 3, wherein the route of administration is oromucosal, and the oromucosal includes sublingual, buccal or gingival.

Embodiment 7. The method of any of embodiments 1 to 6, comprises oromucosally administering about 30 μg of dexmedetomidine or a pharmaceutically acceptable salt thereof one to six times a day at a dosing interval of at least 2 hours. Embodiment 8. The method of any of embodiments 1 to 6, comprises oromucosally administering about 40 μg of dexmedetomidine or a pharmaceutically acceptable salt thereof one to six times a day at a dosing interval of at least 2 hours.

Embodiment 9. The method of any of embodiments 1 to 6, comprises oromucosally administering about 60 μg of dexmedetomidine or a pharmaceutically acceptable salt thereof one to six times a day at a dosing interval of at least 2 hours.

Embodiment 10. The method of any of embodiments 1 to 6, comprises oromucosally administering about 90 μg of dexmedetomidine or a pharmaceutically acceptable salt thereof one to four times a day at a dosing interval of at least 2 hours.

Embodiment 11. The method of any of embodiments 1 to 6, comprises oromucosally administering a single dose of about 30 μg of dexmedetomidine or a pharmaceutically acceptable salt thereof followed by 60 μg dose of dexmedetomidine or a pharmaceutically acceptable salt thereof after 2 hours.

Embodiment 12. The method of any of embodiments 1 to 6, comprises oromucosally administering a single dose of about 30 μg of dexmedetomidine or a pharmaceutically acceptable salt thereof followed by 60 μg dose of dexmedetomidine or a pharmaceutically acceptable salt thereof after 6 hours.

Embodiment 13. The method of embodiment 1 to 3, wherein the elderly patient is about 75 to about 80 years old.

Embodiment 14. The method of embodiment 1 to 3, wherein the elderly patient is about 80 years old or older.

Embodiment 15. A method of treating an acute agitation episode in an elderly dementia patient, comprising administering dexmedetomidine, or a pharmaceutically acceptable salt thereof, to the agitated patient at a dose sufficient to provide AUC_{0-inf} in a range of about 80% to about 125% of about 200 hr*ng/L to about 2200 hr*ng/L; wherein the Cₘₐₓ is in a range of about 80% to about 125% of about 50 ng/L to about 300 ng/L; and the route of administration is selected from oromucosal, intravenous, intramuscular, subcutaneous, and transdermal.

Embodiment 16. The method of embodiment 15, wherein the dexmedetomidine or a pharmaceutically acceptable salt thereof is administered at a dose of about 30 μg to about 130μg.

Embodiment 17. The method of embodiment 15 or 16, wherein AUC₀₋₈ is 200 hr*ng/L to about 1500 hr*ng/L (for example, about 200 hr*ng/L to about 1250 hr*ng/L, about 200 hr*ng/L to about 1000 hr*ng/L, about 200 hr*ng/L to about 750 hr*ng/L, about 200 hr*ng/L to about 500 hr*ng/L, about 500 hr*ng/L to about 1500 hr*ng/L, about 500 hr*ng/L to about 1250 hr*ng/L, about 500 hr*ng/L to about 1000 hr*ng/L, about 500 hr*ng/L to about 750 hr*ng/L, about 750 hr*ng/L to about 1500 hr*ng/L, about 750 hr*ng/L to about 1250 hr*ng/L, about 750 hr*ng/L to about 1000 hr*ng/L, about 1000 hr*ng/L to about 1500 hr*ng/L.).

Embodiment 18. The method of any of embodiments 15 to 17, wherein the dexmedetomidine or a pharmaceutically acceptable salt thereof is administered at a dose of about 30 μg, the Cₘₐₓ is about 50 ng/L to about 150 ng/L and the AUC₀₋₈ range is between about 200 hr*ng/L to about 600 hr*ng/L(for example, about 200 hr*ng/L to about 400 hr*ng/L, about 300 hr*ng/L to about 600 hr*ng/L, about 300 hr*ng/L to about 500 hr*ng/L, about 350 hr*ng/L to about 450 hr*ng/L).

Embodiment 19. The method of any of embodiments 15 to 17, wherein the dexmedetomidine or a pharmaceutically acceptable salt thereof is administered at a dose of about 40 μg, the Cₘₐₓ is about 50 ng/L to about 250 ng/L and the AUC₀₋₈ range is between about 200 hr*ng/L to about 600 hr*ng/L (for example, about 200 hr*ng/L to about 400 hr*ng/L, about 300 hr*ng/L to about 600 hr*ng/L, about 300 hr*ng/L to about 500 hr*ng/L, about 350 hr*ng/L to about 450 hr*ng/L).

Embodiment 20. The method of embodiment 15 to 17, wherein the dexmedetomidine or a pharmaceutically acceptable salt thereof is administered at a dose of about 45 μg, the Cₘₐₓ is about 75 ng/L to about 175 ng/L and the AUC₀₋₈ range is between about 500 hr*ng/L to about 900 hr*ng/L (for example, about 500 hr*ng/L to about 800 hr*ng/L, about 600 hr*ng/L to about 900 hr*ng/L, about 600 hr*ng/L to about 800 hr*ng/L, about 650 hr*ng/L to about 750 hr*ng/L).Embodiment 21. The method of embodiment 15 to 17, wherein the dexmedetomidine or a pharmaceutically acceptable salt thereof is administered at a dose of about 60 μg ,the Cₘₐₓ is about 100 ng/L to about 250 ng/L and the AUC₀₋₈ range is between about 500 hr*ng/L to about 1500 hr*ng/L (for example, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L).

Embodiment 22. The method of embodiment 15 to 17, wherein the dexmedetomidine or a pharmaceutically acceptable salt thereof is administered at a dose of about 90 μg, the Cₘₐₓ is about 100 ng/L to 400 ng/L and the AUC₀₋₈ range is between about 500 hr*ng/L to about 1500 hr*ng/L. (for example, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L).

Embodiment 23. The method of embodiment 18, wherein the administration of dexmedetomidine or a pharmaceutically acceptable salt thereof results in about 80% to about 125% of AUC_{0-inf} range of about 200 hr*ng/L to about 1000 hr*ng/L. (for example, about 200 hr*ng/L to about 900 hr*ng/L, about 300 hr*ng/L to about 800 hr*ng/L, about 300 hr*ng/L to about 750 hr*ng/L, about 350 hr*ng/L to about 750 hr*ng/L).

Embodiment 24. The method of embodiment 19, wherein the administration of dexmedetomidine or a pharmaceutically acceptable salt thereof results in about 80% to about 125% of AUC_{0-inf} range of about 300 hr*ng/L to about 2200 hr*ng/L, (for example, about 400 hr*ng/L to about 2000 hr*ng/L, about 400 hr*ng/L to about 1800 hr*ng/L, about 500 hr*ng/L to about 1500 hr*ng/L, for example, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L).

Embodiment 25. The method of embodiment 20, wherein the administration of dexmedetomidine or a pharmaceutically acceptable salt thereof results in about 80% to about 125% of AUC_{0-inf} range of about 500 hr*ng/L to about 1500 hr*ng/L. (for example, about 500 hr*ng/L to about 1400 hr*ng/L, about 500 hr*ng/L to about 1000 hr*ng/L, about 600 hr*ng/L to about 1400 hr*ng/L, about 600 hr*ng/L to about 1200 hr*ng/L, about 600 hr*ng/L to about 1000 hr*ng/L, about 800 hr*ng/L to about 1400 hr*ng/L, or about 800 hr*ng/L to about 1000 hr*ng/L)

Embodiment 26. The method of embodiment 21, wherein the administration of dexmedetomidine or a pharmaceutically acceptable salt thereof results in about 80% to about 125% of AUC_{0-inf} range of about 80% to about 125% of about 500 hr*ng/L to about 2000 hr*ng/L.(for example, about 600 hr*ng/L to about 1900 hr*ng/L, about 700 hr*ng/L to about 1800 hr*ng/L, about 700 hr*ng/L to about 1700 hr*ng/L, about 700 hr*ng/L to about 1600 hr*ng/L, about 700 hr*ng/L to about 1500 hr*ng/L, about 800 hr*ng/L to about 1500 hr*ng/L, about 900 hr*ng/L to about 1500, about 1000 hr*ng/L to about 1500 hr*ng/L, about 1100 hr*ng/L to about 1500 hr*ng/L, about 1200 hr*ng/L to about 1500 hr*ng/L, about 1300 hr*ng/L to about 1500 hr*ng/L, or about 1400 hr*ng/L to about 1500 hr*ng/L).

Embodiment 27. The method of embodiment 18, wherein the Cₘₐₓ is about 80% to about 125% of about 50 ng/L to about 150 ng/L, about 50 ng/L to about 125 ng/L, about 50 ng/L to about 100 ng/L, about 50 ng/L to about 75 ng/L, about 75 ng/L to about 150 ng/L, about 75 ng/L to about 125 ng/L, about 75 ng/L to about 100 ng/L, about 100 ng/L to about 150 ng/L.

Embodiment 28. The method of embodiment 19, wherein the Cₘₐₓ is about 80% to about 125% of about 50 ng/L to about 250 ng/L, for example, about 50 ng/L to about 225 ng/L, about 50 ng/L to about 200 ng/L, about 100 ng/L to about 180 ng/L, about 100 ng/L to about 150 ng/L, about 150 ng/L to about 200 ng/L.

Embodiment 29. The method of embodiment 20, wherein the Cₘₐₓ is about 80% to about 125% of about 75 ng/L to about 150 ng/L, about 75 ng/L to about 125 ng/L, about 75 ng/L to about 100 ng/L, about 100 ng/L to about 150 ng/L.

Embodiment 30. The method of embodiment 21, wherein the Cₘₐₓ is about 80% to about 125% of about 100 ng/L to about 250 ng/L, about 100 ng/L to about 225 ng/L, about 100 ng/L to about 200 ng/L, about 150 ng/L to about 250 ng/L, about 150 ng/L to about 200 ng/L.

Embodiment 31. The method of embodiment 22, wherein the Cₘₐₓ is about 80% to about 125% of about 100 ng/L to about 400 ng/L, about 100 ng/L to about 350 ng/L, about 100 ng/L to about 300 ng/L, about 200 ng/L to about 400 ng/L, about 200 ng/L to about 350 ng/L

Embodiment 32. The method of embodiment 15, wherein the elderly patient has both dementia and Alzheimer's disease.

Embodiment 33. The method of any of embodiments 1 to 32, wherein the patient is not significantly sedated within 60 minutes after dexmedetomidine pharmaceutically acceptable salt thereof administration.

Embodiment 34. The method of any of embodiments 1 to 32, wherein the Cₘₐₓ values and ranges are at least 30% higher as compared to that obtained in schizophrenia and bipolar disorder patients.

Embodiment 35. The method of any of embodiments 1 to 32, wherein the Cₘₐₓ value is about 35% higher as compared to that obtained in schizophrenia and bipolar disorder patients.

Embodiment 36. The method of any of embodiments 1 to 32, wherein the AUC values and ranges are at least 50% higher as compared to that obtained in schizophrenia and bipolar disorder patients.

Embodiment 37. The method of any of embodiments 1 to 32, wherein the AUC values and ranges are about 55% higher as compared to that obtained in schizophrenia and bipolar disorder patients.

Embodiment 38. The method of any of embodiments 1 to 32, wherein the Cₘₐₓ, AUC_{0-∞} and AUC₀₋₈ ranges and values are about 40% and 60% higher compared to that obtained in schizophrenia and bipolar disorder patients.

Embodiment 39. The method of any of embodiments 1 to 38, wherein the reduction of agitation in the elderly dementia patients is assessed using PEC, PAS, ACES, Mod-CMAI, and/or CGI-I.

Embodiment 40. The method of any of embodiments 1 to 39, wherein the agitation or signs of agitation are significantly reduced within 60 minutes of administering dexmedetomidine or a pharmaceutically acceptable salt thereof.

Embodiment 41. The method of embodiment 40, wherein reduction in agitation is maintained for about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

Embodiment 42. The method of any of embodiments 1 to 38, wherein the patient achieves a mean change in PAS score of greater than −2 relative to baseline within 2 hours of dexmedetomidine administration.

Embodiment 43. The method of any of embodiments 1 to 42, wherein the patient achieves a mean change in PEC score of greater than −2 relative to baseline within 2 hours of dexmedetomidine administration.

Embodiment 44. The method of embodiment 43, wherein the decrease in PEC score is maintained for at least 2, 3, 4, 5, 6, 7, 8, 9, 10, 11, or 12, hours following administration of dexmedetomidine.

Embodiment 44. The method of any of embodiments 1 to 41, wherein the patient achieves a mean change in mod-CMAI score of greater than −7 relative to baseline after 2 hours of dexmedetomidine administration.

Embodiment 45. The method of embodiment 44, wherein decrease in mod-CMAI score is maintained for at least 2 (including for e.g. 3, 4, 5, 6, 7, 8, 9, 10, 11, or 12) hours following administration of dexmedetomidine.

Embodiment 46. The method of any of embodiments 1 to 41, wherein the patient achieves a CGI-I score improvement to about 1 (very much improved) or about 2 (much improved).

Embodiment 47. The method of embodiment 46, wherein the score improvement is sustained for a period of about 2 hours to about 6 hours.

Embodiment 48. The method of embodiment 46, wherein the score improvement is sustained for a period of about 12 hours.

Embodiment 49. The method of any of embodiments 1 to 41, wherein the agitation is reduced to a 2 (moderate agitation), 3 (mild agitation) or 4 (normal behavior) 2 hours after administering the composition, as measured by the Agitation-Calmness Evaluation Scale (ACES).

Embodiment 50. The method of embodiment 49, wherein the agitation is reduced to a 3 (mild agitation).

Embodiment 51. The method of embodiment 16, wherein the patient has not received treatment for hypertension within at least 10 hours prior to dexmedetomidine administration.

Embodiment 52. The method of any of embodiments 1 to 51, wherein agitation is acute agitation.

Embodiment 53. The method of any of embodiments 1 to 51, wherein agitation is chronic agitation.

Embodiment 54. A method of administering doses higher than about 90 μg of dexmedetomidine or a pharmaceutically acceptable salt thereof to treat agitation in elderly dementia patients who have not received a hypertension treatment for at least 10 hours, at least 24 hours, at least 48 hours, or at least a week, prior to administering dexmedetomidine.

Embodiment 55. The method of any of embodiments 1 to 54, wherein dexmedetomidine or a pharmaceutically acceptable salt thereof is administered sublingually or buccally.

Embodiment 56. The method of embodiment 55, wherein dexmedetomidine or a pharmaceutically acceptable salt thereof is administered sublingually in the form of a tablet, film, spray, gel or drops.

Embodiment 58. The method of embodiment 56, wherein dexmedetomidine or a pharmaceutically acceptable salt thereof is administered sublingually in the form of a film.

Embodiment 59. The method of any of embodiments 1 to 58, wherein dexmedetomidine or a pharmaceutically acceptable salt thereof is administered buccally.

Embodiment 60. The method of embodiment 59, wherein dexmedetomidine or a pharmaceutically acceptable salt thereof is administered buccally in the form of a tablet, film, spray, gel or drops.

Embodiment 61. The method of embodiment 60, wherein dexmedetomidine or a pharmaceutically acceptable salt thereof is administered buccally in the form of a film.

Embodiment 62. The method of any of preceding embodiments, wherein the patient is treated without also inducing clinically significant cardiovascular effects.

Embodiment 63. The method of any of preceding embodiments, wherein dexmedetomidine or a pharmaceutically acceptable salt thereof is dexmedetomidine hydrochloride.

Embodiment 64. A method of treating agitation or signs of agitation in a human subject with delirium hospitalized in ICU, without also inducing significant sedation, comprising oromucosally administering about 20 μg of dexmedetomidine or a pharmaceutically acceptable salt one to four times within 6 hours of first dose at a dosing interval of at least 30 minutes thereof.

Embodiment 65. A method of treating agitation or signs of agitation in a human subject with delirium hospitalized in ICU, without also inducing significant sedation, comprising oromucosally administering about 20 μg of dexmedetomidine or a pharmaceutically acceptable salt one to four times within 6 hours of first dose at a dosing interval of at least 30 minutes thereof.

Embodiment 66. A method of treating agitation or signs of agitation in a human subject with delirium hospitalized in ICU, without also inducing significant sedation, comprising oromucosally administering about 40 μg of dexmedetomidine or a pharmaceutically acceptable salt one to four times within 6 hours of first dose at a dosing interval of at least 30 minutes thereof.

Embodiment 67. A method of treating agitation or signs of agitation in a human subject with delirium hospitalized in ICU, without also inducing significant sedation, comprising oromucosally administering about 60 μg of dexmedetomidine or a pharmaceutically acceptable salt thereof one to four times within 6 hours of first dose at a dosing interval of at least 30 minutes.

Embodiment 68. A method of treating agitation or signs of agitation in a human subject with delirium hospitalized in ICU, without also inducing significant sedation, comprising oromucosally (e.g. sublingually or buccally) administering about 20 μg to about 300 μg of dexmedetomidine or a pharmaceutically acceptable salt.

Embodiment 69. The method of embodiment 68, wherein the agitation or signs of agitation and delirium severity are significantly reduced as measured by RASS and DRS-R-98 respectively

Embodiment 70. The method of embodiment 68, wherein the subject achieves a 2-point or greater drop in RASS at 2 hours.

Embodiment 71. The method of embodiment 68, wherein dexmedetomidine or a pharmaceutically acceptable salt is administered at a dose of about 20 μg, 60 μg, 80 μg, 90 μg, 100 μg, 120 μg, 150 μg, 180 μg, 210 μg, 240 μg, 270 μg, or 300 μg.

Embodiment 72. The method of embodiment 68, wherein dexmedetomidine or a pharmaceutically acceptable salt is administered at a dose of about 270 μg.

Embodiment 73. The method of embodiment 68, wherein the dose of dexmedetomidine or a pharmaceutically acceptable salt is about 300 μg.

Embodiment 74. The method of embodiment 68, wherein the subject's initial RASS is not less than or equal to −3.

Embodiment 75. The method of embodiment 68, wherein the dexmedetomidine or a pharmaceutically acceptable salt is administered as a single unit dose or multiple unit dose.

Embodiment 76. The method of embodiment 68, wherein dexmedetomidine or a pharmaceutically acceptable salt is administered one to ten times a day at an interval of about 1-6 hours of first dose to produce desired effect.

Embodiment 77. The method of embodiment 68, wherein dexmedetomidine or a pharmaceutically acceptable salt is administered twice a day.

Embodiment 78. The method of embodiment 68, wherein about 120 μg dose of dexmedetomidine or a pharmaceutically acceptable salt is administered two times a day at an interval of 12 hours.

Embodiment 79. The method of embodiment 68, wherein about 120 μg dose of dexmedetomidine or a pharmaceutically acceptable salt is administered seven times a day at an interval of about 1 to 6 hours to produce a maximum cumulative dose of 960 μg.

Embodiment 80. The method of embodiment 68, wherein about 180 μg dose of dexmedetomidine or a pharmaceutically acceptable salt is administered followed by additional six doses of 120 μg at an interval of about 1 to 6 hours

Embodiment 81. The method of embodiment 68, wherein about 240 μg dose of dexmedetomidine or a pharmaceutically acceptable salt is administered followed by an additional six doses of 120 μg in a day at an interval of about 1 to about 6 hours to produce the maximum cumulative dose of 960 μg.

Embodiment 82. The method of embodiment 68, wherein about 300 μg dose of dexmedetomidine or a pharmaceutically acceptable salt is administered followed by an additional five doses of 120 μg in a day at an interval of about 1 to about 6 hours to produce the maximum cumulative dose of 900 μg.Embodiment 83. The method of any of embodiments 64 to 82, wherein dexmedetomidine or a pharmaceutically acceptable salt thereof is administered sublingually in the form of a tablet, film, spray, gel or drops.

Embodiment 84. The method of embodiment 83, wherein dexmedetomidine or a pharmaceutically acceptable salt thereof is administered sublingually as a film.

Embodiment 85. The method of any of embodiments 64 to 82, wherein dexmedetomidine or a pharmaceutically acceptable salt thereof is administered buccally in the form of a tablet, film, spray, gel or drops.

Embodiment 86. The method of embodiment 85, wherein dexmedetomidine or a pharmaceutically acceptable salt thereof is administered buccally as a film.

Embodiment 87. The method of any of embodiments 64 to 82, wherein the subject is 18-64 years old.

Embodiment 88. The method of any of embodiments 64 to 82, wherein the subject is above 64 years old.

Embodiment 89. A method of reducing a period of opioid withdrawal by administering dexmedetomidine or a pharmaceutically acceptable salt thereof (e.g. dexmedetomidine hydrochloride) twice daily to a human subject of at least 18 years old in need thereof for the period of withdrawal, wherein the period of withdrawal is up to 14 days.

Embodiment 90. A method of treating or ameliorating opioid withdrawal symptoms, comprising administering a composition comprising dexmedetomidine or a pharmaceutically acceptable salt thereof to a human patient in need thereof, wherein the patient is at least 18 years old and wherein the period of withdrawal is up to 14 days.

Embodiment 91. The method of embodiment 90, wherein the treatment comprises reducing the period of opioid withdrawal.

Embodiment 92. The method of embodiment 91, wherein the opioid withdrawal symptom is agitation.

Embodiment 93. The method of embodiment 90, wherein the dexmedetomidine or a pharmaceutically acceptable salt thereof is administered at a dose range between 30 μg to about 600 μg.

Embodiment 94. The method of embodiment 93, wherein the composition comprises a dose range of dexmedetomidine or a pharmaceutically acceptable salt thereof between 30 μg to about 300 μg.

Embodiment 95. The method of embodiment 93 or 94, wherein the dexmedetomidine or a pharmaceutically acceptable salt thereof is administered at a unit dose of about 30 μg, 60 μg, 90 μg, 120 μg, 150 μg, 180 μg, 240 μg, 270 μg or 300 μg twice daily.

Embodiment 96. The method of embodiment 93 or 94 wherein the period of withdrawal is up to: 14 days, 13 days, 12 days, 11 days, 10 days, 9 days, 8 days, 7 days, 6 days, 5 days, 4 days, or 3 days.

Embodiment 97. The method of embodiment 93 or 94, wherein the composition is administered twice daily for 7 days.

Embodiment 98. The method of embodiment 93 or 94, wherein the opioid is selected from the group comprising of fentanyl, morphine, codeine, heroin, oxycodone, hydrocodone, alfentanil carfentanil, tramadol, hydromorphone, buprenorphine, naloxone, naltrexone, remifentanil butorphanol, meperidine, methadone, dextropropoxyphene (propoxyphene) thebaine, sufentanil or pentazocine.

Embodiment 99. The method of embodiment 93 or 94, wherein the opioid had been administered for amount of time longer than neonate treatment prior to withdrawal.

Embodiments 100. The method of embodiment 93 or 94, wherein improvement in the subject is assessed using a Clinical Opiate Withdrawal Scale (COWS) and/or the Short Opiate Withdrawal Scale (SOWS) of Gossop (e.g. over a 10-day period) after following the treatment.

Embodiment 101. The method of embodiment 93 or 94, wherein improvement in opioid withdrawal is measured in terms of retention (in days) and percentage of subjects dropping after discontinuation of opioid.

Embodiment 102. The method of embodiment 93 or 94, wherein the dexmedetomidine or a pharmaceutically acceptable salt thereof is administered at a dose of about 240 μg twice a day (in an interval of 12 hours).

Embodiment 103. The method of embodiment 98, wherein the opioid is fentanyl.

Embodiment 104. The method of embodiment 93 or 94, wherein the patient is 18 years old to 64 years old.

Embodiment 105. The method of any of embodiments 90 to 104, wherein retention rate of at least about 40% was observed at Day 6 post treatment with 120 μg dose of dexmedetomidine or a pharmaceutically acceptable salt thereof.

Embodiment 106. The method of any of embodiments 90 to 104, wherein retention rate of at least about 50% was observed at Day 6 post treatment with 180 μg dose of dexmedetomidine or a pharmaceutically acceptable salt thereof.

Embodiment 107. The method of any of embodiments 90 to 104, wherein significant reduction in subjective rating of insomnia is obtained on Day 7 as measured on SOWS scale after administration of about 240 μg dose of dexmedetomidine or a pharmaceutically acceptable salt thereof twice daily

Embodiment 108. The method of any of embodiments 90 to 104, wherein significant reduction in ratings of anxiety or irritability is obtained on Day 8 as measured on COWS scale after administration of about 240 μg dose of dexmedetomidine or a pharmaceutically acceptable salt thereof twice daily.

Embodiment 109. The method of embodiment 92, wherein the agitation is reduced to 3 (mild agitation) or 4 (normal behavior) and 5 (mild calmness) after administering dexmedetomidine or a pharmaceutically acceptable salt thereof as measured by the Agitation-Calmness Evaluation Scale (ACES).

Embodiment 110. The method of any of embodiments 89 to 109, wherein administration of dexmedetomidine or a pharmaceutically acceptable salt thereof provides the mean plasma concentrations in the range of about 40 ng/L to about 500 ng/L on day 6 after 2 hours of administration of dexmedetomidine or a pharmaceutically acceptable salt.

Embodiment 111. The method of any of embodiments 89 to 109, wherein administration of dexmedetomidine or a pharmaceutically acceptable salt thereof provides the mean plasma concentrations in the range of about 20 ng/L to about 200 ng/L on day 6 after 6 hours of administration of dexmedetomidine or a pharmaceutically acceptable salt (e.g. hydrochloride).

Embodiment 112. The method of any of embodiments 89 to 109, wherein administration of dexmedetomidine or a pharmaceutically acceptable salt thereof provides the mean plasma concentrations in the range of about 20 ng/L to about 150 ng/L on day 6 after 12 hours of administration of dexmedetomidine or a pharmaceutically acceptable salt (e.g. hydrochloride).

Embodiment 113. The method of any of embodiments 89 to 109, wherein administration of dexmedetomidine or a pharmaceutically acceptable salt thereof provides the mean plasma concentrations in the range of about 50 ng/L to about 500 ng/L on day 12 after 2 hours of administration of dexmedetomidine or a pharmaceutically acceptable salt (e.g. hydrochloride).

Embodiment 114. The method of any of embodiments 89 to 109, wherein administration of dexmedetomidine or a pharmaceutically acceptable salt thereof provides the mean plasma concentrations in the range of about 20 ng/L to about 250 ng/L on day 12 after 6 hours of administration of dexmedetomidine or a pharmaceutically acceptable salt (e.g. hydrochloride).

Embodiment 115. The method of any of embodiments 89 to 109, wherein administration of dexmedetomidine or a pharmaceutically acceptable salt thereof provides the mean plasma concentrations in the range of about 10 ng/L to 150 ng/L on day 12 after 12 hours of administration of dexmedetomidine or a pharmaceutically acceptable salt.

Embodiment 116. The method of any of embodiments 89 to 109, wherein the mean plasma concentrations are preferably 80% to 125% of these ranges and values.

Embodiment 117. The method of any of embodiment 89 or 90, wherein when the dose is 30 μg, the mean plasma concentrations are in the range of about 20 ng/L to about 50 ng/L (for example about 25 ng/L, about 30 ng/L, about 35 ng/L, about 40 ng/L and about 45 ng/L) post administration of dexmedetomidine or pharmaceutically acceptable salt thereof on day 6.

Embodiment 118. The method of any of embodiment 89 or 90, wherein when the dose is 60 μg, the mean plasma concentrations are in the range of about 25 ng/L to about 150 ng/L (for example about 25 ng/L, about 30 ng/L, about 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L) post administration of dexmedetomidine or pharmaceutically acceptable salt thereof on day 6.

Embodiment 119. The method of any of embodiment 89 or 90, wherein when the dose is 90 μg, the mean plasma concentrations are in the range of about 30 ng/L to about 150 ng/L (for example about 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L) post administration of dexmedetomidine or pharmaceutically acceptable salt thereof on day 6.

Embodiment 120. The method of any of embodiment 89 or 90, wherein when the dose is 120 μg, the mean plasma concentrations are in the range of about 50 ng/L to about 200 ng/L for example, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L and about 195 ng/L) post administration of dexmedetomidine or pharmaceutically acceptable salt thereof on day 6.

Embodiment 121. The method of any of embodiment 89 or 90, wherein when the dose is 180 μg, the mean plasma concentrations are in the range of about 100 ng/L to about 450 ng/L for example, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, about 400 ng/L, about 425 ng/L, about 450 ng/L) post administration of dexmedetomidine or pharmaceutically acceptable salt thereof on day 6.

Embodiment 122. The method of any of embodiment 89 or 90, wherein when the dose is 240 μg, the mean plasma concentrations are in the range of about 100 ng/L to about 400 ng/L for example, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, about 395 ng/L) post administration of dexmedetomidine or pharmaceutically acceptable salt thereof on day 6.

Embodiment 123. The method of any of embodiment 89 or 90, wherein when the dose is 30μg, the mean plasma concentrations are in the range of about 10 ng/L to about 100 ng/L (for example about 15 ng/L, about 20 ng/L, about 25 ng/L, about 30 ng/L, about 35 ng/L, about 40 ng/L, about 45 ng/L, about 50 ng/L, about 60 ng/L, about 70 ng/L, about 75 ng/L, about 80 ng/L, about 90 ng/L, about 95 ng/L) post administration of dexmedetomidine or pharmaceutically acceptable salt thereof on day 12.

Embodiment 124. The method of any of embodiment 89 or 90, wherein when the dose is 60μg, the mean plasma concentrations are in the range of about 10 ng/L to about 150 ng/L (for example 15 ng/L, about 20 ng/L, about 25 ng/L, about 30 ng/L, about 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L) post administration of dexmedetomidine or pharmaceutically acceptable salt thereof on day 12.

Embodiment 125. The method of any of embodiment 89 or 90, wherein when the dose is 90μg, the mean plasma concentrations are in the range of about 25 ng/L to about 150 ng/L (for example about 30 ng/L, about 35 ng/L, about 40 ng/L, about 45 ng/L, about 50 ng/L, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L) post administration of dexmedetomidine or pharmaceutically acceptable salt thereof on day 12

Embodiment 126. The method of any of embodiment 89 or 90, wherein when the dose is 120 μg, the mean plasma concentrations are in the range of about 50 ng/L to about 200 ng/L for example, about 55 ng/L, about 60 ng/L, about 70 ng/L, about 75 ng/L, about 90 ng/L, about 100 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L and about 195 ng/L) post administration of dexmedetomidine or pharmaceutically acceptable salt thereof on day 12.

Embodiment 127. The method of any of embodiment 89 or 90, wherein when the dose is 180 μg, the mean plasma concentrations are in the range of about 100 ng/L to about 400 ng/L for example, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, about 400 ng/L) post administration of dexmedetomidine or pharmaceutically acceptable salt thereof on day 12.

Embodiment 128. The method of any of embodiment 89 or 90, wherein when the dose is 240 μg, the mean plasma concentrations are in the range of about 50 ng/L to about 500 ng/L for example, about 55 ng/L, about 60 ng/L, about 70 ng/L, about 75 ng/L, about 80 ng/L, about 90 ng/L, about 95 ng/L, about 105 ng/L, about 110 ng/L, about 115 ng/L, about 120 ng/L, about 125 ng/L, about 130 ng/L, about 135 ng/L, about 140 ng/L, about 145 ng/L, about 150 ng/L, about 160 ng/L, about 165 ng/L, about 170 ng/L, about 175 ng/L, about 180 ng/L, about 185 ng/L, about 190 ng/L, about 195 ng/L, about 200 ng/L, about 225 ng/L, about 250 ng/L, about 275 ng/L, about 300 ng/L, about 325 ng/L, about 350 ng/L, about 375 ng/L, about 395 ng/L, about 400 ng/L, about 425 ng/L, about 450 ng/L, about 460 ng/L, about 465 ng/L, about 475 ng/L, about 480 ng/L, about 485 ng/L, about 490 ng/L and about 495 ng/L) post administration of dexmedetomidine or pharmaceutically acceptable salt thereof on day 12

Embodiment 129. The method of any of embodiments 117 to 128, wherein the mean plasma concentrations are 80% to 125% of these ranges and values

Embodiment 130. The method of any one of the embodiments 89 to 129, wherein dexmedetomidine or a pharmaceutically acceptable salt thereof is administered oromucosally (e.g. sublingually or buccally) in the form of a tablet, film, spray, gel or drops.

Embodiment 131. The method of embodiment 130, wherein dexmedetomidine or a pharmaceutically acceptable salt thereof is administered sublingually in the form of a tablet, film, spray, gel or drops.

Embodiment 132. The method of embodiment 131, wherein dexmedetomidine or a pharmaceutically acceptable salt thereof is administered sublingually in the form of a film.

Embodiment 133. The method of embodiment 132, wherein dexmedetomidine or a pharmaceutically acceptable salt thereof is administered buccally in the form of a film, patch or tablet.

Embodiment 134. The method of embodiment 130, wherein dexmedetomidine or a pharmaceutically acceptable salt thereof is administered buccally in the form of a film.

Embodiment 135 The method of any of the preceding embodiments, wherein the patient is treated without also inducing clinically significant cardiovascular effects.

Embodiment 136. The method of any one of the embodiments 89 to 129, wherein dexmedetomidine or a pharmaceutically acceptable salt thereof is administered orally, intranasally or parenterally.

Embodiment 137. The method of any of relevant preceding embodiments, where dexmedetomidine or a pharmaceutically acceptable salt thereof is administered as a film, wherein said film is a self-supporting, dissolvable, film, comprising:
(i) dexmedetomidine or a pharmaceutically acceptable salt thereof;
(ii) one or more water-soluble polymers; and, optionally,
(iii) one or more pharmaceutically acceptable carriers.

Embodiment 138. The method of embodiment 137, wherein (ii) comprises a low molecular weight, water-soluble polymer and two high molecular weight, water-soluble polymers.

Embodiment 139. The method of embodiment 138, wherein the low molecular weight, water-soluble polymer has a molecular weight from about 5,000 Daltons to about 49,000 Daltons, and each high molecular weight, water-soluble polymer has a molecular weight of greater than about 60,000 Daltons.

Embodiment 140. The method of embodiment 138, wherein the low molecular weight, water-soluble polymer has a molecular weight of about 40,000 Daltons, one of the two high molecular weight, water-soluble polymers has a molecular weight of about 140,000 Daltons, and the other high molecular weight, water-soluble polymer has a molecular weight of about 370,000 Daltons.

Embodiment 141. The method of any one of embodiments 138 to 140, wherein each water-soluble polymer is hydroxypropyl cellulose.

Embodiment 142. The method of any one of embodiments 138 to 140, wherein the film also comprises polyethylene oxide.

Embodiment 143. The method of embodiment 142, wherein the polyethylene oxide has a molecular weight of about 600,000 Daltons.

Embodiment 144. The method of any of relevant preceding embodiments, where dexmedetomidine or a pharmaceutically acceptable salt thereof is administered as a film, wherein said film is a self-supporting, dissolvable, film, comprising:
(i) dexmedetomidine or a pharmaceutically acceptable salt thereof;
(ii) a low molecular weight, water-soluble polymer having a molecular weight of about 40,000 Daltons;
(iii) a high molecular weight, water-soluble polymer having a molecular weight from about 140,000 Daltons;
(iv) a high molecular weight, water-soluble polymer having a molecular weight from about 370,000 Daltons; and
(v) a water-soluble polyethylene oxide having a molecular weight of about 600,000 Daltons.

Embodiment 145. The method of embodiment 144, wherein the film components excluding dexmedetomidine or a pharmaceutically acceptable salt thereof form a single layer film substrate, and dexmedetomidine or a pharmaceutically acceptable salt thereof is present on the surface of the film substrate.

Embodiment 146 The method of embodiment 145, wherein dexmedetomidine or a pharmaceutically acceptable salt thereof is present on the surface of the film substrate within a composition comprising dexmedetomidine or a pharmaceutically acceptable salt thereof, a low molecular weight, water-soluble polymer having a molecular weight of about 40,000 Daltons, and a high molecular weight, water-soluble polymer having a molecular weight of about 140,000 Daltons. Embodiment 147. A method of treatment of alcohol use disorder (AUD) with comorbid posttraumatic stress disorder (PTSD) comprising administering oromucosally to the subject in need thereof a pharmaceutical composition comprising about 40 μg of dexmedetomidine or a pharmaceutically acceptable salt thereof.

Embodiment 148. A method of treatment of alcohol use disorder (AUD) with comorbid posttraumatic stress disorder (PTSD) comprising administering oromucosally to the subject in need thereof a pharmaceutical composition comprising about 80 μg of dexmedetomidine or a pharmaceutically acceptable salt thereof.

Embodiment 149. A method of reducing alcohol consumption comprising administering oromucosally to the subject in need thereof a pharmaceutical composition comprising dexmedetomidine or a pharmaceutically acceptable salt thereof.

Embodiment 150. The method of embodiment 149, wherein the subject is suffering from alcohol use disorder with comorbid posttraumatic stress disorder (PTSD).

Embodiment 151. The method of embodiment 147 or 148, wherein the dexmedetomidine or a pharmaceutically acceptable salt thereof is administered at a dose of about 40 μg.

Embodiment 152. The method of embodiment 147 or 148, wherein the dexmedetomidine or a pharmaceutically acceptable salt thereof is administered at a dose of about 80 μg.

Embodiment 153. The method of embodiments 147 to 152, wherein dexmedetomidine or a pharmaceutically acceptable salt thereof is administered sublingually or buccally.

Embodiment 154. The method of embodiment 153, wherein dexmedetomidine or a pharmaceutically acceptable salt thereof is administered sublingually in the form of a tablet, film, spray, gel or drops.

Embodiment 155. The method of embodiment 154, wherein dexmedetomidine or a pharmaceutically acceptable salt thereof is administered sublingually in the form of a film.

Embodiment 156. The method of embodiment 153, wherein dexmedetomidine or a pharmaceutically acceptable salt thereof is administered buccally.

Embodiment 157. The method of embodiment 156, wherein dexmedetomidine or a pharmaceutically acceptable salt thereof is administered buccally in the form of a film, patch or tablet.

Embodiment 158. The method of any of the relevant preceding embodiments, wherein dexmedetomidine is present as dexmedetomidine hydrochloride.

Embodiment 159. The method of any of relevant preceding embodiments, wherein the subject is treated without experiencing clinically significant cardiovascular effects.

Embodiment 160. The method of any of embodiments 147 to 159, wherein the subject is treated without experiencing significant sedation.

Embodiment 161. The method of any of embodiments 147 to 159, wherein dexmedetomidine or a pharmaceutically acceptable salt thereof is administered from one to six times a day.

Embodiment 162. The method of any of embodiments 147 to 152, wherein dexmedetomidine or a pharmaceutically acceptable salt thereof is administered once every 3 days.

Embodiment 163. The method of any of embodiments 147 to 149, wherein treatment may further comprise concurrent administration of ethanol infusion.

Embodiment 164. The method of embodiment 163, wherein ethanol is administered using a clamp methodology targeting a breath alcohol concentration (BrAC) of 100 mg.

Embodiment 165. The method of embodiment 147 or embodiment 148, wherein the subject diagnosed with PTSD is determined by Clinician-Administered PTSD Scale for DSM-5 (CAPS-5).

Embodiment 166. The method of embodiment 147 or embodiment 148, wherein the subject with PTSD has a PCL-5 score >33.

Embodiment 167. The method of embodiments 147 or embodiment 148, wherein the subject diagnosed having alcohol use disorder is determined using Mini-International Neuropsychiatric Interview for DSM-5 (MINI-5).

Embodiment 168. The method of any of embodiments 147 to 149, wherein the subject is not previously treated with any other medication.

Embodiment 169. The method of any of embodiments 147 to 149, wherein the subject is suffering from bipolar disorder.

Embodiment 170. The method of embodiments 147 to 149, wherein the subject is not suffering from bipolar disorder.

Embodiment 171. The method of embodiment 147 to 149, wherein the subject is below 65 years old, preferably between 21 years old to 50 years old.

Embodiment 172. The method of embodiment 147 to 149, wherein the subject is above 65 years old.

Embodiment 173. The method of any of embodiments 147 to 152, wherein the subject has a breath alcohol content of less than 0.02 as determined using alcohol breathalyzer.

Embodiment 174. The method of any of embodiments 147 to 173, wherein the subject has a score of less than 4 on clinical Institute Withdrawal Assessment for Alcohol Scale (CIWA-Ar).

Embodiment 175. The method of any of embodiments 147 to 174, wherein changes in stress (PTSD) and alcohol cue reactivity are measured using sSTAI-6, VAS and YCS.

Embodiment 176. The method of any of embodiments 147 to 174, wherein changes in stress (PTSD) and alcohol cue reactivity when administered in combination with alcohol are measured using Biphasic Alcohol Effects Scale (BAES); Number of Drinks Scale (NDS); Cognitive performance as assessed by the Hopkins Verbal Learning Test (HVLT-R), Go No-Go Task, and Rapid Information Processing Task (RVIP) and Motor impairment as assessed by the Grooved Pegboard Test.

Embodiment 177. The method of any of embodiments 147 to 176, where dexmedetomidine or a pharmaceutically acceptable salt thereof is administered as a film, wherein said film is a self-supporting, dissolvable, film, comprising:
(i) dexmedetomidine or a pharmaceutically acceptable salt thereof;
(ii) one or more water-soluble polymers; and, optionally,
(iii) one or more pharmaceutically acceptable carriers

Embodiment 178. The method of any of embodiments 147 to 177 wherein dexmedetomidine hydrochloride is administered as a film, wherein said film is a self-supporting, dissolvable, film, comprising:
(a) a composition comprising:
   (i) dexmedetomidine hydrochloride;
   (ii) hydroxypropyl cellulose (40,000 MW); and
   (iii) hydroxypropyl cellulose (140,000 MW); and
(b) a film substrate comprising:
   (i) hydroxypropyl cellulose (40,000 MW);
   (ii) hydroxypropyl cellulose (140,000 MW);
   (iii) hydroxypropyl cellulose (370,000 MW); and
(iv) polyethylene oxide (600,000 MW);

wherein the composition of part (a) is present on the surface of the film substrate (b).

Embodiment 179. A method of treating agitation or signs of agitation in a pediatric subject without also inducing significant sedation,comprising oromucosally administering a single dose containing about 80 μg of dexmedetomidine or a pharmaceutically acceptable salt thereof, wherein the agitation is associated with schizophrenia.

Embodiment 180. A method of treating agitation or signs of agitation in a pediatric subject without also inducing significant sedation,comprising oromucosally administering a single dose containing about 120 μg of dexmedetomidine or a pharmaceutically acceptable salt thereof, wherein the agitation is associated with schizophrenia.

Embodiment 181. A method of treating agitation or signs of agitation in a pediatric subject without also inducing significant sedation,comprising oromucosally administering a single dose containing about 80 μg of dexmedetomidine or a pharmaceutically acceptable salt thereof, wherein the agitation is associated with bipolar disorder.

Embodiment 182. A method of treating agitation or signs of agitation in a pediatric subject without also inducing significant sedation,comprising oromucosally administering a single dose containing about 120 μg of dexmedetomidine or a pharmaceutically acceptable salt thereof, wherein the agitation is associated with bipolar disorder.

Embodiment 183. The method of embodiment 179 or 180, wherein the subject is about 13-17 years old.

Embodiment 184. The method of embodiment 181 or 182, wherein the subject is about 10-17 years old.

Embodiment 185. The method of embodiment 179 to 184, wherein the agitation is acute.

Embodiment 186. The method of embodiment 179 to 182, wherein the subject is diagnosed with Attenuated Psychosis Syndrome DSM-5 298.8 (F28).

Embodiment 187. The method of embodiment 179 to 182, wherein the subject has a score of >4 on at least 1 of the 5 items on the PEC at baseline.

Embodiment 188. The method of embodiment 179 to 182, wherein dexmedetomidine or a pharmaceutically acceptable salt thereof is administered sublingually or buccally as a film.

Embodiment 189. The method of embodiment 179 to 182, wherein reduction in agitation or signs of agitation is measured by relative change in PEC score after adminsitration of dexmedetomidine or a pharmaceutically acceptable salt thereof.

Embodiment 190. The method of embodiment 179 to 182, wherein the clinical improvement in agitation is measured using PANSS, ACES, and/or CGI-I scales.

Embodiment 191. A method of treating cocaine toxicity and/or symptoms associated with cocaine toxicity comprising administering oromucosally about 30 μg to about 400 μg of dexmedetomidine or a pharmaceutically acceptable salt thereof.

### EXAMPLES

### Example 1: Dexmedetomidine Oromucosal Film formulation

TABLE 6
Dexmedetomidine deposited on the surface
of a polymer matrix film composition

| | | | |
|---|---|---|---|
| | Concen- | Concen- | |
| | tration | tration | |
| | g/100 g | g/100 g | |
| | (10 μg | (20 μg | |
| Ingredients | film) | film) | Function |

Drug-containing composition

| | | | |
|---|---|---|---|
| Dexmedetomidine | 0.136 | 0.267 | Active agent |
| hydrochloride | | | |
| Hydroxypropyl cellulose, | 0.301 | 0.593 | Film former |
| HPC-SSL (MW = 40,000) | | | |
| Hydroxypropyl cellulose | 0.301 | 0.593 | Film former |
| (MW = 140,000) | | | |
| FD&C Blue #1 Granular | 0.002 | 0.004 | Color |
| Ethyl Alcohol as a solvent | qs | qs | Solvent |

Polymer matrix composition

| | | | |
|---|---|---|---|
| Hydroxypropyl cellulose | 4.803 | 4.768 | Film former |
| (MW = 140,000) | | | |
| Hydroxypropyl cellulose, | 4.803 | 4.768 | Film former |
| HPC-SSL (MW = 40,000) | | | |
| Hydroxypropyl cellulose | 28.809 | 28.601 | Film former |
| (MW = 370,000) | | | |
| Fast Emerald Green Shade | 0.129 | 0.128 | Color |
| (NO. 06507) | | | |
| Sucralose, USP-NF Grade | 0.993 | 0.985 | Sweetener |
| Peppermint Oil, NF | 2.104 | 2.089 | Flavor |
| Polyethylene oxide | 57.618 | 57.202 | Film former & |
| (Sentry Polyox WSR 205 | | | Mucoadhesive |
| LEO NF) (MW = 600,000) | | | |
| Water as a solvent | qs | qs | Solvent |

### (A) Process for the Preparation of Polymer Matrix:

Polymer mixture: Polyethylene oxide and fast emerald green shade were mixed in water for at least 180 minutes at about 1400 rpm to about 2000 rpm. Sucralose, hydroxypropyl cellulose (molecular weight 140K), hydroxypropyl cellulose, HPC-SSL (molecular weight 40K) and hydroxypropyl cellulose (molecular weight 370K) were added and mixed for at least 120 minutes at about 1600 rpm to 2000 rpm. Peppermint Oil was added to water and the resultant dispersion was then added to the polymer mixture and mixed for at least 30 minutes. The resultant mixture was further mixed under vacuum (248 torr) for at least for 30 minutes at a speed of 350 rpm and at temperature of 22.9° C.

Coating station: A roll was placed on an unwind stand and the leading edge was thread through guide bars and coating bars. The silicone-coated side of the liner was placed faced up. A gap of 40 millimeters was maintained between the coating bars. The oven set point was adjusted to 70° C. and the final drying temperature was adjusted to 85° C.

Coating/drying process: The polymer mixture was poured onto the liner between the guide bars and the coating bars. The liner was pulled slowly through the coating bar at a constant speed by hand until no liquid was remained on the coating bars. The liner was cut to approximately 12-inch length hand sheets using a safety knife. Each hand sheet was placed on a drying board and was tapped on the corners to prevent curl during drying. The hand sheets were dried in the oven until the moisture content was less than 5% (approximately 30 minutes) and then removed from the drying board. The coating weights were checked against the acceptance criteria, and if met, the hand sheets were then stacked and placed in a 34 inch×40 inch foil bag that was lined with PET release liner.

### (B) Process for the Preparation of Deposition Solution:

FDC blue was dissolved in ethyl alcohol for at least 180 minutes. Dexmedetomidine hydrochloride was added to the ethyl alcohol solution with continuous stirring for 10 minutes at about 400 rpm to about 800 rpm. Hydroxypropyl cellulose (40K) and hydroxypropyl cellulose (140K) were added to the mixture, and stirred for at least 30 minutes until all the materials were dissolved.

### (C) Process for the Preparation of Micro-Deposited Matrix:

The deposition solution obtained in Step (B) above was filled into a pipette to the required volume (determined according to the specific drug product strength of the final product). An appropriate amount (1.5 microliters=approximately 5 μg) of the deposition solution were deposited (e.g. as droplets) onto the polymer matrix obtained in Step (A), and repeated to a total of 10 times (i.e. 10 deposits/droplets) with space between each deposit to prevent merging of the deposits/droplets and allow subsequent cutting of the film into individual drug-containing units. The film was initially die cut in individual units with dimensions of 22 mm×8.8 mm containing a single deposit of the drug-containing composition. The die cut micro-deposited matrixes were then dried in an oven for 70° C. for 10 minutes and further die cut into 10 units with each unit containing a single deposit of the drug-containing composition.

### (D) Packaging:

Each defect-free unit was sealed individually into a foil pouch, which was then heat sealed. If the heat seal was acceptable the package was considered as an acceptable unit for commercial use.

Other unit strengths (e.g. 40 μg and 60 μg films) were similarly prepared by varying the concentrations of drug, polymers and colorant within the drug-containing composition. For example, the 40 μg and 60 μg, films were prepared from drug-containing compositions containing, respectively, approximately 2× and 3×, the amounts of drug, polymers and colorant that appear in the 20 μg drug-containing composition described in table 6 above.

### Example 2

TABLE 7
Dexmedetomidine deposited on the surface
of a polymer matrix film composition

| | | | | |
|---|---|---|---|---|
| | Concen- | Concen- | Concen- | |
| | tration | tration | tration | |
| | mg/unit | mg/unit | mg/unit | |
| | (80 μg | (120 μg | (180 μg | |
| Ingredients | film) | film) | film) | Function |

Drug-containing composition

| | | | | |
|---|---|---|---|---|
| Dexmedetomidine | 0.0945 | 0.142 | 0.213 | Active agent |
| hydrochloride | | | | |
| Hydroxypropyl | 0.0812 | 0.122 | 0.183 | Film former |
| cellulose, HPC-SSL | | | | |
| (MW = 40,000) | | | | |
| Hydroxypropyl cellulose | 0.0812 | 0.122 | 0.183 | Film former |
| (MW = 140,000) | | | | |
| FD&C Blue #1 Granular | 0.0008 | 0.001 | 0.002 | Color |
| Ethyl Alcohol as a | q.s | q.s. | q.s. | Solvent |
| solvent | | | | |

Polymer matrix composition

| | | | | |
|---|---|---|---|---|
| Hydroxypropyl cellulose | 0.627 | 0.627 | 0.627 | Film former |
| (MW = 140,000) | | | | |
| Hydroxypropyl | 0.627 | 0.627 | 0.627 | Film former |
| cellulose, HPC-SSL | | | | |
| (MW = 40,000) | | | | |
| Hydroxypropyl cellulose | 3.763 | 3.763 | 3.763 | Film former |
| (MW = 370,000) | | | | |
| Fast Emerald Green | 0.017 | 0.017 | 0.017 | Color |
| Shade (NO. 06507) | | | | |
| Sucralose, USP-NF | 0.130 | 0.130 | 0.130 | Sweetener |
| Grade | | | | |
| Peppermint Oil, NF | 0.275 | 0.275 | 0.275 | Flavor |
| Polyethylene oxide | 7.526 | 7.526 | 7.526 | Film former & |
| (Sentry Polyox WSR | | | | Mucoadhesive |
| 205 LEO NF) (MW = | | | | |
| 600,000) | | | | |
| Water as a solvent | qs | qs | qs | Solvent |

The formulations (80 μg, 120 μg and 180 μg) in table 7 were prepared using the same manufacturing process as described above in Example 1.

### Example 3: A Phase Ib/II, Multicenter, Randomized, Double Blind, Placebo Controlled, Ascending Dose Finding, Efficacy, Pharmacokinetic and Safety Study of Dexmedetomidine Hydrochloride Oromucosal film in Agitation Associated with Dementia

### Primary Objective:

Describe the safety and tolerability of single doses of dexmedetomidine hydrochloride for study of efficacy in treatment of acute agitation associated with dementia.

### Secondary Objective:

1. Describe the onset and magnitude of calming effects of different doses of dexmedetomidine hydrochloride on symptoms of acute agitation associated with dementia compared to placebo.
2. Describe the duration of calming as measured by PEC and ACES.
3. Describe the tolerability and safety profile of dexmedetomidine hydrochloride sublingual film, as determined by adverse events and vital signs versus placebo.
4. Describe clinical effects as measured by the Clinician Global Impression of Severity of agitation scale (CGI-S) to access agitation and then Improvement (CGI-I) after drug administration.
5. Describe the frequency of agitation using the Cohen Mansfield Agitation Inventory (CMAI) at baseline and 2 hours post dose.
6. Determine the approximate dissolution time of dexmedetomidine hydrochloride oromucosal films in the sublingual space.
7. Assess the local tolerability via buccal examination after dosing dexmedetomidine hydrochloride oromucosal film.
8. Describe the pharmacokinetics and exposure of dexmedetomidine as delivered by sublingual dexmedetomidine hydrochloride oromucosal film dosing.
9. Part B—Describe the duration of calming as measured by the 3 supplementary items of the PANSS.

### Primary Outcome Measure

1. Incidence of AEs
   [Time Frame: 7 days]
2. Incidence of abnormal laboratory test results
   Incidence of abnormal laboratory test results
   [Time Frame: 7 days]
3. Incidence of abnormal vital signs
   Incidence of abnormal vital sign (systolic and diastolic blood pressures, heart rate measured as pulse, respiratory rate, and temperature)
   [Time Frame: 7 days]
4. Incidence of abnormal ECG findings
   Incidence of abnormal ECG findings
   [Time Frame: 7 days]

### Secondary Outcome Measure

1. Magnitude of calming effects
   Magnitude of calming effects of different doses of dexmedetomidine oromucosal film on symptoms of acute agitation associated with dementia as measured by the Pittsburgh Agitation Scale (PAS) (Minimum Score value: 0. Maximum score value 16. Higher scores mean a worse outcome)
   [Time Frame: 24 hours]

### Methodology

This was an adaptive Phase Ib/2 trial design. It was a randomized, double-blind, placebo-controlled, multiple ascending dose study assessing efficacy, pharmacokinetics, safety and tolerability of dexmedetomidine hydrochloride dosing in adult (65 years old and older) males and females with acute agitation associated with dementia.

The study attempted to characterize a safe and tolerable dose range that result in calming effect as measured using the Pittsburgh Agitation Scale (PAS) by evaluating at least 10 subjects, (4:1 randomization to active: placebo), at each of the three dose levels(Table 8).

Patients in Part B of this study were seniors aged 65 and above, who were semi-independent, and able to carry out many of their activities of daily living under minimal supervision, such as those who resided in assisted living facilities. The study consisted of a pre-screening/screening period, treatment period, and a follow-up period. Evaluation of three doses of 30 μg, 60 μg and 90 μg (Cohort 1, Cohort 2, and Cohort 3, respectively) were planned, with an option to test different doses based on tolerability and safety. This was an adaptive design as doses selected for testing might be different from these, based upon safety reviews. Doses lower or higher might be chosen to test, and repeated, up to 180 μg within each cohort and additional subjects may be added to a cohort. Dexmedetomidine hydrochloride films might be divided in half if needed to deliver half-dose strengths. Except for the cohort 1 (30μg), each subsequent dose level was authorized after a safety review of the previous dosing cohort. Dosing might be repeated in the case of persistent or recurrent agitation, if there was no significant improvement (CGI-I of 1 or 2 as ‘very much’ or ‘much improved’) and no safety events evident. Dosing might be repeated up to a total of two repeat doses (at the same randomization group Active: Placebo) for all cohorts except for 90 μg dose which could only be repeated once (total 180 μg) if necessary, at 2 hours post first dose but only after the 2-hour assessments were conducted and only within 12 hours post first dose. Patients could only be re-dosed if they were hemodynamically stable, not hypotensive (must be greater than 90/60 diastolic/systolic) and not bradycardic (must be greater than 60 bpm). Patients also could not be re-dosed if they were orthostatic (a drop of 20 points in either SBP or DBP) or if they were experiencing an AE. Not only does this determine individual response to a single dose but determined if a given subject was responsive to a second dose, and might respond to a greater dose, or could be categorized as a non-responder to dexmedetomidine hydrochloride despite being exposed to a greater total dose.

Periodic safety data reviews were undertaken on an ongoing basis to review all subjects assigned and dosed, as data and analyses became available. Dose escalation was allowed unless a safety or tolerability issue became evident upon periodic regular safety review.

Patients enrolling at a site were sequentially assigned to the lowest dose cohort (including placebo) followed by enrollment assignment to increasing dose cohorts. This sequential escalating adaptive enrollment ensures subject safety; the lowest dose cohort completed accrual first, higher dose cohorts complete last. In addition, those subjects assessed as requiring a second dose for efficacy provided early evidence of safety/tolerability of higher doses as they were effectively exposed to doses that approximate the next dose cohort. The majority of patients were enrolled and evaluated in lower dose cohorts before a higher dose cohort was initiated. Further, if evidence of intolerability arose from analyses integrating PK, exposure and safety/tolerability of all subjects and doses, the dose regimen might be altered, or a different dose might be selected to test the hypothesis that a (typically lower) dose regimen was better tolerated. Eligible patients (those with any type of dementia) might be identified in SNIFFs, mental health, psychiatric or medical emergency services, including medical/psychiatric observation units, or as newly admitted to a hospital setting for acute agitation or already in hospital for chronic underlying conditions. Subjects remained in their facility while undergoing screening procedures to assess eligibility. Upon confirmation of eligibility, subjects were randomized to a single 30 μg, 60 μg, or 90 μg dose, respectively, of dexmedetomidine hydrochloride or placebo film. For Part B of this study, subjects were randomized to receive a single dose of dexmedetomidine hydrochloride 40 μg or matching placebo film.

At the beginning of each study session, a single dose of dexmedetomidine hydrochloride film was administered sublingually by the patient if able with instructions from an unblinded staff member who does not participated in evaluation of safety or efficacy. The drug film was retained in the sublingual cavity until dissolved. Participants were also evaluated for local irritation around the area where the film was placed. Efficacy and safety assessments were conducted periodically before and after dosing. The next cohort was dosed after completing accrual of most prior panels, in accord with regular ongoing periodic safety and PK review as eligible subjects were assigned, dosed, and data became available.

Vital signs and ECGs were conducted at the time points indicated in the schedule of events. Participants were allowed water as desired 15 minutes after completion of dosing. Safety and tolerability assessments were continued until the morning of Day 3 (day of discharge) and were repeated on Day 7+2. Smoking was permitted according to the site's policies. After the 4 hr assessments were completed, at the discretion of the PI, rescue therapy might be initiated using standard of care treatment which might include lorazepam 0.5-5 mg po/IM or an antipsychotic medication po/IM.

Any abnormal vital sign measurement, clinical laboratory test, physical examination finding, or ECG parameter deemed clinically significant by the investigator was repeated, including test results obtained on the final study day or upon early termination. For any test abnormality deemed clinically significant, repeat analysis was performed during the follow-up period and until the value returned to baseline (or within normal limits) or the investigator deems the abnormality to be of no clinical significance. Subjects presenting with a clinically significant Urinary Tract Infection (UTI) as determined by clinical laboratory tests were excluded from the study.

### Efficacy Assessments

Efficacy measurements were taken up to and including 24 hours post first dose. The effects of dexmedetomidine hydrochloride on acute agitation were assessed by the following scales: Pittsburgh Agitation Scale (PAS), the PANSS-EC (PEC), CMAI, CGI-Severity (CGI-S) for Agitation and CGI-Improvement (CGI-I) for Agitation. If there was no significant improvement in CGI (CGI-I of 1 or 2 as “very much” or “much improved” respectively) and there were no evident safety concerns, a second film (of same assignment active vs. placebo) may be given.

### Safety and Tolerability Assessments

AEs, clinical laboratory tests, 12-lead ECG, Johns Hopkins Fall Risk Assessment score, and vital signs were monitored, and all observed and volunteered AEs were recorded. Blood pressure, heart rate and ECG were completed per schedule of assessments. Any abnormal clinically significant (investigator determined) vital sign measurement, clinical laboratory test, physical examination finding, or ECG parameter were repeated, until the value returned to baseline (or within normal limits) or the investigator deemed the abnormality to be of no clinical significance. Orthostatic assessments followed the CDC guidelines for the elderly (e.g. blood pressure upon standing for 1, 3 and 5 minutes). Safety and tolerability assessments were continued until the morning of Day 2 and Day 3 and were repeated on Day 7±2 days.

TABLE 8
Arms and Interventions

| Arms | Intervention |
|---|---|
| Active Comparator: | Drug: Oromucosal film containing |
| Cohort 1: 30 μg | dexmedetomidine hydrochloride for the |
| Cohort 1 consists of 10 patients out of whom 8 | treatment of agitation associated with |
| patients receive 30 μg film and the remaining | dementia |
| 2 patients receive a placebo | Drug: Oromucosal placebo film that |
| | matches dexmedetomidine film |
| Active Comparator: | Drug: Oromucosal film containing |
| Cohort 2: 60 μg | dexmedetomidine hydrochloride for the |
| Cohort 2 consists of 10 patients out of whom 8 | treatment of agitation associated with |
| patients receive 60 μg film and the remaining | dementia |
| 2 patients receive a placebo | Drug: Oromucosal placebo film that |
| | matches dexmedetomidine film |
| Active Comparator: | Drug: Oromucosal film containing |
| Cohort 3: 90 μg | dexmedetomidine hydrochloride for |
| Cohort 3 consists of 10 patients out of whom 8 | treatment of agitation associated with |
| patients receive 90 μg film and the remaining | dementia |
| 2 patients receive a placebo | Drug: Oromucosal placebo film that |
| | matched dexmedetomidine film |
| Active Comparator: Part B Cohort | Drug: Oromucosal film containing |
| Part B cohort consists of 46 subjects receiving 40 | dexmedetomidine hydrochloride for |
| μg or placebo | treatment of agitation associated with |
| | dementia |
| | Drug: Oromucosal placebo film |
| | that matches dexmedetomidine film |

### Number of Subjects

At least 30 subjects (10 per cohort) were enrolled and randomized at up to 4 study sites in the United States. Patients were randomized in each dose cohort in increment groups of 5 (ratio=4:1 active:placebo). However, it was possible that the sponsor might opt to expand the number of sites and subjects per dose cohort (up to 90 total patients) as the study progressed. As such, an additional 20 patients were enrolled into the 60-μg cohort. These additional subjects provided significantly more safety data at the 60-μg dose, but also a greater imbalance with respect to placebo (pooled across cohorts) under the original 4:1 randomization ratio (active:placebo). Accordingly, to facilitate more informative comparisons to placebo, the additional 20 subjects were randomized at a 1:1 ratio of active:placebo. This achieved the overall randomization ratio as originally designed. In Part B, 46 patients were randomized 1:1 ratio to receive dexmedetomidine hydrochloride 40 μg oromucosal film or matching placebo film.

Eligible individuals with any form of dementia who had a history of recent agitation (6 months or less) or their legally authorized representative (LAR) signed an informed consent before any study-related procedures were performed. Upon confirmation of eligibility, subjects in Cohorts 1, 2, and 3 were randomized to either dexmedetomidine hydrochloride oromucosal film or placebo film in a 4:1 randomization. The additional 20 subjects to be enrolled in the 60-μg cohort were randomized to either dexmedetomidine hydrochloride oromucosal film or placebo on a 1:1 randomization. Subjects in Cohort 3 first entered the 1-week safety observation to assess eligibility, after which they were screened and randomized to either dexmedetomidine hydrochloride oromucosal film or placebo. Once subjects became agitated, they proceeded with Day 1 assessments.

TABLE 9A
Schedule of Events

| | Activity | | | | | | | |
|---|---|---|---|---|---|---|---|---|
| | Pre- | | Pre- | | | | | |
| | Screening^{8, 9} | Screening | Dose¹ | Treatment Evaluation Day 1 | | | | |
| | Time point | | | | | | | |
| | Pre- | Pre- | −1 hr to | 5 | 10 | 15 | 30 | 1 |
| | treatment | treatment | time 0 | min | min | min | min | hr |
| Informed Consent | X | | | | | | | |
| Medical History | X | X | | | | | | |
| Demographics | X | X | | | | | | |
| Weight | X | | | | | | | |
| Height | X | | | | | | | |
| Mini-Mental State exam | X | | | | | | | |
| Clinical Dementia Rating Score | X | | | | | | | |
| Physical Exam | X | X | | | | | | |
| Safety Laboratory assessments³ | X | | | | | | | |
| UDS¹⁰ | X | X¹¹ | | | | | | |
| UTI and pregnancy | | X | | | | | | |
| Johns Hopkins Fall Risk | X | | | | | | | |
| Assessment (Cohort 3 only) | | | | | | | | |
| ECG with rhythm strip⁷ | X | | X | | | | | |
| Pulse oximetry | | | X | | | | X | X |
| Resting vital signs² | X | X | X | | | | X | X |
| Orthostatic vital signs² | X | X | X | | | | X | X |
| Inclusion/Exclusion criteria | X | X | X | | | | | |
| Randomization | | | X | | | | | |
| CMAI | | X | X | | | | | |
| Study drug administration⁶ | | | X | | | | | |
| PAS | X | X | X | | | | X | X |
| PEC¹² | X | | X | | | | X | X |
| ACES | | | X | | | | | X |
| CGI-Severity Agitation | | | X | | | | | |
| CGI-Improvement/change | | | | | | | X | X |
| in Agitation | | | | | | | | |
| C-SSRS | X | X | | | | | | |
| Buccal (SL) assessment⁵ | | | | X | X | X | X | |
| PK Sampling⁴ | | | | | | | X | X |
| Concomitant Medications | X | X | X | X | X | X | X | X |
| Adverse Events | X | X | X | X | X | X | X | X |

| | Activity | | | | | | |
|---|---|---|---|---|---|---|---|
| | | | | | Day 2 | | |
| | | | | | Follow-Up | | Day 7 |
| | Treatment Evaluation Day 1 | | | | (+1 day) | | (+2 days) |
| | Time point | | | | | | |
| | | | | | 24 hr | | |
| | 2 | 4 | 6 | 8 | (−9/+12 | Day | End of |
| | hr | hr | hr | hr | hr) | 3 | Study |
| Informed Consent | | | | | | | |
| Medical History | | | | | | | |
| Demographics | | | | | | | |
| Weight | | | | | X | | |
| Height | | | | | | | |
| Mini-Mental State exam | | | | | X | | |
| Clinical Dementia Rating Score | | | | | X | | |
| Physical Exam | | | | | X | | |
| Safety Laboratory assessments³ | | | | | | X | X |
| UDS¹⁰ | | | | | | | |
| UTI and pregnancy | | | | | | | |
| Johns Hopkins Fall Risk | | | | | | | |
| Assessment (Cohort 3 only) | | | | | | | |
| ECG with rhythm strip⁷ | X | | | | X | | |
| Pulse oximetry | X | X | X | X | X | | |
| Resting vital signs² | X | X | X | X | X | | |
| Orthostatic vital signs² | X | X | | X | X | | |
| Inclusion/Exclusion criteria | | | | | | | |
| Randomization | | | | | | | |
| CMAI | X | | | | | | X |
| Study drug administration⁶ | | | | | | | |
| PAS | X | X | | X | X | X | X |
| PEC¹² | X | X | | X | X | X | X |
| ACES | X | X | | X | | | |
| CGI-Severity Agitation | X | | | | X | | |
| CGI-Improvement/change | X | X | | X | | | |
| in Agitation | | | | | | | |
| C-SSRS | | | | | X | | |
| Buccal (SL) assessment⁵ | X | X | | | X | | |
| PK Sampling⁴ | X* | X | | X | X | | |
| Concomitant Medications | X | X | X | X | X | X | X |
| Adverse Events | X | X | X | X | X | X | X |

¹Pre-dose assessments had a window of 60 minutes prior to first dose. If possible, Pre-dose CMAI should be performed within 45 min prior to dosing and PAS, PEC and CGI-S should be performed within 15 min prior to dosing. Timing of all subsequent assessments was relative to the first dose. All post-dose assessments had a window of −10/+20 minutes until 2 hours and ±30 minutes until 8 hours. All post-dose efficacy assessments were conducted prior to any other assessments at each time point.
²Resting vital signs (SBP, DBP and HR) were taken upon having the subject recumbent for 5 min at Pre Screening, Screening, Pre-dose and at 30 min, 1, 2, 4 6, 8 and 24 hours post first dose. Triplicate measurements were performed in case of Systolic BP <90 mmHg, Diastolic BP <60 mmHg or Pulse <60 bpm. Orthostatic measurements (SBP, DBP, HR,) will be taken upon having the subject stand, with measurements taken upon standing for 1, 3 and 5 minutes, per CDC guidelines for elderly) at Pre Screening, Screening, Pre-dose visits, 30 min, 1, 2, 4, 8 and 24 hours post first dose. Temperature and respiratory rate will be recorded when orthostatic measurement is indicated in the Schedule of Events and are not required to be measured at resting vital sign timepoints Vital signs were done prior to drawing PK samples.
³Safety Laboratory assesssments included chemistry, hematology and urinalysis,. Laboratory samples drawn within 28 days prior to dosing might suffice with the exception of UDS (urine drug screen).
⁴PK blood samples were collected at 30 min, 1, 2, 4, 8-10 hours (collect one sample between 8 and 10 hours) and 24 hr after first dose. For Part B subjects, an additional sample will be collected if possible, between 10 and 12 hours. The 8 hour sample could have a window of 7-9 hours post dose and the next sample could have a window of 10-12 hours post dose. A sample might not be collected if the Physician indicated in source documents that the patient was in a mental state that is not conducive to PK sample collection. Non-compliance or refusal of all or any PK draw was not be exclusionary nor result in Early termination (ET). All PK collections were had a window of ±10 minutes except for the 24-hour post-dose collection which were had a window of ±1 hour.
*For re-dosed subjects only: PK blood sample were collected at 2.5 hrs. post first dose in addition to the other times.
⁵Buccal exam for local irritation and drug dissolution time was performed by unblinded staff at 5, 10, 15, 30 min, 2 h, 4 h and 24 hours post first dose.
⁶All pre-dose and screening visit assessments should be completed before study treatment is administered. In the investigator's clinical judgement, the same randomized dose and or a lower dose might be repeated after 2 hr post first dose assessments were complete, if there was persistent or recurrent agitation as measured by (improvement on the CGI-I) and in the absence of safety concerns. Doses could be repeated twice in a span of 12 hours, except for the 90 μg cohort, which could only be repeated once. If necessary, placement of study drug might be performed by unblinded research staff member. Antihypertensives or other medications could be held on the day of study drug administration at the discretion of the PI.
⁷12 LED ECGs for pre-dose needed to be collected but if unable to be assessed it did not constitute a protocol deviation. ECGs collected following treatment were to be performed prior to PK assessments.
⁸Pre-Screening Assessments were performed within 28 days before first dose of study treatment.. If subject did not became agitated within the 28-day window, they were considered a pre-screen failure. However, the subject could be rescreened once at the discretion of the investigator.
⁹After completing Pre-Screening assessments and review of labs and ECGs, Cohort 3 subjects, if eligible, entered the 1-week safety observation (Table 9B). After completing the 1-week safety observation, subjects started with Screening visit assessments.
¹⁰UDS was analyzed by a local laboratory.
¹¹UDS required at Screening for Cohort 3. UDS will be re-collected at Screening for Cohorts 1, 2 and subjects in part B of this study if more than 21 days have passed since the Pre-Screening visit.
¹²For Part B, the 3 PANSS Supplementary Items were performed at each PEC assessment.

TABLE 9B
Schedule of Events - 1-week Safety Observation (Cohort 3 Only)

| | Activity | | | | | | | | | | | | | |
|---|---|---|---|---|---|---|---|---|---|---|---|---|---|---|
| | Day O1 | | Day O2 | | Day O3 | | Day O4 | | Day O5 | | Day O6 | | Day O7 | |
| Time point | AM | PM¹ | AM | PM¹ | AM | PM¹ | AM | PM¹ | AM | PM¹ | AM | PM¹ | AM | PM¹ |
| Resting vital | X | X | X | X | X | X | X | X | X | X | X | X | X | X |
| signs² | | | | | | | | | | | | | | |
| Orthostatic vital | X | X | X | X | X | X | X | X | X | X | X | X | X | X |
| signs² | | | | | | | | | | | | | | |
| Recording of | X | X | X | X | X | X | X | X | X | X | X | X | X | X |
| falling and | | | | | | | | | | | | | | |
| syncope | | | | | | | | | | | | | | |
| Concomitant | X | X | X | X | X | X | X | X | X | X | X | X | X | X |
| medications | | | | | | | | | | | | | | |
| Adverse events | X | X | X | X | X | X | X | X | X | X | X | X | X | X |

Notes to the Schedule of Events:
¹PM assessments were performed at least 8 hours after AM assessments.
²Resting vital signs (SBP, DBP and HR) were taken upon having the subject recumbent for 5 min. Triplicate measurements were performed in case of SBP <90 mmHg, DBP <60 mmHg, or pulse <60 bpm. Orthostatic measurements (SBP, DBP, HR) were taken upon having the subject stand, with measurements taken upon standing for 1, 3, and 5 minutes, per CDC guidelines for elderly).

### Diagnosis and Main Criteria for Eligibility

### Inclusion Criteria

1. Male and female patients 65 years old and older.
2. Patients who met DSM-5 criteria for neurocognitive disorder, or dementia who had history of instances of acute agitation.
3. History of agitation (e.g., kick, bite, flailing) to the point that it impairs social activities, requires staffing or medical intervention, or impairs ability for functional activities of daily living.
4. Patients who met IPA diagnostic criterion for agitation.
5. Patients who were judged to be clinically agitated at Pre-dose with a total score of ≥8 on the 4 items (aberrant vocalization, motor agitation, aggressiveness, and resisting care) comprising the Pittsburgh Agitation Scale (PAS).
6. Patients who had a score of ≥2 on at least 1 of the 4 items on the Pittsburgh Agitation Scale (PAS).
7. Patients who read, understood and provided written informed consent, or who have a Legally Authorized Representative (LAR).
8. Patients who were in good general health prior to study participation as determined by a detailed medical history, physical examination, 12-lead ECG, blood chemistry profile, hematology, urinalysis and in the opinion of the Principal Investigator.
9. Female participants, if of child-bearing potential and sexually active, and male participants, if sexually active with a partner of child-bearing potential, who agreed to use a medically acceptable and effective birth control method throughout the study and for one week following the end of the study. Medically acceptable methods of contraception that might be used by the participant and/or his/her partner include abstinence, birth control pills or patches, diaphragm with spermicide, intrauterine device (IUD), condom with foam or spermicide, vaginal spermicidal suppository, surgical sterilization and progestin implant or injection. Prohibited methods include: the rhythm method, withdrawal, condoms alone, or diaphragm alone.

### Exclusion Criteria

1. Patients with agitation caused by acute intoxication must be excluded. Positive identification of non-prescription drugs during urine screening excluded the subject.
2. Patients treated within 4 hours prior to study drug administration with benzodiazepines, other sedatives, hypnotics or oral or short-acting intramuscular antipsychotics must be excluded.
3. Treatment with alpha-1 noradrenergic blockers, alpha adrenergic antagonists within 8 hours prior to dosing.
4. No new chronic medications initiated in the past 14 days prior to screening excluding over-the-counter products taken sporadically.
5. Patients with significant risk of suicide or homicide per the investigator's assessment, or any patient with an answer of “yes” to item 4 or 5 on the CSSRS.
6. Patients who had hydrocephalus, seizure disorder, or history of significant head trauma, subarachnoid bleeding, brain tumor, encephalopathy, meningitis, or focal neurological findings, with a recent large (non-microvascular) stroke, who might be considered medically unstable or in recovery must be excluded. Patients with a remote history of stroke might be included, regardless of size/location.
   History of clinically significant syncope or other syncopal attacks, orthostatic hypotension within the past two years, current evidence of hypovolemia, orthostatic hypotension (following 1, 3, and 5 minutes of standing, ≥20 mmHg drop in systolic BP or >10 mmHg drop in diastolic, or dizziness or lightheadedness) bradycardia or a baseline (Pre-dose) measurements of heart rate of <60 beats per minutes or systolic blood pressure <110 mmHg or diastolic BP <70 mmHg must be excluded.

Note: Subjects in Cohort 3 who have OH on more than 1 instance in the same day during the 1-week safety observation period must be excluded.
7. Patients with laboratory or ECG abnormalities considered clinically significant by the investigator or qualified designee [Advanced heart block (second-degree or above atrioventricular block without pacemaker), diagnosis of Sick sinus syndrome] considered clinically significant by the investigator or qualified designee and that would have clinical implications for the patient's participation in the study must be excluded.
8. Cohort 3 only: Patients who were taking nitrates or beta blockers were excluded. Any other anti-hypertensives should be maintained in the course of the study.
9. Patients with serious or unstable or uncontrolled medical illnesses must be excluded. These included current hepatic (moderate-severe hepatic impairment), renal, gastro-enterologic, respiratory, cardiovascular (including ischemic heart disease, congestive heart failure), endocrinologic, or hematologic disease.
10. Patients who had received an investigational drug within 30 days prior to the current agitation episode must be excluded.
11. Patients who were considered by the investigator, for any reason, to be an unsuitable candidate for receiving dexmedetomidine, or unable to use the oromucosal film, must be excluded; e.g. patients with a history of allergic reactions to dexmedetomidine must be excluded.
12. Patients experiencing clinically significant pain, per Investigator.
13. Cohort 3 only: Patients who were a high fall risk assessed via the Johns Hopkins Fall Risk Assessment (total score >13) or during the 1-week safety observation period were excluded from further study participation.
14. Pregnancy
15. For Part B: Patients with dementia associated with Parkinson's disease and/or Lewy Body Disease, if etiology of dementia is known.

### Method of Assigning Subjects to Treatment Groups

Upon confirmation of eligibility, subjects were randomized to dexmedetomidine hydrochloride or placebo film. In each of the three-dose cohorts, patients were randomized 4:1 dexmedetomidine hydrochloride film: Placebo. If additional patients were added to a cohort (up to approximately 90 total patients), they were randomized 4:1 active drug: placebo. However, after beginning enrollment in the 90-μg cohort, a decision was made not to continue to enroll the 90-μg cohort, but rather to enroll an additional 20 subjects at the 60-μg dose. This could result in significantly more safety data at that dose, but also a greater imbalance with respect to placebo (pooled across cohorts) under the original 4:1 randomization ratio (active drug:placebo). Accordingly, to facilitate more informative comparisons to placebo, the additional 20 subjects were randomized at a 1:1 ratio of active drug: placebo. This achieved the overall randomization ratio as originally designed. In Part B, the inclusion of an additional 46 subjects assessed the efficacy and safety of a 40 μg dose of dexmedetomidine hydrochloride or placebo in a 1:1 randomization ratio. The study randomization was computer generated.

### Treatment Administration

Dosing might be achieved by cutting of a film, widthwise, directly in the middle, to make a half dose. Dosing might also be achieved by administration of 1 to 2 films [e.g., a 120 μg dose might be cut in half and administered to make a 60-μg dose or a 180-μg dose might be cut in half and administered to make a 90 μg dose]. At the beginning of each study session, patients were instructed on how to self-administer the investigational product. If the patient could self-administer, he/she self-administered the dose of dexmedetomidine hydrochloride or placebo film sublingually under supervision of an unblinded staff member who does not participated in evaluation of safety or efficacy. The investigational product was retained in the sublingual cavity until dissolved. If sublingual administration was not possible, the film might be placed inside the lower lip. The location of the placement of the film should be noted in the subject's chart. Objective buccal mucosal examination and time of film dissolution by unblinded study staff per Table 9A was conducted. In Part B, dosing was achieved by cutting of an 80 μg film, widthwise, directly in the middle, to make a 40 μg dose

### Study Procedures

Subjects or their LAR provided written informed consent, and assent as applicable, before any study-related procedures are initiated, including the cessation of prohibited concomitant therapy. The schedule of events to be performed during the study was provided in Table 9A.

Cohort 3 subjects, who participated in the 1-week safety observation period, also followed the assessments provided in Table 9B. For Part B, the 3 PANSS Supplementary Items were done at the times the PEC was conducted, including the different timepoints throughout the study.

### Study Assessments

### Efficacy:

The effect of study drug was evaluated using several validated instruments as given below.

PANSS-Excited Component (PEC)

Agitation-Calmness Evaluation Scale (ACES)

Cohen Mansfield Agitation Inventory (CMAI):

Assessment of drug effect on frequency of acute agitation was done using the CMAI. The CMAI is a rating questionnaire consisting of 29 behaviors each rated on a 7-point scale of frequency. It was possible that all 29 behaviors would not be relevant to a specific patient. Only behaviors manifested by the subject at baseline were assessed throughout the study resulting in a modified CMAI. Behaviors which were present immediately pre-dose were rated throughout the post-dose time-points. At each time-point after pre-dose, the rater noted items (behaviors) which were not manifested prior to dosing had not emerged since last CMAI assessment. Should they emerge, these items shall be included in ratings.

Pittsburg Agitation Scale (PAS):

The Pittsburg Agitation Scale (PAS) is an instrument based on direct observations of the patient that is developed to monitor the severity of agitation associated with dementia. There are four Behavior Groups observed (using a 0 to 4-point scale) in the patient, Aberrant Vocalization, Motor Agitation, Aggressiveness, Resting Care.

CGI-S and CGI-I for Agitation:

Both CGI-I and CGI-S were focused on the severity of agitation rather than the severity of the overall illness of dementia.

Clinical Global Impression of Severity (CGI-S) was rated based upon the severity of agitation at screening and pre-dose (immediately prior to start of dosing). Severity of agitation was assessed based on following scale:
0=Not assessed
1=Normal not at all symptomatic
2=Minimally symptomatic—few or mild symptoms—little interference with patients functioning
3=Mildly symptomatic-low level of symptoms-little interference in social functioning
4=Moderately symptomatic—some prominent symptoms—some interference in functioning
5=Markedly symptomatic—significant symptoms with very substantial interference in functioning
6=Severely symptomatic—very marked symptoms make it difficult for patients to engage with others
7=Among the most extremely symptomatic subjects-extreme symptoms—patient is incapacitated or highly dangerous to self or others requires extra care and supervision

Drug response on agitation was evaluated by the Clinical Global Impressions-Improvement (CGI-I) which was performed after dosing and evaluated relative to pre-dose baseline agitation. The CGI-I scores range from 1 to 7:
0=not assessed (missing),
1=very much improved,
2=much improved,
3=minimally improved,
4=no change,
5=minimally worse,
6=much worse,
7=very much worse

### Clinical Diagnosis and Description of Dementia

The subtype of dementia was determined and recorded based upon clinical neurologic and psychiatric evaluation to included review of all available medical information, medical records, documentation of prior evaluations, family/caretaker interviews, records, laboratory, genetics or other biomarkers, and results of neuroimaging (if available). The following scales were characterized subject's dementia (DSM-5 Major Neurocognitive disorder) in terms of cognitive and functional impairment:

MMSE

The Folstein Mini-Mental State Examination (MMSE) is an examination that tests an elderly person's cognitive ability. Domains measured by the MMSE include orientation to time and place, registration, attention and calculation, recall, naming, repetition, comprehension, reading, writing, and drawing. Total points on this test are 30. Any score of 24 or more (out of 30) indicates a normal cognition. Below this, scores can indicate severe (<9 points), moderate (10-18 points) or mild (19-23 points) cognitive impairment.

CDR® Score

The CDR® (Alzheimer's Disease Research Center, Washington University, St Louis) is a 5-point scale used to characterize six domains of cognitive and functional performance applicable to Alzheimer Disease and related dementias: memory, orientation, judgment & problem solving, community affairs, home & hobbies, and personal care. A score of 0 connotes no cognitive impairment, and then the remaining four points are for various stages of dementia where:

CDR-0=normal
CDR-0.5=very mild dementia
CDR-1=mild
CDR-2=moderate
CDR-3=severe.

### Safety

Safety was assessed during the study by the monitoring and recording of AEs, clinical laboratory test results (hematology, biochemistry, and urinalysis), vital sign measurements (systolic and diastolic blood pressures, heart rate measured as pulse, respiratory rate, and temperature), ECG, and physical examination findings.

Adverse events (AEs) were characterized by type, severity, seriousness, and relationship to treatment. Adverse events were coded by preferred term and system organ class using MedDRA version 20.0.

### Pharmacokinetics

Blood samples (4 mL) were collected at 0.5, 1, 2, 4, 8- and 24-hours post-dose per Schedule of Events (Table 9A).

For each subject, up to 6 blood samples (24 mL of blood) were collected during the study for PK analysis. In addition, approximately 15 mL of blood was collected at screening, approximately 15 mL of blood was collected at Day 3 Discharge, and approximately 15 mL of blood was collected at Day 7(+2) for clinical laboratory testing. The total volume of blood collected during the study was expected to be approximately 69 mL.

For re-dosed subjects only: an extra PK blood sample (4 ml) was collected at 2.5 hours post first dose in addition to the other times, totaling approximately 73 ml. All PK sampling occurred only after all the other assessments at that time point were conducted.

### Pharmacokinetic Analyses

All pharmacokinetic parameters were calculated using non-compartmental analysis using WinNonlin. Actual sampling times were used in all pharmacokinetic analyses. Per protocol times were used to calculate mean plasma concentrations for graphical displays. Other PK analyses might be performed as appropriate.

### Results

Interim data: The interim efficacy data was provided in Table 9C. The groups were divided as follows.

Groups: Subjects in 3 dose groups (4:1 ratio initially, then 1:1 in later cohorts)
30 μg [2 groups dosed (n=20)]
60 μg [3 groups dosed (n=30)]
90 μg [4 subjects dosed]

TABLE 9C
Demographics and Baseline Characteristics

| | 30 μg | | 60 μg | | Placebo | | Overall* | |
|---|---|---|---|---|---|---|---|---|
| Parameters | (N = 16) | | (N = 20) | | (N = 14) | | (N = 54) | |
| Mean age (SD) | 75.8 | (8.0) | 77.8 | (6.4) | 75.9 | (8.9) | 76.0 | (7.8) |
| Female (%) | 5 | (31.3) | 10 | (50.0) | 8 | (57.1) | 23 | (42.6) |
| Race (% | 81.3/18.8 | | 70.0/30.0 | | 92.9/7.1 | | 75.9/24.1 | |
| white/non- | | | | | | | | |
| white) | | | | | | | | |
| BMI | 27.5 | (5.7) | 23.6 | (3.8) | 25.1 | (7.0) | 25.4 | (5.4) |

DIAGNOSIS (n %)

| | | | | | | | | |
|---|---|---|---|---|---|---|---|---|
| AD | 14 | (87.5) | 17 | (85.0) | 13 | (92.9) | 47 | (87.0) |

| | | | | | | | |
|---|---|---|---|---|---|---|---|
| FTD | 1 | (6.3) | 1 | (5) | 0 | 2 | (3.7) |
| Vascular | 1 | (6.3) | 2 | (10) | 0 | 4 | (7.4) |

| | | | | | | |
|---|---|---|---|---|---|---|
| Unknown | 0 | 0 | 1 | (7.1) | 1 | (1.9) |

| | | | | | | |
|---|---|---|---|---|---|---|
| PEC baseline | 18.3 | (1.5) | 16.6 | (3.5) | 16.6 | (2.7) |
| (SD) | | | | | | |
| PAS | 8.9 | (0.9) | 9.1 | (1.3) | 8.7 | (0.9) |

Plasma samples were collected at 0.5, 1, 2, (2.5 in re-dose subjects), 4 and 8 hr.

Samples were analyzed for dexmedetomidine and the following samples were collected:
30 μg cohorts: 20 subjects dosed:
   4 Placebos
   8 subjects with few samples and/or no 8 hr sample (no AUC₀₋₈ calculated)
   1 subject was re-dosed (excluded)
   7 subjects with complete data
60 μg cohort: 19 subjects dosed:
   11 Placebos
   11 subjects with few samples and/or no 8 hr sample (no AUC₀₋₈ calculated)
   1 subject with inconsistent data
   7 subjects with complete data
90 μg cohorts: 4 subjects dosed
   2 subjects with few samples and/or no 8 hr sample (AUC₀₋₈ not calculated)
   2 subjects with sufficient samples

TABLE 9D
Pharmacokinetic parameters after administration
of dexmedetomidine film to dementia patients

| Dose | Cₘₐₓ (ng/L) | Cₘₐₓ/Dose | AUC₀₋₈ (hr*ng/L) | AUC_{0-8/}Dose |
|---|---|---|---|---|
| 30 μg | 85.25 [+/−17.34] | 2.84 | 425.2 [+/−105.8] | 14.17 |
| (n = 10 Cmax; =7 AUC) | {57.31-114.53} | | {271.2-569.4} | |
| 60 μg | 176.99 [+/−52.07] | 2.95 | 977.1 [+/−295.0] | 16.28 |
| (n = 9/7) | {126.36-298.98} | | {608.6-1307.4} | |

TABLE 9E
Comparison of pharmacokinetic parameters after administration of dexmedetomidine
film to dementia patients (Example -3) and schizophrenia patients

| | | Schizophrenia | | Schizophrenia |
|---|---|---|---|---|
| | | study - | | study - |
| | | NCT04268303 | | NCT04268303 |
| | Dementia study - | (SERENITY 1) | Dementia study - | (SERENITY 1) |
| | Example 3 | Study | Example 3 | Study |
| Dose | Cₘₐₓ/Dose | | AUC₀₋₈/Dose | |
| 20 μg | | 2.03 | | 9.74 |
| (Schizophrenia- | | | | |
| n = 8) | | | | |
| 30 μg | 2.84 | | 14.17 | |
| (n = 10 Cmax; =7 | | | | |
| AUC) | | | | |
| 60 μg | 2.95 | 2.33 | 16.28 | 9.54 |
| (Schizophrenia: | | | | |
| n = 18) | | | | |
| (Dementia: n = 9/7) | | | | |
| 80 μg | | 2.09 | | 9.89 |
| (Schizophrenia: | | | | |
| n = 18) | | | | |

TABLE 9F
Simulated pharmacokinetic comparison: dementia patients and schizophrenia
(NCT04268303 (SERENITY 1)/bipolar patients (NCT04276883 (SERENITY II))

| | | | Schizophrenia/ | | | | Schizophrenia/ | |
|---|---|---|---|---|---|---|---|---|
| | Dementia patients | | Bipolar patients | | Dementia patients | | Bipolar patients | |
| | | Cₘₐₓ/ | | Cₘₐₓ/ | | AUC_{0-inf}/ | | AUC_{0-inf}/ |
| Dose(μg) | Cₘₐₓ | Dose | Cₘₐₓ | Dose | AUC_{0-inf} | Dose | AUC_{0-inf} | Dose |
| 30 | 81.2 | 2.71 | | | 738 | 24.6 | | |
| 40 | 108 | 2.70 | | | 985 | 24.6 | | |
| 60 | 162 | 2.70 | | | 1480 | 24.7 | | |
| 120 | | | 291 | 2.43 | | | 1700 | 14.2 |
| 180 | | | 436 | 2.42 | | | 2540 | 14.1 |

PK summary—Table 9E shows that higher exposure levels are observed in elderly dementia patients, which can allow for efficacy at lower doses. Dexmedetomidine has broad potential in treating the full spectrum of agitation in patients with dementia. Table 9F shows observed data were consistent with simulated data and lower clearance in elderly subjects, simulated exposures higher in dementia elderly patients than schizophrenia patients.

### Efficacy Data

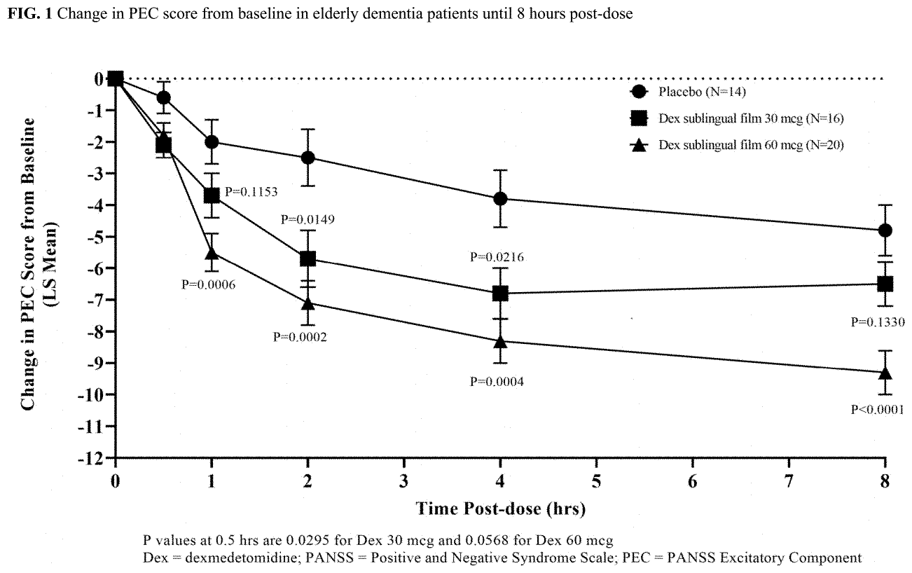

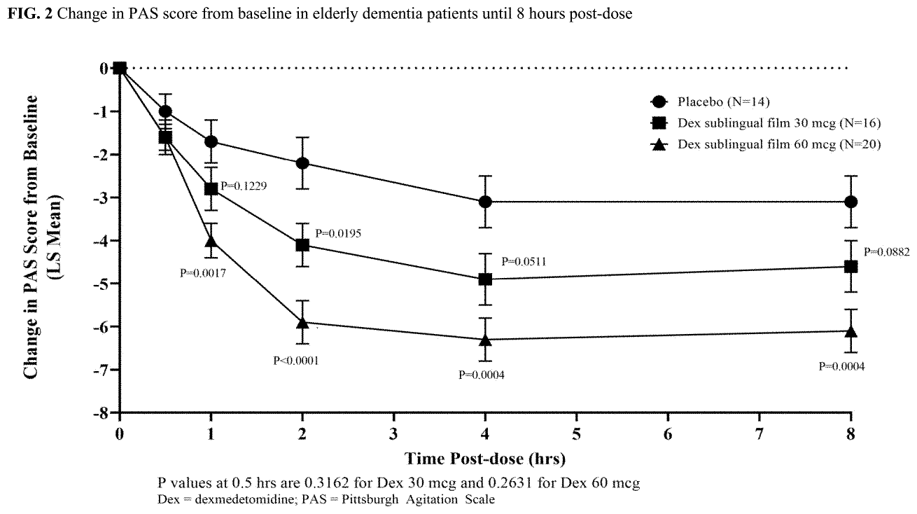

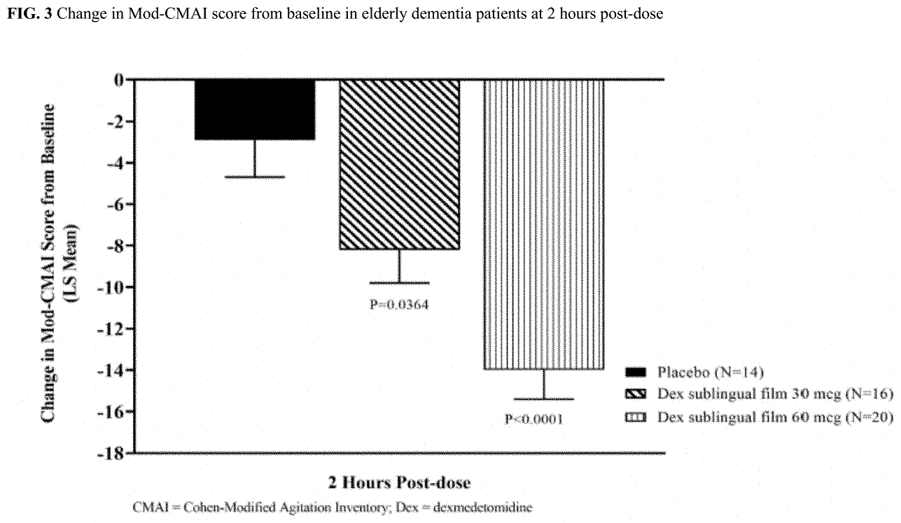

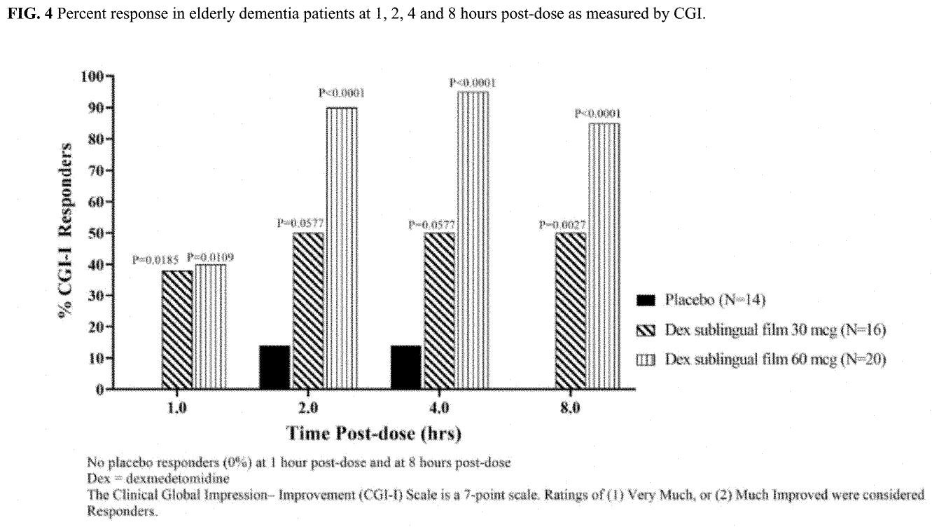

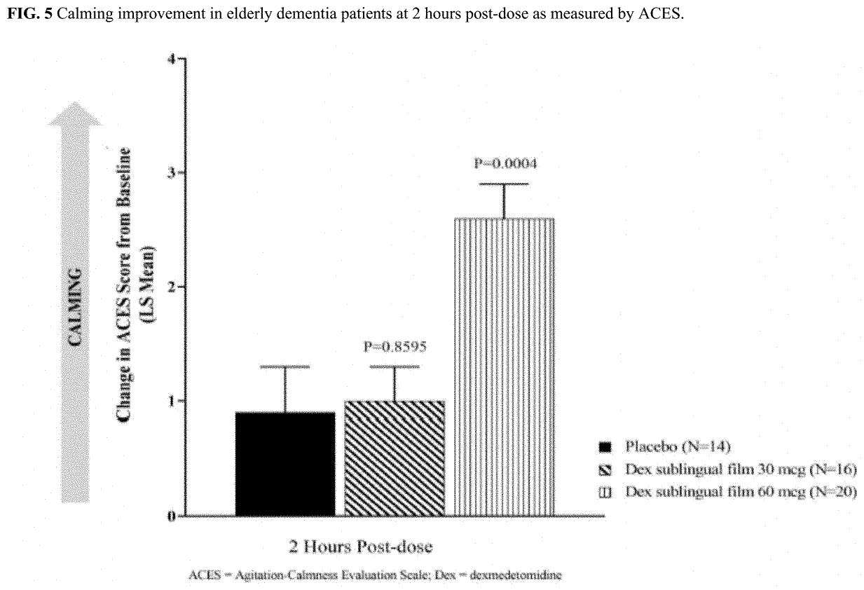

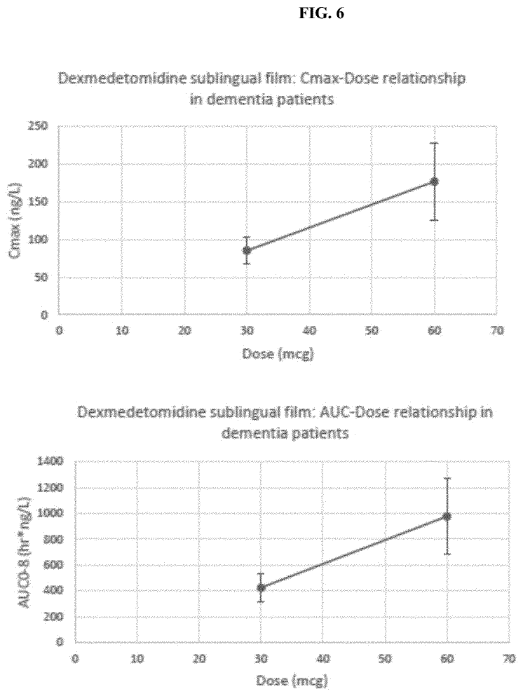

Further interim efficacy data for 30 μg and 60 μg dexmedetomidine oromucosal film can be observed with the measurement of following parameters
PEC Score reduction—PEC score reduction (see FIG. 1 and Table 9G).
PAS score reduction (see FIG. 2 and Table 9H).
Mod-CMAI score reduction (see FIG. 3 and Table 91),
CGI-I improvement—see FIG. 4, and
ACES score improvement—see FIG. 5
These data shows that both 30 μg and 60 μg dexmedetomidine oromucosal films are remarkably efficacious at treating agitation associated with dementia in elderly patients.

TABLE 9G
Statistically significant reduction in PEC scores of dementia patients
at 2 hours after administration of dexmedetomidine film

| | Placebo | 30 μg | 60 μg |
|---|---|---|---|
| PEC Total Score | (N = 14) | (N = 16) | (N = 20) |
| Change from | −2.5 | −5.7 | −7.1 ** |
| Baseline | | | |
| (LS Mean) | | | |
| Response ^{∘} | 0% | 31% | 70% ** |

PANSS-Excitatory Component (PEC) is a 5 items scale: Excitement, Hostility, Tension, Uncooperativeness, Poor Impulse Control, rated 1—Absent to 7—Extreme, ITT analysis, Least Square Means ± SEM

Proportion achieving ≥40% PEC reduction

TABLE 9H
The significant reduction in Agitation score as
confirmed by PAS in dementia patients at 2 hours
after administration of dexmedetomidine film

| | Placebo | 30 μg | 60 μg |
|---|---|---|---|
| PAS Total Score | (N = 14) | (N = 16) | (N = 20) |
| Baseline | 8.7 | 8.9 | 9.1 |
| Change from Baseline | −2.2 | −4.1 | −5.9 **** |
| (LS Mean) | | | |

Pittsburgh Agitation Scale (PAS) measures 4 behavior groups: aberrant vocalization, motor agitation, aggressiveness, and resisting to care rated 0—no agitation present to 4—highest form of agitation.
ITT analysis, Least Square Means ± SEM

TABLE 9I
Reduction in agitation score as confirmed by
modified CMAI in dementia patients at 2 hours
after administration of dexmedetomidine film:

| | Placebo | 30 μg | 60 μg |
|---|---|---|---|
| Mod-CMAI Total Score | (N = 14) | (N = 16) | (N = 20) |
| Baseline | 45.7 | 54.2 | 53.2 |
| Change from Baseline | −2.9 | −8.2 | −14.0 **** |
| (LS Mean) | | | |

Modified Cohen-Mansfield Agitation (Mod-CMAI) is an inventory consisting of 29 behaviors, each rated on a 7-point scale of frequency: 1—never to 7—several times an hour. Only behaviors manifested by the subject at baseline were assessed throughout the study.
ITT analysis, Least Square Means ± SEM

TABLE 9J
Dexmedetomidine film well tolerated with
no severe or serious adverse events

| | | 30 μg | | 60 μg | | Placebo |
|---|---|---|---|---|---|---|
| Parameters | | (N = 16) | | (N = 20) | | (N = 14) |
| Somnolence* | Mild | 9 | (56.3%) | 11 | (55.0%) | 0 |
| | Moderate | 0 | (0%) | 1 | (5.0%) | 0 |
| Hypotension | Mild | 0 | (0%) | 1 | (5.0%) | 0 |
| | Moderate | 0 | (0%) | 1 | (5.0%) | 0 |
| Orthostatic | Mild | 0 | (0%) | 1 | (5.0%) | 0 |
| hypotension | Moderate | 1 | (6.3%) | 0 | | 0 |
| Dizziness | Mild | 1 | (6.3%) | 1 | (5.0%) | 0 |
| | Moderate | 0 | (0%) | 0 | (0%) | 0 |

| | | | | |
|---|---|---|---|---|
| Bradycardia | 0 | 1 | (5.0%) | 0 |
| Dry mouth | 0 | 1 | (5.0%) | 0 |
| Nausea | 0 | 1 | (5.0%) | 0 |
| Headache | 0 | 1 | (5.0%) | 0 |

Verbatim; drowsy or feeling sleepy

### Interim Data Summary

Dexmedetomidine oromucosal film was found to be safe in dementia patient population and well tolerated to elderly patient population at the tested doses with no severe or serious side effects and no cases of syncope and falls reported. Statistically significant reductions in agitation achieved at 2 hours post-dose with both 30 and 60 μg cohort as measured by the PEC, PAS and Mod-CMAI scales, with:
Numerical separation as early as 30 min in PEC score, with statistically significant reductions from baseline observed at 60 min in PEC & PAS scores
Duration of response lasted 8 hrs after treatment with 60 μg dose
All exploratory endpoints demonstrated statistically significant reductions from baseline in agitation with 60 μg dose
CGI-I demonstrated statistically significant improvement from baseline in agitation with 30 μg dose.

### Example 4: Extension of Population Pharmacokinetic Analysis and Exposure-Response Analysis of the Oromucosal Film Formulation of Dexmedetomidine Hydrochloride in Patients with Dementia.

### Objectives

The main obj ectives of the analysis were to characterize the population PKPD of Dexmedetomidine oromucosal film following single and multiple (2 repeated doses) dosing in elderly patients with dementia and to explore the impact of patient characteristics on variability in relevant PK parameters and exposure-response relationships (ERRs).

This was achieved through the following sub-objectives:
(i). Extension of the existing PopPK model by inclusion of data from the recent phase 1b/2 study among elderly patients with dementia
(ii). Evaluation of the impact of age and different disease type on the pharmacokinetic (PK) of dexmedetomidine oromucosal film
(iii). Extension and adaption of the existing population PKPD models to describe the efficacy and occurrence of adverse event as a function of predicted dexmedetomidine oromucosal film exposure for dementia patients
(iv). Evaluation of the intended dosing regimen of dexmedetomidine oromucosal film in dementia patients by the use of simulations

This analysis represents an extension of previously developed PKPD model framework to include data from placebo-controlled Phase 1b/2 clinical trial where at least 50 elderly patients suffering from dementia were included. The relationships between dexmedetomidine oromucosal film exposure and efficacy and safety endpoints in elderly populations with agitation suffering from dementia were evaluated.

The efficacy modelling framework that involved PEC and ACES was adapted and extended to include the additional endpoints, Pittsburgh Agitation Scale (PAS) and modified Cohen Mansfield Agitation Inventory Scale (m-CMAI). In addition, the safety endpoint, somnolence, was modelled using logistic regression. The model was subsequently used to simulate the expected safety and efficacy response under a range of different dosing regimens in a manner similar to what was done previously.

### Data

Dexmedetomidine hydrochloride was given as a thin film formulation for sublingual (SL) administration in doses of 30, 60, or 90 μg. Data from 39 patients were included in the previously created PopPK analysis data set. One subject each in the 30 and 60 μg dose group received a redose 2 h after the first dose based on limited efficacy, determined as a reduction in PEC score of less than 40%.

### Methods

The existing PK model was fit to the combined data set, with a special focus on differences in PK parameters among elderly patients and patients suffering from dementia. The extended model was then used to derive exposure estimates for use in the development of the models for efficacy and safety, for which separate PKPD models were developed. Efficacy was quantified using an integrated analysis of the exposure-response relationship (ERR) between sublingual dexmedetomidine PK and two excitement/agitation scales, PEC and PAS. In addition, a similar, but separate model was developed for a third excitement/agitation endpoint, m-CMAI. Characterization of the safety profile of oromucosal film of dexmedetomidine hydrochloride involved a logistic regression analysis of the incidence of somnolence, as well as an extension of the longitudinal model of the ERR between oromucosal film of dexmedetomidine hydrochloride and the safety endpoints systolic blood pressure (SBP), diastolic blood pressure (DBP) and HR developed previously.

### Model Development, Diagnostics ad Qualification

During model development, the level of significance was P<0.01 for an effect to be included in the structural model development and forward inclusion of covariates. For retention of a covariate relation during backward elimination P<0.001 was used. Model stability was assessed throughout the model development and especially for the base model before covariate exploration.

Model development was carried out using importance sampling method assisted by mode a posteriori estimation (IMP-MAP). Diagnostic plots of observed data vs. population prediction (PRED) and individual prediction (IPRED) were examined for adequate fit. Plots of conditional weighted residual (CWRES) versus PRED and versus time (time after last dose as well as time after first dose) were inspected for evidence of systematic lack of fit, and to confirm the absence of bias in the error distributions.

### PK Simulations

Dexmedetomidine hydrochloride oromucosal film PK and ERR were simulated for the intended target population. The following dosing regimens were simulated: single doses of 30, 40, 60 and 90 mcg; multiple dose regimen: 30 mcg followed by 60 mcg after 2 hours and 30 mcg followed by 60 mcg after 6 hours. A simulation dataset was created for simulations in elderly patients (>65 years old). The dataset included 1000 subjects consisting of 500 males and 500 females to allow for a representative body weight (WT) distribution withinthis age range. Patient level characteristics, such as age, were simulated from the observed covariate distribution in the popPK data set.

Exposure and ERR for safety and efficacy endpoints were simulated every 0.05 hours and at 0.0833, 0.166, 0.25, 0.333, 0.5, 1, 1.5, 2, 3, 4, 5, 6, 8, 10, 12 and 24 hours after administration of the first dose of oromucosal film of dexmedetomidine hydrochloride.

### Efficacy Model Development

PEC model development was performed incrementally, starting with the development of a model to describe the placebo data. In a second step, data from the active treatment arms were added. Subsequent efficacy model development steps included optimization of the exposure-response relationship, variability structures and covariate effect evaluations. Once a satisfactory PKPD model was available describing the totality of the PEC response data, PAS and m-CMAI response data were added to estimate the relationship between both scales in a joint fit. The impact of patient and study-dependent covariates on the PKPD model parameters was graphically assessed throughout the model development.

### Simulations

Dexmedetomidine oromucosal film ERR Simulations using final models were performed in order to investigate a number of intended Phase 3 dosing regimens on the efficacy and safety of dexmedetomidine oromucosal film.

The following dosing regimens were investigated:
Single sublingual dose of 30 μg dexmedetomidine oromucosal film
Single sublingual dose of 40 μg dexmedetomidine oromucosal film
Single sublingual dose of 60 μg dexmedetomidine oromucosal film
Single sublingual dose of 30 μg followed by a sublingual dose of 60 μg administered 2 hrs after the initial dose.
Single sublingual dose of 30 μg followed by a sublingual dose of 60 μg administered 6 hrs after the initial dose.

Efficacy simulation results included full profiles of the PEC, PAS and m-CMAI vs. time, simulated with a delta time of 0.05h, and derived metrics such as % responders based on reaching a reduction from baseline PEC scoreof more than 40%. 90% confidence intervals were obtained by simulating 1000 individuals per treatment regimen using the estimated IIV (variance-covariance matrix).

### Model Diagnostics and Qualification

The final popPK model, PEC and PAS ERR models demonstrated good agreement between predicted and observed data. The CWRES are generally randomly scattered around the predicted concentration range and across time. The normal quantile-quantile plot and density plot of the CWRES indicated that the residuals were normally distributed with a mean and variance of approximately zero and one, respectively.

### Results

Summary statistics of the final model derived post hoc PK parameter estimates for first occasion and single dose (non-redosed) treatment in elderly dementia patients are tabulated in Table 10

TABLE 10
Summary of Model (post-hoc)- derived PK Metrics by Single Dose Treatment in Elderly Dementia Patients

| Dose | | | |
|---|---|---|---|
| 30 μg | 60 μg | 90 μg | All |
| (N = 16) | (N = 19) | (N = 4) | (N = 39) |

Fabs %

| | | | | | | | | |
|---|---|---|---|---|---|---|---|---|
| Mean (SD) | 47.2 | (9.6) | 45.0 | (16) | 42.7 | (14) | 45.7 | (13) |
| Median (range) | 49.5 | (28.5-65.4) | 46.8 | (11.9-69.9) | 42.9 | (25.5-59.6) | 46.8 | (11.9-69.9) |

CL (L/h)

| | | | | | | | | |
|---|---|---|---|---|---|---|---|---|
| Mean (SD) | 21.5 | (5.3) | 19.6 | (5.3) | 25.9 | (7.5) | 21.0 | (5.7) |
| Median (range) | 20.0 | (15.3-32.3) | 19.2 | (6.68-30.0) | 23.5 | (19.9-36.8) | 19.9 | (6.68-36.8) |

V1 (L)

| | | | | | | | | |
|---|---|---|---|---|---|---|---|---|
| Mean (SD) | 94.2 | (9.6) | 96.0 | (8.6) | 99.0 | (11) | 95.6 | (9.2) |
| Median (range) | 93.4 | (79.7-112) | 96.3 | (79.3-113) | 103 | (83.2-108) | 96.3 | (79.3-113) |

V2 (L)

| | | | | | | | | |
|---|---|---|---|---|---|---|---|---|
| Mean (SD) | 37.8 | (4.5) | 39.0 | (6.1) | 36.8 | (3.6) | 38.3 | (5.3) |
| Median (range) | 36.9 | (32.4-49.2) | 37.9 | (31.6-49.6) | 35.9 | (33.7-41.7) | 36.9 | (31.6-49.6) |

T_{1/2 elimination} (h)

| | | | | | | | | |
|---|---|---|---|---|---|---|---|---|
| Mean (SD) | 4.63 | (1.1) | 5.44 | (2.3) | 3.93 | (0.92) | 4.95 | (1.9) |
| Median (range) | 4.69 | (2.82-6.67) | 4.90 | (3.48-14.2) | 3.81 | (2.94-5.17) | 4.69 | (2.82-14.2) |

V_{z} (L)

| | | | | | | | | |
|---|---|---|---|---|---|---|---|---|
| Mean (SD) | 136 | (8.8) | 139 | (11) | 141 | (15) | 138 | (10) |
| Median (range) | 137 | (121-149) | 137 | (123-161) | 143 | (121-156) | 137 | (121-161) |

Cₘₐₓ(ng/L)

| | | | | | | | |
|---|---|---|---|---|---|---|---|
| Mean (SD) | 90.6 | (44) | 165 | (70) | 208 | (99) | — |
| Median (range) | 81.6 | (43.3-237) | 165 | (34.8-276) | 191 | (107-344) | — |

Tₘₐₓ(h)

| | | | | | | | |
|---|---|---|---|---|---|---|---|
| Mean (SD) | 1.92 | (0.74) | 1.80 | (0.47) | 1.58 | (0.53) | — |
| Median (range) | 1.80 | (1.12-4.03) | 1.71 | (1.15-2.89) | 1.43 | (1.15-2.28) | — |

AUC₀₋ₗₐₛₜ (pg · h/mL)

| | | | | | | | |
|---|---|---|---|---|---|---|---|
| Mean (SD) | 381 | (3.0e+02) | 853 | (9.9e+02) | 674 | (7.9e+02) | — |
| Median (range) | 330 | (38.1-1.34e+03) | 662 | (49.4-4.24e+03) | 375 | (108-1.84e+03) | — |

AUC_{0-Inf} (pg · h/mL)

| | | | | | | | |
|---|---|---|---|---|---|---|---|
| Mean (SD) | 709 | (2.7e+02) | 1.65e+03 | (1.3e+03) | 1.63e+03 | (7.6e+02) | — |
| Median (range) | 682 | (352-1.21e+03) | 1.45e+03 | (286-6.29e+03) | 1.75e+03 | (624-2.42e+03) | — |

Simulation results for the different efficacy and safety endpoints and treatment scenarios are discussed below.

### Longitudinal PK-PEC-PAS-CMAI Profiles

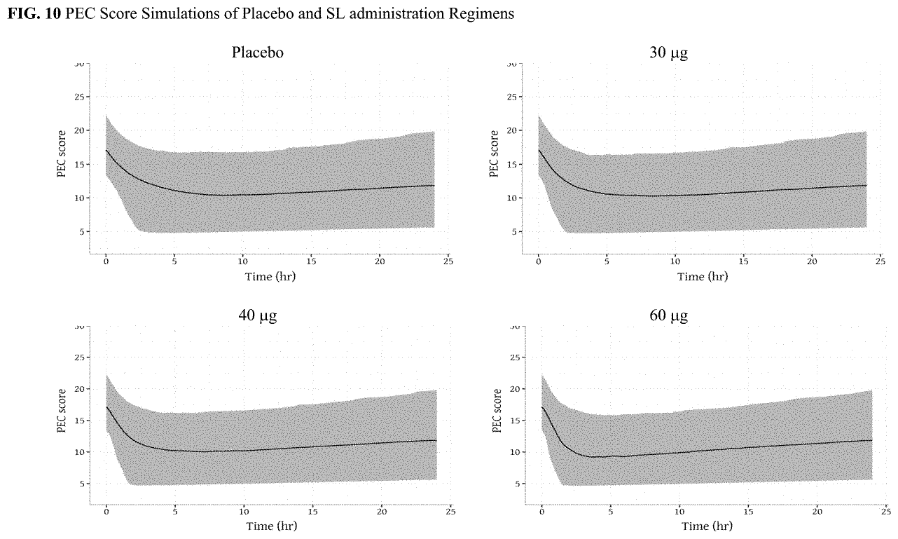

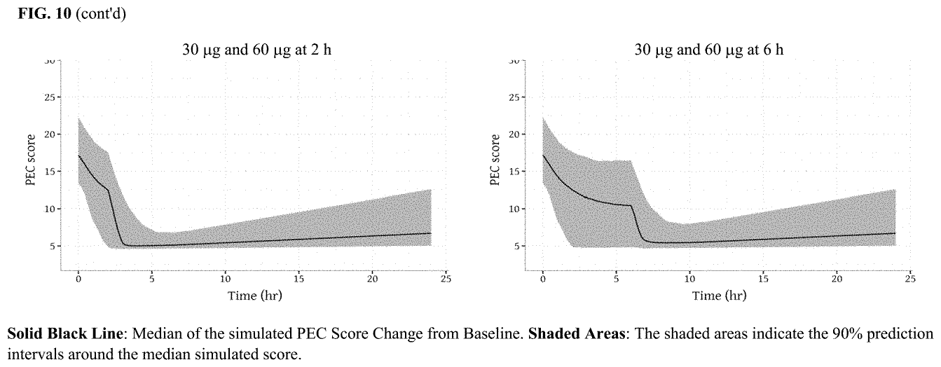

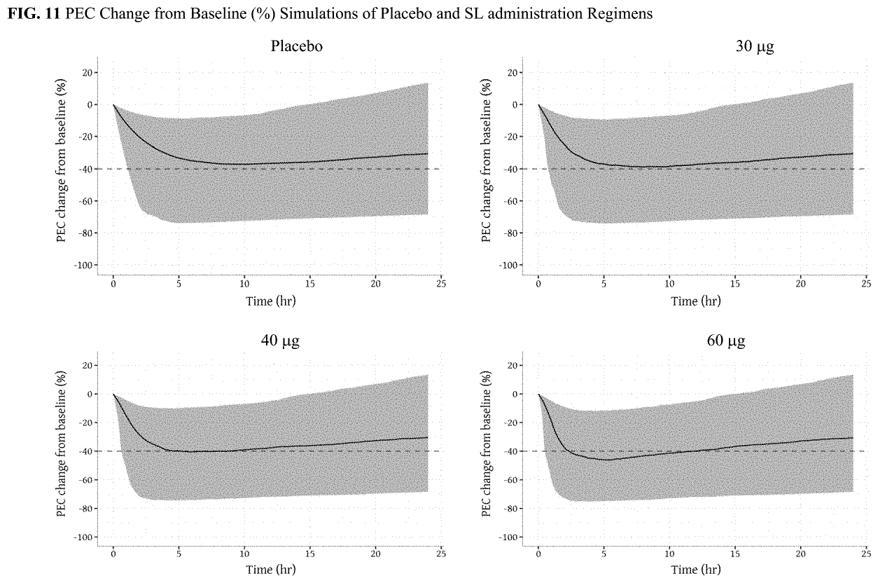

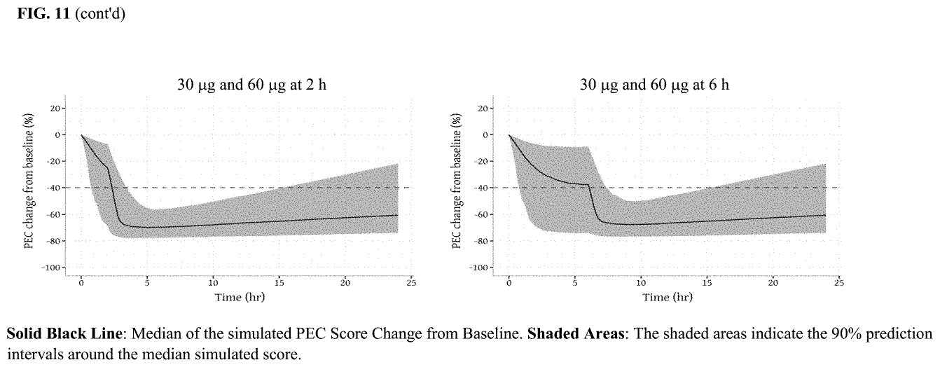

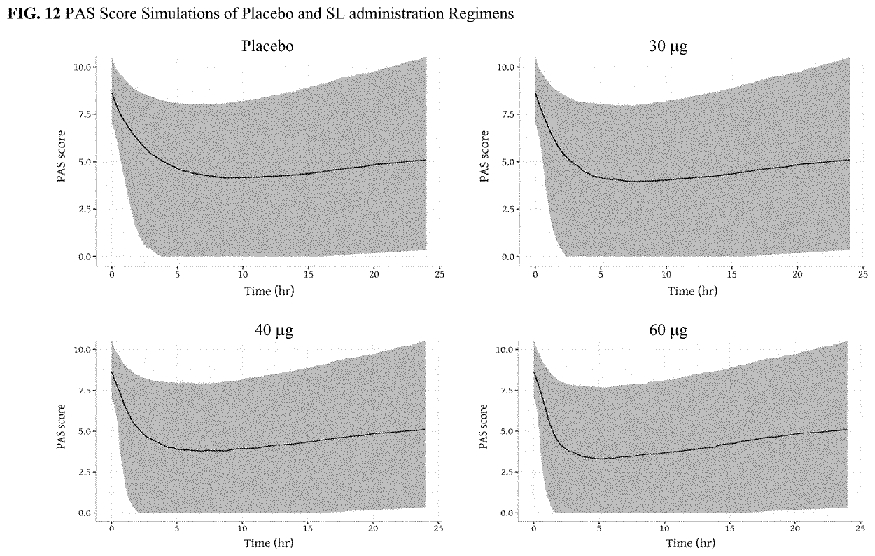

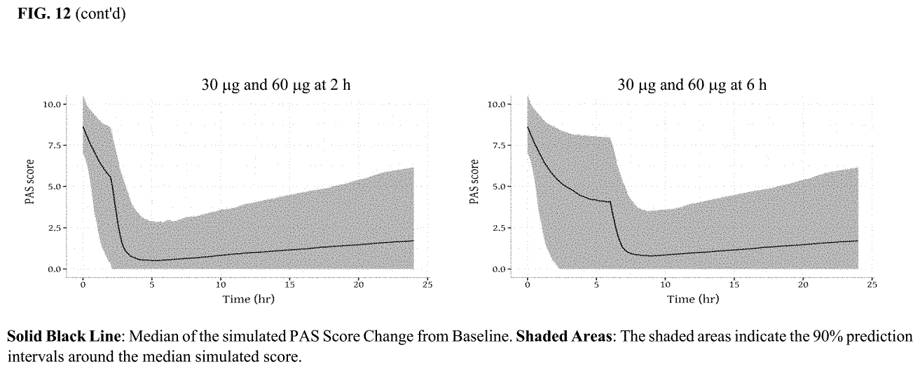

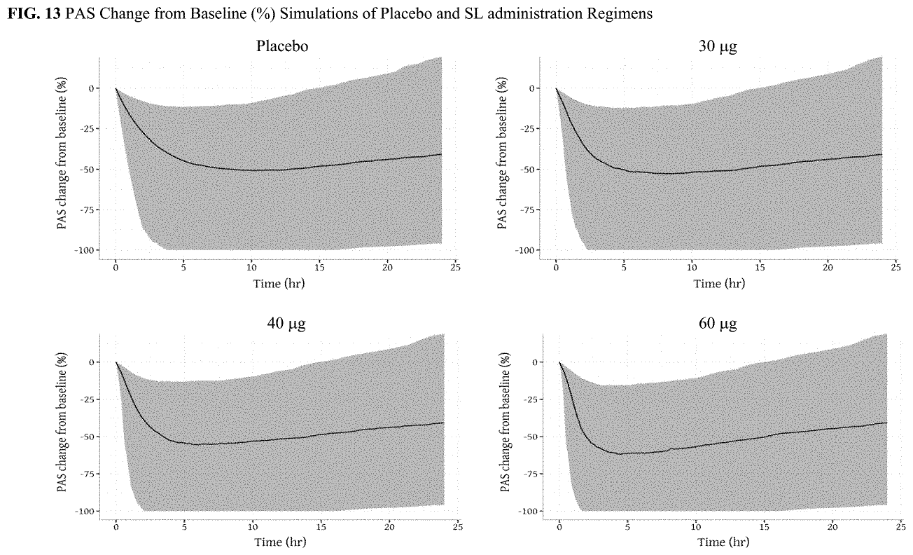

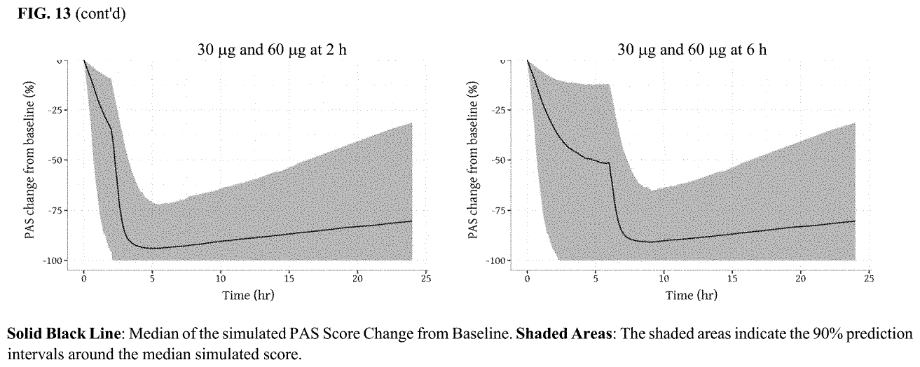

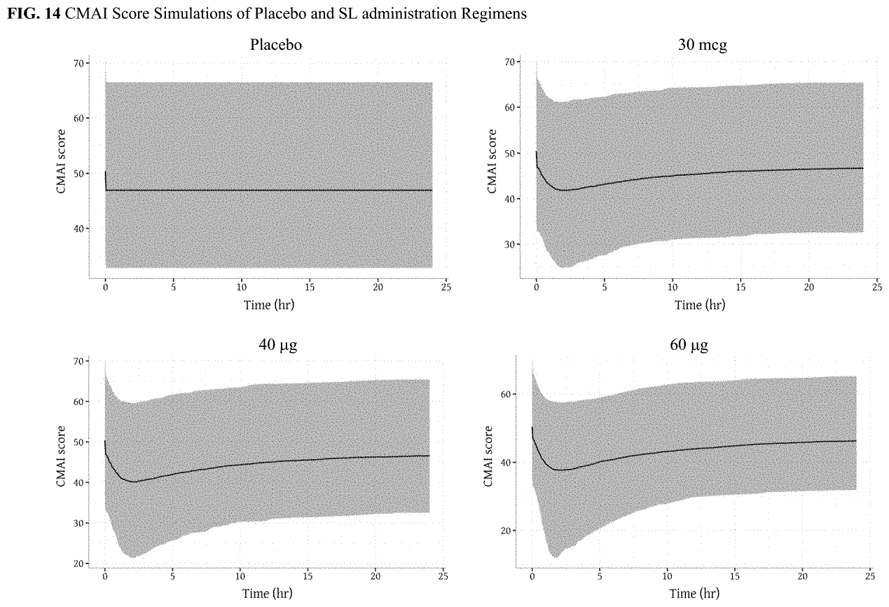

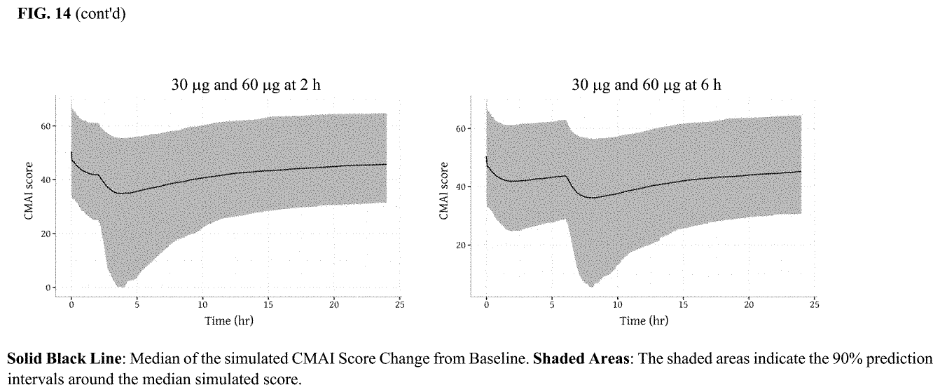

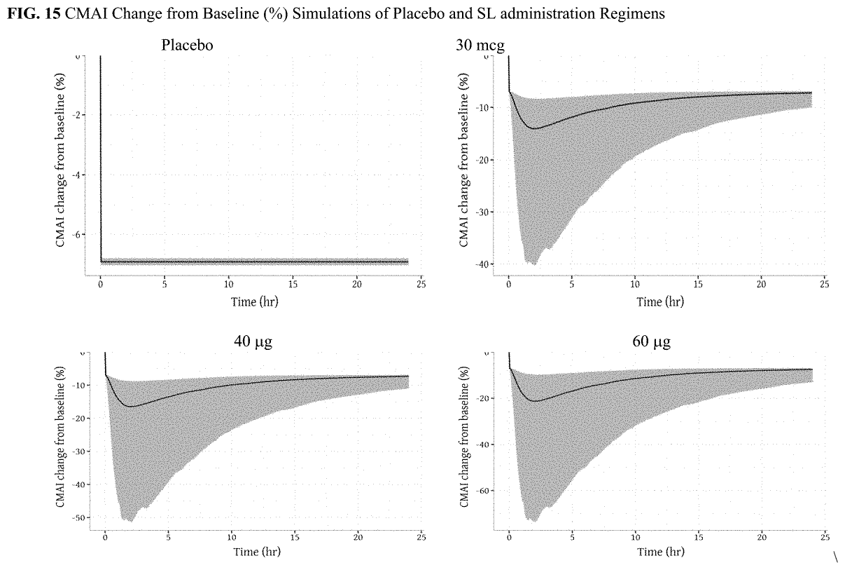

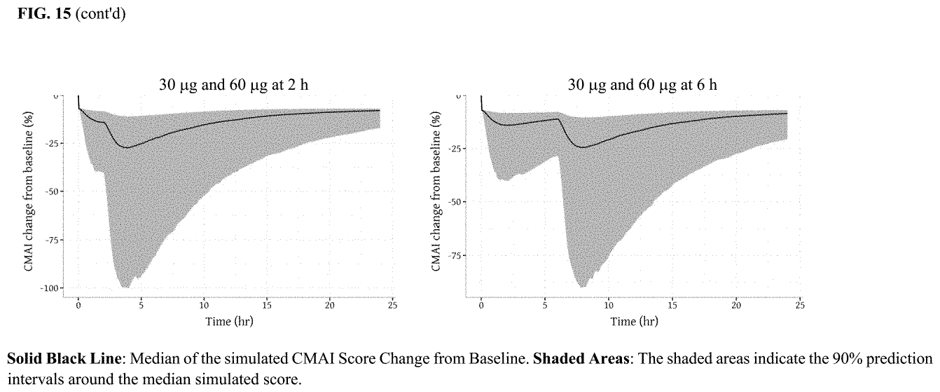

Summary statistics of the corresponding Cₘₐₓ and exposure are shown in Table 11. FIGS. 10-15 depict the simulated longitudinal profiles for PEC score, PEC change from baseline, PAS score, PAS change from baseline and m-CMAI score and m-CMAI change from baseline for the different regimens.

TABLE 11
Summary Statistics SL PK Simulation

| | | | Median | | Median | |
|---|---|---|---|---|---|---|
| | Median | 5^{th}-95^{th} | AUC_{0-24 h} | 5th-95th | AUC_{0-Inf} | 5^{th}-95^{th} |
| Regimen | Cₘₐₓ (ng/L) | % | (ng*h/L) | % | (ng*h/L) | % |
| 30 μg | 81.3 | 36-147 | 676 | 267-1400 | 714 | 271-1660 |
| 40 μg | 108.0 | 49-196 | 901 | 355-1860 | 952 | 361-2210 |
| 60 μg | 163.0 | 73-294 | 1350 | 533-2790 | 1430 | 541-3320 |
| 90 μg | 244.0 | 109-441 | 2030 | 800-4190 | 2140 | 812-4970 |
| 30 μg + | 228.0 | 103-410 | 2010 | 797-4130 | 2140 | 812-4970 |
| 60 μg at 2 h | | | | | | |
| 30 μg + | 198.0 | 90-359 | 1950 | 790-3930 | 2140 | 812-4970 |
| 60 μg at 6 h | | | | | | |

Summary statistics of the simulated effects on PEC and PAS in elderly dementia patients can be found in Table 12

TABLE 12
Simulated 2-hour Efficacy Response [Median (5^{th} 95th %)] - Elderly Dementia Patients

| | Concentration | | | | | | CFB PEC | |
|---|---|---|---|---|---|---|---|---|
| Dose | (ng/L) | | PEC | | CFB PEC | | (%) | |
| placebo | 0 | (0-0) | 13.2 | (6.18-18.2) | −3.52 | (−10.7-−0.991) | −20.4 | (−63.4-−6.05) |
| 30 μg | 77.9 | (33.6-142) | 12.5 | (4.95-17.6) | −4.39 | (−12.7-−1.13) | −25.1 | (−68.7-−7.03) |
| 40 μg | 104 | (44.8-189) | 11.8 | (4.78-17.4) | −4.97 | (−13.4-−1.28) | −28.5 | (−71.4-−8.21) |
| 60 μg | 156 | (67.2-283) | 10.5 | (4.68-17) | −6.29 | (−14.1-−1.51) | −37.7 | (−72.8-−9.07) |
| 30 μg + | 77.9 | (33.6-142) | 12.5 | (4.95-17.6) | −4.39 | (−12.7-−1.13) | −25.1 | (−68.7-−7.03) |
| 60 μg at 2 h | | | | | | | | |
| 30 μg + | 77.9 | (33.6-142) | 12.5 | (4.95-17.6) | −4.39 | (−12.7-−1.13) | −25.1 | (−68.7-−7.03) |
| 60 μg at 6 h | | | | | | | | |

| | | | | | CFB PAS | |
|---|---|---|---|---|---|---|
| Dose | PAS | | CFB PAS | | (%) | |
| placebo | 6.13 | (1.22-8.71) | −2.38 | (−7.17-−0.703) | −27.4 | (−86.6-−8.07) |
| 30 μg | 5.57 | (0.384-8.6) | −2.95 | (−7.89-−0.818) | −34.6 | (−95.4-−9.38) |
| 40 μg | 5.2 | (0.0434-8.42) | −3.33 | (−8.07-−0.961) | −38.3 | (−99.5-−10.8) |
| 60 μg | 4.22 | (0-8.07) | −4.39 | (−8.24-−1.06) | −50.5 | (−100-−12.4) |
| 30 μg + | 5.57 | (0.384-8.6) | −2.95 | (−7.89-−0.818) | −34.6 | (−95.4-−9.38) |
| 60 μg at 2 h | | | | | | |
| 30 μg + | 5.57 | (0.384-8.6) | −2.95 | (−7.89-−0.818) | −34.6 | (−95.4-−9.38) |
| 60 μg at 6 h | | | | | | |

Summary statistics of the simulated change in blood pressure and heart rate at Cₘₐₓ for the simulated dosing regimens in elderly dementia patients is found in Table 13 and Table 14

TABLE 13
Simulated Blood Pressure Response [Median (5^{th}-95^{th} %)] - Elderly Dementia Patients

| | | | Systolic BP | | CFB Systolic BP | | Diastolic BP | | CFB Diastolic BP | |
|---|---|---|---|---|---|---|---|---|---|---|
| Dose | Cₘₐₓ (ng/L) | | (mmHg) | | (mmHg) | | (mmHg) | | (mmHg) | |
| 30 μg | 81.3 | (36.4-147) | 125 | (97.8-145) | −6.05 | (−31.7-0.495) | 74.2 | (57.2-86.1) | −3.52 | (−18.7-0.257) |
| 40 μg | 108 | (48.6-196) | 123 | (94.7-145) | −8.56 | (−35.7-0.567) | 73.1 | (55.9-85.1) | −4.99 | (−21.6-0.309) |
| 60 μg | 163 | (72.8-294) | 120 | (91.4-143) | −12.2 | (−39.7-0.679) | 71.1 | (53.9-84.3) | −7.25 | (−23.9-0.375) |
| 90 μg | 244 | (109-441) | 117 | (88.4-142) | −15.9 | (−43.6-0.998) | 69.3 | (51.7-83.9) | −9.49 | (−25.3-0.527) |
| 30 μg + | 228 | (103-410) | 117 | (89-142) | −15.2 | (−42.7-0.882) | 69.7 | (51.9-83.9) | −9.11 | (−24.8-0.46) |
| 60 μg at 2 h | | | | | | | | | | |
| 30 μg + | 198 | (90.3-359) | 119 | (89.5-142) | −14.2 | (−41.2-0.731) | 70.4 | (52.2-83.9) | −8.48 | (−24.3-0.427) |
| 60 μg at 6 h | | | | | | | | | | |

TABLE 14
Simulated Heart Rate Response [Median (5th-95th %)]- Elderly Dementia Patients

| | | | Heart Rate | | CFB Heart | | Heart Rate | | CFB Heart Rate | |
|---|---|---|---|---|---|---|---|---|---|---|
| Dose | Cₘₐₓ (ng/L) | | (1/min) | | Rate (1/min) | | Placebo (1/min) | | Placebo (1/min) | |
| 30 μg | 81.3 | (36.4-147) | 71.3 | (55.7-87.9) | −4 | (−16-0.413) | 74.3 | (60.4-88.3) | −2.62 | (−3.26-−1.97) |
| 40 μg | 108 | (48.6-196) | 70.5 | (54.3-88.3) | −4.64 | (−17.8-1.29) | 74.3 | (60.4-88.3) | −2.62 | (−3.26-−1.97) |
| 60 μg | 163 | (72.8-294) | 70 | (53.1-88.8) | −5.74 | (−20-2.75) | 74.3 | (60.4-88.3) | −2.62 | (−3.26-−1.97) |
| 90 μg | 244 | (109-441) | 68.9 | (51.2-88.7) | −6.7 | (−21.4-3.75) | 74.3 | (60.4-88.3) | −2.62 | (−3.26-−1.97) |
| 30 μg + | 228 | (103-410) | 69 | (51.3-88.7) | −6.67 | (−21.3-3.59) | 74.2 | (60.3-88.2) | −2.7 | (−3.33-−2.06) |
| 60 μg at 2 h | | | | | | | | | | |
| 30 μg + | 198 | (90.3-359) | 69.4 | (52.2-88.8) | −6.3 | (−20.6-3.12) | 74.2 | (60.4-88.2) | −2.67 | (−3.29-−2.05) |
| 60 μg at 6 h | | | | | | | | | | |

### Conclusion

The model was able to adequately describe the observed data and there was good correlation between the observed and predicted data. The model was used to predict PK and efficacy and safety under different dosing scenarios and showed a dose-dependent change in exposure with corresponding changes in efficacy and safety measures. The model and simulations were able to build on the observed data and demonstrate the potential utility of dexmedetomidine oromucosal film in treating agitation associated with dementia.

### Example 5: A Phase Ib/II, Multicenter, Randomized, Double Blind, Placebo Controlled, Ascending Dose Finding, Efficacy, Pharmacokinetic and Safety Study of dexmedetomidine hydrochloride oromucosal film to treat symptoms of acute Opioid Withdrawal in patients with opioid use disorder who are physically dependent on opioids

### Primary Objective

Establish the safety and tolerability of ascending doses of dexmedetomidine hydrochloride oromucosal film relative to placebo in subjects with opioid use disorder who were physically dependent on opioids and maintained on oral morphine.

### Secondary Objectives

Establish the efficacy of dexmedetomidine hydrochloride oromucosal film relative to placebo in improving the following:
1. Opioid withdrawal symptoms:
   Short Opiate Withdrawal Scale of Gossop (SOWS-GOSSOP) and
   Clinical Opiate Withdrawal Scale (COWS)
2. Time to dropout after opioid discontinuation
3. Percentage of subjects dropping out after opioid discontinuation
4. Assessment of safety reflected by scores on the Agitation and Calmness Evaluation Scale (ACES) assessment

### Exploratory Objective

Evaluation of pharmacokinetics in subjects undergoing opiate withdrawal.

### Study Design

This inpatient Phase lb study assessed the safety, pharmacokinetics, and early signs of efficacy of escalating doses of dexmedetomidine hydrochloride oromucosal film versus placebo following discontinuation of morphine maintenance. The opioid maintenance phase (“morphine stabilization”) occurred during Study Days 1-5; the randomized dexmedetomidine hydrochloride oromucosal film/placebo phase (“randomized phase”) occurred on Study Days 6-12. The randomized phase was followed by 2 days of placebo oromucosal film (for dexmedetomidine) and morphine-placebo treatment for all remaining subjects on days 13-14.

After screening, eligible male and female adult subjects with OUD who were physically dependent on opioids and were not seeking treatment for OUD were admitted to an inpatient unit.

Morphine Stabilization (Days 1-5): At the start of the opioid maintenance phase (Study Days 1-5), subjects (n=225 enrollers) received oral morphine (30 mg) four times a day (QID) [8 am, 1 pm, 6 pm, and 11 pm (±30 minutes)] approximately every 4-6 hours or up to 5 times per day as needed with an additional 30 mg rescue dose available each day.

The total dose of morphine during Study Days 1-5) could be varied at the discretion of the investigator, between 120 mg-150 mg per day depending on patient's opioid use history and need for higher dose to stabilize withdrawal symptoms. In addition, all subjects received placebo films (B.I.D: 8 am and 8 pm), approximately 12 hours apart during this opioid maintenance phase (i.e., Days 1-5) to simulate and thus blinded treatment with dexmedetomidine oromucosal film during Days 6-12. During the stabilization phase participants had access to concomitant medications for: anxiety/restlessness, nausea, upset stomach, diarrhea, insomnia, muscle pain, and general discomfort. The use of benzodiazepines was limited to a standardized clonazepam taper. On Day 1:0.5 mg of clonazepam was available every 3-4 hrs, up to 2.0 mg total. Using this same schedule of availability, on Day 2, a total of 1.5 mg was available, Day 3, 1.0 mg total, and on Day 4, only one 0.5 mg dose was available. On Day 5, no clonazepam was available.

Morphine Discontinuation (Days 6-12): Starting on the morning of Day 6, blinded abrupt discontinuation of active morphine begun by replacing active morphine with placebo morphine. Placebo morphine capsules were identical in appearance to the morphine capsules taken during the opioid maintenance period. On this day (Study Day 6), subjects were randomized (within each cohort, 20 subjects received active dexmedetomidine oromucosal film and 5 subjects received placebo) to receive either placebo or dexmedetomidine hydrochloride films administered twice a day (BID), approximately 12 hours apart at approximately 8 am and 8 pm which continued throughout the inpatient period. For safety, randomization occurred in cohorts of escalating doses of dexmedetomidine oromucosal film. Placebo oromucosal film or dexmedetomidine oromucosal film administered on Days 6-12 along with placebo morphine (QID). Throughout the inpatient period, opioid withdrawal was assessed using the clinical opiate withdrawal scale (COWS) and short opiate withdrawal scale (SOWS) immediately prior to the 8 am dexmedetomidine oromucosal film or placebo dose, 2 hours post-dose, immediately prior to the 8 pm dexmedetomidine oromucosal film or placebo dose, and 2 hours post-dose. Agitation and sedation were also assessed using the Agitation and Calmness Evaluation Scale.

Post-Treatment Phase (Days13-14): On days 13 and 14, all remaining subjects received placebo morphine capsules (QID) and placebo oromucosal films (BID). During this time assessments of withdrawal and AEs continued. Additionally, a physical examination was conducted on the day of discharge.

Six cohorts were tested (n=25 per cohort; n=5 placebo and n=20 active dexmedetomidine oromucosal film) with potential to add cohorts or select different doses/schedule of dosing based on ongoing safety review and medical monitoring. The following doses were administered: 30 μg (Cohort 1), 60μg (Cohort 2), 90 μg (Cohort 3), 120 μg (Cohort 4), 180 μg (Cohort 5) and 240 μg (Cohort 6). The dose for Cohort 6 (240 μg) was determined from data acquired from cohorts 1-5 and cohort 6 was activated prior to the end of the study. Safety and tolerability were monitored continuously and summarized upon completion of each cohort by medical safety review. Studies of opioid withdrawal with placebo arms were likely to have high dropout rates, thus, the dropouts prior to Day 6 might be replaced to ensure enough sample size entering the treatment phase. The study was intended to be flexible and adaptable and as such, the dosing frequency, the doses and the number of cohorts of dexmedetomidine hydrochloride oromucosal film might be changed as a result of review of safety, tolerability and efficacy data.

Opioid withdrawal symptoms (SOWS-Gossop and COWS) were measured throughout the inpatient period at predose, 2 hours post dose, pre 2nd dose and 2 hours post second dose. Additional/SOWS-Gossop/COWS might be administered at investigator discretion. Transition to treatment for opioid use disorder was offered prior to patients leaving the unit.

Vital Signs, SOWS-Gossop, COWS, pulse oximetry and electrocardiogram (ECG) with rhythm strip were measured as per the schedule of assessments. Dexmedetomidine oromucosal film or placebo was self-administered sublingually under staff observation and subjects were allowed fluids as desired 15 minutes after completion of dosing. Safety and tolerability assessments continued until the morning of Day 14 (day of discharge).

Any abnormal vital sign measurement, clinical laboratory test, physical examination finding, or ECG parameter deemed clinically significant by the investigator were repeated, including test results obtained on the final study day or upon early termination. For any test abnormality deemed clinically significant, repeat analysis was performed during the follow-up period and until the value returns to baseline (or within normal limits) or the investigator deems the abnormality to be stable and no longer of clinical concern.

One-week Follow-up (Day 21): Participants returned to the study site one week after discharge to complete a follow-up visit that included multiple assessments of health.

### Diagnosis and Main Criteria for Eligibility

### Inclusion Criteria

Male and female subjects who were 18 years old to less than 65 years old.

Mets criteria for moderate to severe opioid use disorder as per Diagnostic and Statistical Manual of Mental Disorders, Fifth Edition (DSM-5) criteria and confirmed by the Mini-International Neuropsychiatric Interview (MINI) with physiological dependence as evidenced by a Clinical Opiate Withdrawal (COWS) score of >5 or a positive naloxone challenge upon admission on Day 1.

Subjects who could read, understand and provide written informed consent.

Women of childbearing potential must have a negative pregnancy test and agreed to be abstinent or use an acceptable method of contraception for the duration of the study.

### Exclusion Criteria

Positive urine pregnancy test at screening or when tested or currently breast feeding.
1. Clinically significant history of cardiac disease, screening and baseline heart rate of <55 beats per minutes or systolic blood pressure <110 mmHg or diastolic blood pressure <70 mmHg.
2. History or presence of a significant medical disease or disorder which, in the opinion of the investigator, increases the risk or may confound the interpretation of study measures, as confirmed by screening laboratory results.
3. Hepatic dysfunction (marked by ascites, or bilirubin >10% above the upper limit of normal [ULN] or liver function tests >3×ULN) at the screening visit.
4. Acute active Hepatitis B or C as evidenced by positive serology and aspartate aminotransferase (AST)/alanine aminotransferase (ALT) >2×ULN.
5. Clinically significant abnormal ECG findings such as second or third degree heart block, uncontrolled arrhythmia, or QTc (Fridericia correction formula) interval >450 msec for males, and >470 msec for females at screening or prior to dosing.
6. Any psychiatric disorder that would compromise ability to complete study requirements.
7. Currently meets DSM-5 criteria for substance abuse disorder, moderate or severe for any substance other than opioids, caffeine, or nicotine and/or current physical dependence on drugsthat pose risk of withdrawal that requires medical management such as alcohol or benzodiazepines.
8. History of suicidal behavior within the last 1 year prior to screening.
9. Participation in a clinical trial of a non-FDA-approved pharmacological agent within 30 days prior to screening.
10. Use of any excluded medication at screening or anticipated/required use during the study period.
11. Subjects with a history of intolerance to morphine.
12. Any finding that, in the view of the principal investigator, would compromise the subject's ability to fulfill the protocol visit schedule or visit requirements.

### Study Treatments

### Test Product, Dose, and Mode of Administration:

Dexmedetomidine hydrochloride oromucosal film 30 μg, 60 μg, 90 μg, 120 μg, 180 μg, and 240 μg doses as a thin film formulation of dexmedetomidine for sublingual (SL) administration. The product was a small, solid-dose film formulation designed to completely dissolve in the SL space within 1-3 minutes.

### Reference Therapy, Dosage and Mode of Administration:

Matching placebo films were taken sublingually as described above.

### Duration of Treatment

30 mg QID or 5×/day Morphine and placebo oromucosal film (of dexmedetomidine): 5 days;

BID dexmedetomidine hydrochloride oromucosal film or placebo oromucosal film and morphine placebo: 7 days, placebo oromucosal film (of dexmedetomidine) and morphine placebo: 2 days.

### Study Procedures

Subjects were provided written informed consent before any study-related procedures are initiated, including the cessation of prohibited concomitant therapy.

TABLE 15A
Schedule of Visits and Assessments

| | | Inpatient Admission (14 days) | | | | |
|---|---|---|---|---|---|---|
| | | Detoxification | | | | |
| | | | Randomization | | Post | |
| | | | & first | | Treatment | One-week |
| | | Morphine | day of | Treatment | phase or | Follow-up |
| | Screening¹ | maintenance | treatment | Phase | ET | Day 21 (±3 |
| Day | −2 to −1 | Days 1-5 | Day 6 | Days 7-12 | Days 13-14 | days) |
| Naloxone Challenge² | | X | | | | |
| Informed Consent | X | | | | | |
| Inclusion/Exclusion | X | | X | | | |
| Criteria³ | | | | | | |
| Mini International | X | | | | | |
| Neuropsychiatric Inventory | | | | | | |
| (MINI) | | | | | | |
| Columbia Suicide Severity | X | | | | | |
| Rating Scale (C-SSRS) | | | | | | |
| Randomization (Day 6) | | | X | | | |
| Demographics | X | | | | | |
| Medical and Psychiatric | X | | | | | |
| History | | | | | | |
| Concomitant Medications | X | X | X | X | X | X |
| 12 - LeadECG⁴ | X | X | X | X | X | |
| Physical Exam⁵ | X | X | X | X | X | |
| Clinical labs | X | | X | | | X |
| Vital Signs Measurements⁶ | X | X | X | X | X | X |
| HIV testing | X | | | | | |
| Buccal SL assessment⁷ | | X | X | X | X | |
| Rapid Urine Pregnancy | X | X | X | | | |
| Testing⁸ | | | | | | |
| AE Monitoring | X | X | X | X | X | X |
| Urine Toxicology/BAL⁹ | X | X | X | X | | X |
| Urinalysis | X | | X | | | X |
| Timeline Followback | X | | | | | X |
| Pharmacokinetics¹⁰ | | | X | X¹¹ | | |
| SOWS & COWS¹² | X | X | X | X | X | |
| Administration of | | X | | | | |
| Morphine | | | | | | |
| Administration of | | X | X | X | X | |
| Dexmedetomidine | | | | | | |
| oromucosal film or | | | | | | |
| Placebo¹³ | | | | | | |
| Administration of | | | X | X | X | |
| Morphine Placebo¹⁴ | | | | | | |
| | | | | | X | |
| Agitation and Calmness | | X | X | X | | |
| Evaluation Scale (ACES)¹⁵ | | | | | | |

¹All procedures must be completed prior to subject randomization and within 30 days of signing informed consent.
²Subjects had the option of naloxone challenge on admission if not displaying signs/symptoms of withdrawal. If naloxone is administered, subjects might receive morphine to alleviate the opioid withdrawal symptoms that may be present at the end of the challenge test.
³Inclusion/Exclusion criteria evaluated at Screening and pre dose on Randomization Day 6.
⁴ECG was done on Screening, pre-morning dose of oromucosal film -placebo (of dexmedetomidine) on Day 1-5, pre-morning dose on day 6, 8, 10 and pre-morning dose of placebo on Days 13-14.
⁵The Physical Exam were performed at Screening, day 1, day 8, day 10, and day 14/discharge. Height to be collected at Screening only. Weight to be collected at Screening and Day 14/discharge only.
⁶Vital sign measurements included orthostatic blood pressure, pulse, and measurement of oxygen saturation. Resting (recumbent) vital signs (SBP, DBP and HR) were taken upon having the subject recumbent for 5 min at Screening, prior to each dexmedetomidine film on days 1-14, and Day 21/follow-up. Orthostatic measurements (SBP, DBP, HR, respiratory rate) were taken upon having the subject stand for 5 minutes, with measurements taken at Screening, pre-dose and 2 hours post-dose each dexmedetomidine film on days 1-14, and Day 21/follow-up with windows of −5 minutes and +15 minutes. Triplicate measurements were performed in case of Systolic BP <90 mmHg, Diastolic BP <60 mmHg, or Pulse <60 bpm.
⁷Buccal exam at 30 min post first dose for signs of local irritation on Days 1, 6, & 12. Buccal exam was done prior to discharge on Day 14. Additional buccal exam may be done at investigator's discretion or in case of a relevant adverse event.
⁸Arapid urine pregnancy test was performed at screening, Day 1 and 6.
⁹Urine drug testing included opioids (fentanyl), buprenorphine, methadone, , benzodiazepines, cocaine (benzoylecgonine), amphetamines, and other drugs. Breath alcohol levels (BAL) assessed at screening, Day 1 and follow-up.
¹⁰Blood samples for pharmacokinetic analyses took place on inpatient Study Days 6 and 12 at 0, 2, , 6, and 12 hr after the first dose of dexmedetomidine oromucosal film of the day. The 12 hr. sample will be taken just before the administration of the next dose of dexmedetomidine oromucosal film or placebo oromucosal film (for dexmedetomidine). A window of +/−5 mins per PK sample were allowed without deviation.
¹¹PK samples were collected on Day 12 only at the scheduled times.
¹²COWS and SOWS-Gossop assessments were performed Pre-dose, 2 hour post-dose, pre-2^{nd} dose, and 2 hour post 2^{nd} dose. Additional assessments done at investigator's discretion.
¹³Administration of dexmedetomidine oromucosal film or Placebo was given BID (approximately 8am and 8pm [+/−30 minutes]). On Days 1-5, subjects received placebo oromucosal films (of dexmedetomidine) approximately 12 hours apart, besides their morphine treatments.
¹⁴Morphine placebo was administered at approximate same time as Day 1-5.
¹⁵Administration of the Agitation and Calmness Evaluation Scale (ACES) occurred at approximately two hours after (−5/+15 mins) dexmedetomidine oromucosal film or placebo dosing (approximately 10am and 10pm).

### Criteria for Evaluation

Treatment-Emergent Adverse Events and Serious Adverse Events were the primary dependent measures of the safety and tolerability of dexmedetomidine oromucosal film. An adverse event was defined as any unfavorable and unintended symptom or laboratory finding. A Treatment-Emergent Adverse Event (TEAE) was defined as an adverse event with an onset equal to or after randomization (Day 6). TEAEs were coded using Medical Dictionary for Regulatory Activities (MedDRA) terminology and summarized descriptively by treatment condition (i.e., placebo vs active dexmedetomidine oromucosal film), relationship to study drug, and severity (i.e., mild, moderate, or severe).

Short Opiate Withdrawal Scale (Gossop et al. 1990) is a 10-item self-report measure designed to quantify the severity of opioid withdrawal. Participants are asked to rate the following 10 items as “None,” “Mild,” “Moderate,” or “Severe.” “Feeling Sick,” “Stomach Cramps,” “Muscle Spasms/Twitching,” “Feeling of Coldness,” “Heart Pounding,” “Muscular Tension,” “Aches and Pains,” “Yawning,” “Runny Eyes,” and “Insomnia/Problems Sleeping.”

The score on the SOWS-Gossop ranges from 0 to 30, with higher scores indicating greater severity of withdrawal symptoms.

Clinical Opiate Withdrawal Scale (Wesson and Ling, 2003) is an 11-item clinician-administered measure designed to quantify the severity of opioid withdrawal. The COWS evaluates the severity of the following symptoms on a scale of 0-4 or 5: resting pulse rate, sweating, restlessness, pupil size, bone or joint aches, runny nose or tearing, gastrointestinal upset, tremor, yawning, anxiety or irritability, and gooseflesh. The scores on the COWS ranges from 0 to 48 with withdrawal ratings between 5 to 12 considered mild, 13 to 24 as moderate, 25 to 36 as moderately severe, and >36 considered severe withdrawal.

The Agitation and Calmness Scale consist of a single item in which a clinician rates acute agitation and sedation of the participant/patient on the following scale: 1=marked agitation, 2=moderate agitation, 3=mild agitation, 4=normal, 5=mild calmness, 6=moderate calmness, 7=marked calmness, 8=deep sleep and 9=unarousable.

### Pharmacokinetics

Blood samples (4 ml) were collected for pharmacokinetic analysis on inpatient Study Days 6 and 12 at 0, 2, 6, and 12 hours after the first dose of dexmedetomidine hydrochloride oromucosal film on Study Days 6 and 12. The 12 hr. sample was taken just before the administration of the next dose of dexmedetomidine oromucosal film or placebo.

Statistical Analysis: The study sought to enroll a total of 150 participants (25 per dosing cohort). This sample size was estimated to provide at least 10 completers (i.e., thru Day 12) per dosing cohort, ensuring precise 95% confidence intervals, with a margin of error of <2.7. Demographics data were summarized descriptively by treatment condition (e.g., number, percentage, mean, standard deviation, and range. All comparative statistical tests were two-sided, and the significance level was set at p<0.05. Analyses were conducted on all subjects who completed at least one post-Day 6 (i.e., randomization) assessment of the outcome measures above. The distributions of all continuous variables were checked for normality before utilizing the parametric tests described below. Statistical analyses were performed using SAS version 9.3 (SAS Institute).

### Primary Outcomes

The safety and tolerability of dexmedetomidine oromucosal film was measured by the number of TEAEs (defined above) by treatment condition. The number and percentage of subjects experiencing 1 or more TEAEs was summarized by treatment condition and severity. Chi-square tests were used to compare the frequencies of TEAEs across the treatment conditions.

### Secondary Outcomes

COWS and SOWS scores during Days 6-12. COWS and SOWS scores on Days 6-12 were analyzed using a mixed-effects, repeated-measures model. These data were examined using average change scores from pre-dose. For these comparisons, “average” refers to the average of the change in score across the two administrations of dexmedetomidine oromucosal film (i.e., 2-hour post-dose from the respective pre-dose scores). Planned comparisons for each “Day” were used to identify which treatment groups (i.e., dexmedetomidine oromucosal film dose) were significantly different from placebo.
Days of retention following discontinuation of active morphine (days 6-12). Kaplan-Meier estimates were used to generate survival curves for each treatment group. A logistic regression model was used to compare the numbers of subjects dropping out after opioid discontinuation as a function of dexmedetomidine oromucosal film maintenance condition.
Mean agitation and calmness as measured by the ACES (days 6-12). Differences in mean agitation and sedation were compared between the treatment groups using the same mixed-effects, repeated-measures model described above.

TABLE 15B
Time to Dropout After Morphine Discontinuation (FAS Population)

| Time to dropout for | 30 μg | | 60 μg | | 90 μg | | 120 μg | | 180 μg | | 240 μg | | Placebo | |
|---|---|---|---|---|---|---|---|---|---|---|---|---|---|---|
| Days 6-12 (in days) | (N = 17) | | (N = 17) | | (N = 21) | | (N = 19) | | (N = 21) | | (N = 15) | | (N = 25) | |
| Event, n (%) | 10 | (58.8) | 11 | (64.7) | 15 | (71.4) | 11 | (57.9) | 13 | (61.9) | 9 | (60.0) | 19 | (76.0) |
| Censored, n (%) | 7 | (41.2) | 6 | (35.3) | 6 | (28.6) | 8 | (42.1) | 8 | (38.1) | 6 | (40.0) | 6 | (24.0) |
| Q1 | 2.0 | | 1.0 | | 2.2 | | 1.1 | | 2.1 | | 1.1 | | 0.5 | |
| Median (95% CI) | 3.1 | (0.7, NE) | 1.5 | (0.0, NE) | 3.1 | (2.2, 6.0) | 5.1 | (1.1, NE) | 6.1 | (2.1, NE) | 3.5 | (1.0, NE) | 2.0 | (0.7, 5.1) |

| | | | | | | | |
|---|---|---|---|---|---|---|---|
| Q3 | NE | NE | NE | NE | NE | NE | 5.2 |

Abbreviations: CI = confidence interval, Q1 = 1st quartile, Q3 = 3rd quartile, NE = Not Estimable.
Subjects not discontinued by Day 12 are censored at 7 days.

Kaplan-Meier estimates were also used to generate survival curves over time in each treatment group. Logistic regression model was used to compare the dexmedetomidine hydrochloride oromucosal film and placebo groups on the numbers of subjects dropping out after opioid discontinuation.

### Safety Analyses

Safety data analyses were conducted on all subjects receiving at least 1 dose of study drug. The number and percentage of subjects experiencing 1 or more AEs were summarized by treatment, relationship to study drug, and severity. AEs were coded using Medical Dictionary for Regulatory Activities (MedDRA) terminology. Listings of subjects who withdraw from the study due to an AE, serious AEs and/or death or lack of treatment effect presented.

Laboratory parameters were summarized by treatment using descriptive statistics and data listings of clinically significant abnormalities. Vital signs and ECG data were summarized by changes from baseline values using descriptive statistics. Chi-square (or Fisher's exact) tests were used to compare the frequencies of AEs or serious AEs on blood pressure, heart rate, or respiratory drive between dexmedetomidine hydrochloride oromucosal film and placebo, at the beginning of Day 6 and then daily during the remainder of this study.

### Pharmacokinetic Analyses

Plasma concentration data for dexmedetomidine were analyzed by a popPK approach and reported separately. The plasma concentration data from this study were combined with PK data from other studies to develop the population PK model. The model was used to estimate exposure measures in subjects in this study through a modeling and simulation approach.

Interim data: The demographic data and baseline characteristics are given in Table 15C.

### Participants

356 participants were screened, and 225 participants were enrolled in the study. Discontinuation during the morphine stabilization phase (Days 1-5) was high (40%) but did not significantly vary among the six cohorts: Cohort 1: 36%, Cohort 2: 46%, Cohort 3: 28%, Cohort 4: 36%, Cohort 5: 43%; Cohort 6: 49% (p=0.19). Post dexmedetomidine oromucosal film randomization/morphine discontinuation (Day 6) data were available from 135 participants; detailed demographics of this sample are presented in Table 15C.

TABLE 15C
Sample Demographics and Baseline Characteristics

| | | | 30 μg | | 60 μg | | 90 μg | | 120 μg | |
|---|---|---|---|---|---|---|---|---|---|---|
| Dexmedetomidine | | | BID | | BID | | BID | | BID | |
| oromucosal Dose | | | (N = 17) | | (N = 17) | | (N = 21) | | (N = 19) | |
| Age (Years) | | Mean | 41.6 | (13.9) | 45.5 | (12.6) | 39.3 | (9.64) | 41.3 | (9.55) |
| | | (SD) | | | | | | | | |
| Sex | Male | n (%) | 11 | (64.7) | 9 | (52.9) | 19 | (90.5) | 15 | (78.9) |
| | Female | | 6 | (35.3) | 8 | (47.1) | 2 | (9.5) | 4 | (21.1) |
| Race | White | n (%) | 10 | (58.8) | 7 | (41.2) | 16 | (76.2) | 12 | (63.2) |
| | Black or African | | 7 | (41.2) | 10 | (58.8) | 4 | (19.0) | 6 | (31.6) |
| | American | | | | | | | | | |
| | Native Hawaiian or | | 0 | | 0 | | 0 | | 0 | |
| | Other Pacific | | | | | | | | | |
| | Islander | | | | | | | | | |
| | Asian | | 0 | | 0 | | 0 | | 0 | |
| | American Indian or | | 0 | | 0 | | 0 | | 0 | |
| | Alaska Native | | | | | | | | | |
| | Multiple | | 0 | | 0 | | 0 | | 1 | (5.3) |
| | Other | | 0 | | 0 | | 1 | (4.8) | 0 | |
| Ethnicity | Hispanic or Latino | n (%) | 1 | (5.9) | 0 | | 5 | (23.8) | 2 | (10.5) |
| | Not Hispanic | | 16 | (94.1) | 17 | (100.0) | 16 | (76.2) | 17 | (89.5) |
| | or Latino | | | | | | | | | |
| Body Mass | | Mean | 24.65 | (4.14) | 22.95 | (3.22) | 25.05 | (3.82) | 28.16 | (7.86) |
| Index | | (SD) | | | | | | | | |
| (kg/m2) | | | | | | | | | | |
| Past 7 Day | | Mean | 2.9 | (3.27) | 1.3 | (2.59) | 1.8 | (2.78) | 3.8 | (3.39) |
| Opioid Use | | (SD) | | | | | | | | |
| Screening | Opioids | n (%) | 4 | (23.5) | 4 | (23.5) | 7 | (33.3) | 7 | (38.9) |
| Urine | Fentanyl | | 13 | (76.5) | 14 | (82.4) | 17 | (81.0) | 17 | (89.5) |
| Toxicology | Buprenorphine | | 3 | (17.6) | 3 | (17.6) | 2 | (9.5) | 3 | (15.8) |
| | Methadone | | 0 | | 2 | (11.8) | 0 | | 2 | (10.5) |
| | Cocaine | | 10 | (58.8) | 10 | (58.8) | 11 | (52.4) | 11 | (57.9) |
| | Cannabinoids | | 7 | (41.2) | 6 | (35.3) | 8 | (38.1) | 10 | (52.6) |
| | Benzodiazepines | | 6 | (35.3) | 2 | (11.8) | 2 | (9.5) | 1 | (5.3) |
| | Amphetamines | | 1 | (5.9) | 2 | (11.8) | 1 | (4.8) | 3 | (15.8) |
| | Phencyclidine | | 0 | | 2 | (11.8) | 1 | (4.8) | 0 | |
| | Ketamine | | 0 | | 0 | | 0 | | 0 | |
| | | | 180 μg | | 240 μg | | Placebo | | | |
| Dexmedetomidine | | | BID | | BID | | BID | | Total | |
| oromucosal Dose | | | (N = 21) | | (N = 15) | | (N = 25) | | (N = 135) | |
| Age (Years) | | Mean | 42.9 | (9.57) | 42.0 | (11.9) | 42.1 | (10.9) | 42.0 | (10.9) |
| | | (SD) | | | | | | | | |
| Sex | Male | n (%) | 15 | (71.4) | 11 | (73.3) | 19 | (76.0) | 99 | (73.3) |
| | Female | | 6 | (28.6) | 4 | (26.7) | 6 | (24.0) | 36 | (26.7) |
| Race | White | n (%) | 13 | (61.9) | 10 | (66.7) | 16 | (64.0) | 84 | (62.2) |
| | Black or African | | 7 | (33.3) | 5 | (33.3) | 9 | (36.0) | 48 | (35.6) |
| | American | | | | | | | | | |
| | Native Hawaiian or | | 0 | | 0 | | 0 | | 0 | |
| | Other Pacific | | | | | | | | | |
| | Islander | | | | | | | | | |
| | Asian | | 0 | | 0 | | 0 | | 0 | |
| | American Indian or | | 0 | | 0 | | 0 | | 0 | |
| | Alaska Native | | | | | | | | | |
| | Multiple | | 0 | | 0 | | 0 | | 1 | (0.7) |
| | Other | | 1 | (4.8) | 0 | | 0 | | 2 | (1.5) |
| Ethnicity | Hispanic or Latino | n (%) | 4 | (19.0) | 4 | (26.7) | 2 | (8.0) | 18 | (13.3) |
| | Not Hispanic | | 17 | (81.0) | 11 | (73.3) | 23 | (92.0) | 117 | (86.7) |
| | or Latino | | | | | | | | | |
| Body Mass | | Mean | 28.05 | (8.25) | 27.47 | (6.59) | 26.21 | (7.72) | 26.12 | (6.49) |
| Index | | (SD) | | | | | | | | |
| (kg/m2) | | | | | | | | | | |
| Past 7 Day | | Mean | 3.5 | (3.35) | 3.4 | (3.40) | 2.8 | (3.31) | 2.9 | (3.27) |
| Opioid Use | | (SD) | | | | | | | | |
| Screening | Opioids | n (%) | 8 | (38.1) | 3 | (20.0) | 10 | (41.7) | 4 | (23.5) |
| Urine | Fentanyl | | 19 | (90.5) | 13 | (86.7) | 22 | (91.7) | 13 | (76.5) |
| Toxicology | Buprenorphine | | 2 | (9.5) | 3 | (20.0) | 3 | (12.5) | 3 | (17.6) |
| | Methadone | | 0 | | 0 | | 1 | (4.2) | 0 | |
| | Cocaine | | 10 | (47.6) | 7 | (46.7) | 14 | (58.3) | 10 | (58.8) |
| | Cannabinoids | | 9 | (42.9) | 2 | (13.3) | 10 | (41.7) | 7 | (41.2) |
| | Benzodiazepines | | 4 | (19.0) | 2 | (13.3) | 2 | (8.3) | 6 | (35.3) |

| | | | | | | |
|---|---|---|---|---|---|---|
| Amphetamines | 0 | 0 | 1 | (4.2) | 1 | (5.9) |

| | | | | | |
|---|---|---|---|---|---|
| Phencyclidine | 1 | (4.8) | 0 | 0 | 0 |

| | | | | |
|---|---|---|---|---|
| Ketamine | 0 | 0 | 0 | 0 |

### Safety and Tolerability

A total of 36 subjects reported 1 or more Treatment-emergent adverse event (TEAE). Higher doses of dexmedetomidine oromucosal film (i.e., 180 and 240 mg BID) significantly increased the frequency of hypotension, orthostatic hypotension, and somnolence. Among the TEAEs that were determined to be probably or definitely related to dexmedetomidine oromucosal film, the severity of each occurrence was rated as “mild” or “moderate” by a site physician. However, three “severe” TEAEs of orthostatic hypotension, bradycardia, and back/musculoskeletal pain, were noted as “severe.” All severe TEAEs occurred within the same participant who was randomized to the 120 mg BID condition.

The frequency of all TEAEs as a function of dexmedetomidine oromucosal film dose (Days 6-12) is presented in Table 15D. No TEAEs that were found during the Day 14 physical examination or from the Day 21 clinical laboratory tests occurred at a greater frequency among participants who received active versus placebo dexmedetomidine oromucosal film. Finally, sublingual administration of the dexmedetomidine oromucosal film did not produce any reports of local irritation of the oral mucosa.

See table below (Table 15D) with focus on cardio-vascular and nervous system treatment emergent adverse events.

TABLE 15D
Treatment-Emergent Adverse Events by System Organ Class

| | | | | | | | | |
|---|---|---|---|---|---|---|---|---|
| | 30 μg | 60 μg | 90 μg | 120 μg | 180 μg | 240 μg | Placebo | |
| System Organ | BID | BID | BID | BID | BID | BID | BID | |
| Class*& | (N = 17) | (N = 17) | (N = 21) | (N = 19) | (N = 21) | (N = 15) | (N = 25) | X2 |
| MedDRA Term | n (%) | n (%) | n (%) | n (%) | n (%) | n (%) | n (%) | p-value |

Cardiac disorders

| | | | | | | | | | |
|---|---|---|---|---|---|---|---|---|---|
| Bradycardia | 0 | 0 | 0 | 0 | 0 | 1 | (6.7) | 0 | 0.5 |

Eye disorders

| | | | | | | | | |
|---|---|---|---|---|---|---|---|---|
| Lacrimation | 0 | 0 | 0 | 1 (5.3) | 0 | 0 | 0 | 0.5 |

Gastrointestinal disorders

| Abdominal pain | 0 | 0 | | 0 | 1 (5.3) | 1 | (4.8) | 1 | (6.7) | 0 | | 0.7 |
|---|---|---|---|---|---|---|---|---|---|---|---|---|
| Diarrhea | 1 (5.9) | 2 | (11.8) | 0 | 1 (5.3) | 0 | | 0 | | 3 | (12.0) | 0.2 |
| Nausea | 0 | 1 | (5.9) | 1 (4.8) | 1 (5.3) | 2 | (9.5) | 1 | (6.7) | 1 | (4.0) | 0.7 |

| Toothache | 0 | 0 | | 0 | 1 (5.3) | 0 | | 0 | | 0 | 0.5 |
|---|---|---|---|---|---|---|---|---|---|---|---|
| Vomiting | 0 | 1 | (5.9) | 0 | 1 (5.3) | 1 | (4.8) | 3 | (20.0) | 0 | 0.2 |

General disorders

| Chills | 0 | 0 | 0 | 1 (5.3) | 0 | | 0 | 0 | 0.5 |
|---|---|---|---|---|---|---|---|---|---|
| Fatigue | 0 | 0 | 0 | 0 | 1 | (4.8) | 0 | 0 | 0.5 |

Infections & infestations

| | | | | | | | | | |
|---|---|---|---|---|---|---|---|---|---|
| Furuncle | 0 | 0 | 0 | 0 | 1 | (4.8) | 0 | 0 | 0.5 |

| | | | | | | | | | |
|---|---|---|---|---|---|---|---|---|---|
| Urinary tract | 0 | 0 | 0 | 0 | 0 | 0 | 1 | (4.0) | 0.5 |

Metabolism & nutrition disorders

| | | | | | | | | | |
|---|---|---|---|---|---|---|---|---|---|
| Dehydration | 0 | 0 | 0 | 0 | 1 | (4.8) | 0 | 0 | 0.5 |

Musculoskeletal & connective tissue disorders

| Back pain | 0 | 0 | 0 | 1 (5.3) | 0 | | 0 | 0 | | 0.5 |
|---|---|---|---|---|---|---|---|---|---|---|
| Groin pain | 0 | 0 | 0 | 0 | 0 | | 0 | 1 | (4.0) | 0.5 |
| Myalgia | 0 | 0 | 0 | 1 (5.3) | 1 | (4.8) | 0 | 1 | (4.0) | 0.7 |

Nervous system disorders

| Dizziness | 0 | 0 | 0 | 0 | 1 | (4.8) | 0 | | 0 | 0.5 |
|---|---|---|---|---|---|---|---|---|---|---|
| Presyncope | 0 | 0 | 0 | 0 | 0 | | 1 | (6.7) | 0 | 0.5 |
| Somnolence | 0 | 0 | 0 | 0 | 2 | (9.5) | 7 | (46.7) | 0 | <0.001 |

Psychiatric disorders

| | | | | | | | | | | |
|---|---|---|---|---|---|---|---|---|---|---|
| Anxiety | 0 | 2 | (11.8) | 0 | 0 | 1 | (4.8) | 0 | 0 | 0.2 |

| Insomnia | 0 | 0 | | 0 | 1 (5.3) | 0 | 0 | 0 | 0.5 |
|---|---|---|---|---|---|---|---|---|---|
| Irritability | 0 | 2 | (11.8) | 0 | 0 | 0 | 0 | 0 | 0.06 |

| | | | | | | | | |
|---|---|---|---|---|---|---|---|---|
| Restlessness | 0 | 0 | 1 (4.8) | 0 | 0 | 0 | 0 | 0.5 |

Respiratory, thoracic, & mediastinal disorders

| | | | | | | | | | | |
|---|---|---|---|---|---|---|---|---|---|---|
| Cough | 0 | 0 | 0 | 1 (5.3) | 3 | (14.3) | 0 | 1 | (4.0) | 0.1 |

Vascular disorders

| Hypertension | 0 | 0 | | 0 | 0 | 0 | 1 | (6.7) | 0 | 0.5 |
|---|---|---|---|---|---|---|---|---|---|---|
| Hypotension | 0 | 1 | (5.9) | 0 | 0 | 0 | 5 | (33.3) | 0 | <0.001 |

| | | | | | | | | | | |
|---|---|---|---|---|---|---|---|---|---|---|
| Orthostatic | 0 | 0 | 0 | 0 | 2 | (9.5) | 4 | (26.7) | 0 | <0.01 |

*Participants are counted once within each system organ class and adverse event.

TABLE 15E
Key Safety Advantages of dexmedetomidine oromucosal film vs. lofexidine (Fishman et al. 2019)

| | | Dexmedetomidine | | | | | | | | | |
|---|---|---|---|---|---|---|---|---|---|---|---|
| | | oromucosal film | | | | | Lofexidine* (Fishman et. Al, | | | | |
| | Placebo | 120 μg | 180 μg | | 240 μg | | 2019) | | | | |
| | Placebo | BID | BID | | BID | | Placebo | 2.16 mg | | 2.88 mg | |
| Adverse | (N = 25) | (N = 19) | (N = 21) | | (N = 15) | | (N = 229) | (N = 229) | | (N = 229) | |
| Event | n (%) | n (%) | n (%) | | n (%) | | n (%) | n (%) | | n (%) | |
| Orthostatic | 0 (0) | 0 (0) | 2 | (9.5) | 4 | (27) | 7 (4.6) | 67 | (29) | 94 | (42) |
| hypotension | | | | | | | | | | | |
| Bradycardia | 0 (0) | 0 (0) | 0 | (0) | 1 | (6.7) | 8 (5.3) | 54 | (24) | 70 | (32) |
| Dizziness | 0 (0) | 0 (0) | 1 | (4.8) | 0 | (4.8) | 4 (2.6) | 44 | (19) | 51 | (23.0) |
| Somnolence | 0 (0) | 0 (0) | 2 | (9.5) | 7 | (46.7) | 8 (5.3) | 25 | (10.9) | 29 | (13.1) |

*Fewer than half of the participants in the lofexidine trial completed the study in the active dose groups.

### Effects of Dexmedetomidine Hydrochloride Oromucosal Film on Opioid Withdrawal and Retention

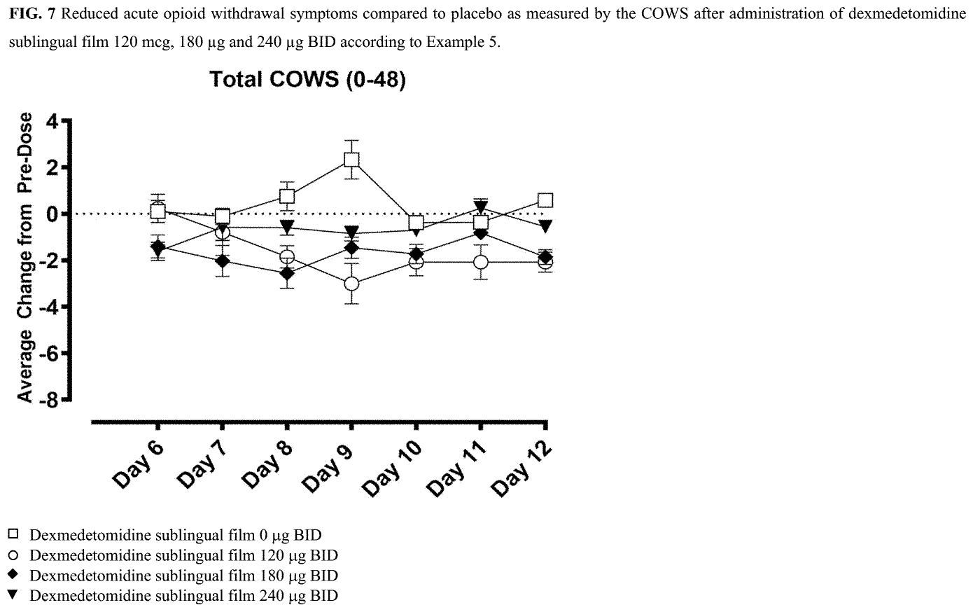

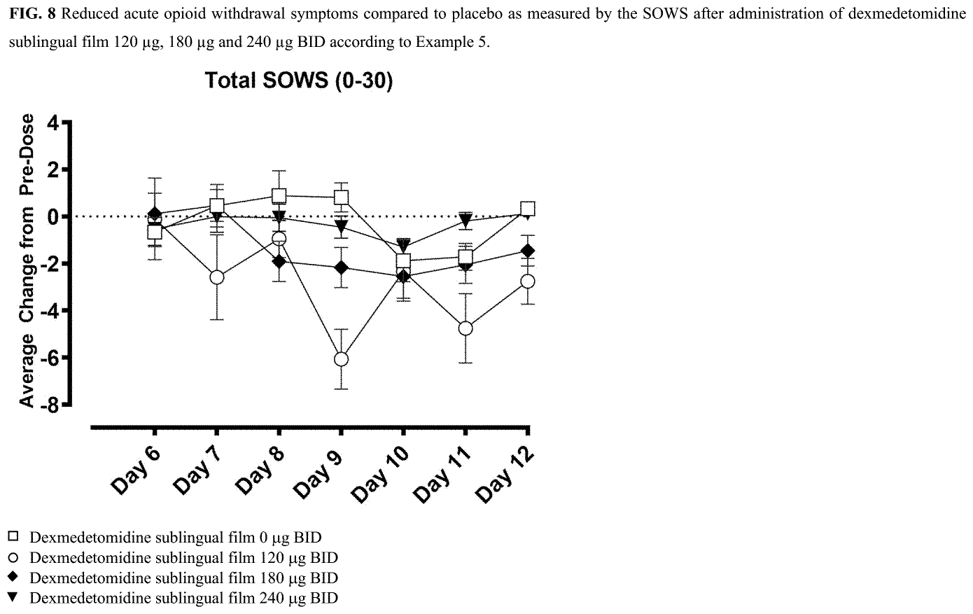

COWS and SOWS data were examined as a function of average change from pre-dose (i.e., 2-hour post-dose score subtracted from the respective pre-dose score and then averaged across the morning and evening dosing times). A significant reduction from pre-dose SOWS and COWS scores were observed on various days for the 120, 180, and 240 mg BID dose conditions (FIGS. 7 and 8). Average change for each of the individual SOWS and COWS items is presented in Table 15F.

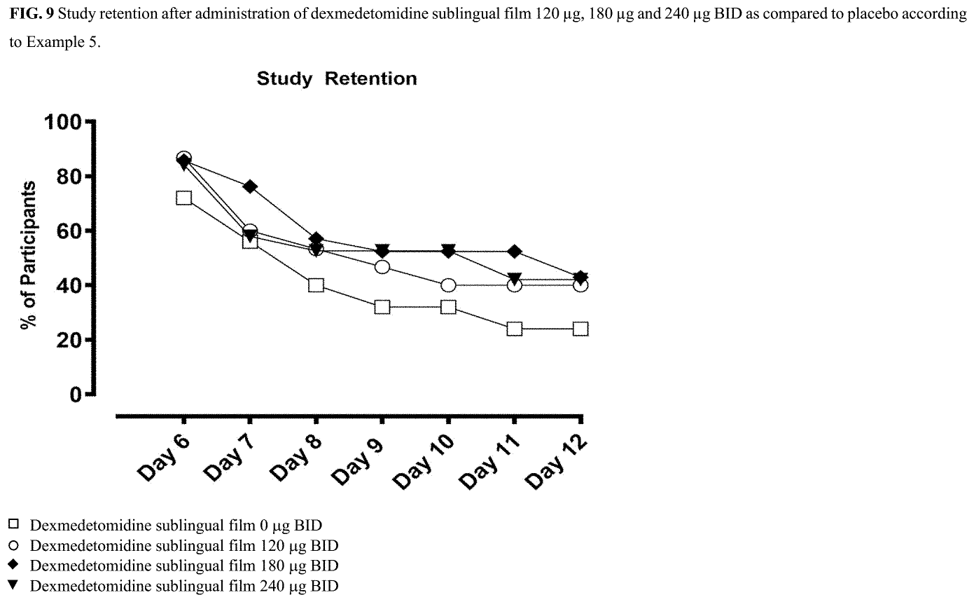

Overall dropout rates (i.e., % retention) during days 6-12 did not significantly vary across the dose conditions (p=0.6). However, there is a trend towards improved retention with higher doses of dexmedetomidine hydrochloride oromucosal film compared to placebo (FIG. 9)

TABLE 15F
Average Change from Pre-Dose Score*

| | Dexmedetomidine hydrochloride oromucosal dose | | | | | | |
|---|---|---|---|---|---|---|---|
| | 30 μg | 60 μg | 90 μg | 120 μg | 180 μg | 240 μg | Placebo |
| Outcome | (N = 17) | (N = 17) | (N = 21) | (N = 19) | (N = 21) | (N = 15) | (N = 25) |
| Measure | n(SD) | n(SD) | n(SD) | n(SD) | n(SD) | n(SD) | n(SD) |

SOWS
Feeling Sick

| | | | | | | | | |
|---|---|---|---|---|---|---|---|---|
| Day 6 | −0.21 | 0.22 | 0.00 | 0.03 | 0.19 | 0.03 | −0.04 | (0.576) |
| Day 7 | −0.19 | −0.04 | 0.14 | 0.08 | 0.22 | −0.33 | 0.14 | (0.819) |
| Day 8 | −0.15 | −0.14 | 0.28 | 0.20 | −0.47 | −0.20 | 0.31 | (0.805) |
| Day 9 | 0.00 | 0.07 | 0.20 | −0.20 | −0.25 | −0.31 | 0.19 | (0.843) |
| Day 10 | −0.29 | 0.00 | 0.00 | −0.10 | 0.00 | −0.08 | −0.13 | (0.582) |
| Day 11 | 0.07 | 0.30 | 0.00 | 0.06 | −0.18 | −0.08 | 0.00 | (0.289) |
| Day 12 | 0.00 | 0.20 | −0.33 | 0.06 | 0.14 | −0.17 | 0.50 | (0.775) |

Stomach Cramps

| | | | | | | | | |
|---|---|---|---|---|---|---|---|---|
| Day 6 | 0.00 | 0.13 | −0.13 | −0.03 | −0.10 | −0.07 | −0.28 | (0.579) |
| Day 7 | −0.27 | −0.13 | −0.17 | −0.12 | 0.06 | −0.33 | 0.14 | (0.795) |
| Day 8 | −0.08 | 0.21 | 0.00 | 0.15 | −0.38 | −0.45 | 0.12 | (0.893) |
| Day 9 | −0.06 | −0.21 | 0.20 | −0.10 | −0.58 | −0.63 | 0.06 | (0.623) |
| Day 10 | −0.14 | −0.17 | 0.07 | −0.35 | −0.45 | −0.17 | −0.19 | (0.458) |
| Day 11 | −0.14 | −0.10 | −0.07 | 0.13 | −0.18 | −0.67 | −0.14 | (0.378) |
| Day 12 | −0.07 | 0.00 | −0.17 | −0.19 | 0.00 | −0.50 | 0.17 | (0.258) |

Muscle Spasm/Twitching

| | | | | | | | | |
|---|---|---|---|---|---|---|---|---|
| Day 6 | 0.12 | 0.06 | −0.05 | −0.24 | −0.02 | 0.13 | −0.08 | (0.553) |
| Day 7 | 0.04 | 0.04 | −0.17 | 0.12 | −0.25 | −0.21 | −0.18 | (0.464) |
| Day 8 | 0.04 | −0.29 | 0.16 | −0.20 | −0.44 | 0.05 | −0.08 | (0.572) |
| Day 9 | 0.00 | −0.07 | 0.00 | −0.05 | −0.38 | −0.44 | 0.19 | (0.259) |
| Day 10 | −0.07 | 0.00 | −0.14 | −0.15 | 0.14 | 0.00 | −0.38 | (0.582) |
| Day 11 | −0.14 | 0.10 | 0.07 | −0.06 | −0.23 | −0.33 | −0.07 | (0.189) |
| Day 12 | −0.21 | 0.00 | −0.25 | −0.06 | −0.64 | 0.00 | 0.17 | (0.683) |

Feelings of Coldness

| | | | | | | | | |
|---|---|---|---|---|---|---|---|---|
| Day 6 | −0.21 | 0.38 | −0.20 | −0.21 | 0.12 | 0.23 | −0.06 | (0.464) |
| Day 7 | −0.08 | −0.67 | −0.08 | 0.12 | 0.06 | −0.21 | 0.04 | (0.692) |
| Day 8 | 0.23 | −0.14 | 0.00 | −0.15 | −0.19 | −0.10 | 0.23 | (0.881) |
| Day 9 | −0.11 | −0.36 | −0.30 | −0.05 | −0.42 | −0.94 | −0.13 | (0.354) |
| Day 10 | 0.00 | 0.00 | 0.07 | 0.00 | −0.50 | −0.25 | −0.31 | (0.594) |
| Day 11 | −0.14 | 0.20 | −0.07 | 0.19 | −0.36 | −0.25 | −0.07 | (0.345) |
| Day 12 | 0.00 | −0.20 | −0.17 | 0.00 | −0.27 | −0.17 | 0.08 | (0.204) |

Heart Pounding

| | | | | | | | | |
|---|---|---|---|---|---|---|---|---|
| Day 6 | −0.24 | 0.16 | −0.08 | −0.05 | −0.19 | −0.10 | −0.08 | (0.607) |
| Day 7 | −0.12 | −0.21 | 0.00 | 0.04 | 0.16 | −0.25 | −0.04 | (0.634) |
| Day 8 | −0.23 | −0.07 | 0.13 | 0.00 | −0.06 | −0.05 | 0.00 | (0.500) |
| Day 9 | 0.06 | −0.14 | 0.10 | −0.05 | −0.04 | −0.31 | 0.00 | (0.000) |
| Day 10 | −0.57 | −0.33 | −0.07 | −0.15 | −0.41 | −0.25 | 0.00 | (0.000) |
| Day 11 | −0.14 | −0.10 | 0.00 | −0.06 | −0.27 | −0.25 | −0.14 | (0.378) |
| Day 12 | 0.07 | −0.20 | −0.33 | 0.00 | −0.32 | −0.33 | −0.08 | (0.376) |

Muscle Tension

| | | | | | | | | |
|---|---|---|---|---|---|---|---|---|
| Day 6 | 0.00 | 0.16 | −0.05 | −0.18 | 0.02 | 0.17 | −0.12 | (0.696) |
| Day 7 | −0.04 | −0.04 | 0.11 | 0.00 | 0.00 | −0.21 | −0.07 | (0.852) |
| Day 8 | 0.15 | 0.14 | −0.06 | −0.35 | −0.16 | 0.30 | 0.00 | (0.500) |
| Day 9 | 0.17 | −0.07 | 0.15 | 0.05 | 0.08 | −0.56 | 0.19 | (0.372) |
| Day 10 | −0.07 | −0.17 | −0.07 | −0.10 | −0.41 | −0.08 | −0.31 | (0.594) |
| Day 11 | −0.21 | 0.10 | 0.14 | 0.06 | 0.00 | −0.67 | −0.14 | (0.627) |
| Day 12 | 0.00 | −0.10 | −0.42 | −0.06 | −0.36 | 0.08 | −0.42 | (0.585) |

Aches and Pains

| | | | | | | | | |
|---|---|---|---|---|---|---|---|---|
| Day 6 | −0.03 | 0.03 | −0.13 | 0.03 | 0.00 | −0.03 | −0.12 | (0.650) |
| Day 7 | 0.08 | 0.25 | −0.22 | −0.08 | −0.13 | −0.42 | 0.14 | (0.770) |
| Day 8 | 0.12 | −0.21 | −0.25 | −0.10 | −0.25 | 0.25 | −0.04 | (1.108) |
| Day 9 | −0.28 | −0.07 | 0.00 | 0.00 | −0.21 | −0.88 | 0.19 | (0.458) |
| Day 10 | −0.36 | 0.00 | 0.00 | −0.15 | −0.14 | −0.50 | −0.13 | (0.354) |
| Day 11 | 0.07 | 0.00 | 0.07 | −0.31 | −0.27 | −1.00 | −0.14 | (0.476) |
| Day 12 | 0.00 | 0.00 | −0.50 | 0.06 | −0.32 | −0.33 | −0.17 | (0.683) |

Yawning

| | | | | | | | | |
|---|---|---|---|---|---|---|---|---|
| Day 6 | 0.06 | 0.22 | 0.05 | 0.00 | −0.05 | −0.07 | 0.04 | (0.498) |
| Day 7 | 0.19 | −0.04 | −0.11 | −0.12 | −0.06 | −0.21 | −0.14 | (0.691) |
| Day 8 | 0.00 | 0.00 | −0.06 | 0.10 | 0.16 | −0.15 | −0.04 | (0.721) |
| Day 9 | −0.28 | 0.00 | −0.15 | 0.00 | −0.08 | −0.81 | −0.13 | (0.231) |
| Day 10 | −0.07 | −0.17 | −0.07 | 0.00 | −0.23 | −0.33 | −0.31 | (0.594) |
| Day 11 | 0.00 | 0.00 | 0.00 | 0.00 | −0.09 | −0.50 | −0.43 | (0.535) |
| Day 12 | −0.07 | −0.30 | −0.25 | 0.06 | −0.18 | −0.42 | 0.17 | (0.258) |

Runny Eyes

| | | | | | | | | |
|---|---|---|---|---|---|---|---|---|
| Day 6 | 0.15 | 0.00 | 0.05 | −0.03 | 0.00 | 0.00 | 0.04 | (0.320) |
| Day 7 | 0.00 | 0.13 | 0.36 | 0.00 | 0.19 | 0.25 | 0.25 | (0.643) |
| Day 8 | 0.15 | −0.07 | 0.31 | 0.00 | 0.28 | −0.15 | 0.31 | (0.480) |
| Day 9 | 0.00 | −0.07 | 0.20 | 0.10 | 0.08 | −0.31 | −0.06 | (0.563) |
| Day 10 | 0.21 | −0.17 | 0.14 | 0.00 | −0.27 | −0.08 | −0.31 | (0.651) |
| Day 11 | −0.29 | −0.20 | −0.14 | 0.00 | −0.05 | −0.58 | −0.29 | (0.567) |
| Day 12 | 0.00 | −0.20 | −0.17 | 0.00 | 0.18 | −0.08 | 0.00 | (0.000) |

Insomnia or Problems Sleeping

| | | | | | | | | |
|---|---|---|---|---|---|---|---|---|
| Day 6 | −0.12 | 0.13 | 0.08 | 0.13 | 0.14 | −0.40 | 0.04 | (1.050) |
| Day 7 | −0.04 | −0.33 | 0.39 | −0.04 | 0.22 | −0.67 | 0.18 | (0.639) |
| Day 8 | 0.15 | −0.07 | 0.31 | 0.30 | −0.41 | −0.45 | 0.08 | (1.017) |
| Day 9 | 0.28 | −0.14 | 0.20 | −0.15 | −0.38 | −0.88 | 0.31 | (0.651) |
| Day 10 | 0.14 | 0.00 | 0.07 | −0.30 | −0.27 | −0.58 | 0.19 | (0.594) |
| Day 11 | −0.14 | −0.20 | −0.36 | −0.19 | −0.41 | −0.42 | −0.29 | (0.699) |
| Day 12 | −0.07 | −0.70 | −0.33 | 0.25 | 0.27 | −0.83 | −0.08 | (0.665) |

COWS
Resting Pulse Rate

| | | | | | | | | |
|---|---|---|---|---|---|---|---|---|
| Day 6 | 0.15 | −0.09 | −0.08 | −0.05 | −0.26 | −0.13 | 0.32 | (0.690) |
| Day 7 | 0.12 | −0.21 | 0.06 | −0.08 | −0.06 | −0.13 | −0.11 | (0.881) |
| Day 8 | −0.31 | 0.00 | −0.13 | −0.20 | −0.19 | 0.05 | 0.35 | (1.197) |
| Day 9 | 0.11 | −0.21 | 0.05 | −0.10 | 0.00 | 0.19 | 0.56 | (0.982) |
| Day 10 | 0.00 | −0.17 | −0.14 | 0.15 | 0.05 | 0.00 | 0.19 | (0.651) |
| Day 11 | 0.64 | −0.30 | −0.29 | 0.13 | −0.23 | −0.08 | −0.07 | (0.787) |

Day 12

Restlessness

| | | | | | | | | |
|---|---|---|---|---|---|---|---|---|
| Day 6 | −0.03 | 0.25 | −0.13 | −0.55 | −0.31 | −0.17 | −0.02 | (0.699) |
| Day 7 | 0.46 | −0.08 | −0.14 | 0.04 | −0.25 | −0.42 | −0.14 | (0.886) |
| Day 8 | 0.08 | −0.14 | 0.03 | −0.20 | −0.78 | −0.28 | −0.27 | (0.665) |
| Day 9 | 0.06 | 0.14 | −0.10 | −0.15 | −0.25 | −0.94 | −0.06 | (0.464) |
| Day 10 | −0.14 | −0.33 | −0.07 | −0.25 | −0.14 | −0.17 | −0.19 | (0.259) |
| Day 11 | 0.00 | −1.00 | −0.25 | −0.13 | −0.50 | −0.25 | 0.17 | (0.516) |

Day 12

Pupil

| | | | | | | | | |
|---|---|---|---|---|---|---|---|---|
| Day 6 | −0.06 | 0.00 | −0.13 | 0.00 | 0.05 | 0.20 | 0.00 | (0.144) |
| Day 7 | 0.04 | −0.08 | −0.08 | −0.12 | −0.13 | 0.17 | 0.04 | (0.237) |
| Day 8 | 0.04 | −0.14 | 0.16 | −0.10 | −0.13 | 0.00 | −0.08 | (0.277) |
| Day 9 | 0.06 | 0.00 | −0.05 | −0.10 | −0.08 | −0.19 | 0.22 | (0.667) |
| Day 10 | −0.07 | 0.00 | 0.00 | 0.00 | −0.05 | 0.00 | −0.13 | (0.354) |
| Day 11 | 0.00 | 0.10 | 0.00 | 0.00 | 0.00 | 0.00 | 0.00 | (0.000) |
| Day 12 | 0.00 | 0.00 | 0.00 | 0.00 | −0.09 | −0.17 | 0.00 | (0.000) |

Bone and Joint Pain

| | | | | | | | | |
|---|---|---|---|---|---|---|---|---|
| Day 6 | −0.09 | 0.00 | −0.25 | −0.08 | 0.05 | 0.03 | −0.04 | (0.455) |
| Day 7 | 0.12 | 0.00 | −0.22 | −0.04 | −0.09 | −0.08 | 0.04 | (0.571) |
| Day 8 | 0.35 | −0.07 | −0.13 | −0.15 | −0.25 | 0.00 | −0.08 | (0.760) |
| Day 9 | −0.33 | −0.07 | −0.20 | −0.30 | −0.08 | −0.06 | 0.44 | (0.726) |
| Day 10 | 0.00 | 0.17 | 0.21 | 0.00 | −0.09 | −0.33 | 0.06 | (0.320) |
| Day 11 | −0.07 | 0.10 | 0.07 | −0.13 | 0.00 | −0.33 | 0.00 | (0.500) |
| Day 12 | 0.00 | 0.00 | −0.33 | 0.13 | −0.36 | −0.25 | 0.08 | (0.204) |

Runny Nose

| | | | | | | | | |
|---|---|---|---|---|---|---|---|---|
| Day 6 | 0.00 | −0.19 | −0.13 | −0.29 | 0.00 | 0.03 | 0.08 | (0.493) |
| Day 7 | 0.12 | −0.17 | 0.03 | −0.42 | −0.03 | 0.04 | 0.21 | (0.642) |
| Day 8 | 0.19 | −0.07 | −0.03 | −0.05 | −0.16 | 0.06 | 0.31 | (0.778) |
| Day 9 | −0.11 | −0.36 | −0.25 | −0.05 | −0.25 | −0.38 | 0.11 | (0.220) |
| Day 10 | 0.00 | −0.50 | −0.21 | −0.05 | −0.23 | −0.17 | 0.00 | (0.535) |
| Day 11 | −0.14 | −0.10 | −0.14 | 0.19 | 0.00 | −0.25 | −0.07 | (0.189) |
| Day 12 | −0.29 | 0.00 | −0.08 | −0.13 | 0.00 | 0.00 | 0.00 | (0.000) |

Gastrointestinal Upset

| | | | | | | | | |
|---|---|---|---|---|---|---|---|---|
| Day 6 | 0.24 | 0.06 | −0.20 | −0.24 | −0.36 | −0.07 | 0.04 | (0.519) |
| Day 7 | −0.12 | 0.00 | −0.17 | −0.08 | −0.44 | 0.29 | −0.11 | (0.738) |
| Day 8 | 0.35 | −0.21 | −0.13 | −0.10 | −0.47 | −0.50 | 0.19 | (0.663) |
| Day 9 | 0.28 | −0.86 | 0.35 | −0.10 | −0.08 | −0.25 | 0.33 | (1.199) |
| Day 10 | −0.29 | −0.08 | 0.07 | −0.30 | −0.23 | −0.50 | −0.13 | (0.991) |
| Day 11 | −0.07 | −0.30 | −0.36 | 0.06 | 0.00 | −0.33 | −0.07 | (0.345) |
| Day 12 | −0.14 | −0.10 | −0.42 | −0.19 | 0.09 | −0.50 | 0.17 | (0.516) |

Tremor

| | | | | | | | | |
|---|---|---|---|---|---|---|---|---|
| Day 6 | −0.18 | −0.03 | −0.10 | −0.08 | −0.14 | 0.07 | 0.00 | (0.323) |
| Day 7 | −0.08 | −0.50 | −0.19 | −0.12 | −0.25 | −0.04 | 0.00 | (0.196) |
| Day 8 | 0.23 | −0.07 | −0.19 | −0.15 | −0.19 | −0.50 | −0.04 | (0.139) |
| Day 9 | 0.00 | −0.07 | −0.10 | 0.00 | −0.33 | −0.50 | 0.06 | (0.167) |
| Day 10 | 0.14 | −0.08 | 0.07 | −0.05 | −0.14 | −0.17 | 0.06 | (0.177) |
| Day 11 | 0.00 | −0.10 | −0.07 | 0.13 | −0.18 | −0.33 | 0.00 | (0.289) |
| Day 12 | 0.07 | 0.00 | 0.00 | 0.00 | −0.09 | −0.25 | 0.00 | (0.000) |

Yawning

| | | | | | | | | |
|---|---|---|---|---|---|---|---|---|
| Day 6 | 0.03 | 0.06 | 0.00 | −0.11 | −0.02 | 0.13 | 0.10 | (0.323) |
| Day 7 | 0.08 | −0.08 | −0.06 | 0.08 | −0.09 | −0.08 | 0.04 | (0.365) |
| Day 8 | 0.23 | −0.14 | 0.00 | 0.10 | −0.16 | 0.06 | −0.04 | (0.380) |
| Day 9 | −0.22 | 0.00 | 0.10 | −0.10 | −0.17 | −0.25 | 0.00 | (0.250) |
| Day 10 | 0.00 | 0.00 | 0.07 | 0.00 | 0.00 | 0.00 | 0.00 | (0.267) |
| Day 11 | 0.07 | 0.20 | −0.07 | −0.06 | 0.00 | 0.00 | −0.14 | (0.244) |
| Day 12 | 0.07 | 0.10 | −0.25 | 0.06 | 0.00 | −0.33 | −0.08 | (0.204) |

Anxiety

| | | | | | | | | |
|---|---|---|---|---|---|---|---|---|
| Day 6- | −0.03 | −0.06 | −0.18 | −0.05 | −0.29 | 0.00 | −0.20 | (0.722) |
| Day 7 | 0.12 | −0.04 | 0.06 | 0.12 | 0.06 | −0.38 | 0.07 | (0.385) |
| Day 8 | −0.23 | −0.29 | 0.00 | −0.05 | −0.13 | −0.33 | 0.19 | (0.435) |
| Day 9 | 0.28 | 0.14 | −0.45 | −0.10 | 0.00 | −0.38 | 0.11 | (0.333) |
| Day 10 | −0.07 | 0.00 | −0.07 | −0.20 | −0.09 | −0.17 | −0.06 | (0.320) |
| Day 11 | −0.07 | 0.00 | −0.14 | 0.00 | 0.14 | −0.17 | 0.07 | (0.189) |
| Day 12 | 0.07 | −0.40 | −0.25 | −0.06 | −0.14 | −0.33 | 0.25 | (0.418) |

Gooseflesh

| | | | | | | | | |
|---|---|---|---|---|---|---|---|---|
| Day 6 | −0.09 | 0.00 | −0.23 | −0.08 | −0.14 | 0.10 | −0.12 | (0.415) |
| Day 7 | 0.46 | −0.46 | −0.08 | −0.23 | −0.47 | 0.00 | −0.11 | (0.401) |
| Day 8 | 0.58 | −0.43 | 0.19 | 0.15 | −0.19 | −0.33 | 0.11 | (0.924) |
| Day 9 | −0.83 | −0.21 | 0.00 | 0.15 | −0.08 | −0.38 | 0.33 | (1.000) |
| Day 10 | −0.21 | 0.00 | 0.00 | 0.00 | −0.55 | −0.50 | 0.00 | (0.000) |
| Day 11 | 0.00 | 0.00 | 0.00 | 0.00 | −0.27 | 0.00 | 0.00 | (0.000) |
| Day 12 | 0.00 | −0.30 | 0.00 | 0.00 | −0.55 | 0.00 | 0.00 | (0.000) |

Agitation and Calmness Scale (ACES) Total Score
Post First Dose

| | | | | | | | | | | | | | | |
|---|---|---|---|---|---|---|---|---|---|---|---|---|---|---|
| Day 6 | 4.6 | (1.27) | 3.4 | (1.71) | 4.4 | (1.35) | 4.9 | (1.31) | 4.7 | (1.35) | 4.9 | (1.73) | 4.3 | (1.40) |
| Day 7 | 4.3 | (0.95) | 3.6 | (1.83) | 3.8 | (1.29) | 3.9 | (0.76) | 4.1 | (1.48) | 4.4 | (1.73) | 4.1 | (0.86) |
| Day 8 | 3.8 | (1.17) | 4.1 | (1.07) | 4.0 | (1.26) | 4.6 | (1.43) | 4.2 | (1.17) | 5.0 | (1.83) | 4.1 | (0.83) |
| Day 9 | 3.0 | (0.87) | 4.1 | (1.46) | 3.7 | (0.67) | 4.5 | (1.18) | 3.5 | (0.80) | 4.7 | (2.06) | 3.8 | (0.46) |
| Day 10 | 4.1 | (1.77) | 3.8 | (0.98) | 4.1 | (0.38) | 4.3 | (1.06) | 4.3 | (1.01) | 4.3 | (1.37) | 4.4 | (0.92) |
| Day 11 | 4.7 | (1.50) | 4.0 | (0.71) | 4.1 | (0.38) | 4.1 | (0.64) | 3.9 | (0.94) | 4.2 | (0.41) | 4.6 | (1.13) |
| Day 12 | 4.3 | (0.76) | 4.4 | (1.14) | 4.7 | (1.21) | 4.0 | (0.00) | 4.1 | (0.83) | 4.8 | (1.33) | 4.0 | (1.10) |

Post Second Dose

| | | | | | | | | | | | | | | |
|---|---|---|---|---|---|---|---|---|---|---|---|---|---|---|
| Day 6 | 3.9 | (0.73) | 3.5 | (0.78) | 3.7 | (0.75) | 3.6 | (0.61) | 4.1 | (1.00) | 3.6 | (0.77) | 3.8 | (0.73) |
| Day 7 | 3.8 | (0.38) | 3.2 | (0.75) | 3.7 | (0.70) | 3.9 | (1.04) | 3.8 | (0.41) | 3.5 | (0.71) | 4.1 | (0.27) |
| Day 8 | 3.8 | (0.67) | 3.9 | (0.38) | 3.7 | (0.63) | 3.6 | (0.70) | 4.0 | (0.43) | 3.9 | (0.35) | 3.7 | (0.90) |
| Day 9 | 4.3 | (0.49) | 4.0 | (0.58) | 4.1 | (0.38) | 3.8 | (0.42) | 3.8 | (0.60) | 4.4 | (1.06) | 3.9 | (0.83) |
| Day 10 | 4.0 | (0.00) | 3.6 | (0.89) | 4.0 | (0.00) | 3.7 | (0.48) | 3.9 | (0.54) | 4.2 | (0.41) | 3.9 | (0.35) |
| Day 11 | 4.0 | (0.00) | 3.2 | (1.30) | 4.0 | (0.00) | 3.8 | (0.46) | 3.7 | (0.47) | 4.3 | (1.37) | 3.8 | (0.41) |
| Day 12 | 3.9 | (0.69) | 4.0 | (0.00) | 4.2 | (0.41) | 4.0 | (0.00) | 3.9 | (0.33) | 3.7 | (0.82) | 3.6 | (0.89) |

*Average change refers to the average of the two change scores at 2-hour post-dose from the respective pre-dose scores. Bolded numbers indicate a statistically significant difference from placebo (p < .05).

Agitation and Calmness Scale (ACES): ACES total score during Days 6-12, following the first and second dexmedetomidine hydrochloride oromucosal film is shown in Table 15F. Mean observer ratings were between 3 (mild agitation), 4 (normal), and 5 (mild calmness), with few significant differences being observed as a function of dexmedetomidine hydrochloride oromucosal film dose

### Drug Concentration and Pharmacokinetics

Dexmedetomidine was quantifiable in plasma samples collected on days 6 and 12, at selected time points (pre-dose, 2 hours, 6 hours, and 12 hours post dose). The results in Table 15G showed a general trend of dose dependent increases in concentration as the dose increased from 30 μg to 240 μg. The mean concentrations declined over time between the 2-hour and 12-hour timepoints at all doses.

TABLE 15G1
Plasma concentrations of dexmedetomidine hydrochloride oromucosal film (Safety Population)

| | | Time | | | | | | |
|---|---|---|---|---|---|---|---|---|
| Parameter | | Point | 30 μg | 60 μg | 90 μg | 120 μg | 180 μg | 240 μg |
| (Unit) | Visit | Statistics | (N = 17) | (N = 17) | (N = 21) | (N = 19) | (N = 21) | (N = 15) |
| | | Pre Dose 1 | | | | | | |
| Dexmedetomidine | Day 6 | n | 17 | 17 | 19 | 19 | 21 | 15 |
| HCl oromucosal | | Mean | 0.00 | 0.00 | 2.48 | 0.36 | 1.36 | 1.68 |
| film (ng/L) | | (SD) | (0.000) | (0.000) | (8.955) | (1.588) | (6.210) | (6.504) |
| | | Gₘₑₐₙ | | | 18.23 | 6.92 | 28.46 | 25.19 |
| | | Median | 0.00 | 0.00 | 0.00 | 0.00 | 0.00 | 0.00 |
| | | Min, Max | 0.0, 0.0 | 0.0, 0.0 | 0.0, 38.6 | 0.0, 6.9 | 0.0, 28.5 | 0.0, 25.2 |
| | | CV (%) | | | 360.7 | 435.9 | 458.3 | 387.3 |
| | | 2 Hours Post Dose 1 | | | | | | |
| | | n | 17 | 16 | 19 | 18 | 21 | 15 |
| | | Mean | 44.56 | 105.96 | 138.90 | 169.05 | 362.59 | 346.79 |
| | | (SD) | (16.852) | (52.558) | (69.779) | (61.609) | (103.417) | (127.803) |
| | | Gmean | 41.69 | 91.38 | 121.38 | 161.14 | 349.12 | 324.23 |
| | | Median | 41.23 | 100.85 | 133.67 | 160.73 | 322.52 | 326.09 |
| | | Min, Max | 18.0, 80.9 | 17.2, 202.0 | 33.7, 330.8 | 89.3, 373.3 | 223.8, 544.7 | 155.4, 620.3 |
| | | CV (%) | 37.8 | 49.6 | 50.2 | 36.4 | 28.5 | 36.9 |
| | | 6 Hours Post Dose 1 | | | | | | |
| | | n | 14 | 14 | 19 | 19 | 21 | 15 |
| | | Mean | 21.60 | 46.97 | 45.67 | 70.66 | 152.47 | 161.09 |
| | | (SD) | (17.530) | (30.117) | (38.153) | (39.983) | (108.590) | (88.372) |
| | | Gmean | 16.85 | 39.07 | 33.88 | 61.12 | 123.59 | 140.80 |
| | | Median | 13.94 | 40.61 | 40.31 | 61.78 | 132.56 | 138.58 |
| | | Min, Max | 7.2, 59.5 | 10.6, 124.7 | 9.1, 157.3 | 18.6, 185.0 | 31.3, 502.7 | 52.0, 330.8 |
| | | CV (%) | 81.2 | 64.1 | 83.5 | 56.6 | 71.2 | 54.9 |
| | | 12 Hours Post Dose 1 | | | | | | |
| Dexmedetomidine | | n | 7 | 8 | 7 | 14 | 17 | 14 |
| oromucosal film | | Mean | 29.99 | 28.67 | 41.99 | 78.13 | 102.57 | 120.01 |
| (ng/L) | | (SD) | (30.637) | (24.967) | (36.820) | (87.334) | (129.498) | (141.859) |
| | | Gmean | 17.80 | 21.72 | 31.38 | 36.67 | 50.61 | 57.95 |
| | | Median | 10.66 | 19.88 | 33.00 | 34.71 | 30.66 | 50.07 |
| | | Min, Max | 5.9, 81.7 | 8.5, 83.6 | 8.3, 119.3 | 6.8, 281.7 | 10.5, 473.4 | 10.0, 431.4 |
| | | CV (%) | 102.2 | 87.1 | 87.7 | 111.8 | 126.3 | 118.2 |
| | | Pre Dose 1 | | | | | | |
| Dexmedetomidine | Day | n | 3 | 4 | 4 | 8 | 9 | 6 |
| oromucosal | 12 | Mean | 52.38 | 28.12 | 11.96 | 35.72 | 23.26 | 60.70 |
| film (ng/L) | | (SD) | (27.710) | (26.864) | (2.514) | (35.834) | (15.268) | (32.058) |
| | | Gmean | 45.70 | 21.13 | 11.74 | 25.01 | 19.41 | 52.12 |
| | | Median | 66.37 | 17.11 | 12.48 | 31.78 | 18.34 | 61.85 |
| | | Min, Max | 20.5, 70.3 | 10.2, 68.0 | 8.6, 14.3 | 7.4, 119.7 | 6.2, 56.9 | 17.9, 100.2 |
| | | CV (%) | 52.9 | 95.6 | 21.0 | 100.3 | 65.7 | 52.8 |
| | | 2 Hours Post Dose 1 | | | | | | |
| | | n | 7 | 5 | 6 | 8 | 9 | 6 |
| | | Mean | 66.63 | 149.99 | 106.80 | 193.19 | 324.33 | 469.96 |
| | | (SD) | (25.386) | (57.523) | (42.913) | (35.391) | (156.251) | (67.792) |
| | | Gmean | 63.10 | 142.66 | 99.07 | 190.34 | 287.69 | 466.27 |
| | | Median | 66.75 | 131.36 | 104.93 | 191.48 | 271.47 | 446.20 |
| | | Min, Max | 40.6, 117.6 | 98.5, 248.6 | 47.0, 175.2 | 144.8, 250.0 | 93.4, 566.1 | 405.7, 600.4 |
| | | CV (%) | 38.1 | 38.4 | 40.2 | 18.3 | 48.2 | 14.4 |
| | | 6 Hours Post Dose 1 | | | | | | |
| | | n | 7 | 5 | 6 | 8 | 9 | 5 |
| | | Mean | 22.25 | 71.33 | 31.83 | 76.33 | 89.57 | 180.25 |
| | | (SD) | (22.731) | (32.487) | (36.700) | (48.282) | (41.688) | (155.707) |
| | | Gmean | 17.16 | 66.00 | 21.55 | 63.56 | 81.75 | 145.57 |
| | | Median | 14.78 | 64.33 | 18.79 | 65.44 | 74.53 | 116.69 |
| | | Min, Max | 10.8, 73.5 | 37.1, 124.2 | 7.3, 105.6 | 21.8, 173.4 | 49.6, 166.6 | 81.0, 456.8 |
| | | CV (%) | 102.2 | 45.5 | 115.3 | 63.3 | 46.5 | 86.4 |
| | | 12 Hours Post Dose 1 | | | | | | |
| Dexmedetomidine | | n | 3 | 5 | 3 | 6 | 8 | 6 |
| oromueosal film | | Mean | 13.25 | 12.32 | 76.83 | 57.48 | 90.40 | 51.33 |
| (ng/L) | | (SD) | (12.293) | (6.769) | (49.915) | (40.580) | (195.986) | (64.802) |
| | | Gmean | 10.12 | 10.88 | 66.21 | 44.29 | 24.73 | 33.04 |
| | | Median | 6.46 | 12.33 | 64.88 | 51.25 | 17.58 | 25.97 |
| | | Min, Max | 5.9, 27.4 | 6.0, 22.5 | 34.0, 131.6 | 16.6, 112.2 | 5.6, 573.8 | 13.0, 182.3 |
| | | CV (%) | 92.8 | 54.9 | 65.0 | 70.6 | 216.8 | 126.3 |

CV = coefficient of variation;
max = maximum.
min = minimum;
SD = standard deviation

### Conclusions:

The primary aim of the current trial was to assess the safety of a novel oromucosal dexmedetomidine formulation (i.e., dexmedetomidine oromucosal film). The secondary aim was to assess the preliminary efficacy of dexmedetomidine oromucosal film to treat the symptoms of opioid withdrawal. Concerning safety, both self-reported and clinician-observed (e.g., abnormal laboratory tests) TEAEs were infrequent, mild, and none resulted in participant discontinuation from the trial. Compared to placebo, higher dexmedetomidine doses increased the incidence of hypotension, orthostatic hypotension, and somnolence.

Cardiovascular AEs were mild and transient, with none requiring medical intervention. Incidences of somnolence did not result in any participants who were un-arousable or required medical intervention. Furthermore, there was no clinically meaningful observer-rated sedation, as assessed by the ACES.

The frequency and severity of these TEAEs were lower than what has been reported in previous studies with lofexidine, the only FDA-approved drug to treat opioid withdrawal.

The safety profile observed for the dose range of dexmedetomidine oromucosal film tested in the current study suggests that higher doses could be explored for greater efficacy. Nonetheless, the current study did support the utility of dexmedetomidine oromucosal film for the treatment of opioid withdrawal. In comparison to placebo, higher dexmedetomidine oromucosal film doses (120, 180, and 240 mg BID) reduced COWS and SOWS scores. Furthermore, closer examination of the individual COWS and SOWS items revealed improvements in withdrawal symptomology not typically seen with lofexidine including insomnia, anxiety, and irritability The safety, tolerability, and preliminary efficacy observed in the current trial support the further development of dexmedetomidine oromucosal films (oromucosal dexmedetomidine) in the treatment of opioid withdrawal. Currently, only one medication is FDA-approved for this indication, lofexidine. Lofexidine's clinical utility was partially based on its improved safety profile in comparison to clonidine, which had been used off-label for this indication for years. In the current study, dexmedetomidine oromucosal film reduced the severity of opioid withdrawal at doses that produced less cardiovascular adverse effects that are major concerns for lofexidine treatment (e.g., orthostatic hypotension, bradycardia, dizziness, somnolence). Furthermore, unique treatment effects included reductions in insomnia, anxiety, and irritability, which often necessitate the co-prescribing of sleep medication and anxiolytics with lofexidine. In sum, in opioid-dependent individuals undergoing managed opioid withdrawal, dexmedetomidine oromucosal film was safe, well-tolerated, and reduced withdrawal severity. Thus, dexmedetomidine oromucosal film may prove to be an effective medication with novel treatment benefits so further testing and clinical development are warranted.

### Example 6: A Phase Ib/II Randomized, Double Blind, Placebo Controlled, Dose Finding, Efficacy and Safety Study of Dexmedetomidine Hydrochloride Oromucosal Film to Treat Patients Hospitalized in the ICU with Delirium and Agitation

### Key Objectives

1. To assess the impact of dexmedetomidine oromucosal film on cardiovascular parameters, including blood pressure, heart rate, and QTc interval, in hospitalized patients with hyperactive delirium.
2. To assess the incidence of other side effects following the administration of dexmedetomidine oromucosal film in the same patient group.
3. To explore the impact of dexmedetomidine oromucosal film on agitation and delirium severity
4. To identify the optimal dose of dexmedetomidine that is effective at reducing agitation and delirium severity without causing significant side effects.

### Diagnosis and Main Criteria for Eligibility:

### Inclusion Criteria

1. Adults hospitalized on a medical or surgical intensive care unit at MGH
2. Diagnosis of delirium, assessed according to DSM-5 criteria (DSM-5) by a licensed psychiatrist
3. Body mass index (BMI) between 18 and 30 kg/m², inclusive, at screening
4. Weight at least 60 kg (132 pounds), at screening
5. Sufficiently physically healthy in the opinion of the study and clinical teams to receive dexmedetomidine oromucosal film

### Exclusion Criteria:

1. Per medical record or team report, diagnoses of:
   Dementia
   Significant traumatic brain injury
   History of stroke, with persistent neurologic deficits
2. Presence of any of the following cardiovascular comorbidities
   Sick sinus syndrome
   A resting heart rate of <55 beats per minutes or systolic blood pressure <100 mmHg or >160 mmHg or diastolic BP <70 mmHg or >95 mmHg at enrollment and prior to dosing.
   Evidence of cardiac ischemia on a 12-lead electrocardiogram (ECG)
   Corrected QT interval of >450 msec
   Presence of a permanent pacemaker device
3. Per medical record (notes, current medications, flowsheets):
   Second degree (or greater) AV block without a pacemaker
   Known allergy or adverse reaction to dexmedetomidine
   Current use of dexmedetomidine
4. Inability to take oromucosal dexmedetomidine due to severe agitation, neurological impairment, NPO status, or other cause.
5. Hepatic impairment (liver function tests >3 times the upper limit of normal)
6. Severe renal impairment (GFR <30 ml/min or on dialysis)
7. Weight <60 kg
8. Pregnancy (in women; tested with serum or urine hCG)
9. Non-fluency in English
10. Prior enrollment in the study, with receipt of study medication, during the current or a previous hospitalization

### Assessment for Inclusion Criteria:

Inclusion and exclusion criteria will be assessed in a stepwise fashion. Prior to approaching participants/surrogates, study staff will review the electronic medical record to assess for exclusionary conditions and will discuss with clinical teams if the presence or absence of such conditions is unclear. Only those patients who do not clearly meet exclusion criteria will be evaluated further for inclusion and exclusion criteria by the study psychiatrist.

Specifically, the study psychiatrist will confirm a diagnosis of delirium through review of the medical record, evaluation of the potential participant, and discussion with the inpatient team if any questions about diagnosis remain following the evaluation. Delirium will be diagnosed using DSM-5 criteria:
1. There is a disturbance in attention and awareness.
2. The disturbance develops over a short period of time, represents an acute change from baseline, and tends to fluctuate over the course of the day.
3. There is an additional disturbance in cognition.
4. The disturbances are not better explained by an underlying neurocognitive disorder (e.g., dementia).
5. The disturbances do not occur in the context of a severely reduced level of arousal (e.g., coma).
6. There is evidence that the disturbance is a direct physiological consequence of another medical condition, substance intoxication or withdrawal, or exposure to a toxin, or is due to multiple etiologies.

This clinical diagnosis will be made in the context of a psychiatric and cognitive evaluation, including bedside tests of orientation, attention, and memory.

If a patient meets criteria for delirium, the study psychiatrist will then review the patient's medical record further and speak with the inpatient team to determine if any exclusion criteria are present. To ensure the clinical team feels that the participant is medically able to tolerate dexmedetomidine, they will be asked whether they would consider the participant to be suitable to receive dexmedetomidine intravenously if he/she were to become agitated. If participants meet diagnostic criteria for delirium and do not meet any exclusion criteria, they will be considered eligible for inclusion in the study.

Following enrolment in the study, laboratory testing and obtainment of an ECG will be performed to confirm clinical stability prior to medication administration. If a participant is experiencing agitation immediately following enrollment and confirmation of clinical stability, the participant will be eligible to be randomized to receive the study medication. If the participant does not have evidence of agitation, the participant will be monitored on a daily basis for the development of agitation (RASS 1) and will be randomized only if/once agitation has developed. While patients with or without agitation will be eligible for enrollment in the study, only those participants who become agitated will receive dexmedetomidine film.

### Study Procedures

### A. Data Collection and Monitoring

Initial screening and data collection. As noted, prior to enrollment, study psychiatrists will assess the patient for inclusion and exclusion criteria via brief medical record review, discussion with primary treatment team, diagnostic evaluation (DSM-5 criteria), and assessment for decision-making capacity. For enrolled subjects, chart reviews will be performed to gather baseline characteristics (Table 17—schedule of events).

Further screening for eligibility to receive study medication. Following enrollment, laboratory studies, including comprehensive metabolic panel (glucose, sodium, potassium, chloride, bicarbonate, calcium, carbon dioxide, magnesium, blood urea nitrogen, creatinine, uric acid, inorganic phosphorus) and liver function tests (alkaline phosphatase, aspartate aminotransferase [AST], alanine aminotransferase [ALT], gamma-glutamyl transferase, total bilirubin) will be performed.

Additionally, serum human chorionic gonadotropin (HCG; female participants only) will be obtained. If these laboratory studies were already drawn during this admission, new samples will not be drawn unless a change in clinical status that might affect them has occurred. Finally, a standard, 12-lead ECG will be obtained. The ECG parameters assessed will include heart rate and PR, QRS, QT, and QTc using Bazett's (QTcB) and Fridericia's (QTcF) correction methods. The QTcF will be considered the standard QTc interval to evaluate any changes in QTc in response to the study medication. The ECGs will be interpreted by the investigator, and if needed in the medical opinion of the principal investigator or designee, the findings will be confirmed by a cardiologist, critical care physician, or anesthesiologist. Study medication administration will not occur until all necessary laboratory studies have resulted and the participant's eligibility for medication administration has been confirmed.

Monitoring for the development of agitation: On a daily basis during the work week, the participant will be evaluated by a study team member using the Richmond Agitation Sedation Scale (RASS). If/when a participant is found to have significant agitation (defined as a RASS score 1), the participant will undergo baseline monitoring procedures and will be randomized to one of the two treatment conditions.

Randomization: Participants will be randomized to receive either 20 μg or 60 μg of dexmedetomidine sublingually. As dexmedetomidine is available in 10 and 60 μg films, those receiving 20 μg will receive two 10 μg films, while those receiving 60 μg will receive a 60 μg film and a placebo film to ensure blinding of staff and participants. Participants will receive repeat dosing every 30 minutes for up to three additional doses, leading to maximum doses of 80 μg and 240 μg, respectively. Both investigators and clinicians will be blind to the participant's group, with only the study pharmacist aware of the dose of medication on the films.

Baseline monitoring. Following enrollment, the study team will record baseline measures of heart rate, blood pressure, and oxygen saturation. An ECG will be performed and QTcF measured. Next, the physician will evaluate the patient using the RASS and DRS-R-98 to measure baseline agitation and delirium severity, respectively.

Medication administration. Dexmedetomidine oromucosal film will be administered by the study physician or study nurse as per the manufacturer's instructions. Specifically, the film will be placed into the participant's mouth. The participants will be instructed to keep the film in their mouth until it dissolves. If the participant is unable to hold the medication in their mouth (e.g., they spit it out), the participant will not be re-dosed. Dexmedetomidine administration will be repeated every 30 minutes if the participant continues to have agitation (RASS 1) and does not meet any cardiovascular stopping criteria. Dosing times and maximum medication doses are as follows in table 16.

TABLE 16
Dosing times and maximum medication doses

| | Administration Time (minutes) | | | | |
|---|---|---|---|---|---|
| Group | 0 | 30 | 60 | 90 | Total Dose |
| 20 μg | 20 μg | 20 μg | 20 μg | 20 μg | 80 μg |
| 60 μg | 60 μg | 60 μg | 60 μg | 60 μg | 240 μg |

Monitoring for side effects. Heart rate, blood pressure, oxygen saturation, use of supplemental oxygen, and use of pressors will be monitored continuously and recorded every 30 minutes from the participant's Epic flowsheet or using the telemetry monitor/automated blood pressure cuff for the 6 hours following the initial medication administration (baseline; Time 0). An ECG will be performed at 1.5, 3, 4.5, and 6 hours, and QTc will be calculated using the Fridericia formula, as described above. Study staff will also monitor the participant and speak with nursing staff at 6 hours to assess for any other side effects/complaints the patient may have had during the time since medication administration. These will be assessed formally using a list of side effects reported for dexmedetomidine in post-marketing surveillance.

Monitoring of agitation and delirium severity. The RASS will be performed every 30 minutes (up to 6 hours post-baseline) by study staff (research coordinator, nurse, or study psychiatrist) to monitor for agitation. The DRS-R-98 will be performed by the study physician at 1, 2, 3, 4, and 6 hours post-baseline to assess for changes in delirium severity. If patients require additional treatments to manage agitation following medication administration within the 6-hour monitoring period, all efficacy and safety assessments from that point forward will continue, but those time points for those patients will not be analyzed in the primary data analysis.

Dosing will be stopped at any time if any of the following occurs:
1. There is >30 mm Hg decrease in systolic or diastolic blood pressure.
2. There is an isolated drop in Systolic BP <95 mmHg.
3. There is an isolated drop Diastolic BP <55 mmHg.
4. There is a decrease in heart rate of 20 beats per minute or a drop below 55 beats per minute.
5. ECG reveals QTc >500 msec.
6. Attainment of a RASS of −1.

TABLE 17
Schedule of study events.

| | Pre- | | Med | 30 | 1 | 1.5 | 2 | 2.5 | 3 | 3.5 | 4 | 4.5 | 6 |
|---|---|---|---|---|---|---|---|---|---|---|---|---|---|
| Measure | enrollment | Baseline | Admin. | min | h | h | h | h | h | h | h | h | h |
| Delirium Screening | X | | | | | | | | | | | | |
| (CAM-ICU) | | | | | | | | | | | | | |
| Delirium Diagnosis | X | | | | | | | | | | | | |
| (DSM-5) | | | | | | | | | | | | | |
| Agitation (RASS) | X | X | X | X | X | X | X | X | X | X | X | X | X |
| Delirium Severity | | X | | | X | | X | | X | | X | | X |
| (DSR-R-98) | | | | | | | | | | | | | |
| Heart Rate | | X | X | X | X | X | X | X | X | X | X | X | X |
| Blood Pressure | | X | X | X | X | X | X | X | X | X | X | X | X |
| Oxygen Saturation | | X | X | X | X | X | X | X | X | X | X | X | X |
| Electrocardiogram | | X | | | | X | | | X | | | X | X |
| Other side effects | | | | | | | | | | | | | X |

### Study Drugs

In this study, we will administer the medication as a oromucosal film formulation (absorbed sublingually) at a dose of 20 or 60 μg (with repeated administration, participants may receive a total of up to 80 μg or 240 μg, respectively).

Data collection

Baseline data. Prior to administration of dexmedetomidine, the study physician will perform the RASS and DRS-R-98 to assess level of agitation and delirium severity. Heart rate, blood pressure, and oxygen saturation will be recorded from the participant's telemetry monitor and using an automated blood pressure cuff. Finally, an ECG will be performed, and the study physician will calculate a QTc interval.

Cardiovascular parameters and side effects. Following administration of dexmedetomidine, heart rate, blood pressure and oxygen saturation will be recorded every 30 minutes from the telemetry monitor or Epic flowsheet. Consistent with prior research, we will monitor for the following side effects:
1. Bradycardia—heart rate <55 beats per minute (or >20% reduction in heart rate from baseline if baseline heart rate is <70 beats per minute)
2. Hypotension—systolic blood pressure <95 mmHg (or ≥20% decrease in systolic blood pressure from baseline if baseline systolic blood pressure is <120 mm Hg), or the addition or increase of vasopressors
3. Tachycardia—heart rate >100 beats per minute (or ≥20% increase in heart rate from baseline if baseline heart rate is >83 beats per minute)
4. Hypertension—systolic blood pressure >160 mmHg (or ≥20% increase in systolic blood pressure from baseline if baseline systolic blood pressure is >133 mm Hg)
5. Hypoxia—oxygen saturation <90% (or ≥5% decrease in absolute value of oxygen saturation if baseline oxygen saturation is less than 95%), or increase in the amount of supplemental oxygen required to maintain oxygen saturation >90%
6. ECG changes (including QTc prolongation)—The ECG parameters will include heart rate, PR interval, QRS interval, QT interval, and QTc interval (rate correction using QTcB and QTcF methods). Summaries of ECG results and the change from baseline will be presented. The numbers and percentages of participants with a QTc increase from baseline to 90-minute ECG time point will be summarized using the following change categories:
   a. QTc interval increase from baseline >30 to ≤60 msec
   b. QTc interval increase from baseline >60 msec

In addition, QTc values will be summarized by gender as normal to prolonged in accordance with Committee for Proprietary Medicinal Products (CPMP) Points to Consider regarding the assessment of the potential for QT interval prolongation shown in the Table 18.

TABLE 18
QTcF Intervals: Upper and Lower Limits
of Normal, Borderline, and Prolonged

| Intervals | Males | Females |
|---|---|---|
| Normal | ≤430 msec | ≤450 msec |
| Borderline | >430 to ≤450 msec | >450 to ≤470 msec |
| Prolonged | >450 msec | >470 msec |

Note:
In accordance with CPMP guidelines.

Similarly, the numbers and percentages of participants with absolute QTc interval values above certain thresholds will be summarized by gender using the following limits in accordance with International Conference on Harmonisation (ICH) E14 guidance:
QTc interval >450 to ≤480 msec
QTc interval >480 msec
QTc interval ≥500 msec

A listing of participants with QTc change from baseline between 30 to 60 msec and ≥60 msec will be provided. A listing of participants with abnormal QTc interval values (>450 msec to <500 msec and ≥500 msec) will be provided to the Sponsor as well.

Non-cardiovascular side effects. During the final assessment (6 hours after the initial dose of the medication), participants will be asked the following question: “Have you noticed any new physical problems or side effects since receiving the study medication?” In addition, the participant's primary nurse will be asked whether the patient had signs or symptoms of new medical conditions or side effects (e.g., muscle stiffness, tremor, rash) since the administration of the medication, using a checklist of side effects reported in post-marketing surveillance.

### Impact on Agitation and Delirium Severity

Agitation: Agitation will be measured using the RASS, a 10-point scale to quantify levels of consciousness and agitation. This validated measure can be administered in less than one minute and has clear definitions for levels of arousal/agitation. The RASS will be administered at baseline and every 30 minutes (up to 6 hours post-baseline) by a research coordinator, study nurse, or study psychiatrist.

Delirium severity: The DRS-R-98 will be used as a measure of delirium severity. This 16-item scale can be used to screen for/diagnose delirium, but it also includes 13 items to assess delirium severity. It is reliable and has been validated in patients with delirium. This scale will be administered by a study physician at baseline, then 1, 2, 3, 4, and 6 hours after the initial dexmedetomidine administration.

### Example 7: A Phase 2, Multicenter, Randomized, Double-Blind, Placebo-Controlled, Ascending Starting Dose Finding, Safety, and Efficacy Study of Dexmedetomidine Hydrochloride Oromucosal Film in Agitation Associated with Delirium in ICU Patients

### Objectives

### Primary Objective

To determine the optimal dexmedetomidine hydrochloride oromucosal film starting dose (StartD) that will safely and effectively reduce agitation associated with delirium in ICU subjects using the Richmond Agitation Sedation Scale (RASS).
Primary Endpoint: Identification of the dose leading to a 2-point or greater drop in RASS at 2 hours after starting dose administration, with initial RASS not ≤−3.

### Secondary Objectives:

To identify the earliest time when a significant effect on agitation is seen.
Secondary Endpoint: Earliest time at which a 2-point drop is seen in RASS after starting dose administration.
Evaluate the effect of dexmedetomidine hydrochloride oromucosal film on overall delirium improvement as measured by the CAM-ICU-7 Total Score during ICU stay
Determine the safety & tolerability profile of dexmedetomidine hydrochloride oromucosal film compared to placebo as measured by hemodynamic/CV parameters (including blood pressure, heart rate, and QTc interval) and adverse events
To assess the PK of dexmedetomidine hydrochloride oromucosal film in these subjects.

### Exploratory Objective

To determine the overall clinical improvement after drug administration as measured by the Clinical Global Impression-Improvement Scale (CGI-I).

### Study Design

This study is designed to determine and evaluate the optimal dexmedetomidine hydrochloride oromucosal film starting dose (StartD) that safely and effectively reduces agitation associated with delirium in ICU patients using the Richmond Agitation Sedation Scale (RASS).

This is an ascending adaptive dose study evaluating the safety and efficacy of four potential doses of dexmedetomidine hydrochloride oromucosal film (120 μg, 180 μg, 240 μg, and 300 μg) in reducing agitation levels in adult ICU patients with delirium. For subjects 65 years old and older, the potential doses will be reduced 50% to reduce the chance of AEs in this population. The purpose of this clinical trial is to identify an optimally safe and effective dexmedetomidine hydrochloride oromucosal film starting dose.

### Ascending Starting Dose Adaptive Design

This is a randomized, double-blind, placebo-controlled, ascending starting dose finding study assessing safety, efficacy, tolerability and PK of dexmedetomidine hydrochloride oromucosal film in four potential starting dose cohort groups to reduce agitation levels associated with delirium within the ICU setting. Each cohort will consist of 20 male and female subjects >18 years old.

Cohorts will be enrolled sequentially in this dose escalating design. The initial cohort will be administered a starting dose of 120 μg and subsequent cohorts will be enrolled after thorough review of the safety data from the previous cohorts to ensure acceptable safety and tolerability. In addition, the patients to be enrolled in this trial are studied in the ICU setting and are carefully monitored.

Evaluation of four dexmedetomidine hydrochloride oromucosal film starting doses compared to placebo will be conducted according to the following ascending dose plan:

Cohort 1: 120 μg (one 120 μg film) or placebo (1 placebo film)

Cohort 2: 180 μg (one 180 μg film) or placebo (1 placebo film)

Cohort 3: 240 μg (two 120 μg films) or placebo (2 placebo films)

Cohort 4: 300 μg (one 120 μg film and one 180 μg film) or placebo (2 placebo films)

For subjects 65 years old and older enrolled in each of those cohorts, the dose will be reduced by 50% in line with the Precedex (reference drug) label. For subjects 65 years and older, evaluation of four starting doses compared to placebo will be conducted according to the following ascending dose plan:

Cohort 1: 60 μg (120 μg film cut in half) or placebo (½ placebo film)

Cohort 2: 90 μg (180 μg film cut in half) or placebo (½ placebo film)

Cohort 3: 120 μg (one 120 μg film) or placebo (1 placebo film)

Cohort 4: 150 μg (one 180 μg film cut in half and one 120 μg film cut in half) or placebo (2 half placebo films)

The ratio of dexmedetomidine to placebo in each cohort is 3:1.

Escalation of dose will occur only after results from the previous dose have been reviewed by the internal safety committee comprised of PI, sponsor medical monitor, and Chief Development Officer. Patients to be enrolled in this trial are studied in the ICU setting and are closely monitored.

This is an ascending adaptive design. Doses selected for testing may differ from these, based upon safety reviews. In addition, a cohort or cohorts may be repeated or added, to provide more information about a starting dose with respect to tolerability or impact in decreasing RASS scores as a function of baseline RASS score or other factors.

Subjects 18 years old and older (or their legally appointed representative) will be asked to provide written informed consent (or assent) at the time of admission to ICU. Upon confirmation of eligibility through Inclusion and Exclusion Criteria, subjects will receive the first starting dose (dexmedetomidine hydrochloride oromucosal film or placebo, 3:1 randomization manner respectively) when baseline RASS score is ≥+1. Subsequent doses may start in increments of 120 every 3 to 6 hours post first dose (StartD) only if RASS is still ≥+1, to a maximum cumulative dose of 960 μg in the 24 hours treatment period.

For subjects 18-64 years old, re-dosing as needed if RASS is still ≥+1 per cohort reflects the following maximum levels (cohorts):
maximum of seven 120 μg doses at the 120 μg starting dose level (cohort 1)
maximum of six additional 120 μg doses at the 180 μg starting dose level (cohort 2)
maximum of six additional 120 μg doses at the 240 μg starting dose level (cohort 3)
maximum of five additional 120 μg doses at the 300 μg starting dose level (cohort 4)

For subjects 65 years and older, subsequent doses may start in increments of 60 μg every 3 to 6 hours post first dose only if RASS was still ≥+1, to a maximum cumulative dose of 480 in the 24 hour treatment period. Re-dosing as needed for subjects 65 years and older reflects the following maximum levels:
maximum of seven additional 60 μg doses at the 60 μg starting dose level (cohort 1)
maximum of six additional 60 μg doses at the 90 μg starting dose level (cohort 2)
maximum of six additional 60 μg doses at the 120 μg starting dose level (cohort 3)
maximum of five additional 60 μg doses at the 150 μg starting dose level (cohort 4)

Subjects can only be titrated (re-dosed) if they are hemodynamically stable, not hypotensive (must be greater than 90/60 systolic/diastolic), not bradycardic (must be greater than 60 bpm), and not experiencing an AE. Except for the first starting dose cohort (Cohort 1: 120 μg), each subsequent dose level will be authorized after a safety review of the results from the previous dosing cohort.

Haloperidol can be used as a rescue medication per PI decision, preferably not before 4 hours post starting dose (StartD), with an initial IV haloperidol bolus of 2.5 to 5 mg. Haloperidol dosing may be repeated every 30 minutes until desired calmness (RASS −2 to 0) is achieved. Subjects can only be re-dosed if hemodynamically stable, no abnormalities seen on ECG, and not experiencing an AE. Except for the first starting dose cohort (Cohort 1: 120 μg), each subsequent dose level is authorized after a safety review of the results from the previous dosing cohort.

Agitation severity will be assessed with the RASS at Baseline and at the following timepoints post initial dose (StartD): 10 minutes; 20 minutes; 30 minutes; and then every 30 minutes for 2 hours post-dose then every 4 hours post-dose for the 24-hour treatment period.

Delirium presence and severity will be assessed with the CAM-ICU and CAM-ICU-7, respectively, which will be administered at Screening, pre-dose, and post-dose every 4 hours post initial dose (StartD). If subject is re-dosed, RASS evaluation should be performed 2 hours after each re-dosing.

Safety, efficacy, and tolerability will be assessed throughout the treatment period at various timepoints.

Safety monitoring will follow administration of the first dose (StartD) and continue for the duration of the treatment period (24 hours) and for an additional 24-hour period without study drug. During this safety monitoring period the following assessments will be conducted:

Vital signs monitoring every 30 minutes, pulse oximetry every hour

ECG will be monitored on telemetry per ICU protocol, formal complete strip ECG will be done at screening, 2, 4 and 8 hours post-dose, and end of treatment

AE monitoring at 4-hour intervals

After completion of the lowest dose cohort (Cohort 1: 120 μg or 60 μg for subjects 65 years old and older), a safety and tolerability review will be performed by the PI, sponsor medical monitor and Chief Development Officer. At dose escalation meetings safety and tolerability data will be reviewed to determine the next dose to be tested. PK data will be reviewed as it becomes available. A safety review will also be conducted in the event of a Serious Adverse Event (SAE).

### Stopping Criteria for Study:

One or more deaths or 5 or more nonfatal SAEs; both considered to be attributable to dexmedetomidine hydrochloride oromucosal film by the PI and Sponsor.

### Stopping Criteria for Subjects:

Significant oral mucosal reaction (necrosis, bleeding, infection) considered by the PI to be due to film application during the study.

Persistent hemodynamic instability defined as SBP <90 mmHg or heart rate <40 bpm if not responding to dose reduction/hold

Any AE Grade 3 or greater considered to be related to the study drug by the PI and in the opinion of the PI poses an unacceptable risk for the subject.

Treatment emergent clinically significant ECG changes from baseline, brady-and tachyarrhythmias, QTcF >500 msecs

Subject unable to self-administer the dexmedetomidine hydrochloride oromucosal film or placebo film

### Number of Subjects

Twenty subjects per cohort will be randomized 3:1—active:placebo

### Diagnosis and Main Criteria for Eligibility

### Inclusion Criteria for Enrollment (Informed Consent)

ICU admitted male and female patients, ≥18 years old, COVID 19 (+) and (−)

With or without mechanical ventilation

Subject or legally appointed representative (LAR) able to read, understand and provide informed consent, or to provide assent

### Inclusion Criteria for Randomization

Positive CAM-ICU

RASS score ≥+1

Subject judged to be likely capable of self-administration of sublingual or buccal film

### Exclusion Criteria

Second degree Type II AV block or complete heart block, or bradycardia with hemodynamic instability; hemodynamic instability defined as: SBP <90 mmHg and heart rate ≤50 bpm; moderate to high vasopressor (this exclusion criterion does not apply to subjects receiving low dose norepinephrine [5 μg /kg/min or less], epinephrine [0.1 μg /kg/min or less], or phenylephrine [100 μg /kg/min or less], or those in the process of weaning off moderate to high dose vasopressor); severe ventricular dysfunction

Hyperkalemia defined as serum potassium level of >5.0 mEq/L

Adrenal suppression based on clinical symptoms or measured cortisol levels (Morning cortisol level of <10 μg /DL)

Polyuria (>3 liters urine output within 24 hrs)

Clinically significant ECG changes, brady- and tachyarrhythmias, QTc prolongation >480 msec

Hepatic dysfunction, defined as ascites, bilirubin >10% above the upper limit of normal, or liver function tests >3× upper limit of normal

Pregnancy

Known allergy to dexmedetomidine or haloperidol.

Repeat laboratory testing per PI discretion.

### Study Treatments

### Method of Assigning Subjects to Treatment Groups

Upon confirmation of eligibility, subjects will be randomized to dexmedetomidine hydrochloride oromucosal film or placebo film.

In each of four cohorts, twenty (20) new participants will be enrolled, randomized to 3:1 dexmedetomidine hydrochloride oromucosal film: Placebo film, i.e., 15 receiving dexmedetomidine hydrochloride oromucosal film and 5 receiving Placebo film per cohort. Study randomization will be computer generated.

### Test Product, Dose, and Mode of Administration

Dexmedetomidine hydrochloride will be in an orally dissolving film formulation for sublingual (SL) and buccal administration provided in two strengths: 120 μg and 180 μg. Dosing delivers 180 μg or 120 μg of DEX sublingually or behind the lower lip, between the gum and lip. The 120 μg dose and 180 μg dose are administered as one film; the 240 μg and 300 μg doses are administered as a combination of two films placed side by side, not overlapping. Half doses are achieved by cutting either the 120 μg or 180 μg films in half so that the 120 μg film if cut in half result in a 60 μg dose when administered and the 180 μg film if cut in half result in a 90 μg dose when administered.

The product is a small, solid-dose film formulation, designed to completely solubilize in the SL space or buccal space (behind the lower lip between the gum and lip) within 1-3 minutes. Drinking or eating should be prohibited for 15 minutes immediately following administration. Participants will also be evaluated for sublingual or buccal irritation around the area where the films are placed.

### Treatment Administration

At the beginning of each study session, subjects are verbally instructed on how to administer the investigational product along with a visual demonstration of where to place the film within the buccal cavity. Dosing delivers 180 μg or 120 μg of DEX sublingually or behind the lower lip, between the gum and lip. The 120 μg dose is administered as one film; the 180 μg dose is administered as one film, the 240 μg and 300 μg doses are administered as a combination of two films placed side by side, not overlapping. For subjects age 65 years old and older, a 50% reduction in dose will be applied, such that the 120 μg film or the 180 μg film is cut in half prior to administration (i.e., the 60 μg dose is half of 120 μg film; the 90 μg dose is one half of the 180 μg film; the 120 μg dose is one 120 μg film; the 150 μg dose is one half of the 120 μg film alongside one half of the 180 μg film). The investigational product will be retained in the sublingual cavity or lower lip until dissolved.

### Reference Therapy, Dosage and Mode of Administration:

Matching placebo films are administered sublingually or behind the lower lip between the gum and lip.

### Duration of Treatment

24 hours

### Study Procedures

Subjects or their LAR will provide written informed consent, and assent as applicable, before any study-related procedures are initiated, including the cessation of prohibited concomitant therapy.

The schedule of events to be performed during the study is provided in Table 19.

TABLE 19
Schedule of Events

| | | | Post Dose Time | | | | | | | | | | |
|---|---|---|---|---|---|---|---|---|---|---|---|---|---|
| | | Pre- | | | | | | | | | | | Post-24 |
| | Screening | Dose | | | | | | | | | | | Hour |
| Activity | Pre- | −1 hr to | 10 | 20 | 30 | 1 | 1.5 | 2 | 4 | 6 | 8 | 24 hr | Treatment |
| Time point | treatment | time 0 | min | min | min | hr | hr | hr | hr | hr | hr | Day 1 | Day 2⁸ |
| Informed Consent¹ | X | | | | | | | | | | | | |
| Medical History | X | | | | | | | | | | | | |
| Demographics | X | | | | | | | | | | | | |
| Weight | X | | | | | | | | | | | | |
| Height | X | | | | | | | | | | | | |
| BMI | X | | | | | | | | | | | | |
| Physical Exam ¹⁶ | X | | | | | | | | | | | X | |
| Safety Labs³ | X | X | | | | | | | | | | | X |
| ECG with rhythm strip⁹ | X | | | | | | | X | X | | X | X | |
| Clinical Labs | X | | | | | | | | | | | X | X |
| Pregnancy test² | X | | | | | | | | | | | | |
| Pulse oximetry¹⁰ | | X | | | | X | | X¹⁰ | X | X | | X | X |
| Resting vital signs¹¹ | X | X | | | X | X | X | X¹¹ | X | X | | X | X |
| Admit to ICU Date | X | | | | | | | | | | | | |
| CAM-ICU⁴ | X | X | | | | | | | X⁴ | | | X | |
| Inclusion/Exclusion | X | X | | | | | | | | | | | |
| criteria | | | | | | | | | | | | | |
| Randomization⁵ | | X | | | | | | | | | | | |
| Starting Dose | | X | | | | | | | | | | | |
| Study drug | | X | | | | | | | | | | | |
| administration | | | | | | | | | | | | | |
| RASS⁶ | X | X | X | X | X | X | X | X | X⁶ | | | X | |
| CAM-ICU-7⁷ | X | X | | | | | | | X⁷ | | | X | |
| Clinical Global | X | X | | | | | | | | | | | |
| Impression Scale - | | | | | | | | | | | | | |
| Severity ¹⁴ | | | | | | | | | | | | | |
| Clinical Global | | | | | X | | | X | | | | X | |
| Impression Scale - | | | | | | | | | | | | | |
| Improvement ¹⁵ | | | | | | | | | | | | | |
| Buccal (SL) assessment | | | | | | | | | | | | X | |
| for local irritation | | | | | | | | | | | | | |
| Pharmacokinetic | | X | | | X | | | X | X | | X | X | |
| Sampling¹³ | | | | | | | | | | | | | |
| Prior Meds | X | | | | | | | | | | | | |
| Readmission to ICU | | | | | | | | | | | | | |
| Concomitant Meds | X | X | X | | | | | | | | | | |
| Adverse Events¹² | X | X | X | | | | | | | | | | |

¹Written informed consent will be obtained from the subject or LAR, and assent if applicable, upon admission to ICU and PI identifies risk for delirium. Symptoms, understanding of study, and appropriateness must be documented in source. No study procedures may be performed prior to completion of the ICF process.
²Urine pregnancy test for all females of childbearing potential will be conducted at Screening.
³Safety Labs will include hematology, clinical chemistry, urinalysis, and urine drug screen to be collected at Screening visit and specified intervals pre-dose, post-treatment, and per PI discretion. Safety and tolerability assessment will include hemodynamic/cardiovascular parameters (blood pressure; heart rate; QTc interval).
⁴The CAM-ICU will be administered to assess presence of delirium at Screening, Pre-Dose, and Post-Dose every 4 horns thereafter for the 24 hour treatment period.
⁵Randomization will occur upon completion of pre-randomization procedures, at which time the subject will be randomized to study treatment (dexmedetomidine hydrochloride oromucosal film or placebo SL film).
⁶The RASS will be administered to assess agitation severity at Screening, Pre-Dose, and Post-Dose at 10 min, 20 min, 30 min, and every 30 min for the first 2 hours after treatment then every 4 hours for the 24 hour treatment period. If subject is re-dosed, the RASS will be administered every 2 hours after each re-dose.
⁷The CAM-ICU-7 will be administered at Screening to assess severity of delirium Pre-Dose and Post-Dose every 4 hours for the 24 hour treatment period.
⁸Day 2: 24 hr safety monitoring period without study medication
⁹ECG monitored by telemetry per ICU protocol; formal complete strip ECG will be completed at Screening, 2 hours post-dose, 4 hours post-dose, 8 hours post-dose, and at end of treatment. In addition, to minimize variability across participating sites, ECG documentation may be collected and submitted to be read centrally.
¹⁰Pulse oximetry taken every 1 hr post dose
¹¹Vital signs taken every 30 minutes post dose
¹²AE monitored in 4 hr intervals post dose. Adverse events will contribute to assessment of safety and tolerability.
¹³PK samples will be collected pre-dose and at 30 min, 2 hrs, 4 hrs, 8 hrs, and 24 hrs post dose. If additional dexmedetomidine hydrochloride oromucosal film doses were administered, additional PK samples will be collected just before and at 2 hr post-dose for each additional dose.
¹⁴ CGI-S will be completed at Screening and pre-dose
¹⁵ CGI-I will be completed at 30 minutes post-dose, 2 hours post-dose, and 24 hours post-dose.
¹⁶ Physical examination will be performed at Screening and 24 hours post-dose.

### Example 8: Effect of Oromucosal formulation of Dexmedetomidine HCl on Ethanol in Heavy Drinkers with PTSD—Alcohol Interaction Study

### Objectives

The overall objective of the proposed study is to determine if dexmedetomidine HCl is safe for treatment of alcohol use disorder (AUD) with comorbid posttraumatic stress disorder (PTSD) and also shows potential signals of efficacy thereby supporting the conduct of later phase clinical trials. The specific aims of this study are:
1. to evaluate whether pretreatment with dexmedetomidine HCl 40 μg and 80 μg attenuates stress (PTSD) reactivity in individuals with AUD and PTSD;
2. to evaluate whether pretreatment with dexmedetomidine HCl 40 μg and 80 μg attenuates alcohol cue reactivity in individuals with AUD and PTSD; and
3. to evaluate whether pretreatment with dexmedetomidine HCl 40 μg and 80 μg alters subjective effects of ethanol in a laboratory setting
4. to evaluate whether pretreatment with dexmedetomidine HCl 40 μg and 80 μg increases side effect associated with ethanol administration including sedation and vital signs.

### Study Design

This laboratory study is a phase 1, double-blind, placebo-controlled, within subjects study. This study will consist of 3 laboratory test sessions following pretreatment with dexmedetomidine HCl/placebo for 10 heavy drinker participants with comorbid PTSD. Participants (n=10) will participate in a laboratory study with 3 test days (minimum of 2 days, but no longer than 2 weeks between each test session); for each test day they will be assigned to receive oromucosal dexmedetomidine hydrochloride 40 μg, 80 μg and placebo in a randomized fashion. Test sessions will be conducted to evaluate stress (PTSD) reactivity and alcohol cue reactivity. Participants will also receive IV ethanol administered via “clamp methodology” to assess for the effects of dexmedetomidine HCl in combination with ethanol.

Since this is the first time dexmedetomidine HCl is being tested in combination with alcohol administration, we will be using a modified randomization where participants will not receive the 80 μg dose until they have received the 40 μg dose.

### Primary Endpoints

The various assessments used to assess the safety and effect of dexmedetomidine HCl on stress (PTSD) and alcohol cue reactivity include:
Orthostatic vital signs
State Trait Anxiety Inventory (STAI-6)
Visual analog scales (VAS)
Yale Craving Scale (YCS)

Likewise, the assessments used to assess the safety and effect of dexmedetomidine HCl when taken concurrently with alcohol include those listed above as well as:
Orthostatic vital signs
Biphasic Alcohol Effects Scale (BAES)
Number of Drinks Scale (NDS)
Cognitive performance as assessed by the Hopkins Verbal Learning Test (HVLT-R), Go No-Go Task, and Rapid Information Processing Task (RVIP)
Motor impairment as assessed by the Grooved Pegboard Test

### Participant Inclusion Criteria

To be enrolled in this study, participants must meet the following criteria.
1. Male or female, Veterans and non-Veterans, ages 21 to 50;
2. Able to read and write in English and sign the informed consent;
3. Willing to comply with all study procedures and be available for the duration of the study;
4. ECG that demonstrates no clinically significant conduction issues or arrhythmias;
5. Have no clinically significant contraindications, in the judgement of the PI/study physician, for study participation (based on self-reported medical history and brief physical examination);
6. Have a current diagnosis of Alcohol use disorder (AUD) (mild, moderate, or severe) as determined by MINI-5 and PTSD as determined by the Clinician-Administered PTSD Scale for DSM-5 (CAPS-5);
7. PCL-5 score ≥33;
8. Must have >1 heavy drinking episodes (>4 standard drink units (SDU) for men; >3 SDU for women) in the last 30 days (assessed by the Timeline Follow Back (TLFB)).
9. Not be treatment seeking for AUD
10. Females of childbearing potential (not surgically sterilized (tubal ligation/hysterectomy) or not post-menopausal (no menstrual period for >6 months) must be willing to use a medically acceptable and effective birth control method for 3 months before the study and while participating in the study. Medically acceptable methods of contraception that may be used by the participant include abstinence, birth control pills or patches, birth control implants, diaphragm, intrauterine device (IUD), or condoms.

### Participant Exclusion Criteria

To be enrolled in this study, participants must not meet the following criteria.
1. Current bipolar disorder or psychotic disorders as determined by MINI-5;
2. Current diagnosis of a substance use disorder (other than alcohol, nicotine, or marijuana) as determined by MINI-5;
3. Females who are pregnant, nursing, or planning to become pregnant during study participation;
4. Current physiological alcohol dependence requiring a higher level of care (e.g. detox) as determined by study physician conducting physical examination and CIWA score. Tolerance to alcohol will be allowed.
5. Recent history of complicated alcohol withdrawal, alcohol withdrawal seizures, or delirium tremens (DTs);
6. Score >4 on Clinical Institute Withdrawal Assessment for Alcohol Scale (CIWA-Ar) at randomization;
7. History of major medical illnesses including liver disease, heart disease, chronic pain or other medical conditions that the physician investigator deems contraindicated for the participant to be in the study;
8. Clinically significant history of cardiac disease including (a) chronic hypertension (even if adequately controlled by antihypertensive medications); (b) history of syncope or other syncopal attacks; (c) current evidence of orthostatic hypotension (defined as a decrease in systolic BP of 20 mm Hg or decrease in diastolic BP of 10 mm Hg within 3 minutes); (d) resting heart rate of <60 beats per minute; (e) systolic blood pressure <110 mmHg or diastolic BP <70 mmHg; or (f) participants with a QTC interval >440 msec (males) or >460 msec (females).
9. Clinically significant medical conditions including hepatic ascites (bilirubin >10% above the upper limit of normal [ULN] or liver function tests [LFT] >3>ULN);
10. Renal impairment as measured by BUN/Creatinine;
11. Currently taking the following medications: a) medications for alcoholism (e.g. naltrexone, disulfiram, topiramate, acamprosate); b) psychotropic medications that promote sedation including sedative/hypnotics, barbiturates, antihistamines, sedative antidepressants (e.g. doxepin, mirtazapine, trazodone), and triptans (e.g., sumatriptan); c) antihypertensive medications; d) alpha-2-adrenergic agonists (clonidine, guanfacine, lofexidine); or adrenergic agents prescribed for other reasons are excluded (prazosin). (Permitted Concomitant Medications: The concomitant medications allowed in the study include non-sedative antidepressants used to treat PTSD);
12. History of allergic reactions to dexmedetomidine or known allergy to dexmedetomidine;
13. Participation in a clinical trial of a pharmacological agent within 30 days prior to screening;
14. Any finding that, in the view of the principal investigator, would compromise the subject's ability to fulfill the study visit schedule or requirements

### Strategies for Recruitment and Retention

### Multipronged Recruitment Strategy

The recruitment strategies include direct recruitment from the investigator's clinics, social media and news advertisement, flyers in the community, self-referral through word-of-mouth interactions with others, and integrated outreach to mental health and primary care outpatient clinics, substance rehabilitation programs, community, and inpatient units. Under a HIPAA waiver, IRB-approved letters of invitation can be mailed to prospective participants with a past diagnosis of AUD or PTSD or who otherwise might meet eligibility criteria.

### Retention Strategies

Adherence and attendance are vital to the success of the study. Patient education on study procedures in the study is the cornerstone. As a primary way to minimize drop-out from the study, the investigators and/or Clinical Research Coordinators (CRCs) provide thorough pre-enrollment education for all prospective participants during the informed consent process and confirm the participant's commitment to and feasibility for follow-up. Screening and baseline visits are separate from day of randomization so that any ambivalence can manifest, and participants can back-out prior to randomization. The investigators and CRCs also provide ongoing support and education during the study to reinforce the participants' commitment and resolve logistical barriers to keeping the appointments. Participant burden is kept to a minimum (i.e. small number of assessments and low frequency).

After consenting to participate, a urine drug screen and breathalyzer will be conducted at each visit. If the drug screen is positive for any exclusionary drugs and/or if the participant has a breathalyzer level >0.02 at any visit, the appointment may be canceled and rescheduled (at the discretion of the PI), and the participant will not be paid for the canceled appointment. The appointment may be rescheduled for a later date.

### Reasons for Withdrawal or Termination

Participants are free to withdraw from participation in the study at any time, or the investigator may terminate a participant's participation; however, these are two different actions that result in different study follow-up paths.

The investigator may discontinue study procedures for a participant or withdraw participation in the study if:
Any clinical adverse event (AE), laboratory abnormality, or other medical condition or situation occurs such that continued receipt of study medication or participation in the study would not be in the best interest of the participant;
The participant exhibits extreme post-dose vital sign changes, significant orthostasis, a syncopal event, or other observed intolerance to dexmedetomidine hydrochloride;
The participant has a newly developed, or not previously recognized, medical condition that precludes further study participation;
The participant demonstrates an inability to comply with the verbal and written study instructions and/or procedures or exhibits difficult behaviors (e.g. abusive, violent, aggressive);
The investigator decides that continuing in the study would be harmful to the participant;
The participant needs to take medications that are not allowed while in this study;
The participant is unable to keep appointments or complete study procedures as instructed;
The participant has a bad reaction to the study drug such that s/he can no longer continue to take them;
The study is canceled by the PASA Consortium/DOD, sponsor or Yale

### Study Design

### Study Agent(s)

Dexmedetomidine HCl oromucosal film and the matching placebo

### Study Drug Description:

Dexmedetomidine HCl is a thin film formulation of dexmedetomidine (DEX) for oromucosal administration. Dosing delivers 40 μg or 80 μg of DEX sublingually. Matching placebo films will also be taken sublingually. The product is a small, solid-dose film formulation, approximately 286 mm² in area and 0.7 mm thick, designed to solubilize in the SL space within 1-3 minutes.

Dexmedetomidine HCl oromucosal films are packaged as individual 80 μg or placebo films in a heat-sealed, white foil pouch with a drug product label. The 80 μg or placebo film can be removed from the pouch and cut into two 40 μg or equivalent placebo films. The pouch has a white colored outer layer with foil colored inner layer. There is a label on one side of the pouch. Each label has a colored border.

### Drug Administration

At the time of dosing, subjects will be verbally instructed on how to take the investigational product sublingually, and that they should retain the investigational product in the sublingual cavity until dissolved.

### Dosing

During the laboratory study, participants will receive the study agent following the completion of baseline assessments. Participants will be randomized to receive dexmedetomidine HCl oromucosal film 40 μg, 80 μg or placebo on 3 separate test days. All participants will be randomized using a modified scheme and will not receive the 80 μg dose until they have received the 40 μg dose.

### Route of Administration

Dexmedetomidine HCl will be administered orally, as individual films in the SL space. Ethanol will be administered intravenously (IV) using a clamp procedure, targeting a breath alcohol concentration (BrAC) of 100 mg % (100 mg/dL). This procedure will achieve steady-state blood alcohol levels without the individual variation from oral alcohol administration.

### Duration of Therapy

Participants will receive 3 separate doses of study agent, during three different test sessions.

### Ethanol Infusion

Alcohol Clamp Procedure: In this study, we will be using a modified alcohol-IV clamp procedure developed and standardized by Subramanian and colleagues (Subramanian, Heil et al. 2002), that has been used previously and in ongoing studies by the Co-PI (Kerfoot, Pittman et al. 2013). We will use the latest state-of-the art infusion method CAIS that uses a computerized control of the alcohol infusion that includes real-time pharmacokinetic modeling to optimize the reliability and standardization of the procedure (Zimmermann, O'Connor et al. 2013). The infusion will be performed using a 6% ethanol solution in 0.9% saline. The computer assisted administration program automatically calculates and corrects the infusion rate based on real-time BrAC data entry by staff, based on the pharmacokinetic profile of each subject.

### Study Specific Procedures

Procedures and assessments specific to the study include Medical, Cognitive and Motor Assessments, Clinician Administered Assessments, Self-Assessments, and Alcohol Cue and Stress (PTSD) Reactivity.

### Medical Procedures/Assessments

### Breathalyzer

Breathalyzer tests are conducted at each test visit during the laboratory phase in the research clinic by a trained clinical research coordinator and used to measure participants' breath alcohol concentration (BrAC). Samples >0.01 g/dl are considered positive. Results are kept in research records and not entered in medical records.

### Prior/Concomitant Medications (Con-Meds)

Concomitant medications (con-meds) are other prescription medications, over the counter (OTC) drugs or dietary supplements that a study participant takes in addition to the drug under investigation. Participants are asked to bring all their current medications with them at the time of their screening appointment.

### Medical History

This measure is a broad screening tool to collect information on a participants' medical history and check for conditions such as diabetes, liver disease, and kidney disease. It is comprised of questions to cover the following areas: allergies, asthma, head, ears, eyes, nose, throat (HEENT), cardiovascular, renal, hepatic, pulmonary, gastrointestinal, musculoskeletal, neurologic, psychiatric, dermatologic, metabolic, hematologic, endocrine, genitourinary, reproductive system, seizure, infectious disease, inflammatory or auto-immune disorders, and diabetes.

### Physical Examination (PE)

A targeted PE will include assessments of the head, eyes, ears, nose, throat, skin, thyroid, neurological system, lungs, cardiovascular system, abdomen (liver and spleen), lymph nodes, and extremities. Height and weight measurements will be taken during screening.

### Orthostatic Vital Signs

Orthostatic vital signs (blood pressure, pulse, and symptoms) will be obtained and recorded prior to medication administration, post medication administration, at the time of expected maximum effect of study drug, and prior to discharge from the study clinic. If the patient is unable to stand, orthostatic vitals may be taken while the patient is sitting with feet dangling. Measurements are to be repeated if clinically significant changes are observed or a machine error occurs.

### Oxygen Saturation (SpO2)

Oxygen saturation will be measured using a pulse oximeter prior to administering Dexmedetomidine HCl and approximately every 15 minutes after the study drug has been administered and through the end of the alcohol infusion. After the alcohol infusion ends oxygen saturation will be checked approximately every 30-60 minutes until the participant is discharged. If SpO₂ drops to <90% after administration, the study team will immediately discontinue any medication if indicated (e.g. alcohol infusion) and the participant will be monitored until it rises over threshold. The PI will determine whether the subject should be withdrawn from participation.

### Electrocardiogram (ECG)

12-lead ECGs will be obtained using a machine that automatically calculates heart rate and determines intervals for PR, QRS, QT/QTc and PRT axes.

### Clinical Institute Withdrawal Assessment of Alcohol Scale

Clinical Institute Withdrawal Assessment of Alcohol Scale, Revised (CIWA-Ar) is an interviewer-driven measure of alcohol withdrawal used to identify withdrawal symptoms requiring medical treatment. All study physicians have extensive experience in assessing and triaging patients who may be experiencing alcohol withdrawal. Participants who show signs or symptoms of alcohol withdrawal and who need detoxification will be referred for detoxification at an outpatient clinic or an inpatient unit (depending on the level of care needed).

### Adverse Events

All AEs occurring during the clinical trial will be collected, documented, and reported by the Principal Investigator/designated study staff according to the specific procedures. Additionally, study staff will assess patients for any medical or psychiatric side effects by asking the participant “How have you been feeling since I saw you last?” and in a manner consistent with Systematic Assessment for Treatment of Emergent Events (SAFTEE) guidelines (Johnson, Ait-Daoud et al. 2005). Study staff will also review the previous Adverse Event Form and inquire whether any of those events are continuing. Each new or unresolved adverse event will be recorded on the Adverse Event Case Report Form using a brief verbatim term, a severity ranking, and any additional description, according to the procedures. If an adverse event is reported that requires medical attention, it will be reported to a study clinician immediately for review. The PI/trained staff member will review each AE to assess their possible relationship to study medications and expectedness.

### Side Effects

A list of side effects associated with taking dexmedetomidine oromucosal film will be reviewed by the study nurse and specific symptoms, as well as severity, will be recorded. The Side Effect questionnaire will be administered during each test session. The side effects checklist will include items assessing the local tolerability (oromucosal) of dexmedetomidine HCl including (1) signs of local irritation at the application site of the oromucosal preparation (buccal mucosa); (2) swelling of tongue, lip, mouth, and throat; (3) shortness of breath; and (4) anaphylaxis.

### Cognitive and Motor Assessments

### Cognitive Performance Assessments

The Rapid Information Processing Task (RVIP) is a widely used task to assess sustained attention, with a working memory component. These cognitive functions are sensitive to acute alcohol administration (Howland, Rohsenow et al. 2011, Ralevski, Perry et al. 2012). In this task, a series of single digits is presented on a computer screen at a rate of 100 digits per minute for 4 min. Targets are defined as three consecutive odd digits (e.g., 7-9-3) or three consecutive even digits (e.g., 2-8-6). The percentage of targets correctly detected will be the main outcome measure. A Go No-Go task (Sofuoglu, Waters et al. 2008) will assess the ability to withhold responses to an infrequently occurring target (No-Go trials). Alcohol has been shown to impair responses to Go No-Go task (Weafer and Fillmore 2012). A series of blue and green rectangular shapes are presented every 1150 ms and participants are instructed to press a spacebar every time the green rectangular shape appeared, and to give equal importance to speed and accuracy. The primary outcome is the number of errors on the No-Go trials. The Hopkins Verbal Learning Test-Revised (HVLT-R) is a word list learning test of verbal memory (Brandt 1991). Verbal memory is sensitive to the acute alcohol effects (Ray and Bates 2006, Schweizer, Vogel-Sprott et al. 2006) and has the advantage of 6 different versions that permit multiple episodes of testing. The main outcomes for the HVLT-R are percent correct immediate and delayed recalls).

### Motor Impairment

The Grooved Pegboard Test (Lafayette Instrument Company) is a manipulative dexterity test, consisting of a board with randomly positioned slots in which subjects insert pegs. It is an eye-to hand coordination test that has been used in research studies, including pharmacotherapy studies (Rosen, Beauvais et al. 2003). Because of the nature of the challenge paradigm in this study (IV infusion), other tests, such as body sway, which require the subject to stand during the infusion, are practically more difficult. This test is accomplished with the subject sitting down.

### Clinician Administered Assessments

### Demographics

The interviewer asks the respondent for his/her date of birth, race, if he or she considers himself or herself to be Hispanic or Latino, and highest-grade level or degree received at the time of the interview.

### Richmond Agitation Sedation Scale (RASS)

The RASS is a 10 point Likert scale [+4 (combative) to -5 (unarousable)] which will be administered by a study staff member to measure participants' level of sedation during the study. During the study it will be conducted prior to administration of dexmedetomidine hydrochloride and approximately every 30 minutes after. Participants must at least have arousable sedation that can be reversed temporarily by verbal stimulation in order for dosing to continue (Sessler C N et al., 2002; Ely E W et. al, 2003).

### Agitation-Calmness Evaluation Scale (ACES)

The ACES uses a 9 point Likert scale [1, marked agitation; 2, moderate agitation; 3, mild agitation; 4, normal behavior; 5, mild calmness; 6, moderate calmness; 7, marked calmness; 8, deep sleep; and 9, unarousable] will be administered in addition to the RASS to further assess participants' sedation during the study. During the study it will be conducted prior to administration of dexmedetomidine hydrochloride and approximately every 30 minutes after.

### Clinician Administered PTSD Scale for DSM-5 (CAPS-5)

Gold-standard in PTSD assessment. The CAPS-5 is a 30-item structured interview that can be used to make current (past month) diagnosis of PTSD, make lifetime diagnosis of PTSD, and assess PTSD symptoms over the past week (Weathers, F. W., Blake, D. D., Schnurr, P. P., Kaloupek, D. G., Marx, B. P., & Keane, T. M., 2013).

### Mini-International Neuropsychiatric Interview for DSM-5

Assessment used to determine psychiatric diagnoses. This brief interview is structured and assesses DSM-5 current and lifetime psychiatric diagnoses (Sheehan, Lecrubier et al. 1998).

### Time-Line Follow-Back Assessment Method (TLFB)

Interview technique that will be used to obtain quantity/frequency of alcohol consumption data for each day during the 90-day period prior to the study, throughout the period of study participation (measuring drinking on days outside of test session days) and the follow-up. (Sobell and Sobell 1992) Participants are given a blank calendar covering the time interval to be re-constructed and are asked to reconstruct retrospectively their drinking behavior over that interval. The process is facilitated by establishing anchor points (e.g., holidays, anniversaries, major national events, etc.). It can be scored to provide the number of days on which various levels of consumption occurred. The time-line method has good test-retest reliability and good validity for verifiable events. It has been used in numerous studies to compare pre- to post-treatment drinking.

### Self-Assessments (Mood, Alcohol, and PTSD Symptoms)

### Biphasic Alcohol Effects Scale (BAES)

The BAES is a 14-item self-report adjective rating scale that will be used to measure the stimulant and sedative effects of alcohol during the test sessions (Martin, Earleywine et al. 1993).

This instrument has been found to be a sensitive and reliable measure to study medication influences on alcohol effects (Swift, Whelihan et al. 1994, Kranzler, Modesto-Lowe et al. 2000, Reynolds and Schiffbauer 2004).

### Drug Effects Questionnaire (DEQ)

The DEQ is a 5-item self-report measure used to assess the two key aspects of the subjective experience of substance effects: (1) the strength of substance effects and (2) the desirability of substance effects. (Morean, de Wit et al. 2012) Specifically, the DEQ uses 100-mm VAS anchored by “not at all” and “extremely” to assess the extent to which participants (1) feel any drug effect, (2) feel high, (3) dislike any of the drug effects, (4) like any of the drug effects, and (5) want more of the drug they consumed.

### Emotional Well-Being

The Differential Emotions Scale (DES-R) is a self-report device for assessment of an individual's emotions.

### Life Events Checklist for DSM-5 (LEC-5)

LEC-5 is a self-report that assesses exposure to 16 traumatic events, as a preface to the CAPS-5, to provide an anchor of the index trauma and document category of trauma (Gray, Litz et al. 2004, Weathers, Blake et al. 2013).

### PTSD Checklist for DSM-5 (PCL-5)

The PCL-5 is a 20-item self-report measure that assesses DSM-5-based criteria for PTSD symptoms. (Blevins, Mirshahi et al. 1997) Each item is rated on a 5-point Likert-type scale (0=Not at all; 4=Extremely) that indicates how much the participant has been bothered by an identified “worst” stressful event in the past month.

### Visual Analog Scales (VAS)

The VAS has the following items that will be assessed: high, anxious, drowsy, irritable, and nauseous, sleepy, desire to use alcohol at this moment, buzzed, depressed, tired, sad, angry, and nervous. We will use a 100 mm scale anchored by “not at all” and “extremely” to assess the extent to which participants are experiencing the symptoms listed at the time that the survey is given.

### Number of Drinks Scale (NDS)

NDS is used to assess how many drinks the subject feels he/she has consumed at a specific timepoint.

### State Trait Anxiety Inventory (STAI-6)

The STAI-6 is a 6-item measure designed to assess trait and state aspects of anxiety (Spielberger C D, et. al., 1983).

### Yale Craving Scale (YCS)

A significant advantage of this scale is that following completion of baseline training to match perceived intensity of craving to the perceived brightness from the sun, each assessment time point only consists of a single visual analog scale of craving, making it very easy to administer.

### Alcohol Cue Reactivity

This portion of the laboratory study consists of a presentation of a neutral cue (water) followed by an alcohol cue presentation and is used to assess alcohol craving.

### Neutral Cue Presentation

Individuals are instructed that they will be given a glass of water to handle for three minutes and that they may smell and handle the glass, but that they should not consume the water. In the presence of the research assistant, they are given the drink of water to hold and smell for three minutes. Following the presentation, the research assistant takes the beverage and leaves the room, while the subject completes the assessments.

### Alcohol Cue Presentation

Individuals are instructed that they will be given their drink of choice to handle for three minutes and that they may smell and handle the drink, but that they should not consume the alcohol. In the presence of the research assistant, they are given their drink to hold and smell for three minutes. Following the presentation, the research assistant takes the beverage and leaves the room, while the subject completes the assessments.

### Stress (PTSD) Reactivity

Participants will be exposed to two conditions in random order: PTSD cues and neutral cues. The cues will consist of a 5 minute presentation of the stimulus (trauma or neutral) followed by immediate evaluation of craving and anxiety. There will be a relaxation procedure between each condition. The imagery scripts are developed based on the scene construction questionnaire developed by Lang et al (Lang, Kozak et al. 1980, Lang, Levin et al. 1983) using a standardized format designed by Sinha (Sinha 2001).

The trauma imagery script will be based on the participant's description of a recent personal traumatic event that is self-rated as an 8 or above on a 10-point Likert scale, where 1=“not at all traumatic” and 10=“the most traumatic event in my life”. A ‘script’ or description of each situation will be developed using Scene Development Questionnaires which obtain specific stimulus and response details, including specific physical and interpersonal context details, verbal/cognitive attributions regarding the people involved, and physiological and bodily sensations experienced for the situation being described. The four scripts for each subject will then be recorded on an audio-tape for guided imagery in the laboratory sessions. The order of trauma, and neutral scripts will be assigned randomly, and counterbalanced across subjects.

Participants will be instructed to relax for a few minutes by clearing their minds and focusing on deep breathing. Participants will be instructed that when situation (script) is being read to them, they should try and imagine as though they are in the situation, and as though it is happening at that time. They will be asked to imagine themselves in the situation until they are asked to stop. The experimental procedure will follow the same format for each of the conditions consisting of baseline relaxation, imaging scripts and recovery period.

### Clinical Laboratory Evaluations

### Urine Drug Screen

A Clinical Laboratory Improvement Amendments (CLIA)-waived Multidrug Cup is an immunochromatographic assay for rapid, qualitative detection of drug combinations and their principal metabolites in urine at specified cut-off concentrations. A 12-panel multidrug cup will be used to test several drugs including Marijuana, Cocaine, Opiates (Morphine, Codeine, and Heroin), Methamphetamine, Amphetamines, Benzodiazepines, Barbiturates, Methadone, Buprenorphine, Ecstasy (MDMA), and Oxycodone.

### Urine Pregnancy Test

This assay is used to detect whether female participants are currently pregnant.

### Study Schedule

### Screening Assessments

A deidentified log is kept of all participants who are screened. After a potential participant has demonstrated that s/he understands the study and voluntarily provided written informed consent the assessments and forms outlined in Table 20 are conducted to determine study eligibility.
1. An alcohol breathalyzer test will be performed to determine breath alcohol content (BrAC). The BrAC must be <0.02 before screening can continue.
2. If the BrAC is >0.02, the visit will be rescheduled.
   If the BrAC is ≥0.08, the prospective participant will be discouraged from driving or engaging in other potentially dangerous activities; they will be encouraged to wait at the clinic until levels decrease to <0.08 (the legal limit).
   Prospective participants are allowed to leave if staff can verify that s/he has a responsible adult with them who is willing to be a designated driver or if s/he is taking a bus or taxi.
3. Blood Laboratory Tests will include:
   CBC w/auto differential (WBC, RBC, Hgb, Hct, plt)
   Chem 7 (glucose, BUN, Cr, Na, K, Cl, CO2, est GFR)
   LFTs (albumin, ALP, ALT, AST, total/direct bilirubin)
   GGT
   TSH

In addition to the screening assessments listed and described above, participants enrolled will also need to participate in a script development session as described under study specific procedures prior to the first test session. This can take place on the same day as screening or during a separate visit (based on clinician and participant availability).

TABLE 20
Screening Assessments:

| | |
|---|---|
| Assessment | Screen |

X

Assessments to Qualify for Study and Characterize Population

| | |
|---|---|
| Demographics | X |
| BrAC | X |
| Medical history | X |
| Inclusion/Exclusion Criteria | X |
| Informed Consent | X |
| LEC-5 | X |
| CAPS-5 | X |
| ECG | X |
| Physical and Laboratory Examination | X |
| MINI-5 | X |
| Vital Signs (Orthostatic HR/BP, SpO2) | X |
| Urine Pregnancy Test | X |
| Urine Toxicology | X |
| CIWA-AR | X |

Alcohol and PTSD Related Outcomes

| | |
|---|---|
| Timeline Follow-Back (TLFB) (90 days) | X |
| PCL | X |

Adverse Events and Concomitant Medications

| | |
|---|---|
| Side Effects Checklist | X |
| Concomitant Medications | X |

Other Assessments

| | |
|---|---|
| ACES | X |
| HVLT | X |
| RASS | X |
| Stress (PTSD) Script Development* | X |

### Schedule of Assessments

TABLE 21
Schedule of Test Day Assessments

| Assessment | Test #1 | Telephone | Test #2 | Telephone | Test #3 | Telephone | Telephone |
|---|---|---|---|---|---|---|---|
| Payment | X | | X | | X | | |

Assessments to Qualify for Proceeding with Test Day

| | | | |
|---|---|---|---|
| BrAC | X | X | X |
| Urine Pregnancy Test | X | X | X |
| Urine Toxicology | X | X | X |

Intervention

| | | | |
|---|---|---|---|
| Administration of dexmedetomidine HCl or | X | X | X |
| Placebo | | | |
| ETOH Challenge | X | X | X |

Alcohol Related Outcomes

| | | | |
|---|---|---|---|
| Biphasic Alcohol Effects Scale (BAES) | X | X | X |
| CIWA-AR | X | X | X |
| Timeline Follow-Back (TLFB) | X | X | X |
| Yale Craving Scale (YCS) | X | X | X |
| Alcohol Cue Reactivity | X | X | X |

PTSD Related Outcomes

| | | | |
|---|---|---|---|
| PCL-5 | X | X | X |
| Stress (PTSD) Reactivity | X | X | X |

Adverse Events and Concomitant Medications

| | | | | | | | |
|---|---|---|---|---|---|---|---|
| Adverse Events | X | X | X | X | X | X | X |
| Side Effects Checklist | X | X | X | X | X | X | X |
| Concomitant Medications | X | | X | | X | | |
| Oxygen Saturation | X | | X | | X | | |
| Agitation-Calmness Evaluation Scale (ACES) | X | | X | | X | | |
| Richmond Agitation Sedation Scale (RASS) | X | | X | | X | | |
| Vital Signs (Orthostatic HR/BP)* | X | | X | | X | | |

Other Assessments

| | | | |
|---|---|---|---|
| Drug Effects Questionnaire (DEQ) | X | X | X |
| Visual Analog Scales (VAS) | X | X | X |
| Number of Drinks Scale (NDS) | X | X | X |
| Differential Emotions Scale (DES-R) | X | X | X |
| The Grooved Pegboard Test | X | X | X |
| State Trait Anxiety Inventory (STAI-6) | X | X | X |
| Rapid Information Processing Task (RVIP) | X | X | X |
| Go No-Go task | X | X | X |
| Hopkins Verbal Learning Test-Revised (HVLT-R) | X | X | X |

*Will also occur prior to start of test day; participants must meet the heart rate and blood pressure parameters outlined in other sections of this protocol

Participants will come in for 3 separate test sessions, with a minimum of 2 days in between test sessions but no more than 2 weeks between test sessions. Each session will start around 8:00 AM. Between the test session days, participants will be able to smoke cigarettes, consume alcohol or drink caffeinated beverages as they normally do to minimize the withdrawal effects on study medications and testing procedures. Participants will be provided a standard, light breakfast (prior to this, participants will be asked not to eat after midnight before coming in).

TABLE 22
Laboratory Sessions: Study Procedures Table (times are approximate)

| Time | Measures and Events |
|---|---|
| Baseline | Urine drug screen and BrAC check, urine pregnancy, CIWA, randomization (Test # 1 |
| (−180 min) | only), AEs, Concomitant Medications, HR/BP, SpO2, ACES, RASS, TLFB, PCL, DEQ, |
| | STAI-6, VAS, YCS, BAES, NDS, DES-R, Go No-Go, Grooved Pegboard Test, RVIP |
| −60 min | Dexmedetomidine HCl 40 μg, 80 μg or Placebo administration |
| −40 min | HR/BP, SpO2, ACES, RASS, Side Effects, DEQ, STAI-6, VAS, YCS, BAES, NDS, DES- |
| | R, Go No-Go, Grooved Pegboard Test, RVIP |

Alcohol Cue Reactivity Condition
(Presentation of glass of water followed by alcoholic beverage-not consumed)

| | |
|---|---|
| 0 min | HR/BP, SpO2, ACES, RASS, DEQ, STAI-6, VAS, YCS |
| +10 min | Neutral Cue Presentation (3 minutes for presentation; 2 minutes to set up and remove) |
| +15 min | HR/BP, SpO2, ACES, RASS, DEQ, STAI-6, VAS, YCS |
| +20 min | Alcohol Cue Presentation (3 minutes for presentation; 2 minutes to set up and remove) |
| +25 min | HR/BP, DEQ, STAI-6, VAS, YCS |

Stress (PTSD) Reactivity Condition 1 (10 min break)
(Presentation of 5 minute audio clip of traumatic or neutral event)

| | |
|---|---|
| +30 min | Baseline/Relaxation; HR/BP, SpO2, ACES, RASS, DEQ, STAI-6, VAS, YCS |
| +40 min | Image period 1 (Trauma or Neutral), HR/BP |
| +45 min | HR/BP, SpO2, DEQ, STAI-6, VAS, YCS |
| +50 min | Recovery period. HR/BP |
| +55 min | HR/BP, DEQ, STAI-6, VAS, YCS |

Stress (PTSD) Reactivity Condition 2
(Presentation of 5 minute audio clip of traumatic or neutral event)

| | |
|---|---|
| +60 min | Baseline/Relaxation HR/BP, SpO2, ACES, RASS, DEQ, STAI-6, VAS, YCS |
| +70 min | Image Period 2 (Trauma or Neutral) HR/BP |
| +75 min | HR/BP, SpO2, DEQ, STAI-6, VAS, YCS |
| +80 min | Recovery Period HR/BP |
| +85 min | HR/BP, DEQ, STAI-6, VAS, YCS |

Alcohol Infusion

| | |
|---|---|
| +90 min | HR/BP, SpO2, ACES, RASS, DEQ, STAI-6, VAS, BAES, NDS, YCS |
| +100 min | start of IV alcohol infusion (30 minute to reach target of 0.1 BAL) |
| | HR/BP, SpO2 (Q15 min), ACES, RASS |
| +130 min | Target reached - HR/BP, SnO2 (Q15 min), ACES, RASS, DEQ, STAI-6, VAS, BAES, |
| | NDS, YCS, Go No-Go, Grooved Pegboard, RVIP, HVLT-R (and recall) |
| +170 min | IV alcohol infusion ends HR/BP, SpO2, ACES, RASS |
| +200 min | HR/BP, SpO2, ACES, RASS, DEQ, STAI-6, VAS, BAES, NDS, YCS, Grooved |
| | Pegboard, Lunch |
| +230 min | When BrAC ≤0.04: HR/BP, SpO2, ACES, RASS, Discharge* |

*Discharge will occur when BrAC ≤0.04 and participant will be medically cleared by a physician prior to discharge

### Medication Hold/Stop Procedures

Vital signs and oxygen saturation will be taken prior to administration of dexmedetomidine hydrochloride and prior to the start of the alcohol infusion. If a participant has a systolic BP <100 mmHg; diastolic BP <70 mmHg; HR <60 beats per minute; or pulse oximetry <90% administration at either of these points, the study drug (dexmedetomidine hydrochloride or alcohol depending on the time point) will not be administered. Doses will be on hold for participants who exhibit orthostatic changes (decrease in systolic blood pressure of 20 mm Hg or a decrease in diastolic blood pressure of 10 mm Hg within three minutes at baseline). Dosing will also be stopped if a participant does not have a level of arousable sedation that can be reversed temporarily by verbal stimulation.

### Telephone Follow-Up

A member of the research team will contact the participant after each test session (approximately within the next business day) to collect any adverse events. Following test session 3, the participant will additionally be contacted via telephone approximately 1 week later to record any adverse events. Each telephone follow-up will last approximately 10 minutes.

### Early Termination Visit

If an early exit occurs, the reason(s) for early termination will be documented and a post-study phone call will be conducted the following day to ensure the participant has made or scheduled any needed follow-up appointments to address any clinical problems that may have occurred.

### Assessment of Safety

### Adverse Events (AEs)

Participants will be asked at each visit if they are experiencing any discomfort or symptoms that might indicate potential side effects of the study drug. Any spontaneously reported symptoms or complaints will be recorded and, if serious, will be reported to the IRB and the DSMB.

Any spontaneously reported symptoms or complaints will be recorded and, if serious, will be reported to the IRB and the Food and Drug Administration (FDA).

### Severity of Event

Severity of adverse events will be determined by the study investigators using FDA's Guidance for Industry Toxicity Grading Scale for Healthy Adult and Adolescent Volunteers Enrolled in Preventive Vaccine Clinical Trials as a guide. General parameters for each grade are as follows.

TABLE 23

| | | | | Potentially Life |
|---|---|---|---|---|
| | Mild | Moderate | Severe | Threatening |
| Vital Signs* | (Grade 1) | (Grade 2) | (Grade 3) | (Grade 4) |
| Fever (° C.) ** | 38.0-38.4 | 38.5-38.9 | 39.0-40 | >40 > 104 |
| (° F.) ** | 100.4-101.1 | 101.2-102.0 | 102.1-104 | |
| Tachycardia - | 101-115 | 116-130 | >130 | ER visit or hospitalization |
| beats per minute | | | | for arrhythmia |
| Bradycardia - | 50-54 | 45-49 | <45 | ER visit or hospitalization |
| beats per | | | | for arrhythmia |
| minute*** | | | | |
| Hypertension | 141-150 | 151-155 | >155 | ER visit or hospitalization |
| (systolic) - mm | | | | for malignant hypertension |
| Hg | | | | |
| Hypertension | 91-95 | 96-100 | >100 | ER visit or hospitalization |
| (diastolic) - mm | | | | for malignant hypertension |
| Hg | | | | |
| Hypotension | 85-89 | 80-84 | <80 | ER visit or hospitalization |
| (systolic) - mm | | | | for hypotensive shock |
| Hg | | | | |
| Respiratory Rate - | 17-20 | 21-25 | >25 | Intubation |
| breaths per | | | | |
| minute | | | | |

*Participant should be at rest for all vital sign measurements.
** Oral temperature; no recent hot or cold beverages or smoking.
***When resting heart rate is between 60-100 beats per minute. Use clinical judgement when characterizing bradycardia among some healthy participant populations, for example, conditioned athletes.

TABLE 24

| | | | | Potentially Life |
|---|---|---|---|---|
| Systemic | Mild | Moderate | Severe | Threatening |
| (General) | (Grade 1) | (Grade 2) | (Grade 3) | (Grade 4) |
| Nausea/vomiting | No interference | Some interference | Prevents daily | ER visit or |
| | with activity or 1-2 | with activity or >2 | activity, requires | hospitalization for |
| | episodes/24 | episodes/24 hours | outpatient IV | hypotensive shock |
| | hours | | hydration | |
| Diarrhea | 2-3 loose | 4-5 stools or 400- | 6 or more watery | ER visit or |
| | stools or <400 | 800 g/24 hours | stools or >800 | hospitalization |
| | g/24 hours | | g/24 hours or | |
| | | | requires outpatient | |
| | | | IV hydration | |
| Headache | No interference | Repeated use of | Significant; any | ER visit or |
| | with activity | nonnarcotic pain | use of narcotic | hospitalization |
| | | reliever >24 hours | pain reliever or | |
| | | or some interference | prevents daily | |
| | | with activity | activity | |
| Fatigue | No interference | Some interference | Significant; | ER visit or |
| | with activity | with activity | prevents daily | hospitalization |
| | | | activity | |
| Myalgia | No interference | Some interference | Significant; | ER visit or |
| | with activity | with activity | prevents daily | hospitalization |
| | | | activity | |

TABLE 25

| | | | | Potentially Life |
|---|---|---|---|---|
| | Mild | Moderate | Severe | Threatening |
| Systemic Illness | (Grade 1) | (Grade 2) | (Grade 3) | (Grade 4) |
| Illness or clinical | No interference | Some interference | Prevents daily | ER visit or |
| adverse event (as | with activity | with activity not | activity and | hospitalization |
| defined according | | requiring medical | requires medical | |
| to applicable | | intervention | intervention | |
| regulations) | | | | |

TABLE 26

| | | | | Potentially Life |
|---|---|---|---|---|
| | Mild | Moderate | Severe | Threatening |
| Serum* | (Grade 1) | (Grade 2) | (Grade 3) | (Grade 4) ** |
| Sodium - Hyponatremia | 132-134 | 130-131 | 125-129 | <125 |
| mEq/L | | | | |
| Sodium - Hypernatremia | 144-145 | 146-147 | 148-150 | >150 |
| mEq/L | | | | |
| Potassium - Hyperkalemia | 5.1-5.2 | 5.3-5.4 | 5.5-5.6 | >5.6 |
| mEq/L | | | | |
| Potassium - Hypokalemia | 3.5-3.6 | 3.3-3.4 | 3.1-3.2 | <3.1 |
| mEq/L | | | | |
| Glucose - Hypoglycemia | 65-69 | 55-64 | 45-54 | <45 |
| mg/dL | | | | |
| Glucose - Hyperglycemia | 100-110 | 111-125 | >125 | Insulin |
| Fasting - mg/dL Random - | 110-125 | 126-200 | >200 | requirements or |
| mg/dL | | | | hyperosmolar coma |
| Blood Urea Nitrogen BUN | 23-26 | 27-31 | >31 | Requires dialysis |
| mg/dL | | | | |
| Creatinine - mg/dL | 1.5-1.7 | 1.8-2.0 | 2.1-2.5 | >2.5 or requires |
| | | | | dialysis |
| Calcium - hypocalcemia | 8.0-8.4 | 7.5-7.9 | 7.0-7.4 | <7.0 |
| mg/dL | | | | |
| Calcium - hypercalcemia | 10.5-11.0 | 11.1-11.5 | 11.6-12.0 | >12.0 |
| mg/dL | | | | |
| Magnesium - | 1.3-1.5 | 1.1-1.2 | 0.9-1.0 | <0.9 |
| hypomagnesemia mg/dL | | | | |
| Phosphorous - | 2.3-2.5 | 2.0-2.2 | 1.6-1.9 | <1.6 |
| hypophosphatemia mg/dL | | | | |
| CPK - mg/dL | 1.25-1.5 × ULN*** | 1.6-3.0 × ULN | 3.1-10 × ULN | >10 × ULN |
| Albumin - | 2.8-3.1 | 2.5-2.7 | <2.5 | — |
| Hypoalbuminemia g/dL | | | | |
| Total Protein - | 5.5-6.0 | 5.0-5.4 | <5.0 | — |
| Hypoproteinemia g/dL | | | | |
| Alkaline phosphate - | 1.1-2.0 × ULN | 2.1-3.0 × ULN | 3.1-10 × ULN | >10 × ULN |
| increase by factor | | | | |
| Liver Function Tests -ALT, | 1.1-2.5 × ULN | 2.6-5.0 × ULN | 5.1-10 × ULN | >10 × ULN |
| AST increase by factor | | | | |
| Bilirubin - when | 1.1-1.25 × ULN | 1.26-1.5 × ULN | 1.51-1.75 × ULN | >1.75 × ULN |
| accompanied by any | | | | |
| increase in Liver Function | | | | |
| Test increase by factor | | | | |
| Bilirubin - when Liver | 1.1-1.5 × ULN | 1.6-2.0 × ULN | 2.0-3.0 × ULN | >3.0 × ULN |
| Function Test is normal; | | | | |
| increase by factor | | | | |
| Cholesterol | 201-210 | 211-225 | >226 | — |
| Pancreatic enzymes - | 1.1-1.5 × ULN | 1.6-2.0 × ULN | 2.1-5.0 × ULN | >5.0 × ULN |
| amylase, lipase | | | | |

*The laboratory values provided in the tables serve as guidelines and are dependent upon institutional normal parameters.
** The clinical signs or symptoms associated with laboratory abnormalities might result in characterization of the laboratory abnormalities as Potentially Life Threatening (Grade 4). For example, a low sodium value that falls within a grade-3 parameter (125-129 mE/L) should be recorded as a grade 4 hyponatremia event if the participant had a new seizure associated with the low sodium value.
***“ULN” is the upper limit of the normal range.

TABLE 27

| | | | | Potentially Life |
|---|---|---|---|---|
| | Mild | Moderate | Severe | Threatening |
| Hematology* | (Grade 1) | (Grade 2) | (Grade 3) | (Grade 4) |
| Hemoglobin (Female) - | 11.0-12.0 | 9.5-10.9 | 8.0-9.4 | <8.0 |
| gm/dL | | | | |
| Hemoglobin (Female) | Any decrease-1.5 | 1.6-2.0 | 2.1-5.0 | >5.0 |
| change from baseline value - | | | | |
| gm/dL | | | | |
| Hemoglobin (Male) - gm/dL | 12.5-13.5 | 10.5-12.4 | 8.5-10.4 | <8.5 |
| Hemoglobin (Male) change | Any decrease-1.5 | 1.6-2.0 | 2.1-5.0 | >5.0 |
| from baseline value - gm/d | | | | |
| WBC Increase - cell/mm³ | 10,800-15,000 | 15,001-20,000 | 20,001-25,000 | >25,000 |
| WBC Decrease - cell/mm³ | 2,500-3,500 | 1,500-2,499 | 1,000-1,499 | <1,000 |
| Neutrophils Decrease - | 1,500-2,000 | 1,000-1,499 | 500-999 | <500 |
| cell/mm3 | | | | |
| Eosinophils - cell/mm3 | 650-1500 | 1501-5000 | >5000 | Hypereosinophilic |
| Platelets Decreased - | 125,000-140,000 | 100,000-124,000 | 25,000-99,000 | <25,000 |
| cell/mm3 | | | | |
| PT - increase by factor | 1.0-1.10 × ULN** | 1.11-1.20 × ULN | 1.21-1.25 × ULN | >1.25 ULN |
| (prothrombin time) | | | | |
| PTT - increase by factor | 1.0-1.2 × ULN | 1.21-1.4 × ULN | 1.41-1.5 × ULN | >1.5 × ULN |
| (partial thromboplastin | | | | |
| time) | | | | |
| Fibrinogen increase - mg/dL | 400-500 | 501-600 | >600 | — |
| Fibrinogen decrease - | 150-200 | 125-149 | 100-124 | <100 or |
| mg/dL | | | | associated with |
| | | | | gross bleeding or |
| | | | | disseminated |
| | | | | intravascular |
| | | | | coagulation (DIC) |

*The laboratory values provided in the tables serve as guidelines and are dependent upon institutional normal parameters.
**“ULN” is the upper limit of the normal range.

TABLE 28

| | | | | Potentially Life |
|---|---|---|---|---|
| | Mild | Moderate | Severe | Threatening |
| Urine* | (Grade 1) | (Grade 2) | (Grade 3) | (Grade 4) |
| Protein | Trace | 1+ | 2+ | Hospitalization or dialysis |
| Glucose | Trace | 1+ | 2+ | Hospitalization for |
| | | | | hyperglycemia |
| Blood (microscopic) - red | 1-10 | 11-50 | >50 and/or | Hospitalization or packed |
| blood cells per high power | | | gross blood | red blood cells (PRBC) |
| field (rbc/hpf) | | | | transfusion |

*The laboratory values provided in the tables serve as guidelines and are dependent upon institutional normal parameters.

### Time Period and Frequency for Event Assessment and Follow-Up

For adverse event assessment, AEs will be collected from time of first dose. Study investigators will follow all SAEs clinically until resolved or clinically stable. Adverse events in general will be followed for study data collection purposes only through the final follow-up visit/phone interview. The occurrence of an AE or SAE may come to the attention of study personnel during study visits and interviews of a study participant presenting for medical care, or upon review by a study monitor. At each study visit, the investigator will inquire about the occurrence of AE/SAEs since the last visit. All AEs will be captured on the appropriate CRF.

Information to be collected includes event description, time of onset, clinician's assessment of severity, relationship to study product (assessed only by those with the training and authority to make a diagnosis), and time of resolution/stabilization of the event. All AEs occurring while on study must be documented appropriately regardless of relationship.

Any medical condition that is present at the time that the participant is screened will be considered as baseline and not reported as an AE. However, if the study participant's condition deteriorates at any time during the study, it will be recorded as an AE.

### Statistical Considerations

### Statistical and Analytical Plans

A statistical analysis plan will be developed for this study and finalized prior to locking the study data and unmasking.

### Analysis Datasets

The safety population will include all participants that receive any study drug (including placebo).

### Description of Statistical Methods

### General Approach

All statistical computations will be performed and data summaries will be created using SAS 9.4 or higher. If additional statistical packages are required, these will be discussed in the study report. This is a small sample, exploratory within subject designed study with a primary objective of assessing the impact of dexmedetomidine hydrochloride on stress and alcohol cue reactivity as well as the subjective effects and the effects of alcohol received via alcohol challenges. For summaries of study data, categorical measures will be summarized in tables listing the frequency and the percentage of participants (by dose level of placebo, dexmedetomidine hydrochloride 40 μg, and dexmedetomidine hydrochloride 80 μg where relevant); continuous data will be summarized by presenting mean, standard deviation, median and range; and ordinal data will be summarized by only presenting median and range. Model-based analyses will also be used to obtain point estimates and associated confidence intervals for various measures as well as p-values for comparisons of data between dose levels. P-values presented will be based on two-sided tests unless otherwise specified and generally not adjust for any baseline covariates. For continuous outcomes, checks of normality will be performed and if required, transformations or non-parametric tests will be employed. Additional details for potential covariate adjustments in secondary analyses or handling violations of analytic method assumptions will be detailed in the statistical analysis plan.

### Analysis of the Stress (PTSD) and Alcohol Cue Reactivity Endpoint(s)

All assessment data will be summarized for each time point measured.

Most measures obtained during the stress and alcohol cue reactivity assessments including heart rate, blood pressure, STAI-6 and YCS scores are continuous and obtained at multiple timepoints. For stress (PTSD) reactivity assessments, the trajectory of these measures will be plotted over time by condition (neutral vs. trauma) as well as by study dose (placebo, dexmedetomidine hydrochloride 40 μg, and dexmedetomidine hydrochloride 80μg) within each condition. Repeated measure models with study dose, time in hours relative to start of the session (assuming parametric trends if possible), condition, and the interactions between these parameters as explanatory factors will be constructed if data allow to test for and estimate any differences over time by condition and by study dose (within the stress condition in particular). The models will control for test period/randomization order and where appropriate baseline measures/scores for the outcome being assessed. The primary comparison of interest will be if a difference exists among all the study dose levels. If a difference is observed, then pairwise comparisons between each dexmedetomidine hydrochloride dose and placebo will be assessed as well as presence of a potential dose-response trend. VAS responses are ordinal in nature and values will be summarized over time in both figures and tables with no formal model-based analyses planned. Data during the alcohol cue reactivity assessment will be assessed in a similar manner with values plotted over time by study dose and repeated measures models with study dose, time in hours relative to start of the session, the interaction between these two parameters as explanatory factors constructed if data allow.

### Analysis of the Alcohol Challenge Endpoints(s)

All assessment data will be summarized for each time point measured. Most measures obtained after the alcohol challenge including heart rate, blood pressure, and STAI-6, BAES, YCS, and Grooved Pegboard scores are continuous and obtained at least twice after each alcohol challenge. The trajectory of these measures will be plotted over time and the measures will be analyzed via repeated measure models with study dose (placebo, dexmedetomidine hydrochloride 40μg, and dexmedetomidine hydrochloride 80μg), time in hours relative to start of infusion (assuming parametric trends if possible) and the interaction between these parameters as explanatory factors will be constructed if data allow to test for and estimate any differences over time by study dose. The models will also control for test period/randomization order and where appropriate baseline measures/scores for the outcome will be assessed. The primary comparison of interest will be if a difference exists among all the study dose levels. If a difference is observed, then pairwise comparisons between each dexmedetomidine hydrochloride dose and placebo will be assessed as well as presence of a potential dose-response trend. HVLT-R, Go No Go, and RVIP are only assessed within each alcohol challenge and will be analyzed using similar repeated measure models that account for the correlation of observations within each individual across test period but have no repeated measures within test period. NDS and VAS responses are ordinal in nature and values will be summarized over time in both figures and tables with no formal model-based analyses planned.

### Planned Interim Analyses

### Safety Review

The study will be monitored by the DSMB. Baseline; participant disposition; study drug exposure; and primary and secondary outcome collection data will be summarized for each study review with a focus on participant safety. While there are study halting rules that will trigger a study review by the DSMB, there are no formal study stopping rules based on the safety review that would trigger permanent study termination without the review and recommendation of the DSMB.

### Sample Size

We expect to consent approximately 40 people in order to achieve n=10 completers with the goal of comparing dexmedetomidine hydrochloride 40 μg and 80 μg to placebo on various measures of stress (PTSD) and alcohol cue reactivity as well as of subjective effects of ethanol in a laboratory setting. The majority of measures collected are continuous. Given this is an early phase laboratory study, the entire profile of effects will be assessed in order to determine if there is support for moving into later phase studies of this compound for treatment of AUD with co-morbid with PTSD as opposed to a formal test of a single hypothesis. Therefore calculations focus on potentially detectable effect sizes when comparing each dexmedetomidine hydrochloride dose to placebo and also on the precision that can be obtained for estimating effect sizes.

### Enrollment/Randomization/Masking Procedures

To minimize bias, all participants will be screened for assurance that they meet study eligibility criteria.

A placebo drug will be employed as the comparison group to active study drug and the study will be conducted in a double-masked fashion in that both the participants and the site investigators and staff interacting with participants and assessing study outcomes will be masked to treatment assignment. The only individuals at the site with access to treatment assignment information will be the research pharmacists.

Since this is the first time that alcohol is being given in combination with dexmedetomidine hydrochloride , all participants will be randomized to receive dexmedetomidine hydrochloride 40 μg, 80 μg or placebo in a modified crossover randomization scheme in which all participants will receive each possible study drug assignment but must receive 40 μg before receiving the higher dose (80 μg). Specifically, each participant will be randomized to one of the following orders of study drug across test sessions.

TABLE 29

| Randomization | Study Drug Assignment By Test Session | | |
|---|---|---|---|
| Sequence | 1 | 2 | 3 |
| A | Placebo | Dexmedetomidine | Dexmedetomidine |
| | | HCl 40 μg | HCl 80 μg |
| B | Dexmedetomidine | Placebo | Dexmedetomidine |
| | HCl 40 μg | | HCl 80 μg |
| C | Dexmedetomidine | Dexmedetomidine | Placebo |
| | HCl 40 μg | HCl 80 μg | |

Participants will be randomly allocated among these randomization sequences such that 3 participants will be randomized to sequences A and C and 4 participants randomized to sequence B.

### Example 9: A Randomized, Double-blind, Placebo-controlled Study to Determine Efficacy and Safety of Dexmedetomidine Oromucosal film in Agitation Associated With Pediatric Schizophrenia and Bipolar Disorder

### Objectives

### Primary Objective

To determine if a single dose of dexmedetomidine hydrochloride oromucosal film, compared to placebo, can effectively reduce symptoms of acute agitation associated with pediatric schizophrenia and bipolar disorder.

### Key Secondary Objective

To determine the earliest time where an effect on agitation is apparent.

### Other Secondary Objectives

To further determine the efficacy, safety, tolerability, and pharmacokinetics (PK) of dexmedetomidine hydrochloride oromucosal film for acute agitation associated with pediatric schizophrenia and bipolar disorder.

### Primary Endpoint

The primary efficacy endpoint will be the absolute change from baseline in the PEC total score at 2 hours.

### Secondary Endpoints

### Key Secondary Endpoint

The key secondary efficacy endpoint will be the absolute change from baseline in the PEC total score at 90, 60, 45, 30, 20, and 10 minutes.

The other secondary endpoints will be:
1. Overall clinical improvement after study drug administration as measured by the CGI-I score.
2. Duration of calming effect as described by the change from baseline in PEC total score, and ACES score at 2, 4 and 8 hours after dosing.
3. The effect on overall psychotic symptoms and subscales (PANSS total, positive, negative, and general psychopathology subscales).
4. Change from baseline in total PEC score over time measured from 10 minutes through 24 hours after dosing.
5. PEC responders and CGI-I responders at 2 hours following administration of dexmedetomidine oromucosal compared with placebo:
   a. PEC responders will be defined as those who achieve at least a 40% reduction in PEC total score from baseline at or before 2 hours post-dose.
   b. CGI-I responders will be defined as subjects with a score of 1 or 2 on the CGI-I scale (CGI-I non-responders will be defined as subjects with scores from 3 to 7 at 2 hours post-dose).
6. Time to rescue medication during the entire 24-hour Post-treatment Evaluation Period for subjects receiving dexmedetomidine oromucosal film compared to placebo.
7. Number of subjects per treatment group who received rescue medication within 4 hours after dosing and within 24 hours after study drug administration.
8. The safety profile of dexmedetomidine oromucosal film as measured by reports of vital signs and TEAEs.
9. Characteristics of the patient population as assessed by the YMRS (for bipolar disorder only).
10. The overall tolerability in terms of TEAE reports and local site (sublingual/buccal) tolerability of oral film.
11. Descriptive PK of dexmedetomidine oromucosal film in the subject population.
12. Subject acceptability, taste, and likability of study medication using Drug Likability Scales.

### Study Design

This is a randomized, double-blind, placebo-controlled study assessing the efficacy, safety, tolerability, and PK of dexmedetomidine oromucosal film in male and female children and adolescents (ages 10-17 years old inclusive for patients with bipolar disorder; ages 13-17 years old inclusive for patients with schizophrenia) with acute agitation associated with schizophrenia, schizoaffective disorder, schizophreniform, and bipolar disorder.

The doses of 80 μg and 120 μg were selected based on population PK (PopPK) modeling and simulation as the doses in children and adolescents to produce similar exposures as seen with the safe and efficacious doses in adults (120 μg and 180 μg). This study will randomize subjects 1:1:1 to receive dexmedetomidine oromucosal film 80 μg, 120 μg, or matching placebo film, stratified by disease type (bipolar vs. schizophrenia) and region (US vs. Europe). Study randomization will be computer generated. These doses were selected by modeling and simulation to produce similar exposures and activities as seen with the safe and efficacious doses in adults. In the event of persistent or recurrent agitation after the 2-hour assessments (PEC change from baseline is ≤40%) and in the absence of safety concerns, subjects may be administered a repeat dose at one-half the initial dose level. A maximum of 2 repeat doses are allowed per subject during the 12 hours post-first dose, with each dose separated by at least 2 hours. Subjects may only be re-dosed if they are hemodynamically stable and not hypotensive, as indicated by SBP <90 mmHg. Subjects cannot be re-dosed if they are orthostatic or if they are experiencing an AE that, when assessed by the investigator, precludes re-dosing.

Potentially eligible subjects (acutely agitated subjects diagnosed with schizophrenia, schizoaffective, schizophreniform, or bipolar disorder) may be identified in outpatient clinics, in mental health, psychiatric or medical emergency services, including medical and psychiatric observation units, or in a hospital setting for acute agitation or as patients already hospitalized for chronic underlying conditions. Subjects will be under medical supervision in a clinical research setting or hospital during the screening procedures to assess eligibility

Method of Assigning Subjects to Treatment Groups

Upon confirmation of eligibility, subjects will be randomized to receive dexmedetomidine oromucosal film 80 μg, 120 μg, or matching placebo. At the time of dosing, subjects will be instructed by a trained staff member to self-administer the investigational product sublingually or buccally by retaining the investigational product in the sublingual cavity or buccal space until dissolved. Participants will be allowed fluids as desired at least 15 minutes after completion of dosing. In the event of persistent or recurrent agitation after the 2-hour assessments (PEC change from baseline is ≤40%) and in the absence of safety concerns, subjects may be administered a repeat dose at one-half the initial dose level. A maximum of 2 repeat doses are allowed per subject during the 12 hours post-first dose, with each dose separated by at least 2 hours.

Subjects may only be re-dosed if they are hemodynamically stable and not hypotensive, as indicated by a systolic blood pressure (SBP) <90 mmHg. Subjects cannot be re-dosed if they are orthostatic or if they are experiencing an AE that, when assessed by the investigator, precludes re-dosing. All efficacy and safety assessments listed at the 2-hour post-first dose timepoint will be re-assessed after each re-dose. Additional PK samples may be obtained at 2 hours after each additional dose in subjects who are re-dosed.

Efficacy, safety, and PK will be measured throughout the treatment period. Safety and tolerability assessments, including vital signs and 12-lead electrocardiogram (ECG) data, will be monitored pre-dose, throughout post-dose, and on Day 7 (+2). Usability and tolerability of the oromucosal film formulation in adolescents will also be assessed. Subjects will be evaluated for local irritation around the area where the film is placed.

Any abnormal vital sign measurement, clinical laboratory test, physical examination finding, or ECG parameter deemed clinically significant by the investigator will be repeated. For any test abnormality deemed clinically significant, repeat analyses will be performed during the follow-up period and until the value returns to baseline (or within normal limits), until the investigator deems the abnormality to be stable and no longer of clinical concern, or until the subject is lost to follow-up.

### Number of Subjects (Planned)

Approximately 120 subjects are planned to be enrolled, with a minimum of 40 subjects in each indication of bipolar disorder and schizophrenia.

### Inclusion Criteria

1. Male and female subjects between the ages of 10-17 years old, inclusive, in subjects with bipolar disorder and 13-17 years old, inclusive, in subjects with schizophrenia.
2. Subject or legally acceptable representative (per local regulatory requirements for the legal age of consent for study participation) is able to sign and date written ICF prior to the start of any study-specific qualification procedures.
3. Subject meets DSM-5 criteria for schizophrenia, schizoaffective, or schizophreniform disorder, or other specified/unspecified schizophrenia spectrum and/or other psychotic disorders
4. OR
5. Subject meets DSM-5 criteria for bipolar disorder (bipolar I or II).
6. Subject assessed to be clinically agitated at Screening and Baseline with a total score of ≥14 on the 5 items (poor impulse control, tension, hostility, uncooperativeness, and excitement) comprising the PANSS Excited Component (PEC).
7. Subject has a score of ≥4 on at least 1 of the 5 items on the PEC at Baseline.
8. Subject is in good general health prior to study participation as determined by a detailed medical history, physical examination, 12-lead ECG with rhythm strip, blood chemistry profile, hematology, urinalysis, and in the opinion of the investigator.
9. Female subjects, if of child-bearing potential and sexually active, and male subjects, if sexually active with a partner of child-bearing potential, agree to use a medically acceptable and effective birth control method throughout the study and for 1 week following the end of the study.
10. Medically acceptable methods of contraception that may be used by the participant and/or his/her partner include abstinence, birth control pills or patches, diaphragm with spermicide, intrauterine device, condom with foam or spermicide, vaginal spermicidal suppository, surgical sterilization, and progestin implant or injection. Prohibited methods include the rhythm method, withdrawal, condoms alone, or diaphragm alone.

### Exclusion Criteria

1. Subjects with agitation caused by acute intoxication, including positive identification of alcohol by breathalyzer or drugs of abuse (except for THC) during urine screening.
2. Subjects with ADHD treated with an alpha2-adrenergic agonist (clonidine, guanfacine)
3. Subjects with agitation that cannot be attributed to schizophrenia or bipolar disorder (bipolar I or II) as diagnosed by DSM-5 criteria.
4. Use of benzodiazepines or other hypnotics or oral or short-acting intramuscular antipsychotic drugs in the 4 hours before study treatment.
5. Treatment with alpha-1 noradrenergic blockers (terazosin, doxazosin, tamsulosin, alfuzosin, or
6. prazosin) or other prohibited medications.
7. Subjects with significant risk of suicide or homicide per the investigator's assessment, or any subject with an answer of “yes” to item 4 or 5 on the C-SSRS.
8. Female subjects who have a positive pregnancy test at Screening.
9. Subjects who have hydrocephalus, seizure disorder, or history of significant head trauma, brain tumor, or meningitis.
10. History of syncope or other syncopal attacks, current evidence of hypovolemia, orthostatic hypotension, or a baseline heart rate of <55 beats per minutes (bpm) or a resting systolic/diastolic blood pressure (DBP) of <90/60 mmHg at Screening and before dosing.
11. Subjects with laboratory or ECG abnormalities considered clinically significant by the investigator or qualified designee that would have clinical implications for the subject's participation in the study.
12. Subjects with serious or unstable medical illnesses. These include current hepatic impairment (moderate-severe), or renal, respiratory, endocrinologic, or hematologic disease.
13. Subjects who have received an investigational drug within 30 days prior to the current agitation episode.
14. Subjects who are considered by the investigator, for any reason, to be an unsuitable candidate for receiving dexmedetomidine, e.g., subjects with a history of allergic reactions to dexmedetomidine.
15. Subjects with a history of atrioventricular block.

### Test Product, Dose, and Mode of Administration

Dexmedetomidine Oromucosal film is an orally dissolving thin film formulation of dexmedetomidine for sublingual or buccal administration. The product is a small, solid-dose film formulation, approximately 286 mm² in area and 0.7 mm thick, designed to completely dissolve in the sublingual or buccal space within 1-3 minutes.

### Reference Therapy, Dosage and Mode of Administration

Matching placebo film with the same inactive ingredients to be taken sublingually or buccally.

### Duration of Treatment

1 day

### Criteria for Evaluation

### Efficacy Assessment

Assessment of drug effects on acute agitation will be done by the PEC, which comprises 5 items associated with agitation: poor impulse control, tension, hostility, uncooperativeness, and excitement. Each item is scored from 1 (minimum) to 7 (maximum), for an overall summary score ranging from 5 to 35. To determine the overall clinical improvement after drug administration, the CGI-I will also be used.

Overall agitation and sedation will be evaluated with the Agitation-Calmness Evaluation Scale (ACES), where 1 indicates marked agitation; 2—moderate agitation; 3—mild agitation; 4—normal behavior; 5—mild calmness; 6—moderate calmness; 7—marked calmness; 8—deep sleep; and 9—unarousable.

### Safety and Tolerability Assessments

Adverse events (AEs), clinical laboratory tests, 12-lead ECG with rhythm strip, and vital signs will be monitored. All observed and volunteered AEs will be recorded and graded. Vital signs, including SBP, DBP, and heart rate will be monitored. Pulse oximetry and respiratory rate will be assessed at various timepoints. The application site of the oromucosal film preparation (buccal mucosa) will be inspected for any signs of local irritation.

At the discretion of the investigator or designee, rescue therapy with lorazepam po/IM may be initiated as a standard of care treatment for acute agitation. Other medications may be used in accordance with the institution's standard of care. When rescue therapy is administered, the medication, time, dose and indication must be clearly recorded as “for agitation” in the CRF and source documents.

### Additional Assessments

Demographics, medical and psychiatric history, psychotic symptoms (PANSS), smoking history, prior and concomitant medication, physical examination, and pregnancy.

### Pharmacokinetics

PK will be assessed by PopPK analysis with sparse samples collected at 2, 4, 8, and 24 hours post-dose and reported separately. A PopPK/pharmacodynamic (PD) analysis of plasma concentration vs. clinical response and key safety parameters will be explored and reported using a separate SAP and report. A graphical assessment of PK vs. vital signs and other potential PD parameters may be included.

TABLE 30
Schedule of Events

| | | Treatment Evaluation Day 1 | | | | | | | | | | | | | |
|---|---|---|---|---|---|---|---|---|---|---|---|---|---|---|---|
| | | Pre- | | | | | | | | | | | Day 2 | | |
| | SCR | Dose¹ | | | | | | | | | | | Follow-up | | Day 7 |
| Activity | ≤28 | −1 hr | 10 | 20 | 30 | 45 | 1 | 1.5 | 2 | 4 | 6 | 8 | 24 hrs | Day 3 | (+2 days) |
| Time Point | days | to 0 | mins | mins | mins | mins | hr | hrs | hrs | hrs | hrs | hrs | (−9/+12 | (+1 day) | End of |
| Informed | X | | | | | | | | | | | | | | |
| consent/assent | | | | | | | | | | | | | | | |
| Inclusion/Exclusion | X | X | | | | | | | | | | | | | |
| criteria | | | | | | | | | | | | | | | |
| Demographics | X | | | | | | | | | | | | | | |
| Medical history | X | | | | | | | | | | | | | | |
| Physical exam | X | | | | | | | | | | | | X | | |
| Weight | X | | | | | | | | | | | | X | | |
| Height | X | | | | | | | | | | | | | | |
| BMI | X | | | | | | | | | | | | | | |
| Resting vital signs⁴ | X | X | | | X | | X | | X | X | X | X | X | X | X |
| Orthostatic vital | X | X | | | | | | | X | X | | X | X | X | X |
| signs⁴ | | | | | | | | | | | | | | | |
| Pulse oximetry | | X | | | X | | X | | X | X | | X | | | |
| ECG with rhythm | X | X | | | | | | | X | | | | X | | |
| strip³ | | | | | | | | | | | | | | | |
| Alcohol breathalyzer | X | | | | | | | | | | | | | | |
| MINI | X | | | | | | | | | | | | | | |
| Clinical laboratory | X | | | | | | | | | | | | | X | X |
| assessments² | | | | | | | | | | | | | | | |
| CGI-Severity⁶ | X | X | | | | | | | | | | | | | |
| C-SSRS | X | X | | | | | | | | | | | X | X | X |
| Admit to unit | X | | | | | | | | | | | | | | |
| PCRS⁵ | X | X | | | | | | | X | | | | X | | |
| PEC⁵ | X | X | X | X | X | X | X | X | X | X | X | X | X | | |
| YMRS (bipolar-only) | | X | | | | | | | | | X | | X | | |
| PANSS | | X | | | | | | | | | X | | X | | |
| (schizophrenia- | | | | | | | | | | | | | | | |
| only)⁹ | | | | | | | | | | | | | | | |
| ACES⁵ | | X | | | | | | | X | X | | X | | | |
| Randomization | | X | | | | | | | | | | | | | |

TABLE 31
Schedule of Events

| | | | | | | | | | | | | | Day 2 | | |
|---|---|---|---|---|---|---|---|---|---|---|---|---|---|---|---|
| | | Treatment Evaluation Day 1 | | | | | | | | | | | Follow-up | | |
| | | Pre- | | | | | | | | | | | (+1 day) | | Day 7 |
| | SCR | Dose¹ | Post-dose Time¹ | | | | | | | | | | 24 hrs | | (+2 days) |
| Activity | ≤28 | −1 hr | 10 | 20 | 30 | 45 | 1 | 1.5 | 2 | 4 | 6 | 8 | (−9/+12 | Day 3 | End of |
| Time Point | days | to 0 | mins | mins | mins | mins | hr | hrs | hrs | hrs | hrs | hrs | hrs) | (+1 day) | Study |

| | | | | | | | | |
|---|---|---|---|---|---|---|---|---|
| Study drug | X | | | | | | | |
| administration¹⁰ | | | | | | | | |
| Drug Likability | | X | | | | | | |
| Scales | | | | | | | | |
| Likability | | X | | | | | | |
| Questionnaire | | | | | | | | |
| CGI-Improvement⁶ | | | X | X | X | X | | |
| Buccal (SL) | | | X | | X | X | | X |
| assessment for local | | | | | | | | |
| irritation⁷ | | | | | | | | |
| PK sampling⁸ | | | | | X | X | X | X |

| | | | | | | |
|---|---|---|---|---|---|---|
| Concomitant | X | X | X | X | X | X |
| medications | | | | | | |
| Adverse events | X | X | X | X | X | X |

ACES = Agitation-Calmness Evaluation Scale;
C-SSRS = Columbia-Suicide Severity Rating Scale;
CGI = Clinical Global Impression;
CLIA = Clinical Laboratory Improvement Amendment;
DBP = diastolic blood pressure;
ECG = electrocardiogram;
hr = hour;
min = minute;
MINI = Mini-International Neuropsychiatric Interview;
PANSS = Positive and Negative Syndrome Scale;
PCRS = Placebo-Control Reminder Script;
PEC = Positive and Negative Syndrome Scale - Excited Component;
PK = pharmacokinetic;
Pre-trt = Pre-treatment;
SBP = systolic blood pressure;
SCR = Screening;
SL = sublingual;
YMRS = Young Mania Rating Scale
¹Pre-dose assessments will have a window of 60 minutes prior to dose with the exception of PEC and ACES which will be performed within 15 minutes of dosing (15 to 0 min). All post-dose assessments will have a window of −5/+15 minutes through the 1.5-hour assessments, −5/+25 minutes for the 2-hour assessments (with the exception of the PEC which will have a ±5 minute window) and ±30 minutes for the 4-, 6-, and 8-hour assessments and full PANSS can be performed at any time.
²Safety laboratory assessments will include chemistry, hematology, electrolyte panel, urinalysis, urine drug screen (local lab, only conducted at screening), alcohol breathalyzer (only conducted at screening), urine pregnancy (conducted at screening), and serum pregnancy (Visit 7/End of Study).
³Screening/enrollment laboratory assessments: Local laboratory assessments drawn within 7 days prior to screening may suffice with the exception of urine drug screen. If results not available on the same day, a non-CLIA test may be performed; to confirm, results from a CLIA-certified laboratory should be recorded once available. Central laboratory assessments should be performed on screening, Day 3 and Day 7.
⁴ECG for pre-dose does not need to be repeated if screening ECG is conducted on the day of dosing. ECGs collected following study drug administration are to be performed prior to PK assessments.
⁵Resting (recumbent) vital signs (SBP, DBP, and heart rate) will be taken upon having the subject recumbent for 5 min at Screening, Pre-dose and at 0.5, 1, 2, 4,6, 8, and 24 hours post-dose, as well as on Day 3 and Day 7. Triplicate measurements are to be performed in case of SBP <90 mmHg, DBP <60 mmHg, or heart rate <60 bpm. Orthostatic measurements (SBP, DBP, heart rate, and respiratory rate) will be taken at 1, 3, and 5 minutes after standing and temperature will be taken at Screening, Pre-dose, and 2, 4, 8, and 24 hours post-first dose, as well as on Day 3 and Day 7.
⁶PEC will be performed at Screening, Pre-dose (within 15 min prior to dose) and at 10, 20, 30, 45 min; 1, 1.5, 2, 4, 6, 8 and 24 hours post dose. The PCRS must be performed prior to PEC rating, when required. At 6 and 24 hours the PEC rating must be performed before the PANSS interview. ACES will be performed at Pre-dose (within 15 min of dose), 2, 4 and 8 hours post dose. The 3 PANSS Supplementary Items will be performed at each PEC assessment.
⁷CGI-Severity will be performed at Screening and pre-dose. CGI-Improvement will be performed at 0.5, 1, 2, and 4 hours post dose. 7 Buccal exam at 0.5, 2, 4, and 24 hours post-dose for local irritation.
⁸Sparse PK blood samples will be collected 2, 4, 8, and 24 hours (while awake) after dose. For the 2 hour time point, samples for PK will be collected after all other assessments have been collected. A sample may not be collected if the investigator indicates in source documents that the subject is in a mental state that is not conducive to PK sample collection. Non-compliance or refusal of all or any PK draw will not be exclusionary nor result in early termination. Vital signs are to be done prior to PK sample draws, when performed at the same timepoints.
⁹Pre-dose PANSS may be administered at any time prior to dosing on the day of dosing and 6 and 24 hours (−1/+2 hour) post-dose. At 6 and 24 hours, PANSS interview must be performed after PEC rating. The 6-hour and 24-hour PANSS is conducted with reference to the time of dosing.
¹⁰The investigator may choose to re-dose the subject after the 2-hour post-dose assessments are performed if the PEC change from baseline is ≤40%. Patients can be re-dosed after completing the 2-hour post-first dose assessments. Repeat dosing administers half of a film. Patients can be re-dosed twice in the 12-hour period post first dose. All assessments listed in this Schedule of Events at the 2-hour post-first dose timepoint should be repeated at 2 hours post every re-dose. Assessments at 4, 6, or 8 hours post-first dose that occur within 1 hour of a post-re-dose assessment are not required to be performed. Additional PK samples may be obtained at 2 hours after each additional dose in subjects who are re-dosed.

Study assessments

### Diagnostic

The Mini International Neuropsychiatric Interview for children and adolescents (MINI Kid) is a short, structured diagnostic interview for DSM-5.

### Efficacy

The effect of study drug will be evaluated using several validated instruments as described below.

### Positive and Negative Syndrome Scale (Structured Clinical Interview PANSS)

The SCI-PANSS is a tool that measures the severity of schizophrenia symptoms. It contains 4 basic domains: positive, negative, general psychopathology, and a composite scale. The composite scale is derived by subtracting the negative scale score from the positive scale score and can indicate the ratio of positive to negative symptoms. The SCI-PANSS contains Yes/No questions as well as open-ended questions. Each of the 30 items of the SCI-PANSS can be scored or rated. The 7 rating points represent increasing levels of severity. The rating points are labeled as: 1—Absent; 2—Minimal; 3—Mild; 4—Moderate; 5—Moderate Severe; 6—Severe; 7—Extreme. The PANSS will be administered only to subjects with schizophrenia.
PANSS—Excitatory Component (PEC)
Young Mania Rating Scale (YMRS)
Agitation-Calmness Evaluation Scale (ACES)
Clinical Global Impressions of Severity (CGI-S) and Improvement (CGI-I)
Placebo-Control Reminder Script (PCRS)
Drug Likability Scales
Drug Likability Questionnaire

### Pharmacokinetics

Blood samples (4 mL) will be collected per the Schedule of Events (Table 30).

For each subject, up to 6 blood samples (up to 24 mL of blood) will be collected during the study for PK analysis. In addition, approximately 30 mL of blood will be collected at screening, approximately 15 mL of blood will be collected at day 3, and approximately 15 mL of blood will be collected at Day 7 (+2) for clinical laboratory testing. The total volume of blood collected during the study is expected to be approximately 84 mL. Whenever possible for all SAEs, a blood sample for PK analysis will be drawn to evaluate a potential relationship with exposure.

### Statistical Analyses

Data will be summarized by treatment using descriptive statistics (number of subjects, mean, median, standard deviation, minimum, and maximum) for continuous variables and summarized by treatment using frequencies and percentages for categorical variables

### Safety Analyses

All safety analyses will be performed using the Safety Population. All subjects who received at least one dose of study drug will be included in the population for safety analysis.

Adverse events will be characterized by type, severity, seriousness, and relationship to treatment. Adverse events will be coded by preferred term and system organ class using the most current version of MedDRA. Incidence of AEs will be summarized by treatment overall, by severity, and by relationship to study drug. Serious AEs and AEs leading to discontinuation of study drug will also be presented.

Vital sign, ECG with rhythm strip, and clinical laboratory results will be summarized by treatment. Physical examination findings will be listed.

### Brief Summary

This is a study of the efficacy and safety of dexmedetomidine oromucosal film in children and adolescents with acute agitation and either bipolar disorder or schizophrenia.

### DETAILED DESCRIPTION

The study will enroll approximately 120 subjects randomized to dose regimens of 80 μg or 120 μg dexmedetomidine oromucosal film or placebo. Subjects with acute agitation will include male and female children and adolescents who are either newly admitted to a hospital setting or already admitted and experiencing acute agitation. Subjects will be domiciled in a clinical research setting or hospitalized to remain under medical supervision while undergoing screening procedures to assess eligibility. Efficacy and safety assessments will be conducted periodically before and after dosing.

### Study Design

Study Type: Interventional
Primary Purpose: Treatment
Study Phase: Phase 1
Interventional Study Model: Parallel Assignment
Study will randomize subjects 1:1:1 to receive dexmedetomidine oromucosal film 80 μg
dexmedetomidine oromucosal film 120 μg
μg, or matching placebo film,
Number of Arms: 3
Masking: Double (Participant, Investigator)
Double-Blind, Placebo controlled
Allocation: Randomized
Enrollment: 120

TABLE 32
Arms and Intervention

| Arms | Assigned Interventions |
|---|---|
| Experimental: 80 micrograms | Drug: Dexmedetomidine oromucosal film |
| oromucosal film containing 80 micrograms | containing 80 micrograms |
| dexmedetomidine | |
| Experimental: 120 micrograms | Drug: Dexmedetomidine |
| oromucosal film containing 120 micrograms | oromucosal film containing 120 |
| dexmedetomidine | micrograms |
| Placebo Comparator: Placebo | Drug: Placebo film |
| oromucosal film | matching oromucosal film |

### Outcome Measures

### Primary Outcome Measure

1. Primary End Point
Absolute change from baseline in the Positive and Negative Syndrome Scale—Excited
Component (PEC) total score
[Time Frame: 120 minutes]

### Secondary Outcome Measure

2. Key Secondary End Point
Absolute change from baseline in the PEC total score
[Time Frame: 90, 60, 45, 30, 20, 10 minutes]

### Eligibility

Minimum Age: 10 years old
Maximum Age: 17 years old
Sex: All
Gender Based: No
Accepts Healthy Volunteers: No

### Inclusion Criteria

1. Male and female subjects between the ages of 10-17 years old, inclusive, with bipolar disorder (DSM-5 criteria) and 13-17 years old, inclusive, in subjects with schizophrenia (DSM-5 criteria).
2. Patients who are judged to be clinically agitated at Screening and Baseline with a total score of ≥14 on the 5 items comprising the PANSS Excited Component (PEC).
3. Patients who have a score of ≥4 on at least 1 of the 5 items on the PEC at Baseline. Participants who agree to use a medically acceptable and effective birth control method.

### Exclusion Criteria

1. Patients with agitation caused by acute intoxication, including alcohol or drugs of abuse (with the exception of THC) during urine screening
2. Use of benzodiazepines or other hypnotics or oral or short-acting intramuscular antipsychotic drugs in the 4 hours before study treatment.
3. Patients who are judged to be at significant risk of suicide.
4. Patients with serious or unstable medical illnesses.
5. Patients who have received an investigational drug within 30 days prior to the current agitation episode
6. Patients who are considered by the investigator, for any reason, to be an unsuitable candidate for receiving the study drug.

### Example 9. Toxicology Study

The objective of this study was to evaluate the potential toxicity of the of a oromucosal strip containing dexmedetomidine as active ingredient, when given sublingually twice daily (approximately 8 hours apart) for 13 consecutive weeks in beagle dogs and to evaluate the potential reversibility of any findings following a 4-week recovery period. In addition, any signs of local irritation, cardiotoxicity, neurotoxicity or gastrointestinal safety, and the toxicokinetic characteristics of dexmedetomidine compositions was also determined.

### Study Design

TABLE 33
Dog Toxicology Study - Experimental Design

| | | | Dose | Dose | Main Study | | Recovery Study | |
|---|---|---|---|---|---|---|---|---|
| Group | Test | Dose Level | Level^{a} | Concentration | No. of | No. of | No. of | No. of |
| No. | Material | (ug/day) | (ug/dose) | (mg/strip) | Males | Females | Males | Females |
| 1 | Vehicle | 0 | 0 | 0 | 4 | 4 | 2 | 2 |
| 2 | oromucosal | 120 | 0 | 0 | 4 | 4 | — | — |
| | strip | | 60 | 60 | | | | |
| 3 | oromucosal | 240 | 0 | 0 | 4 | 4 | — | — |
| | strip | | 120 | 120 | | | | |
| 4 | oromucosal | 280 | 60 | 60 | 4 | 4 | 2 | 2 |
| | strip | | 80 | 80 | | | | |

^{a}Dose level per dose indicates the dose level receives on each sublingual side, respectively.

The following parameters and endpoints were evaluated in this study: mortality, clinical signs, application site observations, body weights, body weight gains, food consumption, ophthalmology, blood pressure, electrocardiographic examinations, clinical pathology parameters (hematology, coagulation, clinical chemistry, and urinalysis), toxicokinetic parameters, organ weights, and macroscopic and microscopic examinations.

Administration of a dexmedetomidine composition sublingually twice daily (BID) resulted in adverse alterations in ECG endpoints, and in non-adverse clinical observations, body weight increases, and food consumption increases.

In general, the groups showed low to moderate inter-animal variability upon toxicokinetic analysis. Dosed groups showed little difference in exposures between sexes and demonstrated greater than dose proportional increases in AUC and Cₘₐₓ values with respect to dose. After multiple days of dosing, exposures, AUCs and Cₘₐₓ decreased between Day 1 and Day 90. The sublingual administration of a dexmedetomidine composition was associated with an adverse dose-related slowing of the heart rate and lengthening of the RR interval at the Day 1 postdose interval that was accompanied by instances of sinus bradycardia following the 240 and 280 μg/day doses. The slower heart rate was accompanied by a physiologically anticipated prolongation of the PR and QT intervals and QRS duration. The QTc interval was also prolonged. Quantitative ECG changes differed significantly from vehicle following the 240 and 280 μg/day doses. Except for the QTc interval, the quantitative ECG changes resolved at the terminal phase. The test article effect on the QTc interval was greater at Day 1 than at the terminal phase. The magnitude of the QTc change at Day 1 following the 280 μg/day dose exceeded the 10% change seen in the Japanese QT PRODACT telemetry studies in dogs of drugs known to cause QT prolongation in people. The QTc interval changes were reversible, not being evident following the recovery phase.

Clinical observations of sedation (including those findings related to sedation such as decreased activity), salivation, skin lesion (tongue), and/or skin discoloration (gums) were present at all dose levels and were reversible during the recovery phase.

All treated groups had increased body weight and/or body weight gain with correlative increases in food consumption during the treatment period, when compared to controls. At recovery, the increased body weights persisted; however, food consumption was below that of control animals.

There was no effect on vital signs, ophthalmology, clinical pathology parameters, or anatomic pathology endpoints.

In conclusion, twice daily sublingual administration of dexmedetomidine composition to dogs at doses of 120, 240, and 280 μg/day resulted in adverse effects on ECG endpoints at 280 μg/day, which demonstrated reversibility. Non-adverse dexmedetomidine-related finings included reversible clinical observations, body weight increases, and food consumption increases at ≥120 μg/day. The No-Observed-Adverse-Effect-Level (NOAEL) was 240 μg/day. The AUC₀₋₈ and Cₘₐₓ for males and females combined at the NOAEL on Day 90 were 27150 hr*pg/mL and 23957 pg/mL; respectively

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A method of treating agitation associated with dementia in patient in need thereof comprising administering a composition comprising dexmedetomidine or pharmaceutically acceptable salt thereof to the oral mucosa of the patient, wherein the oromucosal administration of the composition results in a Cₘₐₓ from about 50 ng/L to about 300 ng/L and an AUC_{0-inf} from about 200 hr*ng/L to about 2200 hr*ng/L, and wherein the patient is at least about 65 years old.

2. The method of claim 1, wherein the Cmaxis about 50 ng/L, about 60 ng/L, about 70 ng/L, about 80 ng/L, about 90 ng/L, about 100 ng/L, about 110 ng/L, about 120 ng/L, about 130 ng/L, about 140 ng/L, about 150 ng/L, about 160 ng/L, about 170 ng/L, about 180 ng/L, about 190 ng/L, about 200 ng/L, about 220 ng/L, about 240 ng/L, about 260 ng/L, about 280 ng/L, or about 300 ng/L.

3. The method of claim 1, wherein the Cₘₐₓ is about 80% to about 125% of about 108 ng/L.

4. The method of claim 1, wherein the AUC_{0-inf} is about 200 hr*ng/L, 300 hr*ng/L, 400 hr*ng/L, about 450 hr*ng/L, about 500 hr*ng/L, about 550 hr*ng/L, about 600 hr*ng/L, about 650 hr*ng/L, about 700 hr*ng/L, about 750 hr*ng/L, about 800 hr*ng/L, about 850 hr*ng/L, about 900 hr*ng/L, about 950 hr*ng/L, about 1000 hr*ng/L, about 1050 hr*ng/L, about 1100 hr*ng/L, about 1150 hr*ng/L, about 1200 hr*ng/L, about 1250 hr*ng/L, about 1300 hr*ng/L, about 1350 hr*ng/L, about 1400 hr*ng/L, about 1450 hr*ng/L, or about 1500 hr*ng/L.

5. The method of claim 1, wherein the AUC_{0-inf} is about 80% to about 125% of about 985 hr*ng/L.

6. The method of claim 1, wherein the dementia patient is about 65 or older.

7. The method of claim 1, wherein the administration to the oral mucosa is buccal or sublingual administration.

8. The method of claim 1, wherein the administration to the oral mucosa achieves a mean change in PEC or PAS score greater than −2 relative to baseline within 2 hours of administration.

9. The method of claim 1, wherein oral mucosa administration results in a 2-point or greater reduction in RASS score from the baseline value within 2 hours of administration.

10. The method of claim 1, wherein the administration to the oral mucosa achieves a mean change in Mod-CMAI score of greater than −7 relative to baseline within 2 hours of administration.

11. The method of claim 1, wherein the administration to the oral mucosa results in a CGI-I score improvement to about 1 (very much improved) or about a 2 (much improved) within 2 hours of administration.

12. The method of claim 1, wherein the administration to the oral mucosa results in Agitation-Calmness Evaluation Scale (ACES) score improvement to 2 (moderate agitation), 3 (mild agitation) or 4 (normal behavior) within 2 hours of administration.

13. The method of claim 1, wherein the composition is administered one to six times a day.

14. The method of claim 1, wherein the composition is a film a tablet, film, spray, gel or drops.

15. The method according to any of claims 1, wherein the composition is a film.

16. The method of claim 1, wherein dosage of dexmedetomidine or pharmaceutically acceptable salt thereof is about 30 μg to about 90 μg.

17. The method of claim 1, wherein dosage of dexmedetomidine or pharmaceutically acceptable salt thereof is about 30 μg.

18. The method of claim 1, wherein dosage of dexmedetomidine or pharmaceutically acceptable salt thereof is about 40 μg.

19. The method of claim 1, wherein dosage of dexmedetomidine or pharmaceutically acceptable salt thereof is about 60 μg.

20. The method of claim 1, wherein the patient is older than 80 years old.

21. The method of claim 1, wherein the patient is not significantly sedated within 60 minutes after administration.

22. The method of claim 1, wherein the agitation is acute.

23. The method of claim 1, wherein the agitation is chronic.

24. The method of claim 1, wherein the dementia patient has Alzheimer's disease.
